(19) United States
(12) Patent Application Publication
(10) Pub. No.: US 2019/0085024 A1
(43) Pub. Date: Mar. 21, 2019

(54) Alpha-Ketoamide Inhibitors Of Cysteine Proteases

(71) Applicant: Phelix Therapeutics, Philadelphia, PA (US)

(72) Inventors: Doron Greenbaum, Philadelphia, PA (US); Russ Addis, Philadelphia, PA (US); Janet Gaboury, Philadelphia, PA (US); Swapnil Kulkami, Philadelphia, PA (US); Nataline Meinhardt, Philadelphia, PA (US)

(21) Appl. No.: 16/132,794

(22) Filed: Sep. 17, 2018

Related U.S. Application Data
(60) Provisional application No. 62/559,200, filed on Sep. 15, 2017.

(51) Int. Cl. C07K 5/062; C07K 5/078; C07C 271/18; C07D 211/62; C07D 205/04; C07D 213/56; C07C 237/22; C07C 279/12; C07D 209/20; C07D 233/64; C07C 275/24; C07D 403/12

(52) U.S. Cl. CPC C07K 5/06052; C07K 5/06165; C07C 271/18; C07D 211/62; C07D 205/04; C07D 213/56; C07C 2601/02; C07C 279/12; C07D 209/20; C07D 233/64; C07C 275/24; C07D 403/12; C07C 237/22

(57) ABSTRACT

The disclosure provides compounds comprising a α-ketoamide linkage that is terminated on each end by an amino acid, such as compounds of Formula (I), wherein RA-RC are defined herein. Also provided are compositions containing these compounds and methods of inhibiting calpain activity, treating a calpain-mediated disorder, inhibiting cathepsin-B, cathepsin-L, cathepsin-S, or cathepsin-L activity, and methods of treating a cathepsin-B, cathepsin-L, cathepsin-S, or cathepsin-L mediated disorder using these compounds and compositions.

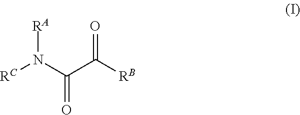

### CROSS REFERENCE TO RELATED APPLICATIONS

This application claims the benefit of U.S. Provisional Application No. 62/559,200 (filed Sep. 15, 2018), the entirety of which is incorporated by reference herein for any and all purposes.

### GOVERNMENT RIGHTS

This invention was made with government support under Contract Nos. R44AI118063 and R01AI097273 awarded by the National Institutes of Health. The government has certain rights in the invention.

### TECHNICAL FIELD

The disclosure provides compounds comprising an α-ketoamide linkage that is terminated on each end by an amino acid, compositions containing these compounds, and methods of inhibiting calpain activity, treating a calpain-mediated disorder, inhibiting cathepsin-B, cathepsin-L, cathepsin-S, or cathepsin-L activity, and methods of treating a cathepsin-B, cathepsin-L, cathepsin-S, or cathepsin-L mediated disorder in using these compounds and compositions.

### BACKGROUND

The papain-like class of cysteine proteases is of pharmaceutical and academic interest due to their roles in a numerous diseases. Papain-like cysteine proteases are primarily intracellular enzymes that cleave peptide bonds using a catalytic triad consisting of cysteine, histidine, and asparagine residues (McGrath, Annu. Rev. Biophys. Biomol. Struct. 1999, 28, 181). Cysteine proteases mediate degradation of peptides and proteins via nucleophilic attack of an amide carbonyl functional group by the active site cysteine sulphhydryl group followed by hydrolysis of a tetrahedral intermediate (Pauly, Biochemistry 2003, 42, 3203).

The papain-like class contains both cathepsins and calpains. Cathepsins are regulated by a prodomain that is cleaved upon a change in pH (Guay, Eur. J. Biochem. 2000, 267, 6311). Cathepsins, are generally located intracellularly, in the acidic environment of the lysosomes where they are responsible for non-specific degradation of protein (Reiser, J Clin Invest 2010, 120, 3421; LaLonde, J. Med. Chem. 1998, 41, 4567). Most cathepsins, including Cathepsin L and B, are expressed ubiquitously in human cells, however some cathepsins are cell specific, such as Cathepsin K and Cathepsin S. Cathepsin K is highly expressed in osteoclasts and most epithelial cells (Turk, Biochimica et Biophysica Acta 2012, 1824, 68). It is excreted to the bone resorption pit where it plays a role in bone remodeling via degradation of collagen fibers. Cathepsin S is expressed in antigen-presenting cells where it is involved in the processing of the invariant chain of the MHC II complex (Riese, Immunity 1996, 4, 357). Like cathepsins, many Calpains, such as Calpains-1 and -2, are ubiquitously expressed. Calpains-1 and -2 are post translationally regulated by an endogenous inhibitor, calpastatin, and require calcium for activation (Siklos, Acta Pharmaceutica Sinica B 2015, 5, 506). Calpains are involved in several biological roles such as the breakdown of cytoskeletal proteins, and are suggested to be involved in cell migration and apoptosis (Goll, J. Physiol. Rev. 2003, 83, 731).

Misregulation or hijacking of cysteine protease activity has been associated with a number of disease states. Both Cathepsin B and Cathepsin L have been implicated in viral entry (Simmons, Antiviral Research 2005, 100, 605). Cathepsin L has also been suggested to contribute to cancer cell proliferation, tumor growth, and resistance to therapy (Biniossek, J. Proteome Res. 2011, 10, 5363). Cathepsins are implicated in processing of the glycoprotein in the viral envelope of corona viruses such as MERS and SARS as well as filo viruses like Ebola and Marburg (Simmons, Antiviral Research 2005, 100, 605; Schornberg, J of Virology 2006, 80, 4174). Excessive Cathepsin K activity has been implicated in osteoporosis and osteoarthritis (LaLonde, J. Med. Chem. 1998, 41, 4567). Cathepsin S has been implicated in cancer (Chen, J. Med. Chem. 2010, 23, 2968). Calpain-1 and -2 contribute to secondary degeneration after acute cellular stress such as myocardial ischemia, cerebral ischemia, or traumatic brain injury (Saatman, Neurotherapeutics 2010, 7, 31; Hong, Stroke 1994, 25, 663). Hyperactivation of Calpain is also correlated with Huntington's disease, Parkinson's disease, Alzheimer's disease, and pulmonary fibrosis (Di Rosa, J. Mol. Neurosci. 2002, 19, 135; Dufty, Am. J. Pathol. 2007, 170, 1725; Vosler, Mol. Neurobiol. 2008, 38, 78; Tabata, Clin. Exp. Immunol. 2010, 162, 560).

Due to the high therapeutic potential of these two classes of proteases, several approaches of inhibiting these two classes of enzymes have been reported in the literature. A common approach is the use of various reactive electrophilic groups called warheads attached to a substrate-like peptide/small molecule that reacts with nucleophilic cysteine thus making it unavailable for hydrolysis of any amide bonds. The warhead can interact with cysteine in a reversible or irreversible fashion, yielding reversible and irreversible inhibitors respectively. Nitrile, aldehyde, and α-ketoamide are examples of reversible warhead groups whereas α,β-unsaturated carbonyls, vinyl sulfones, and epoxides are examples of irreversible groups (Turk, Biochimica et Biophysica Acta 2012, 1824, 68).

What is needed are alternate compounds that are useful for inhibiting calpains and/or various cathepsins.

### SUMMARY

In some embodiments, the disclosure provides compounds comprising an α-ketoamide linkage that is terminated on each end by an amino acid.

In other embodiments, the disclosure provides compounds of Formula (I), wherein R^{A}—R^{C} are defined herein.

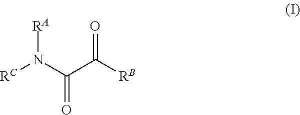

In further embodiments, the disclosure provides compounds of Formula (II), wherein R¹, R³, R^{3′}, R⁴, R^{4′}, R⁵, R⁷, R⁸, R⁹, R¹⁰, x, y, and z are defined herein.

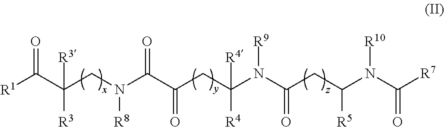

In yet other embodiments, the disclosure provides compounds of Formula (III), wherein R¹, R², R³, R⁴, R⁵, and R⁷ are defined herein.

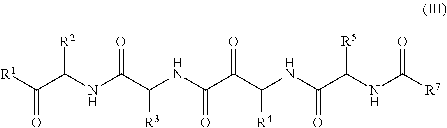

In still further embodiments, the disclosure provides compounds of Formula (IV), wherein R¹, R³, R⁴, R⁵, R⁶, and R⁷ are defined herein.

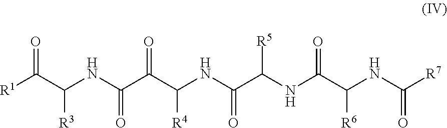

In other embodiments, the disclosure provides compounds of Formula (IV), wherein R¹, R², R³, R⁴, R⁵, R⁶, and R⁷ are defined herein.

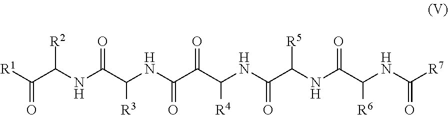

In further embodiment, the disclosure provides composition comprising one or more compounds described herein and a pharmaceutically acceptable carrier and/or excipient.

In still other embodiments, the disclosure provides methods of inhibiting calpain activity, comprising administering one or more compound described herein to a patient in need thereof.

In yet further embodiments, the disclosure provides methods of treating a calpain-mediated disorder in a subject, comprising administering to the subject a therapeutically effective amount of one or more compound described herein to the subject.

In other embodiments, the disclosure provides methods of inhibiting cathepsin-B, cathepsin-L, cathepsin-S, or cathepsin-L activity, comprising administering one or more compound described herein to the subject.

In further embodiments, the disclosure provides methods of treating a cathepsin-B, cathepsin-L, cathepsin-S, or cathepsin-L mediated disorder in a subject, comprising administering to the subject a therapeutically effective amount of one or more compound described herein to the subject.

Other aspects and embodiments of the invention will be readily apparent from the following detailed description of the invention.

### DETAILED DESCRIPTION OF ILLUSTRATIVE EMBODIMENTS

In the present disclosure the singular forms “a”, “an” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about” or “substantially” it will be understood that the particular value forms another embodiment. In general, use of the term “about” or “substantially” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about” or “substantially”. In other cases, the gradations used in a series of values may be used to determine the intended range available to the term “about” or “substantially” for each value. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

Whenever it appears herein, a numerical range such as “1 to 12” refers to each integer in the given range—e.g., “1 to 12 carbon atoms” means that group may consist of 3 carbon atoms, 4 carbon atoms, 5 carbon atoms, etc., up to and including 12 carbon atoms.

“Alkyl,” when used alone or as part of a substituent group, refers to a straight- or branched-chain alkyl having from 1 to 12 carbon atoms (“C₁₋₁₂”), preferably 1 to 6 carbons atoms (“C₁₋₆”), in the chain. Examples of alkyls include methyl (Me, C₁alkyl) ethyl (Et, C₂alkyl), n-propyl (C₃alkyl), isopropyl (C₃alkyl), butyl (C₄alkyl), isobutyl (C₄alkyl), sec-butyl (C₄alkyl), tert-butyl (C₄alkyl), pentyl (C₅alkyl), isopentyl (C₅alkyl), tert-pentyl (C₅alkyl), hexyl (C₆alkyl), or isohexyl (C₆alkyl), among others.

“Alkenyl,” when used alone or as part of a substituent group, refers to a straight- or branched-chain alkyl having from 2 to 12 carbon atoms (“C₁₋₁₂”), preferably 2 to 8 carbons atoms (“C₂₋₈”) or 2 to 6 carbon atoms (“C₂₋₆”) in the chain. Examples of alkyls include ethenyl (C₂alkenyl), n-propenyl (C₃alkenyl), isopropenyl (C₃alkenyl), butenyl (C₄alkenyl), isobutenyl (C₄alkenyl), sec-butenyl (C₄alkenyl), tert-butenyl (C₄alkenyl), pentenyl (C₅alkenyl), isopentenyl (C₅alkenyl), tert-pentenyl (C₅alkenyl), hexenyl (C₆alkenyl), or isohexenyl (C₆alkenyl), among others.

“Cycloalkyl” refers to a cyclic alkyl having from 3 to 12 carbon atoms (“C₁₋₁₂”), preferably 3 to 8 carbons atoms (“C₃₋₈”), in the chain. Examples of cycloalkyls include cyclopropyl (C₃alkyl), cyclobutyl (C₄alkyl), cyclopentyl (C₅alkyl), or cyclohexyl (C₆alkyl), among others.

“Aminoalkyl” as used herein refers to both secondary and tertiary amines where the point of attachment is through the nitrogen-atom and the alkyl is defined above. The alkyls can be the same or different. In some embodiments, the aminoalkyl is NH-(optionally substituted C₁₋₆alkyl). In other embodiments, the aminoalkyl is N(optionally substituted C₁₋₆alkyl)₂,

“Aryl” refers to an aromatic radical with six to ten ring atoms which has at least one ring which is carbocyclic. The term includes monocyclic or fused-ring polycyclic (i.e., rings which share adjacent pairs of ring atoms) groups. Examples of aryls include, without limitation, phenyl, fluorenyl, and naphthyl.

“Heteroaryl” refers to a 5- to 18-membered aromatic radical (e.g., (C₅₋₁₈)heteroaryl) that includes one or more ring heteroatoms that are nitrogen, oxygen and sulfur, and which may be a monocyclic, bicyclic, tricyclic or tetracyclic ring system. Whenever it appears herein, a numerical range such as “5 to 18” refers to each integer in the given range, e.g., “5 to 18 ring atoms” means that the heteroaryl may contain 5 ring atoms, 6 ring atoms, etc., up to and including 18 ring atoms. The heteroatom(s) in the heteroaryl are optionally oxidized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl).

“Ester” refers to a chemical radical of formula —COOR^{e}, where R^{e} includes, but is not limited to, alkyl, alkenyl, alkynyl, cycloalkyl, heterocyclyl, aryl, or heteroaryl.

“Heteroalkyl” refers to an alkyl comprising at least one heteroatom in the backbone of the alkyl as defined herein. In some embodiments, the heteroalkyl contains at least one oxygen, sulfur, or nitrogen heteroatom. In some preferred embodiments, the heteroalkyl contains at least one oxygen atom, for example one, two, three, four, or five oxygen atoms. An alkoxy or oxyalkyl groups are examples of a heteroalkyl containing one oxygen atom. In other preferred embodiments, the heteroalkyl contains at least one sulfur atom, for example one, two, three, four, or five sulfur atoms. In other preferred embodiments, the heteroalkyl contains at least one nitrogen atom, for example one, two, three, four, or five nitrogen atoms.

“Alkoxy” refers to O-alkyl, where the point of attachment is at least through the carbon-atom and the alkyl is defined above. In some embodiments, the alkoxy is a terminal group and is bound through the carbon atom of the alkyl. In other embodiments, the alkoxy is an internal group and is bound through two carbon atoms. In further embodiments, the alkoxy is an internal group and is bound through one carbon atom and one oxygen atom. In some preferred embodiments, the alkoxy contains from 1 to 12 carbon atoms of a straight, branched, cyclic configuration and combinations thereof attached to the parent structure through an oxygen. Examples of alkoxy include, but are not limited to, methoxy, ethoxy, propoxy, isopropoxy, cyclopropyloxy and cyclohexyloxy.

“Oxyalkyl” refers to O-alkyl, where the point of attachment is at least through the oxygen-atom and the alkyl is defined above. In some embodiments, the oxyalkyl is a terminal group and is bound through the oxygen atom. In other embodiments, the oxyalkyl is an internal group and is bound one oxygen atom and one carbon atom. The oxyalkyl contains from 1 to 12 carbon atoms of a straight, branched, cyclic configuration and combinations thereof attached to the parent structure through an oxygen. In some preferred embodiments, the oxyalkyl contains 1 to 12 carbon atoms. Examples of oxyalkyl include, but are not limited to, —O-methyl, O-ethyl, O-propyl, O-cyclopropyl, O-butyl, O-pentyl, or O-cyclohexyl, among others.

“Heterocyclyl” refers to any three to ten membered or four to ten membered monocyclic or bicyclic, saturated ring structure containing at least one heteroatom that is O, N, or S. The heterocyclyl group may be attached at any heteroatom or carbon atom of the ring such that the result is a stable structure. Examples of heterocyclyls include, but are not limited to, azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, quinuclidinyl, tetrahydrofuranyl, tetrahydropyranyl, piperazinyl, and the like.

Unless stated otherwise specifically in the specification, any of the R¹—R⁷ substituents may be optionally substituted by one or more substituents which are independently alkyl, heteroalkyl such as alkoxy, alkenyl, alkynyl, cycloalkyl, heterocyclyl, aryl, heteroaryl, hydroxy, halo, cyano, nitro, oxo, thioxo, —OC(O)R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, where each R^{a} is independently hydrogen, alkyl, cycloalkyl, aryl, heterocyclyl, or heteroaryl.

When a range of carbon atoms is used herein, for example, C₁₋₆, all ranges, as well as individual numbers of carbon atoms are encompassed. For example, “C₁₋₃” includes C₁₋₃, C₁₋₂, C₂₋₃, C₁, C₂, and C₃.

As used herein, the term “compound(s) of formula (I)” includes those compounds of “formula (I),” as well as compounds of any of the formula (I) subgenera such as the compounds of formula (II), (III), (IV), and (V). The term “compound(s) of formula (II)” includes those compounds of “formula (II),” as well as compounds of any of the formula (II) subgenera including the compounds of formula (III), (IV), and (V).

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.”

All isotopic variants of the compounds of the disclosure, radioactive or not, are intended to be encompassed within the scope of the disclosure.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of this disclosure may possess one or more asymmetric centers; such compounds can therefore be produced as individual (R)-or (S)-stereoisomers or as mixtures thereof.

Unless indicated otherwise, the description or naming of a particular compound in the specification and claims is intended to include both individual enantiomers and mixtures, racemic or otherwise, thereof. Within the present disclosure, any open valency appearing on a carbon, oxygen, or nitrogen atom in any structure described herein indicates the presence of a hydrogen atom. Where a chiral center exists in a structure, but no specific stereochemistry is shown for that center, both enantiomers, separately or as a mixture, are encompassed by that structure. The methods for the determination of stereochemistry and the separation of stereoisomers are well-known in the art.

I. The Compounds

The inventors recognized that the full potential of the α-ketoamide functional group is largely unrecognized. Thus, the compounds described herein comprise an α-ketoamide linkage that is terminated on each end by an amino acid. In some embodiments, the compounds contain at least one α-ketoamide linkage.

The term “α-ketoamide linkage” as used herein refers to the following group, where the [symbol] moiety indicates attachment of the linkage to another atom or molecule.

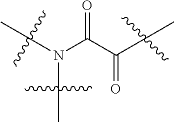 [SMILES: *C(=O)C(=O)N(*)*]

The compounds described herein contain at least one amino acid. In some embodiments, the compounds contain one to about 40 amino acids. In other embodiments, the compounds contain 1 to about 20 amino acids. In further embodiments, the compounds contain 1 to about 10 amino acids. In yet other embodiments, the compounds contain 1 to about 5 amino acids. In still further embodiments, the compounds contain 1, 2, 3, 4, 5, 6, 7, 8, 9, 10, 11, 12, 13, 14, 15, 16, 17, 18, 19, or 20 amino acids.

“Amino acid” as used herein includes natural and unnatural amino acids. In some embodiments, the compound contains one or more natural amino acid. In other embodiments, the compound contains one or more unnatural amino acid. In further embodiments, the compound contains at least one natural amino acid and at least one unnatural amino acid.

Natural amino acids include those that occur in nature, where no part of the amino acid backbone or side chain has been modified. Natural amino acids are proteinogenic, i.e., naturally incorporated into polypeptides. Thus, a natural amino acid may be selected from those known in the art including, without limitation, alanine (Ala), arginine (Arg), asparagine (Asn), aspartic acid (Asp), cysteine (Cys), glutamic acid (Glu), glutamine (Gln), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), valine (Val), selenocysteine (Sec), or pyrrolysine (Pyl).

Unnatural amino acids may occur naturally or may be chemically synthesized. As in a natural amino acid, an unnatural amino acid contains an amine and carboxylic moiety. Unnatural amino acids as used herein refer a natural amino acid that has been modified at one or more points in the amino acid backbone. In some embodiments, the modification is a carboxylation, hydroxylation, or combinations thereof on an amino acid. In other embodiments, the unnatural amino acid includes, without limitation carnitine, GABA, levothyroxine, hydroxyproline, selenomethionine, carboxylated glutamate, hypusine, 2-aminoisobutyric acid, gamma-aminobutyric acid, ornithine, citrulline, or beta alanine, among others.

The natural or unnatural amino acid may be in the L- or D-form. In some embodiments, the natural or unnatural amino acid is in the L-form. In other embodiments, the natural or unnatural amino acid is in the D-form.

In some aspects, the compounds described herein are of Formula (I):

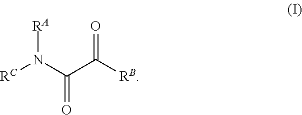

In these compounds, R^{A} is H or C₁₋₆alkyl. In some embodiments, R^{A} is H. In other embodiments, R^{A} is C₁₋₆alkyl such as methyl, ethyl, propyl, butyl, pentyl, or hexyl.

R^{B} and R^{C} are, independently, an amino acid. In some embodiments, R^{B} is Ala, Arg, Asn, Asp, Cys, Glu, Gln, Gly, His, Ile, Leu, Lys, Met, Phe, Pro, Ser, Thr, Trp, Tyr, Val, Sec, or Pyl. In some aspects, R^{B} is Ala. In other aspects, R^{B} is Arg. In further aspects, R^{B} is Asn. In yet other aspects, R^{B} is Asp. In still further aspects, R^{B} is Cys. In other aspects, R^{B} is Glu. In further aspects, R^{B} is Gln. In yet other aspects, R^{B} is Gly. In still further aspects, R^{B} is His. In other aspects, R^{B} is Ile. In further aspects, R^{B} is Leu. In yet further aspects, R^{B} is Lys. In still other aspects, R^{B} is Met. In yet other aspects, R^{B} is Phe. In yet further aspects, R^{B} is Pro. In other aspects, R^{B} is Ser. In still other aspects, R^{B} is Thr. In still further aspects, R^{B} is Trp. In yet other aspects, R^{B} is Tyr. In further aspects, R^{B} is Val. In other aspects, R^{B} is Sec. In yet further aspects, R^{B} is Pyl. In other embodiments, R^{C} is Ala, Arg, Asn, Asp, Cys, Glu, Gln, Gly, His, Ile, Leu, Lys, Met, Phe, Pro, Ser, Thr, Trp, Tyr, Val, Sec, or Pyl. In some aspects, R^{C} is Ala. In other aspects, R^{C} is Arg. In further aspects, R^{C} is Asn. In yet other aspects, R^{C} is Asp. In still further aspects, R^{C} is Cys. In other aspects, R^{C} is Glu. In further aspects, R^{C} is Gln. In yet other aspects, R^{C} is Gly. In still further aspects, R^{C} is His. In other aspects, R^{C} is Ile. In further aspects, R^{C} is Leu. In yet further aspects, R^{C} is Lys. In still other aspects, R^{C} is Met. In yet other aspects, R^{C} is Phe. In yet further aspects, R^{C} is Pro. In other aspects, R^{C} is Ser. In still other aspects, R^{C} is Thr. In still further aspects, R^{C} is Trp. In yet other aspects, R^{C} is Tyr. In further aspects, R^{C} is Val. In other aspects, R^{C} is Sec. In yet further aspects, R^{C} is Pyl.

In other aspects, the compounds described herein are of Formula (II):

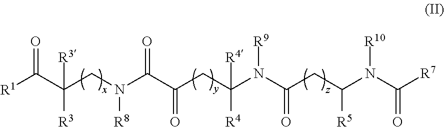

In these compounds, x, y, and z are, independently, 0 or 1. In some embodiments, x is 0 or 1. In other embodiments, x is 0. In further embodiments, x is 1. In yet other embodiments, y is 0 or 1. In still further embodiments, y is 0. In other embodiments, y is 1. In further embodiments, z is 0 or 1. In still other embodiments, z is 0. In yet further embodiments, z is 1. In other embodiments, x, y, and z are 0. In further embodiments, x, y, and z are 1.

R¹ is (i) absent, (ii) OH, (iii) NH₂, (iv) optionally substituted heterocyclyl, (v) NH-(optionally substituted C₁₋₆alkyl), (vi) N-(optionally substituted C₁₋₆alkyl)₂, (vii) NH-(optionally substituted C₁₋₆alkyl-optionally substituted aryl)C(O)NH₂, (viii) NH-(optionally substituted C₁₋₆alkyl-optionally substituted aryl)C(O)N(optionally substituted C₁₋₆alkyl)₂, (ix) NH-(optionally substituted C₁₋₆alkyl-optionally substituted aryl)C(O)NH-optionally substituted C₁₋₆alkyl-optionally substituted aryl, (x) NH-(optionally substituted C₃₋₈cycloalkyl), (xi) NH(optionally substituted C₁₋₆alkyl-optionally substituted aryl), (xii) NH(optionally substituted C₁₋₆alkyl-optionally substituted heterocyclyl), (xiii) NH-(optionally substituted C₁₋₆alkyl)₂C(O)NH-(optionally substituted C₁₋₆alkyl)-optionally substituted heterocyclyl, (xiv) NH-(optionally substituted C₁₋₆alkyl)C(O)NH-(optionally substituted C₁₋₆alkyl)-(optionally substituted aryl), (xv) NH-(optionally substituted C₁₋₆alkoxy)-(optionally substituted C₂₋₆alkenyl)-C(O)NH(optionally substituted C₁₋₆alkylC(O)NH₂)-optionally substituted C₁₋₆alkyl-optionally substituted aryl, or (xvi) NHCH(optionally substituted C₁₋₆alkyl)(C(O)NH(C(O)NH₂)(optionally substituted C₁₋₆alkyl-optionally substituted guanidinyl).

In some embodiments, R¹ is absent.

In other embodiments, R¹ is OH or NH₂. In further embodiments, R¹ is OH. In still other embodiments, R¹ is NH₂.

In yet other embodiments, R¹ is optionally substituted heterocyclyl such as pyrrolidinyl or thiazolyl. In some aspects, R¹ is optionally substituted thiazolyl such as 2-COOH-thiazolyl or 2-COOH-thiazol-one. In other aspects, R¹ is optionally substituted pyrrolidinyl such as prolinyl. In further aspects, R¹ is optionally substituted proline such as proline substituted with an amide, i.e., 2-CONH₂-proline.

In still further embodiments, R¹ is NH-(optionally substituted C₁₋₆alkyl). In some aspects, R¹ is NH-(optionally substituted C₁₋₆alkyl) such as NH(methyl), NH(ethyl), NH(propyl), NH(butyl), NH(pentyl), or NH(hexyl) or N(optionally substituted C₁₋₆alkyl)₂. In other aspects, R¹ is NH-substituted propyl such as NH-propyl substituted with C(O)NH₂, C(O)OH, or C(O)OCH₃, i.e., NHC(CH₃)₂C(O)NH₂, NHC(CH₃)₂C(O)OH, or NHC(CH₃)₂C(O)OCH₃.

In other embodiments, R¹ is N-(optionally substituted C₁₋₆alkyl)₂. In some aspects, R¹ is N(methyl)₂, N(ethyl)₂, N(propyl)₂, N(butyl)₂, N(pentyl)₂, or N(hexyl)₂. In other aspects, the C₁₋₆alkyl is substituted with one or more of C(O)OH, C(O)O(C₁₋₆alkyl), or C(O)NH₂.

In further embodiments, R¹ is NH-(optionally substituted C₁₋₆alkyl-optionally substituted aryl)C(O)NH₂. In some aspects, R¹ is NH—CH(optionally substituted aryl)C(O)NH₂. In other aspects, R¹ is NH—(C₁₋₆alkyl(optionally substituted phenyl or naphthyl))C(O)NH₂. In further aspects, R¹ is NHCH(CH₂phenyl)C(O)NH₂ or NHCH(CH₂-naphthyl)C(O)NH₂. In yet other aspects, R¹ is NHCH(CH₂-biphenyl)C(O)NH₂. In still further aspects, R¹ is NHCH(CH₂-phenyl substituted with C₁₋₆alkyl or CN))C(O)NH₂ such as NHCH(CH₂-phenyl substituted with tBu)C(O)NH₂. In other aspects, R¹ is NH(CH(CH₂-phenyl substituted with CN)—C(O)NH₂ such as NHCH(CH₂-3-CN-phen-3-yl)-C(O)NH₂. In further aspects, R¹ is NHC.

In still other embodiments, R¹ is NH-(optionally substituted C₁₋₆alkyl-optionally substituted aryl)C(O)N(optionally substituted C₁₋₆alkyl)₂such as NH—(C₁₋₆alkyl-aryl)C(O)N(methyl)₂.

In yet further embodiments, R¹ is NH-(optionally substituted C₁₋₆alkyl-optionally substituted aryl)C(O)NH-optionally substituted C₁₋₆alkyl-optionally substituted aryl such as —NH—(C₁₋₆alkyl-aryl)C(O)NH-benzyl or —NH—(C₁₋₆alkyl-aryl)C(O)NH—CH₂CH₂-phenyl.

In other embodiments, R¹ is NH-(optionally substituted C₃₋₈cycloalkyl) such as NH(optionally substituted cyclopentyl). In some aspects, R¹ is NH(cyclopentyl substituted with C(O)OH). In further aspects, R¹ is NH-1-C(O)OH-cyclopentyl.

In further embodiments, R¹ is NH(optionally substituted C₁₋₆alkyl-optionally substituted aryl) such as NH(benzyl)C(O)NH₂.

In yet other embodiments, R¹ is NH(optionally substituted C₁₋₆alkyl-optionally substituted heterocyclyl) such as NH(optionally substituted C₁₋₆alkyl-pyridyl) or NH(optionally substituted C₁₋₆alkyl-optionally substituted pyrimidinyl). In some aspects, R¹ is NH(C₁₋₆alkyl-optionally substituted pyridyl) such as NH(C₁₋₆alkyl-optionally substituted 2-pyridyl), NH(C₁₋₆alkyl-optionally substituted 3-pyridyl), or NH(C₁₋₆alkyl-optionally substituted pyridyl) such as NH(C₁₋₆alkyl-optionally substituted 4-pyridyl). In other aspects, R¹ is NH(C₁₋₆alkyl-optionally substituted pyrimidinyl).

In still further embodiments, R¹ is NH-(optionally substituted C₁₋₆alkyl)C(O)NH-(optionally substituted C₁₋₆alkyl)-optionally substituted heterocyclyl. In some aspects, R¹ is NH—C₁₋₃alkyl)C(O)NH-(optionally substituted C₁₋₆alkyl)-optionally substituted heterocyclyl. In other aspects, R¹ is NH-(optionally substituted C₁₋₆alkyl)C(O)NH-(optionally substituted C₁₋₆alkyl)-(optionally substituted morpholinyl). In further aspects, R¹ is NH—(C₁₋₆alkyl)₂C(O)NH—C₁₋₆alkylmorpholinyl. In still other aspects, R¹ is NHC(Me)₂C(O)NHCH₂CH₂-morpholinyl.

In other embodiments, R¹ is NH-(optionally substituted C₁₋₆alkyl)C(O)NH-(optionally substituted C₁₋₆alkyl)-(optionally substituted aryl). In some aspects, R¹ is NH-(optionally substituted C₁₋₆alkyl)C(O)NH-(optionally substituted C₁₋₆alkyl-optionally substituted phenyl). In other aspects, aspects, R¹ is NH-(optionally substituted C₁₋₆alkyl)C(O)NH—CH₂CH₂-(optionally substituted phenyl). In further aspects, R¹ is NHC(Me)₂C(O)NHCH₂CH₂Ph.

In yet other embodiments, R¹ is NH-(optionally substituted C₁₋₆alkyl-optionally substituted aryl)C(O)NH-(optionally substituted C₁₋₆alkyl)-(optionally substituted aryl). In some aspects, R¹ is NH-(optionally substituted C₁₋₆alkyl-optionally substituted phenyl)C(O)NH-(optionally substituted C₁₋₆alkyl-optionally substituted phenyl). In further aspects, R¹ is NH-(optionally substituted C₁₋₆alkyl-optionally substituted phenyl)C(O)NH—CH₂CH₂-(optionally substituted phenyl). In further aspects, R¹ is NHCH(Bn)C(O)NHCH₂CH₂Ph.

In further embodiments, R¹ is NH-(optionally substituted C₁₋₆alkoxy)-(optionally substituted C₂₋₆alkenyl)-C(O)NH(optionally substituted C₁₋₆alkylC(O)NH₂)-optionally substituted C₁₋₆alkyl-optionally substituted aryl. In some aspects, R¹ is (optionally substituted C₁₋₆alkoxy(CH═CH₂)C(O)(optionally substituted C₁₋₆alkylC(O)NH₂)-optionally substituted C₁₋₆alkyl-optionally substituted aryl. In other aspects, R¹ is NH-(optionally substituted C₁₋₆alkoxy)-(optionally substituted C₂₋₆alkenyl)-C(O)NH(CHC(O)NH₂)-optionally substituted C₁₋₆alkyl-optionally substituted aryl. In further aspects, R¹ is NH-(optionally substituted C₁₋₆alkoxy)-(optionally substituted C₂₋₆alkenyl)-C(O)NH(optionally substituted C₁₋₆alkylC(O)NH₂)—CH₂-optionally substituted aryl. In yet other aspects, R¹ is NH—CH(CH₂OCH₂CH═CH₂)—C(O)NH(CHC(O)NH₂)—CH₂-optionally substituted aryl. In still further aspects, R¹ is NHCH(CH₂OCH₂CH═CH₂)C(O)NH(CH(C(O)NH₂)(CH₂-naphthyl).

In yet other embodiments, R¹ is NHCH(optionally substituted C₁₋₆alkyl)(C(O)NH(C(O)NH₂)(optionally substituted C₁₋₆alkyl-optionally substituted guanidinyl). In some aspects, R¹ is NHCH(optionally substituted CH₂CH₂)(C(O)NH(C(O)NH₂)(optionally substituted C₁₋₆alkyl-optionally substituted guanidinyl). In other aspects, R¹ is NHCH(optionally substituted C₁₋₆alkyl)(C(O)NH(C(O)NH₂)(optionally substituted CH₂CH₂CH₂-optionally substituted guanidinyl). In further aspects, R¹ is NHCH(optionally substituted CH₂CH₂)(C(O)NH(C(O)NH₂)(CH₂CH₂CH₂-optionally substituted guanidinyl). In still other aspects, R¹ is NHCH(CH₂CH₂COOH)C(O)NH(C(O)NH₂(CH₂CH₂CH₂-guanidinyl).

R³ and R^{3′} are, independently, (i) H, (ii) optionally substituted C₁₋₆alkyl, (iii) optionally substituted C₁₋₆alkyl-optionally substituted aryl, (iv) optionally substituted C₁₋₆alkyl-optionally substituted C₃₋₈cycloalkyl, (v) optionally substituted C₁₋₆alkyl-optionally substituted heteroaryl, (vi) optionally substituted C₁₋₆alkyl-guanidinyl, or (vii) optionally substituted C₁₋₆alkyl-NHC(O)(optionally substituted C₂₋₆alkenyl).

In some embodiments, one or both of R³ and R^{3′} are, independently, H or optionally substituted C₁₋₆alkyl.

In other embodiments, one or both of R³ and R^{3′} are, independently, H.

In further embodiments, one or both of R³ and R^{3′} are, independently, optionally substituted C₁₋₆alkyl such as methyl, ethyl, propyl, butyl, pentyl, or hexyl. In some aspects, one or both of R³ and R^{3′} is methyl. In other aspects, one or both of R³ and R^{3′} is butyl such as n-butyl or i-butyl. In further aspects, one or both of R³ and R^{3′} is propyl such as n-propyl. In still other aspects, the C₁₋₆alkyl is substituted with guanidinyl or citrullinyl.

In other embodiments, one or both of R³ and R^{3′} are, independently, optionally substituted C₁₋₆alkyl-optionally substituted aryl such as optionally substituted C₁₋₆alkyl-phenyl or optionally substituted C₁₋₆alkyl-naphthyl. In some aspects, one or both of R³ and R^{3′} is CH₂-optionally substituted aryl. In other aspects, one or both of R³ and R^{3′} is CH₂-optionally substituted phenyl. In further aspects, one or both of R³ and R^{3′} is CH₂-phenyl substituted with C₁₋₆alkyl such as butyl. In yet other aspects, one or both of R³ and R^{3′} is CH₂-4-butyl-benzyl. In still further aspects, one or both of R³ and R^{3′} is CH₂-phenyl substituted with halo such as chloro, bromo, or fluoro. In other aspects, one or both of R³ and R^{3′} is 2-fluorobenzyl, 3-fluorobenzyl, 4-fluorobenzyl, or CH₂CH₂-4-fluorophenyl. In further aspects, one or both of R³ and R^{3′} is CH₂-phenyl substituted with C₁₋₆haloalkyl such as CF₃. In further aspects, one or both of R³ and R^{3′} is 2-CF₃-benzyl, 3-CF₃-benzyl, or 4-CF₃-benzyl. In other aspects, one or both of R³ and R^{3′} is CH₂-phenyl substituted with phenyl, i.e., CH₂-(bi-phenyl). In yet other aspects, one or both of R³ and R^{3′} is CH₂-naphthyl. In still further aspects, the C₁₋₆alkyl is substituted with guanidinyl or citrullinyl. In further aspects, one or both of R³ and R^{3′} is CH₂CH₂CH₂-guanidine.

In yet further embodiments, one or both of R³ and R^{3′} are, independently, optionally substituted C₁₋₆alkyl-optionally substituted C₃₋₈cycloalkyl such as C₁₋₆alkylcyclopropyl, C₁₋₆alkylcyclobutyl, C₁₋₆alkylcyclopentyl, or C₁₋₆alkylcyclohexyl. In some aspects, one or both of R³ and R^{3′} is CH₂-cyclohexyl. In further aspects, one or both of R³ and R^{3′} is CH₂-cyclopropyl. In other aspects, the C₁₋₆alkyl is substituted with guanidinyl or citrullinyl.

In still other embodiments, one or both of R³ and R^{3′} are, independently, optionally substituted C₁₋₆alkyl-optionally substituted heteroaryl. In some aspects, one or both of R³ and R^{3′} is optionally substituted C₁₋₆alkyl-optionally substituted pyridyl such as CH₂-optionally substituted pyridyl. In other aspects, one or both of R₃ and R^{3′} is CH₂-pyrid-2-yl, CH₂-pyrid-3-yl, or CH₂-pyrid-4-yl. In further aspects, one or both of R³ and R^{3′} is optionally substituted C₁₋₆alkyl-optionally substituted indolyl such as CH₂-optionally substituted indolyl. In yet other aspects, the C₁₋₆alkyl is substituted with guanidinyl or citrullinyl.

In further embodiments, one or both of R³ and R^{3′} are, independently, optionally substituted C₁₋₆alkyl-guanidinyl such as methylene-guanidinyl, ethylene-guanidinyl, propylene-guanidinyl, butylene-guanidinyl, pentylene-guanidinyl, or hexylene-guanidinyl,

In other embodiments, R³ and R^{3′} are, independently, optionally substituted C₁₋₆alkyl-NHC(O)(optionally substituted C₂₋₆alkenyl). In some aspects, one or both of R³ and R^{3′} is as (CH₂)₄NHC(O)OCH₂CH═CH₂.

R³ and R^{3′} may alternatively fuse to form an optionally substituted 3 to 8-membered ring. In some embodiments, R³ and R^{3′} are fused to form an optionally substituted 4 to 6-membered ring. In further embodiments, R³ and R^{3′} are fused to form an optionally substituted 3-membered ring, i.e., cyclopropyl. In other embodiments, R³ and R^{3′} are fused to form an optionally substituted 4-membered ring, i.e., cyclobutyl or azetidinyl. In yet other embodiments, R³ and R^{3′} are fused to form azetidinyl. In yet further embodiments, R³ and R^{3′} are fused to form an optionally substituted 5-membered ring, i.e., cyclopentyl or pyrrolidinyl. In still other embodiments, R³ and R^{3′} are fused to form an optionally substituted 5-membered ring, i.e., cyclohexyl or piperidinyl.

R⁴, R^{4′}, and R⁵ are, independently (i) H, (ii) optionally substituted C₁₋₆alkyl, (iii) optionally substituted C₁₋₆alkoxy-optionally substituted C₂₋₆alkenyl, (iv) optionally substituted C₁₋₆alkyl-optionally substituted aryl, (v) optionally substituted C₁₋₆alkyl-optionally substituted C₃₋₈cycloalkyl, or (vi) optionally substituted C₁₋₆alkyl-optionally substituted heteroaryl.

In some embodiments, any one of R⁴, R^{4′}, and R⁵ are, independently, H or optionally substituted C₁₋₆alkyl such as methyl, ethyl, propyl, butyl, pentyl, or hexyl.

In other embodiments, one or both of R⁴ and R^{4′} are H.

In further embodiments, one or both of R⁴ and R^{4′} are, independently, optionally substituted C₁₋₆alkyl such as methyl, ethyl, propyl, butyl, pentyl, or hexyl. In some aspects, one or both of R⁴ and R^{4′} is methyl. In other aspects, R⁴ and R^{4′} are butyl. In further aspects, one or both of R⁴ and R^{4′} are isobutyl.

In still other embodiments, R⁵ is H or optionally substituted C₁₋₆alkyl such as methyl, ethyl, propyl, butyl, pentyl, or hexyl. In some aspects, R⁵ is H. In other aspects, R⁵ is optionally substituted C₁₋₆alkyl such as methyl, ethyl, propyl, butyl, pentyl, or hexyl. In further aspects, R⁵ is H. In yet other aspects, R⁵ is methyl. In still further aspects, R⁵ is isobutyl.

In further embodiments, any one of R⁴, R^{4′}, and R⁵ are, independently, optionally substituted C₁₋₆alkoxy-optionally substituted C₂₋₆alkenyl such as CH₂O—(C₂₋₆alkenyl) or C₁₋₆alkoxy)CH═CH₂. In some aspects, any one of R⁴, R^{4′}, and R⁵ is CH₂OCH₂CH═CH₂.

In yet other embodiments, any one of R⁴, R^{4′}, and R⁵ are, independently, optionally substituted C₁₋₆alkyl-optionally substituted aryl such as C₁₋₆alkylphenyl. In some aspects, one or both of R⁴ and R^{4′} are, independently, optionally substituted C₁₋₆alkyl-optionally substituted aryl such as C₁₋₆alkylphenyl. In other aspects, R⁵ is optionally substituted C₁₋₆alkyl-aryl such as C₁₋₆alkylphenyl. In further aspects, R⁵ is optionally substituted benzyl (Bn). In yet other aspects, R⁵ is benzyl optionally substituted on the phenyl ring —CH₂-(optionally substituted phenyl). In still further aspects, R⁵ is —CH₂-(phenyl substituted with H, halo such as fluoro, chloro, or bromo, C₁₋₆haloalkyl such as CF₃, C₁₋₆alkyl such as methyl or t-butyl, CN, or aryl such as phenyl or naphthyl). In other aspects, R⁵ is 2-F-Bn, 3-F-Bn, 3-CF₃-Bn, 3-CN-Bn, 3-Me-Bn, 4-Br-Bn, 4-CF₃-Bn, 4-F-Bn, or 4-tBu-Bn. In further aspects, R⁵ is CH₂-naphthyl or CH₂-biphenyl.

In still further embodiments, any one of R⁴, R^{4′}, and R⁵ are, independently, optionally substituted C₁₋₆alkyl-optionally substituted C₃₋₈cycloalkyl such as C₁₋₆alkylcyclopropyl. In some aspects, one or both of R⁴ and R^{4′} are, independently, optionally substituted C₁₋₆alkyl-optionally substituted C₃₋₈cycloalkyl such as C₁₋₆alkylcyclopropyl. In other aspects, R⁵ is optionally substituted C₁₋₆alkyl-optionally substituted C₃₋₈cycloalkyl such as C₁₋₆alkylcyclopropyl.

In other embodiments, any one of R⁴, R^{4′}, and R⁵ are, independently, optionally substituted C₁₋₆alkyl-optionally substituted heteroaryl such as C₁₋₆alkylthiazolyl or C₁₋₆alkylpyridinyl. In some aspects, one or both of R⁴ and R^{4′} are, independently, optionally substituted C₁₋₆alkyl-optionally substituted heteroaryl such as C₁₋₆alkylthiazolyl or C₁₋₆alkylpyridinyl. In other aspects, R⁵ is optionally substituted C₁₋₆alkyl-optionally substituted heteroaryl such as C₁₋₆alkylthiazolyl or C₁₋₆alkylpyridinyl. In further aspects, R⁵ is CH₂-(optionally substituted pyridyl) such as CH₂-pyridinone or CH₂-3-pyridinone. In yet other aspects, R⁵ is CH₂-(optionally substituted thiazolyl) such as CH₂-oxo-thiazolyl.

R⁷ is (i) absent, (ii) optionally substituted C₁₋₆alkyl, (iii) optionally substituted C₁₋₆oxyalkyl-optionally substituted aryl, (iv) optionally substituted C₁₋₆alkyl-optionally substituted aryl, (v) optionally substituted C₁₋₆alkyl-optionally substituted aryl-NHC(O)-(optionally substituted C₁₋₆oxyalkyl-optionally substituted aryl), (vi) optionally substituted aryl, (vii) (optionally substituted C₁₋₆alkyl)-NHC(O)-optionally substituted C₁₋₆alkyl, (viii) -(optionally substituted C₁₋₆alkoxy)-(optionally substituted C₂₋₆alkenyl)-NHC(O)-optionally substituted C₁₋₆alkyl-optionally substituted aryl, (ix) optionally substituted C₁₋₆aminoalkyl, or (x) optionally substituted C₁₋₆alkyl-optionally substituted aryl-optionally substituted C₁₋₆oxyalkyl-optionally substituted C₂₋₆alkenyl)(NHC(O)-optionally substituted C₁₋₆alkyl-optionally substituted aryl).

In some embodiments, R⁷ is absent.

In other embodiments, R⁷ is optionally substituted C₁₋₆alkyl such as methyl, ethyl, propyl, butyl, pentyl, or hexyl. In some aspects, R⁷ is methyl. In other aspects, R⁷ is n-butyl. In further aspects, R⁷ is iso-butyl.

In further embodiments, R⁷ is optionally substituted C₁₋₆alkyl-optionally substituted aryl. In some aspects, R⁷ is optionally substituted C₁₋₆alkyl-optionally substituted phenyl, optionally substituted C₁₋₆alkyl-(optionally substituted bi-phenyl), or optionally substituted C₁₋₆alkyl-(optionally substituted naphthyl). In other aspects, R⁷ is benzyl. In further aspects, R⁷ is CH₂-biphenyl. In still other aspects, R⁷ is CH₂-naphthyl. In yet other aspects, R⁷ is CH₂-phenyl substituted with C₁₋₆alkyl such as methyl or halo such as chloro, fluoro, or bromo. In further aspects, R⁷ is CH₂-phenyl substituted with fluoro such as 2-fluoro-phenyl, 3-fluoro-phenyl, or 4-fluoro-phenyl. In other aspects, R⁷ is C₁₋₆alkyl(phenyl)₂ such as CH(phenyl)₂. In still other aspects, R⁷ is CH₂-tolyl such as CH₂-2-tolyl, CH₂-3-tolyl, or CH₂-4-tolyl.

In yet other embodiments, R⁷ is optionally substituted C₁₋₆oxyalkyl-optionally substituted aryl. In some aspects, R⁷ is C₁₋₆oxyalkyl-phenyl. In other aspects, R⁷ is OCH₂-phenyl (OBn).

In still further embodiments, R⁷ is optionally substituted aryl. In some aspects, R⁷ is optionally substituted phenyl. In other aspects, R⁷ is phenyl. In further aspects, R⁷ is phenyl substituted with halo such as fluoro, bromo, or chloro. In still other aspects, R⁷ is phenyl substituted with fluoro such as 2-fluoro-phenyl or 3-fluoro-phenyl.

In other embodiments, R⁷ is -(optionally substituted C₁₋₆alkyl)NHC(O)(optionally substituted C₁₋₆alkyl). In some aspects, R⁷ is -(optionally substituted C₁₋₆alkyl)NHC(O)(benzyl). In further aspects, R⁷ is —(CH(optionally substituted C₁₋₆alkaryl)NHC(O)(benzyl).

In further embodiments, R⁷ is -(optionally substituted C₁₋₆alkoxy)-(optionally substituted C₂₋₆alkenyl)-NHC(O)-optionally substituted C₁₋₆alkyl-optionally substituted aryl. In some aspects, R⁷ is —(C₁₋₆alkoxy)-(C₂₋₆alkenyl)-NHC(O)benzyl. In other aspects, R⁷ is —CH(CH₂OCH₂—C₂₋₆alkenyl)-NHC(O)benzyl, or —(C₁₋₆alkoxy)-(CH═CH₂)—NHC(O)benzyl.

In still other embodiments, R⁷ is optionally substituted C₁₋₆alkyl-optionally substituted aryl-NHC(O)-(optionally substituted C₁₋₆oxyalkyl-optionally substituted aryl). In some aspects, R⁷ is -(optionally substituted C₁₋₆alkyl)-(optionally substituted phenyl)-NHC(O)—(C₁₋₆oxyalkylphenyl). In further aspects, R⁷ is -(optionally substituted C₁₋₆alkyl)-(optionally substituted phenyl)-NHC(O)O-benzyl. In other aspects, R⁷ is -(substituted CH)-(optionally substituted phenyl)-NHC(O)O-benzyl. In further aspects, R⁷ is —(CH(benzyl)-NHC(O)O-benzyl.

In yet further embodiments, R⁷ is optionally substituted C₁₋₆alkyl-optionally substituted aryl-optionally substituted C₁₋₆oxyalkyl-optionally substituted C₂₋₆alkenyl)(NHC(O)-optionally substituted C₁₋₆alkyl-optionally substituted aryl). In some aspects, R⁷ is —CH(C₁₋₆alkyl-(optionally substituted phenyl)-O—C₂₋₆alkenyl)-NHC(O)benzyl. In yet other aspects, R⁷ is —CH(CH₂-optionally substituted phenyl)-O—C₂₋₆alkenyl)-NHC(O)benzyl. In still further aspects, R⁷ is —CH(CH₂(optionally substituted phenyl)OCH₂CH═CH₂)NHC(O)benzyl. In other aspects, R⁷ is CH(CH₂-phenyl-OCH₂CH═CH₂)NHC(O)Bn.

In further embodiments, R⁷ is optionally substituted C₁₋₆aminoalkyl such as NH(optionally substituted C₁₋₆alkyl) or N(optionally substituted C₁₋₆alkyl)₂.

R⁸, R⁹, and R¹⁰ are, independently, H or optionally substituted C₁₋₆alkyl. In some embodiments, any one of R⁸, R⁹, and R¹⁰ are H. In other embodiments, R⁸ is H. In further embodiments, R⁹ is H. In yet other embodiments, R¹⁰ is H. In still further embodiments, any one of R⁸, R⁹, and R¹⁰ are optionally substituted C₁₋₆alkyl. In other embodiments, R⁸ is optionally substituted C₁₋₆alkyl. In further embodiments, R⁹ is optionally substituted C₁₋₆alkyl. In yet other embodiments, R¹⁰ is optionally substituted C₁₋₆alkyl.

In some embodiments, the compound is of Formula (III):

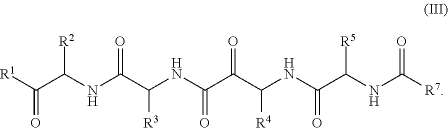

In other embodiments, the compound is of Formula (IV):

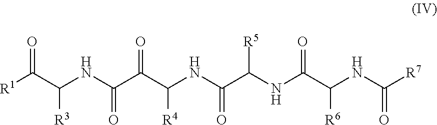

R⁶ in this structure is optionally substituted C₁₋₆alkyl such as methyl, ethyl, propyl, butyl, pentyl, or hexyl.

In further embodiments, the compound is of Formula (V):

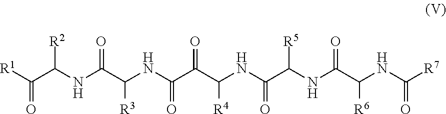

In this structure, R⁶ is optionally substituted C₁₋₆alkyl-optionally substituted C₂₋₆alkenyloxy, optionally substituted C₁₋₆alkyl-optionally substituted aryl-optionally substituted C₂₋₆alkenyloxy, or optionally substituted C₁₋₆alkyl-optionally substituted aryl. In some embodiments, R⁶ is optionally substituted C₁₋₆alkyl-optionally substituted C₂₋₆alkenyloxy. In other embodiments, R⁶ is optionally substituted C₁₋₆alkyl-optionally substituted aryl-optionally substituted C₂₋₆alkenyloxy. In further embodiments, R⁶ is optionally substituted C₁₋₆alkyl-optionally substituted aryl such as benzyl.

In other embodiments, the compound is:

| Phelix # | Structure | IUPAC Name |
|---|---|---|
| PHELIX125 | 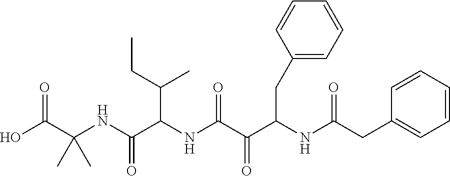 [SMILES: CCC(C)C(NC(=O)C(=O)C(Cc1ccccc1)NC(=O)Cc1ccccc1)C(=O)NC(C)(C)C(=O)O] | 2-methyl-2-(3-methyl-2-(2-oxo-4-phenyl-3-(2-phenylacetamido)butanamido) pentanamido)propanoic acid |
| PHELIX132 | 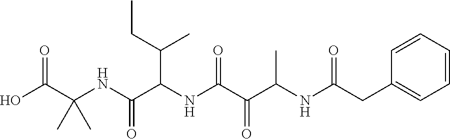 [SMILES: CCC(C)C(NC(=O)C(=O)C(C)NC(=O)Cc1ccccc1)C(=O)NC(C)(C)C(=O)O] | 2-methyl-2-(3-methyl-2-(2-oxo-3-(2-phenylacetamido)butanamido) pentanamido)propanoic acid |
| PHELIX133 | 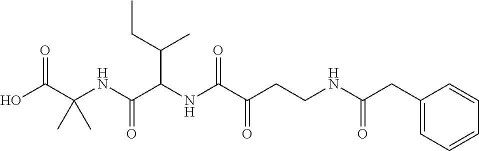 [SMILES: CCC(C)C(NC(=O)C(=O)CCNC(=O)Cc1ccccc1)C(=O)NC(C)(C)C(=O)O] | 2-methyl-2-(3-methyl-2-(2-oxo-4-(2- phenylacetamido)butanamido)pentanamido)propanoic acid |
| PHELIX138 | 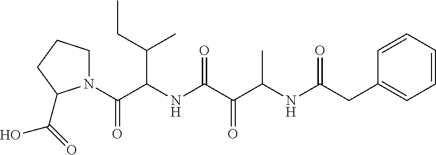 [SMILES: CCC(C)C(NC(=O)C(=O)C(C)NC(=O)Cc1ccccc1)C(=O)N1CCCC1C(=O)O] | (2-oxo-3-(2-phenylacetamido)butanoyl)isoleucylproline |
| PHELIX139 | 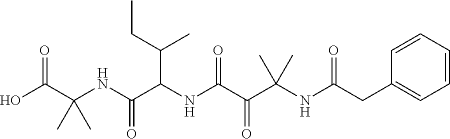 [SMILES: CCC(C)C(NC(=O)C(=O)C(C)(C)NC(=O)Cc1ccccc1)C(=O)NC(C)(C)C(=O)O] | 2-methyl-2-(3-methyl-2-(3-methyl-2-oxo-3-(2- phenylacetamido)butanamido)pentanamido)propanoic acid |
| PHELIX149 | 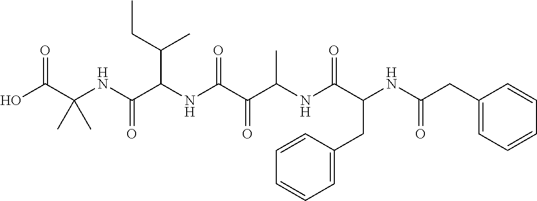 [SMILES: CCC(C)C(NC(=O)C(=O)C(C)NC(=O)C(Cc1ccccc1)NC(=O)Cc1ccccc1)C(=O)NC(C)(C)C(=O)O] | 12-benzyl-5-(sec-butyl)-2,2,9-trimethyl-4,7,8,11,14-pentaoxo-15- phenyl-3,6,10,13-tetraazapentadecanoic acid |
| PHELIX150 | 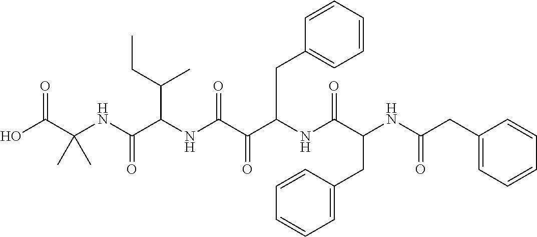 [SMILES: CCC(C)C(NC(=O)C(=O)C(Cc1ccccc1)NC(=O)C(Cc1ccccc1)NC(=O)Cc1ccccc1)C(=O)NC(C)(C)C(=O)O] | 9,12-dibenzyl-5-(sec-butyl)-2,2-dimethyl-4,7,8,11,14-pentaoxo- 15-phenyl-3,6,10,13-tetraazapentadecanoic acid |
| PHELIX155 | 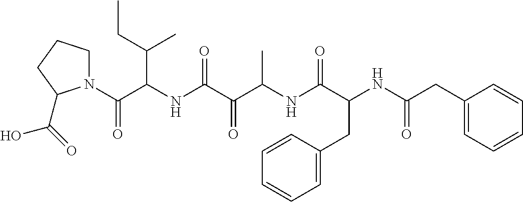 [SMILES: CCC(C)C(NC(=O)C(=O)C(C)NC(=O)C(Cc1ccccc1)NC(=O)Cc1ccccc1)C(=O)N1CCCC1C(=O)O] | (2-oxo-3-(3-phenyl-2-(2- phenylacetamido)propanamido)butanoyl)isoleucylproline |
| PHELIX156 | 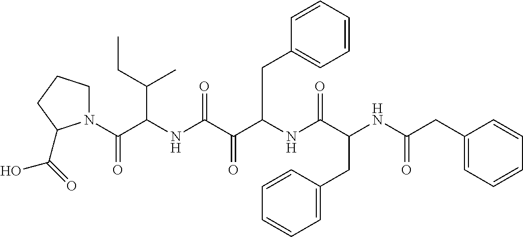 [SMILES: CCC(C)C(NC(=O)C(=O)C(Cc1ccccc1)NC(=O)C(Cc1ccccc1)NC(=O)Cc1ccccc1)C(=O)N1CCCC1C(=O)O] | (2-oxo-4-phenyl-3-(3-phenyl-2-(2- phenylacetamido)propanamido)butanoyl)isoleucylproline |
| PHELIX157 | 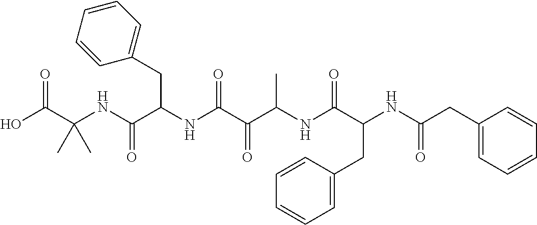 [SMILES: CC(NC(=O)C(Cc1ccccc1)NC(=O)Cc1ccccc1)C(=O)C(=O)NC(Cc1ccccc1)C(=O)NC(C)(C)C(=O)O] | 5,12-dibenzyl-2,2,9-trimethyl-4,7,8,11,14-pentaoxo-15-phenyl- 3,6,10,13-tetraazapentadecanoic acid |
| PHELIX158 | 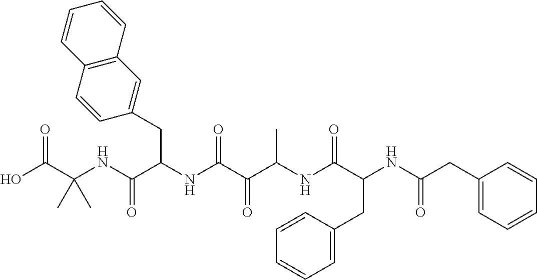 [SMILES: CC(NC(=O)C(Cc1ccccc1)NC(=O)Cc1ccccc1)C(=O)C(=O)NC(Cc1ccc2ccccc2c1)C(=O)NC(C)(C)C(=O)O] | 12-benzyl-2,2,9-trimethyl-5-(naphthalen-2-ylmethyl)-4,7,8,11,14- pentaoxo-15-phenyl-3,6,10,13-tetraazapentadecanoic acid |
| PHELIX159 | 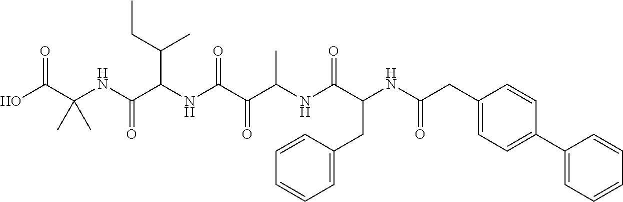 [SMILES: CCC(C)C(NC(=O)C(=O)C(C)NC(=O)C(Cc1ccccc1)NC(=O)Cc1ccc(-c2ccccc2)cc1)C(=O)NC(C)(C)C(=O)O] | 15-([1,1′-biphenyl]-4-yl)-12-benzyl-5-(sec-butyl)-2,2,9-trimethyl- 4,7,8,11,14-pentaoxo-3,6,10,13-tetraazapentadecanoic acid |
| PHELIX160 | 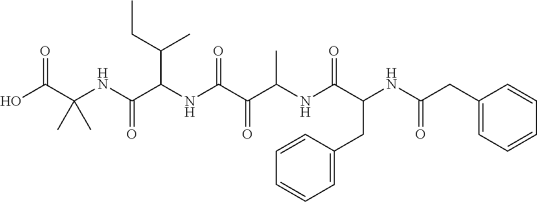 [SMILES: CCC(C)C(NC(=O)C(=O)C(C)NC(=O)C(Cc1ccccc1)NC(=O)Cc1ccccc1)C(=O)NC(C)(C)C(=O)O] | 12-benzyl-5-(sec-butyl)-2,2,9,9-tetramethyl-4,7,8,11,14-pentaoxo- 15-phenyl-3,6,10,13-tetraazapentadecanoic acid |
| PHELIX161 | 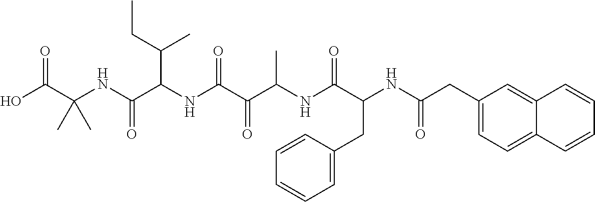 [SMILES: CCC(C)C(NC(=O)C(=O)C(C)NC(=O)C(Cc1ccccc1)NC(=O)Cc1ccc2ccccc2c1)C(=O)NC(C)(C)C(=O)O] | 12-benzyl-5-(sec-butyl)-2,2,9-trimethyl-15-(naphthalen-2-yl)- 4,7,8,11,14-pentaoxo-3,6,10,13-tetraazapentadecanoic acid |
| PHELIX162 | 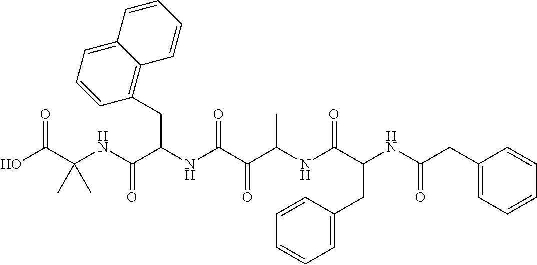 [SMILES: CC(NC(=O)C(Cc1ccccc1)NC(=O)Cc1ccccc1)C(=O)C(=O)NC(Cc1cccc2ccccc12)C(=O)NC(C)(C)C(=O)O] | 12-benzyl-2,2,9-trimethyl-5-(naphthalen-1-ylmethyl)-4,7,8,11,14- pentaoxo-15-phenyl-3,6,10,13-tetraazapentadecanoic acid |
| PHELIX163 | 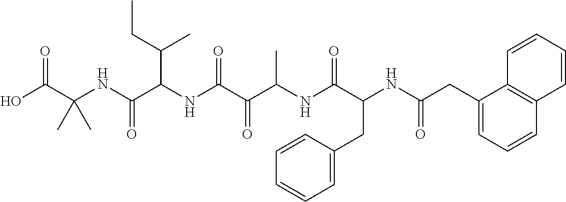 [SMILES: CCC(C)C(NC(=O)C(=O)C(C)NC(=O)C(Cc1ccccc1)NC(=O)Cc1cccc2ccccc12)C(=O)NC(C)(C)C(=O)O] | 12-benzyl-5-(sec-butyl)-2,2,9-trimethyl-15-(naphthalen-1-yl)- 4,7,8,11,14-pentaoxo-3,6,10,13-tetraazapentadecanoic acid |
| PHELIX164 | 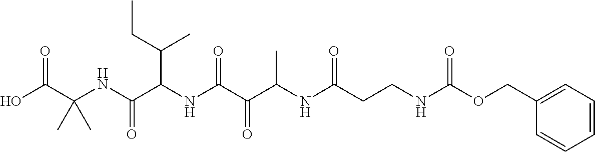 [SMILES: CCC(C)C(NC(=O)C(=O)C(C)NC(=O)CCNC(=O)OCc1ccccc1)C(=O)NC(C)(C)C(=O)O] | 13-(sec-butyl)-9,16,16-trimethyl-3,7,10,11,14-pentaoxo-1-phenyl- 2-oxa-4,8,12,15-tetraazaheptadecan-17-oic acid |
| PHELIX165 | 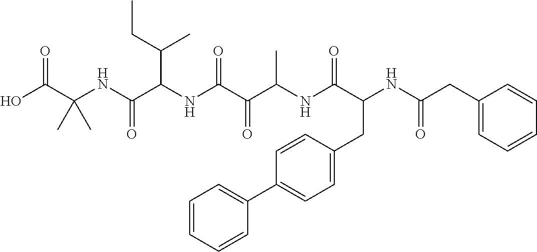 [SMILES: CCC(C)C(NC(=O)C(=O)C(C)NC(=O)C(Cc1ccc(-c2ccccc2)cc1)NC(=O)Cc1ccccc1)C(=O)NC(C)(C)C(=O)O] | 12-([1,1′-biphenyl]-4-ylmethyl)-5-(sec-butyl)-2,2,9-trimethyl- 4,7,8,11,14-pentaoxo-15-phenyl-3,6,10,13-tetraazapentadecanoic acid |
| PHELIX166 | 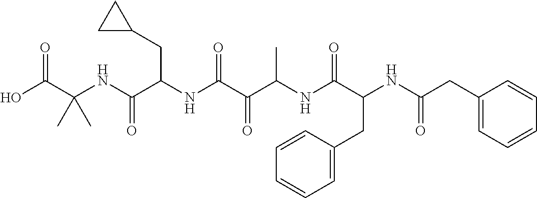 [SMILES: CC(NC(=O)C(Cc1ccccc1)NC(=O)Cc1ccccc1)C(=O)C(=O)NC(CC1CC1)C(=O)NC(C)(C)C(=O)O] | 12-benzyl-5-(cyclopropylmethyl)-2,2,9-trimethyl-4,7,8,11,14-pentaoxo- 15-phenyl-3,6,10,13-tetraazapentadecanoic acid |
| PHELIX167 | 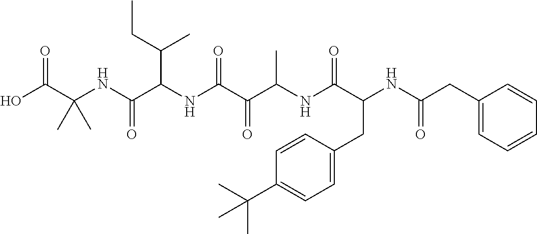 [SMILES: CCC(C)C(NC(=O)C(=O)C(C)NC(=O)C(Cc1ccc(C(C)(C)C)cc1)NC(=O)Cc1ccccc1)C(=O)NC(C)(C)C(=O)O] | 5-(sec-butyl)-12-(4-(tert-butyl)benzyl)-2,2,9-trimethyl- 4,7,8,11,14-pentaoxo-15-phenyl-3,6,10,13-tetraazapentadecanoic acid |
| PHELIX168 | 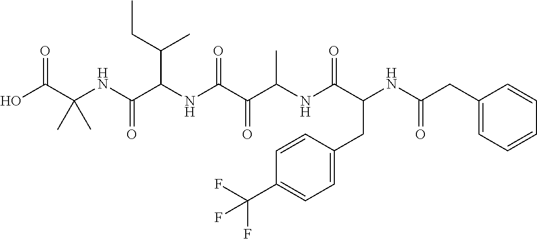 [SMILES: CCC(C)C(NC(=O)C(=O)C(C)NC(=O)C(Cc1ccc(C(F)(F)F)cc1)NC(=O)Cc1ccccc1)C(=O)NC(C)(C)C(=O)O] | 5-(sec-butyl)-2,2,9-trimethyl-4,7,8,11,14-pentaoxo-15-phenyl-12-(4-(trifluoro- methyl)benzyl)-3,6,10,13-tetraazapentadecanoic acid |
| PHELIX169 | 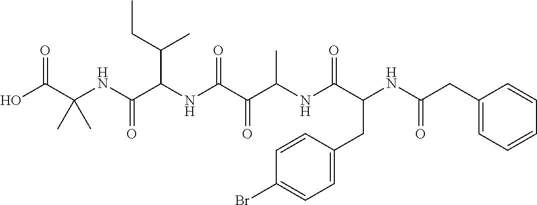 [SMILES: CCC(C)C(NC(=O)C(=O)C(C)NC(=O)C(Cc1ccc(Br)cc1)NC(=O)Cc1ccccc1)C(=O)NC(C)(C)C(=O)O] | 12-(4-bromobenzyl)-5-(sec-butyl)-2,2,9-trimethyl-4,7,11,14-pentaoxo-15- phenyl-3,6,10,13-tetraazapentadecanoic acid |
| PHELIX170 | 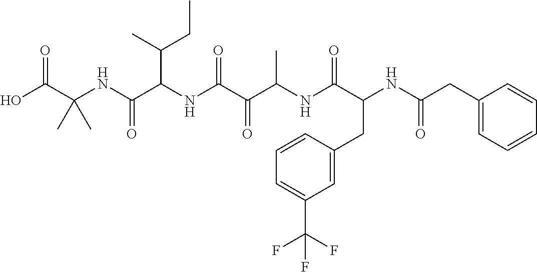 [SMILES: CCC(C)C(NC(=O)C(=O)C(C)NC(=O)C(Cc1cccc(C(F)(F)F)c1)NC(=O)Cc1ccccc1)C(=O)NC(C)(C)C(=O)O] | 5-(sec-butyl)-2,2,9-trimethyl-4,7,8,11,14-pentaoxo-15-phenyl-12-(3-(trifluoro- methyl)benzyl)-3,6,10,13-tetraazapentadecanoic acid |
| PHELIX171 | 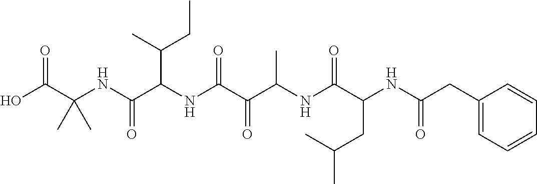 [SMILES: CCC(C)C(NC(=O)C(=O)C(C)NC(=O)C(CC(C)C)NC(=O)Cc1ccccc1)C(=O)NC(C)(C)C(=O)O] | 5-(sec-butyl)-12-isobutyl-2,2,9-trimethyl-4,7,8,11,14-pentaoxo-15-phenyl- 3,6,10,13-tetraazapentadecanoic acid |
| PHELIX172 | 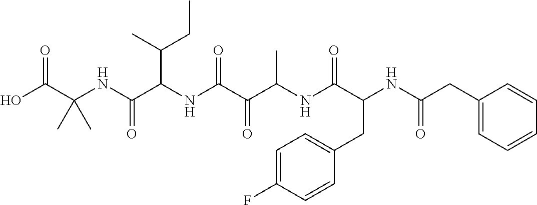 [SMILES: CCC(C)C(NC(=O)C(=O)C(C)NC(=O)C(Cc1ccc(F)cc1)NC(=O)Cc1ccccc1)C(=O)NC(C)(C)C(=O)O] | 5-(sec-butyl)-12-(4-fluorobenzyl)-2,2,9-trimethyl-4,7,8,11,14-pentaoxo-15- phenyl-3,6,10,13-tetraazapentadecanoic acid |
| PHELIX173 | 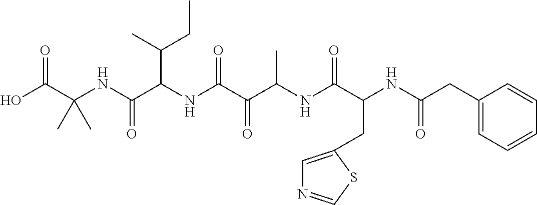 [SMILES: CCC(C)C(NC(=O)C(=O)C(C)NC(=O)C(Cc1cncs1)NC(=O)Cc1ccccc1)C(=O)NC(C)(C)C(=O)O] | (12S)-5-(sec-butyl)-2,2,9-trimethyl-4,7,8,11,14-pentaoxo-15-phenyl-12- (thiazol-5-ylmethyl)-3,6,10,13-tetraazapentadecanoic acid |
| PHELIX174 | 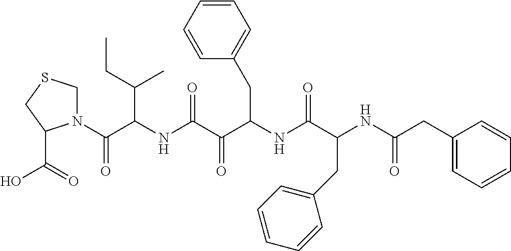 [SMILES: CCC(C)C(NC(=O)C(=O)C(Cc1ccccc1)NC(=O)C(Cc1ccccc1)NC(=O)Cc1ccccc1)C(=O)N1CSCC1C(=O)O] | 3-((2-oxo-4-phenyl-3-(3-phenyl-2-(2- phenylacetamido)propanamido)butanoyl)isoleucyl)thiazolidine-4-carboxylic acid |
| PHELIX175 | 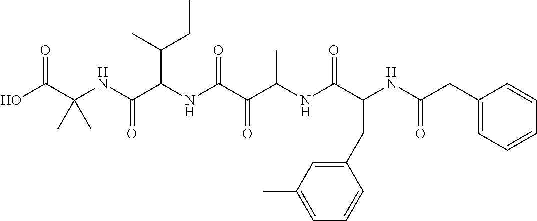 [SMILES: CCC(C)C(NC(=O)C(=O)C(C)NC(=O)C(Cc1cccc(C)c1)NC(=O)Cc1ccccc1)C(=O)NC(C)(C)C(=O)O] | 5-(sec-butyl)-2,2,9-trimethyl-12-(3-methylbenzyl)-4,7,8,11,14-pentaoxo-15- phenyl-3,6,10,13-tetraazapentadecanoic acid |
| PHELIX176 | 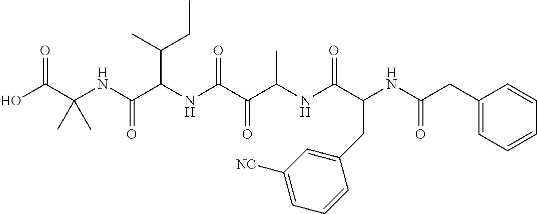 [SMILES: CCC(C)C(NC(=O)C(=O)C(C)NC(=O)C(Cc1cccc(C#N)c1)NC(=O)Cc1ccccc1)C(=O)NC(C)(C)C(=O)O] | 5-(sec-butyl)-12-(3-cyanobenzyl)-2,2,9-trimethyl-4,7,8,11,14-pentaoxo-15- phenyl-3,6,10,13-tetraazapentadecanoic acid |
| PHELIX177 | 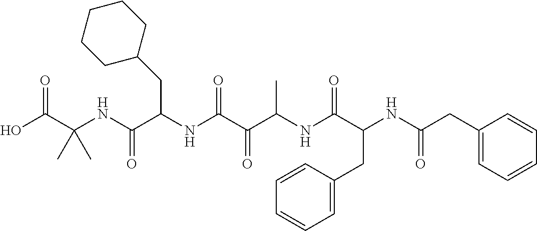 [SMILES: CC(NC(=O)C(Cc1ccccc1)NC(=O)Cc1ccccc1)C(=O)C(=O)NC(CC1CCCCC1)C(=O)NC(C)(C)C(=O)O] | 12-benzyl-5-(cyclohexylmethyl)-2,2,9-trimethyl-4,7,8,11,14-pentaoxo-15- phenyl-3,6,10,13-tetraazapentadecanoic acid |
| PHELIX178 | 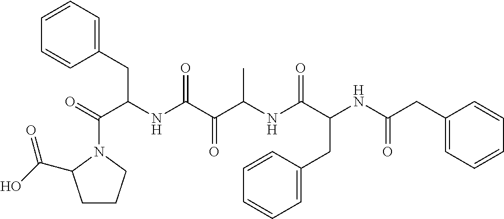 [SMILES: CC(NC(=O)C(Cc1ccccc1)NC(=O)Cc1ccccc1)C(=O)C(=O)NC(Cc1ccccc1)C(=O)N1CCCC1C(=O)O] | (2-oxo-3-(3-phenyl-2-(2-phenylacetamido)propanamido)butanoyl)phenylalanyl proline |
| PHELIX179 | 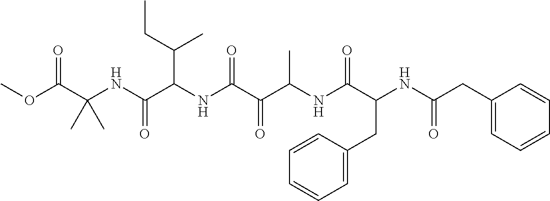 [SMILES: CCC(C)C(NC(=O)C(=O)C(C)NC(=O)C(Cc1ccccc1)NC(=O)Cc1ccccc1)C(=O)NC(C)(C)C(=O)OC] | methyl 12-benzyl-5-(sec-butyl)-2,2,9-trimethyl-4,7,8,11,14-pentaoxo-15- phenyl-3,6,10,13-tetraazapentadecanoate |
| PHELIX180 | 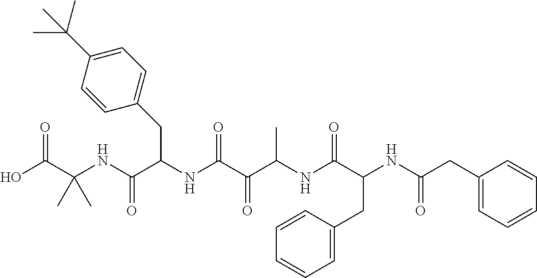 [SMILES: CC(NC(=O)C(Cc1ccccc1)NC(=O)Cc1ccccc1)C(=O)C(=O)NC(Cc1ccc(C(C)(C)C)cc1)C(=O)NC(C)(C)C(=O)O] | (5S,12)S-12-benzyl-5-(4-(tert-butyl)benzyl)-2,2,9-trimethyl- 4,7,8,11,14-pentaoxo-15-phenyl-3,6,10,13-tetraazapentadecanoic acid |
| PHELIX183 | 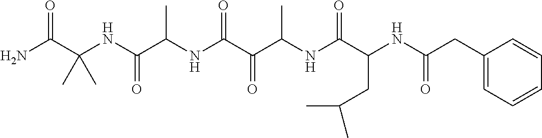 [SMILES: CC(C)CC(NC(=O)Cc1ccccc1)C(=O)NC(C)C(=O)C(=O)NC(C)C(=O)NC(C)(C)C(N)=O] | N-(4-((1-((1-amino-2-methyl-1-oxopropan-2-yl)amino)-1- oxopropan-2-yl)amino)-3,4-dioxobutan-2-yl)-4-methyl-2-(2- phenylacetamido)pentanamide |
| PHELIX184 | 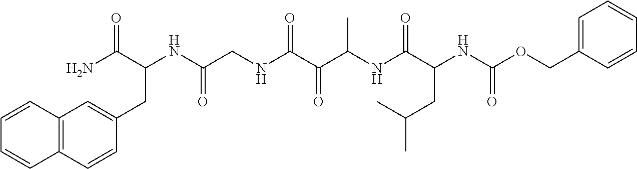 [SMILES: CC(C)CC(NC(=O)OCc1ccccc1)C(=O)NC(C)C(=O)C(=O)NCC(=O)NC(Cc1ccc2ccccc2c1)C(N)=O] | benzyl (1-((4-((2-((1-amino-3-(naphthalen-2-yl)-1-oxopropan-2- yl)amino)-2-oxoethyl)amino)-3,4-dioxobutan-2-yl)amino)-4-methyl-1- oxopentan-2-yl)carbamate |
| PHELIX185 | 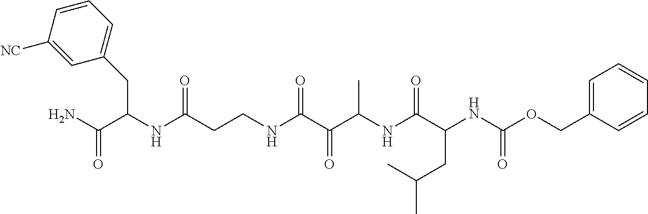 [SMILES: CC(C)CC(NC(=O)OCc1ccccc1)C(=O)NC(C)C(=O)C(=O)NCCC(=O)NC(Cc1cccc(C#N)c1)C(N)=O] | benzyl (1-((4-((3-((1-amino-3-(3-cyanophenyl)-1-oxopropan-2- yl)amino)-3-oxopropyl)amino)-3,4-dioxobutan-2-yl)amino)-4-methyl- 1-oxopentan-2-yl)carbamate |
| PHELIX186 | 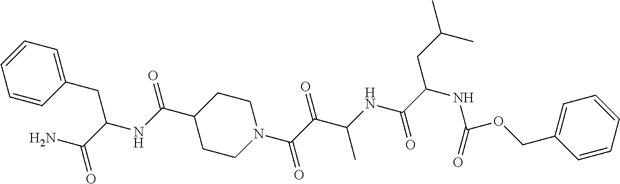 [SMILES: CC(C)CC(NC(=O)OCc1ccccc1)C(=O)NC(C)C(=O)C(=O)N1CCC(C(=O)NC(Cc2ccccc2)C(N)=O)CC1] | benzyl (1-((4-(4-((1-amino-1-oxo-3-phenylpropan-2- yl)carbamoyl)piperidin-1-yl)-3,4-dioxobutan-2-yl)amino)-4-methyl-1-oxopentan- 2-yl)carbamate |
| PHELIX187 | 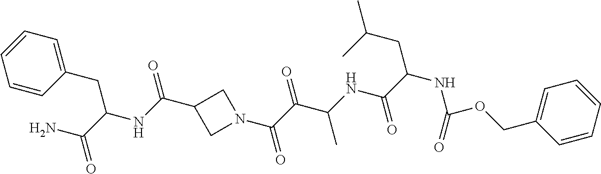 [SMILES: CC(C)CC(NC(=O)OCc1ccccc1)C(=O)NC(C)C(=O)C(=O)N1CC(C(=O)NC(Cc2ccccc2)C(N)=O)C1] | benzyl (1-((4-(3-((1-amino-1-oxo-3-phenylpropan-2- yl)carbamoyl)azetidin-1-yl)-3,4-dioxobutan-2-yl)amino)-4-methyl-1- oxopentan-2-yl)carbamate |
| PHELIX198 | 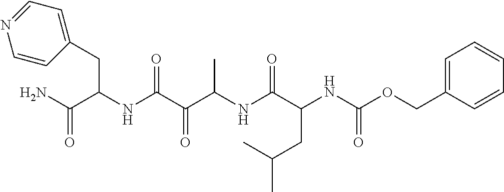 [SMILES: CC(C)CC(NC(=O)OCc1ccccc1)C(=O)NC(C)C(=O)C(=O)NC(Cc1ccncc1)C(N)=O] | benzyl (1-((4-((1-amino-1-oxo-3-(pyridin-4-yl)propan-2- yl)amino)-3,4-dioxobutan-2-yl)amino)-4-methyl-1-oxopentan-2-yl)carbamate |
| PHELIX200 | 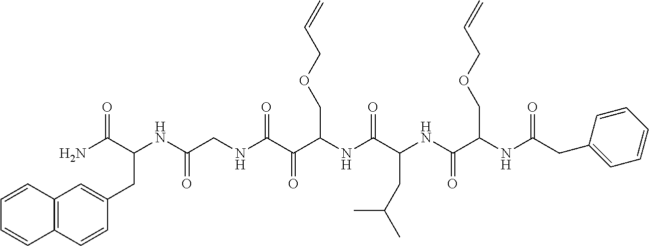 [SMILES: C=CCOCC(NC(=O)Cc1ccccc1)C(=O)NC(CC(C)C)C(=O)NC(COCC=C)C(=O)C(=O)NCC(=O)NC(Cc1ccc2ccccc2c1)C(N)=O] | 2-(3-(allyloxy)-2-(2-phenylacetamido)propanamido)-N-(1- (allyloxy)-4-((2-((1-amino-3-(naphthalen-2-yl)-1-oxopropan-2- yl)amino)-2-oxoethyl)amino)-3,4-dioxobutan-2-yl)-4-methylpentanamide |
| PHELIX202 | 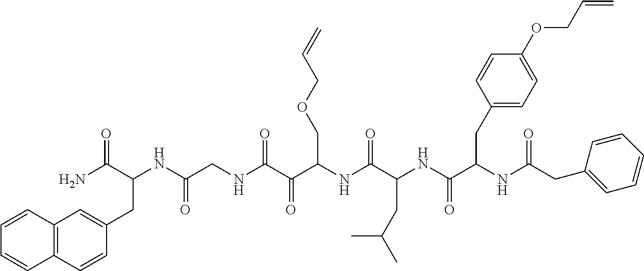 [SMILES: C=CCOCC(NC(=O)C(CC(C)C)NC(=O)C(Cc1ccc(OCC=C)cc1)NC(=O)Cc1ccccc1)C(=O)C(=O)NCC(=O)NC(Cc1ccc2ccccc2c1)C(N)=O] | N-(1-(allyloxy)-4-((2-((1-amino-3-(naphthalen- 2-yl)-1-oxopropan- 2-yl)amino)-2-oxoethyl)amino)-3,4-dioxobutan-2-yl)-2-(3-(4- (allyloxy)phenyl)-2-(2-phenylacetamido)propanamido)-4-methylpentanamide |
| PHELIX204 | 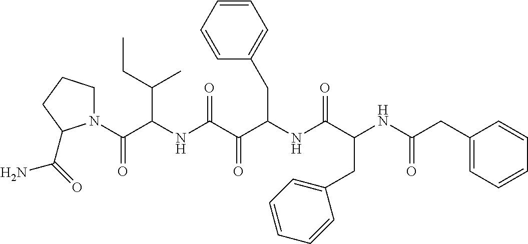 [SMILES: CCC(C)C(NC(=O)C(=O)C(Cc1ccccc1)NC(=O)C(Cc1ccccc1)NC(=O)Cc1ccccc1)C(=O)N1CCCC1C(N)=O] | 1-((2-oxo-4-phenyl-3-(3-phenyl-2-(2- phenylacetamido)propanamido)butanoyl)isoleucyl)pyrrolidine-2-carboxamide |
| PHELIX205 | 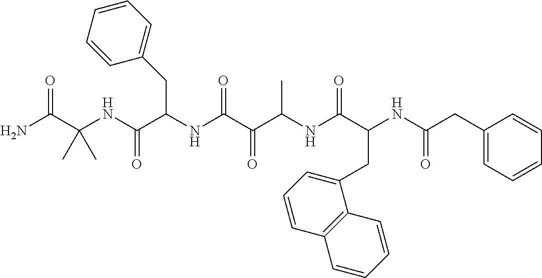 [SMILES: CC(NC(=O)C(Cc1cccc2ccccc12)NC(=O)Cc1ccccc1)C(=O)C(=O)NC(Cc1ccccc1)C(=O)NC(C)(C)C(N)=O] | N-(1-((1-amino-2-methyl-1-oxopropan-2-yl)amino)-1-oxo-3- phenylpropan-2-yl)-3-(3-(napthalen-1-yl)-2-(2-phenylacetamido)propanamido)- 2-oxobutanamide |
| PHELIX206 | 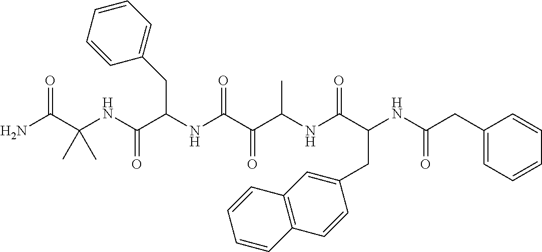 [SMILES: CC(NC(=O)C(Cc1ccc2ccccc2c1)NC(=O)Cc1ccccc1)C(=O)C(=O)NC(Cc1ccccc1)C(=O)NC(C)(C)C(N)=O] | N-(1-((1-amino-2-methyl-1-oxopropan-2-yl)amino)-1-oxo-3- phenylpropan-2-yl)-3-(3-(naphthalen-2-yl)-2-(2-phenylacetamido)propanamido)- 2-oxobutanamide |
| PHELIX207 | 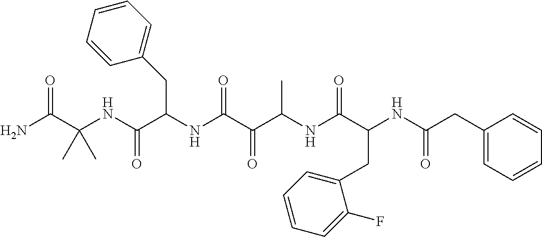 [SMILES: CC(NC(=O)C(Cc1ccccc1F)NC(=O)Cc1ccccc1)C(=O)C(=O)NC(Cc1ccccc1)C(=O)NC(C)(C)C(N)=O] | N-(1-((1-amino-2-methyl-1-oxopropan-2-yl)amino)-1-oxo-3- phenylpropan-2-yl)-3-(3-(2-fluorophenyl)-2-(2-phenylacetamido)propanamido)- 2-oxobutanamide |
| PHELIX208 | 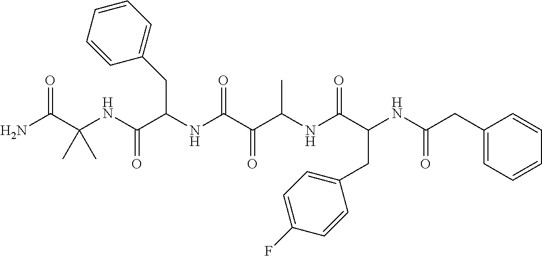 [SMILES: CC(NC(=O)C(Cc1ccc(F)cc1)NC(=O)Cc1ccccc1)C(=O)C(=O)NC(Cc1ccccc1)C(=O)NC(C)(C)C(N)=O] | N-(1-((1-amino-2-methyl-1-oxopropan-2-yl)amino)-1-oxo-3- phenylpropan-2-yl)-3-(3-(4-fluorophenyl)-2-(2-phenylacetamido)propanamido)- 2-oxobutanamide |
| PHELIX209 | 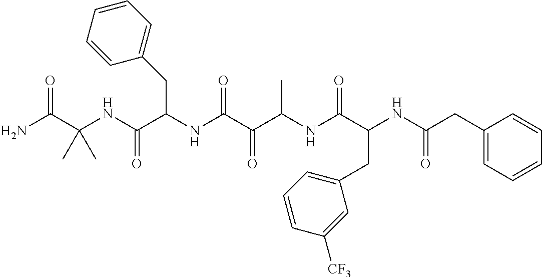 [SMILES: CC(NC(=O)C(Cc1cccc(C(F)(F)F)c1)NC(=O)Cc1ccccc1)C(=O)C(=O)NC(Cc1ccccc1)C(=O)NC(C)(C)C(N)=O] | N-(1-((1-amino-2-methyl-1-oxopropan-2-yl)amino)-1-oxo-3- phenylpropan-2-yl)-2-oxo-3-(2-(2-phenylacetamido)-3-(3- (trifluoromethyl)phenyl)propanamido)butanamide |
| PHELIX211 | 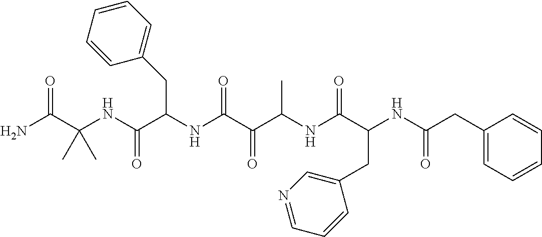 [SMILES: CC(NC(=O)C(Cc1cccnc1)NC(=O)Cc1ccccc1)C(=O)C(=O)NC(Cc1ccccc1)C(=O)NC(C)(C)C(N)=O] | N-(1-((1-amino-2-methyl-1-oxopropan-2-yl)amino)-1-oxo-3- phenylpropan-2-yl)-2-oxo-3-(2-(2-phenylacetamido)-3-(pyridin-3- yl)propanamido)butanamide |
| PHELIX212 | 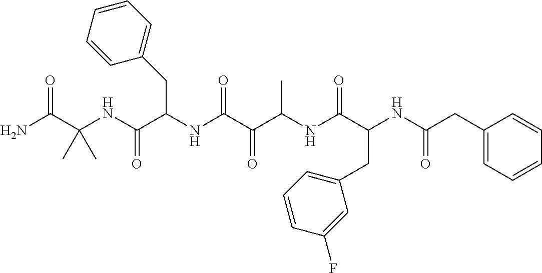 [SMILES: CC(NC(=O)C(Cc1cccc(F)c1)NC(=O)Cc1ccccc1)C(=O)C(=O)NC(Cc1ccccc1)C(=O)NC(C)(C)C(N)=O] | N-(1-((1-amino-2-methyl-1-oxopropan-2-yl)amino)-1-oxo-3- phenylpropan-2-yl)-3-(3-(3-fluorophenyl)-2-(2-phenylacetamido)propanamido)- 2-oxobutanamide |
| PHELIX213 | 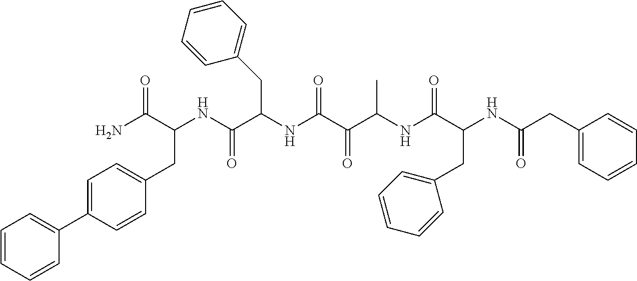 [SMILES: CC(NC(=O)C(Cc1ccccc1)NC(=O)Cc1ccccc1)C(=O)C(=O)NC(Cc1ccccc1)C(=O)NC(Cc1ccc(-c2ccccc2)cc1)C(N)=O] | N-(1-((3-([1,1′-biphenyl]-4-yl)-1-amino-1- oxopropan-2-yl)amino)- 1-oxo-3-phenylpropan-2-yl)-2-oxo-3-(3-phenyl-2-(2- phenylacetamido)propanamido)butanamide |
| PHELIX214 | 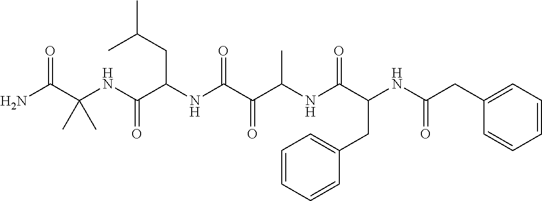 [SMILES: CC(C)CC(NC(=O)C(=O)C(C)NC(=O)C(Cc1ccccc1)NC(=O)Cc1ccccc1)C(=O)NC(C)(C)C(N)=O] | N-(1-amino-2-methyl-1-oxopropan-2-yl)-4-methyl-2-(2- oxo-3-(3-phenyl-2-(2-phenylacetamido)propanamido)butanamido)pentanamide |
| PHELIX215 PHELIX220 | 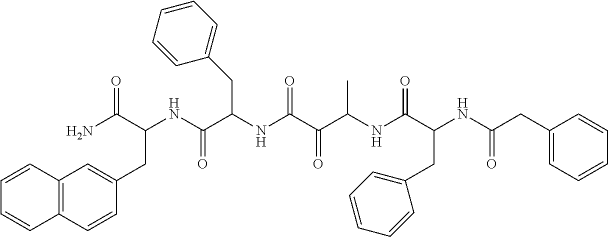 [SMILES: CC(NC(=O)C(Cc1ccccc1)NC(=O)Cc1ccccc1)C(=O)C(=O)NC(Cc1ccccc1)C(=O)NC(Cc1ccc2ccccc2c1)C(N)=O] | N-(1-((1-amino-3-(naphthalen-2-yl)-1-oxopropan-2-yl)amino)- 1-oxo-3-phenylpropan-2-yl)-2-oxo-3-(3-phenyl-2-(2- phenylacetamido)propanamido)butanamide |
| PHELIX216 | 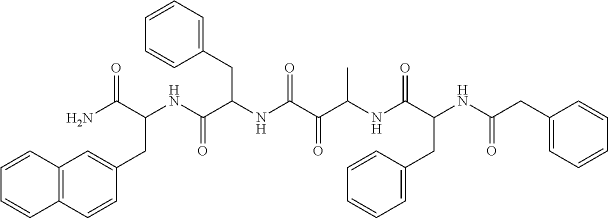 [SMILES: CC(NC(=O)C(Cc1ccccc1)NC(=O)Cc1ccccc1)C(=O)C(=O)NC(Cc1ccccc1)C(=O)NC(Cc1ccc2ccccc2c1)C(N)=O] | N-(1-((1-amino-3-(naphthalen-2-yl)-1- oxopropan-2-yl)amino)-1- oxo-3-phenylpropan-2-yl)-2-oxo-3-(3-phenyl-2-(2- phenylacetamido)propanamido)butanamide |
| PHELIX217 | 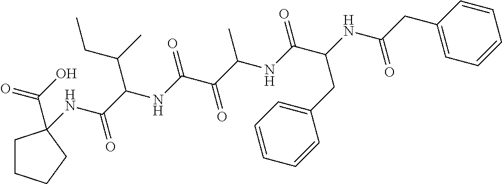 [SMILES: CCC(C)C(NC(=O)C(=O)C(C)NC(=O)C(Cc1ccccc1)NC(=O)Cc1ccccc1)C(=O)NC1(C(=O)O)CCCC1] | 1-(3-methyl-2-(2-oxo-3-(3-phenyl-2-(2- phenylacetamido)propanamido)butanamido)pentanamido)cyclopentane-1-carboxylic acid |
| PHELIX218 | 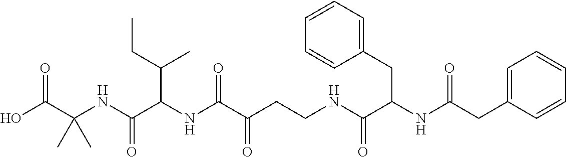 [SMILES: CCC(C)C(NC(=O)C(=O)CCNC(=O)C(Cc1ccccc1)NC(=O)Cc1ccccc1)C(=O)NC(C)(C)C(=O)O] | 13-benzyl-5-(sec-butyl)-2,2-dimethyl-4,7,8,12,15-pentaoxo-16-phenyl- 3,6,11,14-tetraazahexadecanoic acid |
| PHELIX219 | 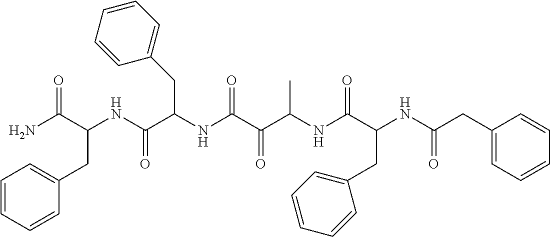 [SMILES: CC(NC(=O)C(Cc1ccccc1)NC(=O)Cc1ccccc1)C(=O)C(=O)NC(Cc1ccccc1)C(=O)NC(Cc1ccccc1)C(N)=O] | N-(1-((1-amino-1-oxo-3-phenylpropan-2-yl)amino)-1-oxo-3- phenylpropan-2-yl)-2-oxo-3-(2-(2-phenylacetamido)-3- (pyridin-2-yl)propanamido)butanamide |
| PHELIX221 | 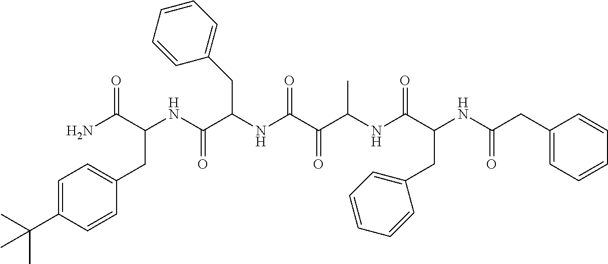 [SMILES: CC(NC(=O)C(Cc1ccccc1)NC(=O)Cc1ccccc1)C(=O)C(=O)NC(Cc1ccccc1)C(=O)NC(Cc1ccc(C(C)(C)C)cc1)C(N)=O] | N-(1-((1-amino-3-(4-(tert-butyl)phenyl)-1-oxopropan-2- yl)amino)-1-oxo-3-phenylpropan-2-yl)-2-oxo-3-(3-phenyl-2-(2- phenylacetamido)propanamido)butanamide |
| PHELIX222 | 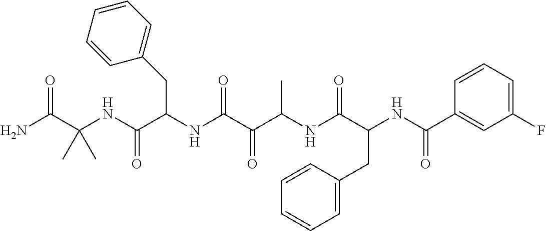 [SMILES: CC(NC(=O)C(Cc1ccccc1)NC(=O)c1cccc(F)c1)C(=O)C(=O)NC(Cc1ccccc1)C(=O)NC(C)(C)C(N)=O] | N-(1-((4-((1-((1-amino-2-methyl-1-oxopropan- 2-yl)amino)-1-oxo-3-phenylpropan- 2-yl)amino)-3,4-dioxobutan-2-yl)amino)-1-oxo-3-phenylpropan-2-yl)-3-fluorobenzamide |
| PHELIX223 | 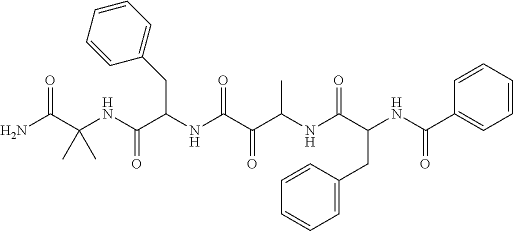 [SMILES: CC(NC(=O)C(Cc1ccccc1)NC(=O)c1ccccc1)C(=O)C(=O)NC(Cc1ccccc1)C(=O)NC(C)(C)C(N)=O] | N-(1-((4-((1-((1-amino-2-methyl-1- oxopropan-2-yl)amino)-1-oxo-3-phenylpropan- 2-yl)amino)-3,4-dioxobutan-2-yl)amino)-1-oxo-3-phenylpropan-2-yl)benzamide |
| PHELIX224 | 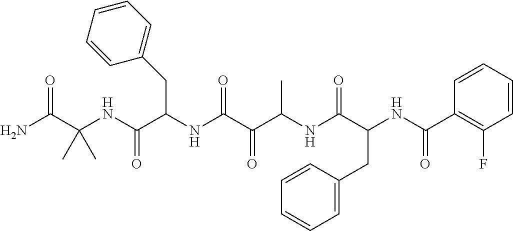 [SMILES: CC(NC(=O)C(Cc1ccccc1)NC(=O)c1ccccc1F)C(=O)C(=O)NC(Cc1ccccc1)C(=O)NC(C)(C)C(N)=O] | N-(1-((4-((1-((1-amino-2-methyl-1-oxopropan- 2-yl)amino)-1-oxo-3-phenylpropan- 2-yl)amino)-3,4-dioxobutan-2-yl)amino)-1-oxo-3-phenylpropan-2- yl)-2-fluorobenzamide |
| PHELIX225 | 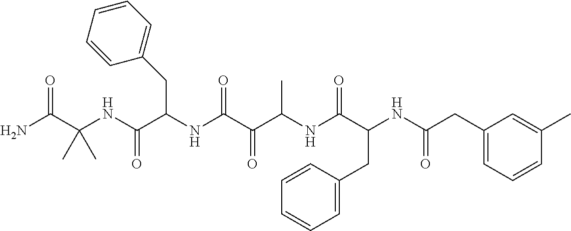 [SMILES: Cc1cccc(CC(=O)NC(Cc2ccccc2)C(=O)NC(C)C(=O)C(=O)NC(Cc2ccccc2)C(=O)NC(C)(C)C(N)=O)c1] | N-(1-((1-amino-2-methyl-1-oxopropan-2-yl)amino)-1-oxo-3- phenylpropan-2-yl)-2-oxo-3-(3-phenyl-2-(2-(m-tolyl)acetamido)propanmido)butanamide |
| PHELIX226 | 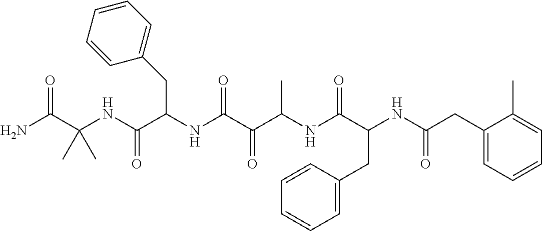 [SMILES: Cc1ccccc1CC(=O)NC(Cc1ccccc1)C(=O)NC(C)C(=O)C(=O)NC(Cc1ccccc1)C(=O)NC(C)(C)C(N)=O] | N-(1-((1-amino-2-methyl-1-oxopropan-2-yl)amino)-1-oxo-3- phenylpropan-2-yl)-2-oxo-3-(3-phenyl-2-(2-(o-tolyl)acetamido)propanamido)butanamide |
| PHELIX227 | 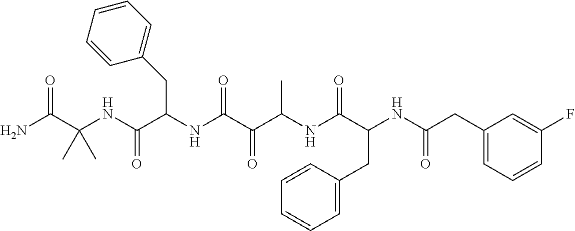 [SMILES: CC(NC(=O)C(Cc1ccccc1)NC(=O)Cc1cccc(F)c1)C(=O)C(=O)NC(Cc1ccccc1)C(=O)NC(C)(C)C(N)=O] | N-(1-((1-amino-2-methyl-1-oxopropan-2-yl)amino)-1-oxo-3- phenylpropan-2-yl)-3-(2-(2-(3-fluorophenyl)acetamido)-3-phenylpropanmido)- 2-oxobutanamide |
| PHELIX228 | 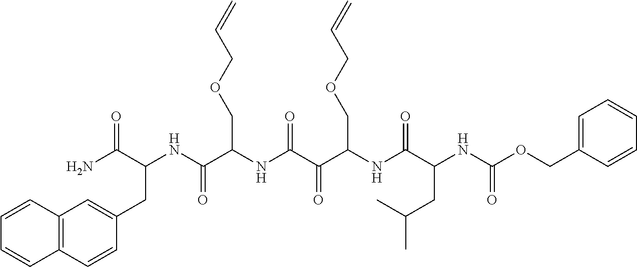 [SMILES: C=CCOCC(NC(=O)C(=O)C(COCC=C)NC(=O)C(CC(C)C)NC(=O)OCc1ccccc1)C(=O)NC(Cc1ccc2ccccc2c1)C(N)=O] | benzyl (1-((1-(allyloxy)-4-((3-(allyloxy)-1-((1-amino-3- (naphthalen-2-yl)-1-oxopropan-2-yl)amino)-1-oxopropan-2- yl)amino)-3,4-dioxobutan-2-yl)amino)-4-methyl-1-oxopentan-2-yl)carbamate |
| PHELIX230 | 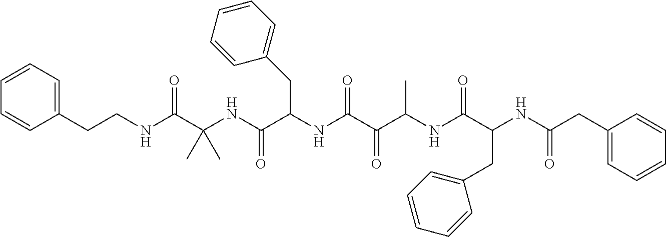 [SMILES: CC(NC(=O)C(Cc1ccccc1)NC(=O)Cc1ccccc1)C(=O)C(=O)NC(Cc1ccccc1)C(=O)NC(C)(C)C(=O)NCCc1ccccc1] | N-(1-((2-methyl-1-oxo-1-(phenethylamino)propan-2-yl)amino)-1- oxo-3-phenylpropan-2-yl)-2-oxo-3-(3-phenyl-2-(2- phenylacetamido)propanamido)butanamide |
| PHELIX231 | 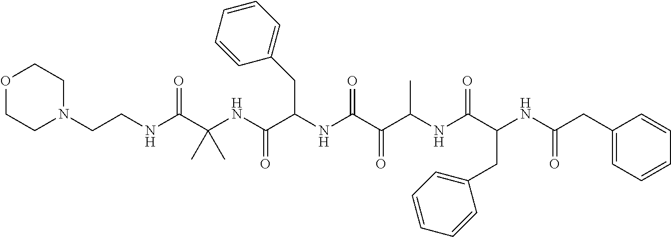 [SMILES: CC(NC(=O)C(Cc1ccccc1)NC(=O)Cc1ccccc1)C(=O)C(=O)NC(Cc1ccccc1)C(=O)NC(C)(C)C(=O)NCCN1CCOCC1] | N-(1-((2-methyl-1-((2-morpholinoethyl)amino)-1-oxopropan-2- yl)amino)-1-oxo-3-phenylpropan-2-yl)-2-oxo-3-(3-phenyl-2-(2- phenylacetamido)propanamido)butanamide |
| PHELIX232 | 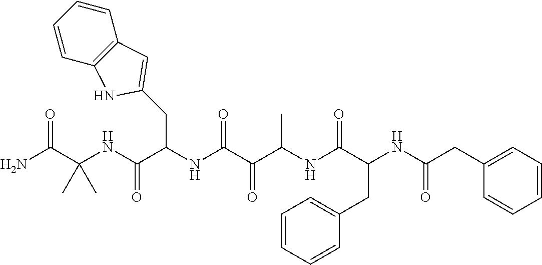 [SMILES: CC(NC(=O)C(Cc1ccccc1)NC(=O)Cc1ccccc1)C(=O)C(=O)NC(Cc1cc2ccccc2[nH]1)C(=O)NC(C)(C)C(N)=O] | N-(1-((1-amino-2-methyl-1-oxopropan-2-yl)amino)-3-(1H-indol- 2-yl)-1-oxopropan-2-yl)-2-oxo-3-(3-phenyl-2-(2- phenylacetamido)propanamido)butanamide |
| PHELIX233 | 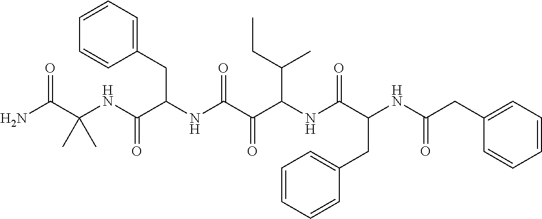 [SMILES: CCC(C)C(NC(=O)C(Cc1ccccc1)NC(=O)Cc1ccccc1)C(=O)C(=O)NC(Cc1ccccc1)C(=O)NC(C)(C)C(N)=O] | N-(1-((1-amino-2-methyl-1-oxopropan-2-yl)amino)-1-oxo-3- phenylpropan-2-yl)-4-methyl-2-oxo-3-(3-phenyl-2-(2- phenylacetamido)propanamido)hexanamide |
| PHELIX234 | 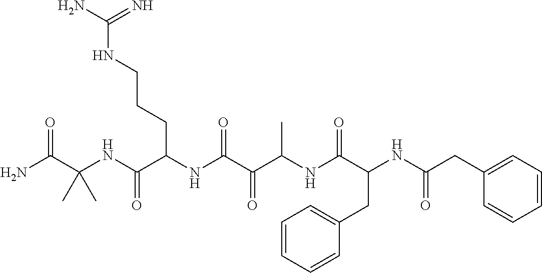 [SMILES: CC(NC(=O)C(Cc1ccccc1)NC(=O)Cc1ccccc1)C(=O)C(=O)NC(CCCNC(=N)N)C(=O)NC(C)(C)C(N)=O] | N-(1-amino-2-methyl-1-oxopropan-2-yl)-5-guanidino-2-(2-oxo- 3-(3-phenyl-2-(2-phenylacetamido)propanamido)butanamido)pentanamide |
| PHELIX235 | 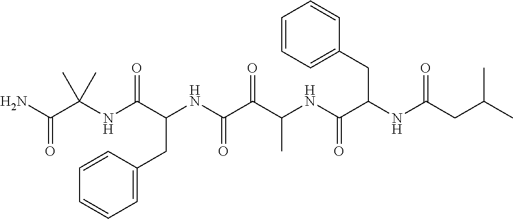 [SMILES: CC(C)CC(=O)NC(Cc1ccccc1)C(=O)NC(C)C(=O)C(=O)NC(Cc1ccccc1)C(=O)NC(C)(C)C(N)=O] | N-(1-((1-amino-2-methyl-1-oxopropan-2-yl)amino)-1-oxo-3- phenylpropan-2-yl)-3-(2-(3-methylbutanamido)-3-phenylpropanamido)- 2-oxobutanamide |
| PHELIX236 | 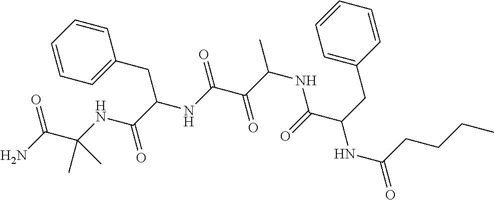 [SMILES: CCCCC(=O)NC(Cc1ccccc1)C(=O)NC(C)C(=O)C(=O)NC(Cc1ccccc1)C(=O)NC(C)(C)C(N)=O] | N-(1-((4-((1-((1-amino-2-methyl-1-oxopropan- 2-yl)amino)-1-oxo-3-phenylpropan- 2-yl)amino)-3,4-dioxobutan-2-yl)amino)-1-oxo-3-phenylpropan- 2-yl)pentanamide |
| PHELIX237 | 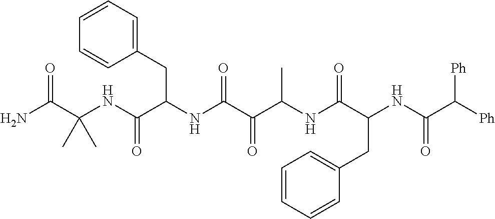 [SMILES: CC(NC(=O)C(Cc1ccccc1)NC(=O)C(c1ccccc1)c1ccccc1)C(=O)C(=O)NC(Cc1ccccc1)C(=O)NC(C)(C)C(N)=O] | N-((S)-1-((1-amino-2-methyl-1-oxopropan- 2-yl)amino)-1-oxo-3- phenylpropan-2-yl)-3-((S)-2-(2,2-diphenylacetamido)-3-phenylpropanamido)- 2-oxobutanamide |
| PHELIX238 | 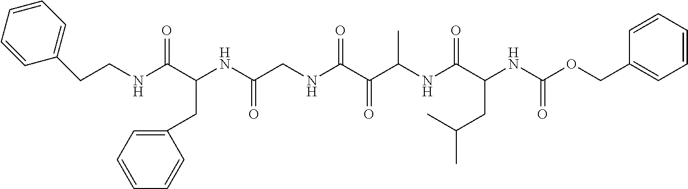 [SMILES: CC(C)CC(NC(=O)OCc1ccccc1)C(=O)NC(C)C(=O)C(=O)NCC(=O)NC(Cc1ccccc1)C(=O)NCCc1ccccc1] | benzyl (5-benzyl-12,17-dimethyl-4,7,10,11,14-pentaoxo-1- phenyl-3,6,9,13-tetraazaoctadecan-15-yl)carbamate |
| PHELIX241 | 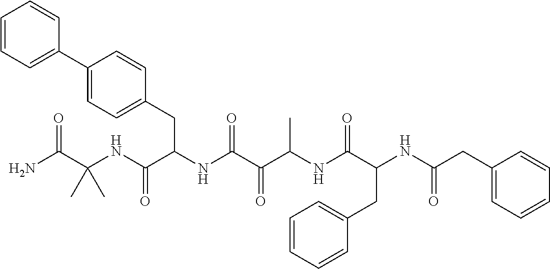 [SMILES: CC(NC(=O)C(Cc1ccccc1)NC(=O)Cc1ccccc1)C(=O)C(=O)NC(Cc1ccc(-c2ccccc2)cc1)C(=O)NC(C)(C)C(N)=O] | N-(3-([1,1′-biphenyl]-4-yl)-1-((1-amino- 2-methyl-1-oxopropan-2- yl)amino)-1-oxopropan-2-yl)-2-oxo-3-(3-phenyl-2-(2- phenylacetamido)propanamido)butanamide |
| PHELIX242 | 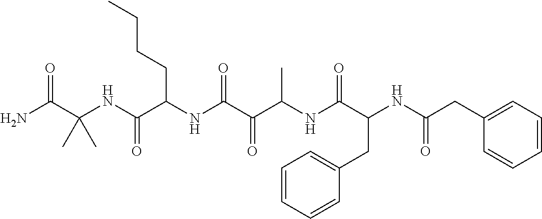 [SMILES: CCCCC(NC(=O)C(=O)C(C)NC(=O)C(Cc1ccccc1)NC(=O)Cc1ccccc1)C(=O)NC(C)(C)C(N)=O] | N-(1-amino-2-methyl-1-oxopropan-2-yl)- 2-(2-oxo-3-(3-phenyl-2-(2- phenylacetamido)propanamido)butanamido)hexanamide |
| PHELIX243 | 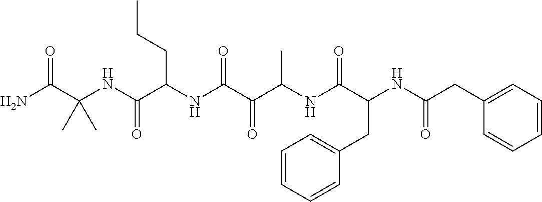 [SMILES: CCCC(NC(=O)C(=O)C(C)NC(=O)C(Cc1ccccc1)NC(=O)Cc1ccccc1)C(=O)NC(C)(C)C(N)=O] | N-(1-amino-2-methyl-1-oxopropan-2-yl)- 2-(2-oxo-3-(3-phenyl-2-(2- phenylacetamido)propanamido)butanamido)pentanamide |
| PHELIX247 PHELIX248 | 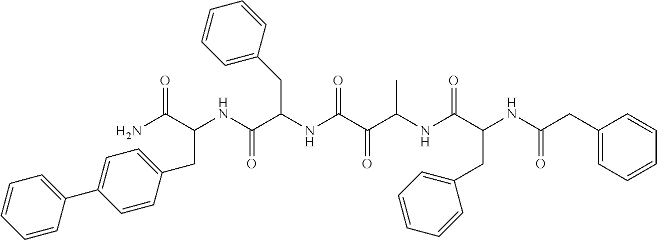 [SMILES: CC(NC(=O)C(Cc1ccccc1)NC(=O)Cc1ccccc1)C(=O)C(=O)NC(Cc1ccccc1)C(=O)NC(Cc1ccc(-c2ccccc2)cc1)C(N)=O] | N-(1-((3-([1,1′-biphenyl]-4-yl)-1-amino- 1-oxopropan-2-yl)amino)- 1-oxo-3-phenylpropan-2-yl)-2-oxo-3-(3-phenyl-2-(2- phenylacetamido)propanamido)butanamide |
| PHELIX249 | 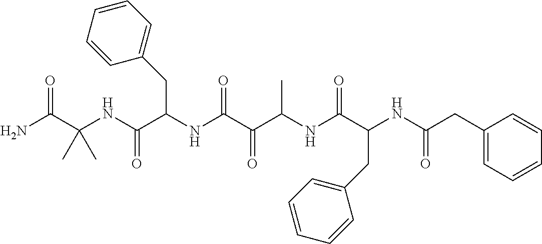 [SMILES: CC(NC(=O)C(Cc1ccccc1)NC(=O)Cc1ccccc1)C(=O)C(=O)NC(Cc1ccccc1)C(=O)NC(C)(C)C(N)=O] | N-(1-((1-amino-2-methyl-1-oxopropan-2-yl)amino)-1-oxo-3- phenylpropan-2-yl)-2-oxo-3-(3-phenyl-2-(2-phenylacetamido)propanamido)butanamide |
| PHELIX251 | 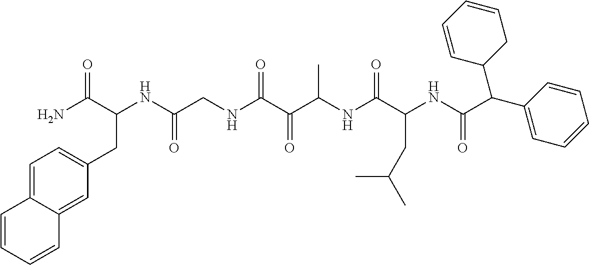 [SMILES: CC(C)CC(NC(=O)C(c1ccccc1)C1C=CC=CC1)C(=O)NC(C)C(=O)C(=O)NCC(=O)NC(Cc1ccc2ccccc2c1)C(N)=O] | N-(4-((2-((1-amino-3-(naphthalen-2-yl)-1- oxopropan-2-yl)amino)- 2-oxoethyl)amino)-3,4-dioxobutan-2-yl)-2-(2,2-diphenylacetamido)- 4-methylpentanamide |
| PHELIX252 | 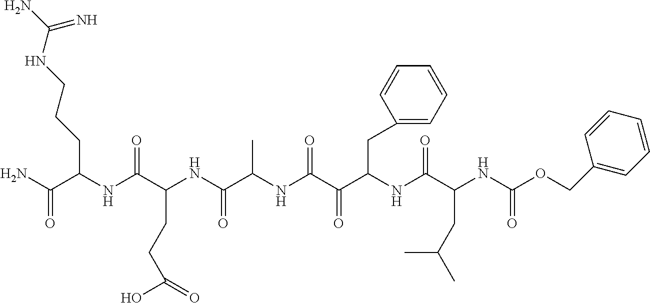 [SMILES: CC(C)CC(NC(=O)OCc1ccccc1)C(=O)NC(Cc1ccccc1)C(=O)C(=O)NC(C)C(=O)NC(CCC(=O)O)C(=O)NC(CCCNC(=N)N)C(N)=O] | 15-((1-amino-5-guanidino-1-oxopentan-2-yl)carbamoyl)-8- benzyl-5-isobutyl-12-methyl-3,6,9,10,13-pentaoxo-1-phenyl- 2-oxa-4,7,11,14-tetraazaoctadecan-18-oic acid |
| PHELIX253 | 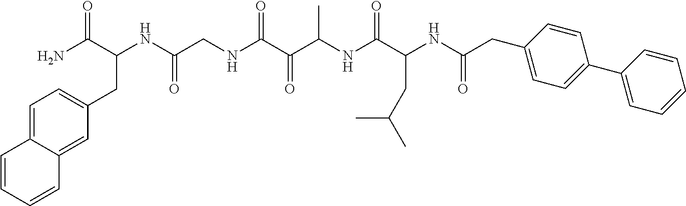 [SMILES: CC(C)CC(NC(=O)Cc1ccc(-c2ccccc2)cc1)C(=O)NC(C)C(=O)C(=O)NCC(=O)NC(Cc1ccc2ccccc2c1)C(N)=O] | 2-(2-([1,1′-biphenyl]-4-yl)acetamido)-N-(4-((2-((1-amino-3- (naphthalen-2-yl)-1-oxopropan-2-yl)amino)-2-oxoethyl)amino)- 3,4-dioxobutan-2-yl)-4-methylpentanamide |
| PHELIX254 PHELIX339 | 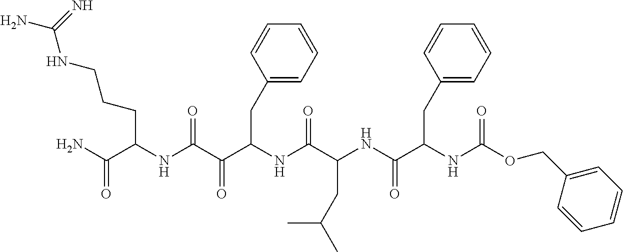 [SMILES: CC(C)CC(NC(=O)C(Cc1ccccc1)NC(=O)OCc1ccccc1)C(=O)NC(Cc1ccccc1)C(=O)C(=O)NC(CCCNC(=N)N)C(N)=O] | benzyl (1-amino-10-benzyl-6-carbamoyl-1-imino-13-isobutyl- 8,9,12,15-tetraoxo-17-phenyl-2,7,11,14-tetraazaheptadecan-16- yl)carbamate |
| PHELIX256 | 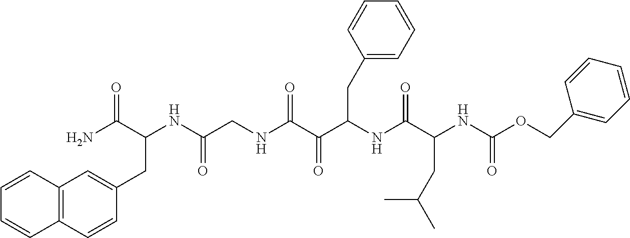 [SMILES: CC(C)CC(NC(=O)OCc1ccccc1)C(=O)NC(Cc1ccccc1)C(=O)C(=O)NCC(=O)NC(Cc1ccc2ccccc2c1)C(N)=O] | benzyl (1-((4-((2-((1-amino-3-(naphthalen- 2-yl)-1-oxopropan-2- yl)amino)-2-oxoethyl)amino)-3,4-dioxo-1-phenylbutan-2-yl)amino)- 4-methyl-1-oxopentan-2-yl)carbamate |
| PHELIX257 | 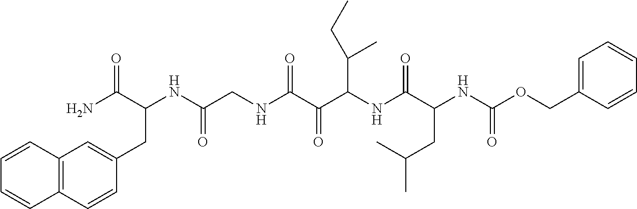 [SMILES: CCC(C)C(NC(=O)C(CC(C)C)NC(=O)OCc1ccccc1)C(=O)C(=O)NCC(=O)NC(Cc1ccc2ccccc2c1)C(N)=O] | benzyl (1-((1-((2-((1-amino-3-(naphthalen- 2-yl)-1-oxopropan-2- yl)amino)-2-oxoethyl)amino)-4-methyl-1,2-dioxohexan-3-yl)amino)- 4-methyl-1-oxopentan-2-yl)carbamate |
| PHELIX258 | 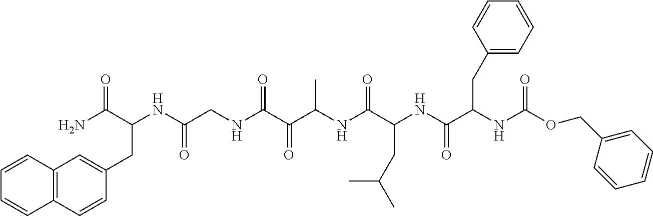 [SMILES: CC(C)CC(NC(=O)C(Cc1ccccc1)NC(=O)OCc1ccccc1)C(=O)NC(C)C(=O)C(=O)NCC(=O)NC(Cc1ccc2ccccc2c1)C(N)=O] | benzyl (1-amino-12-isobutyl-9-methyl- 2-(naphthalen-2-ylmethyl)-1,4,7,8,11,14- hexaoxo-16-phenyl-3,6,10,13-tetraazahexadecan-15-yl)carbamate |
| PHELIX259 | 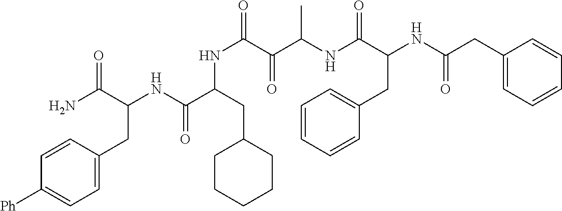 [SMILES: CC(NC(=O)C(Cc1ccccc1)NC(=O)Cc1ccccc1)C(=O)C(=O)NC(CC1CCCCC1)C(=O)NC(Cc1ccc(-c2ccccc2)cc1)C(N)=O] | N-(1-((3-([1,1′-biphenyl]-4-yl)-1-amino- 1-oxopropan-2-yl)amino)- 3-cyclohexyl-1-oxopropan-2-yl)-2-oxo-3-(3-phenyl-2-(2- phenylacetamido)propanamido)butanamide |
| PHELIX260 | 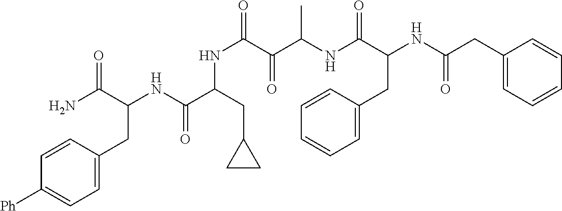 [SMILES: CC(NC(=O)C(Cc1ccccc1)NC(=O)Cc1ccccc1)C(=O)C(=O)NC(CC1CC1)C(=O)NC(Cc1ccc(-c2ccccc2)cc1)C(N)=O] | N-(1-((3-([1,1′-biphenyl]-4-yl)-1-amino-1- oxopropan-2-yl)amino)- 3-cyclopropyl-1-oxopropan-2-yl)-2-oxo-3-(3-phenyl-2-(2- phenylacetamido)propanamido)butanamide |
| PHELIX261 | 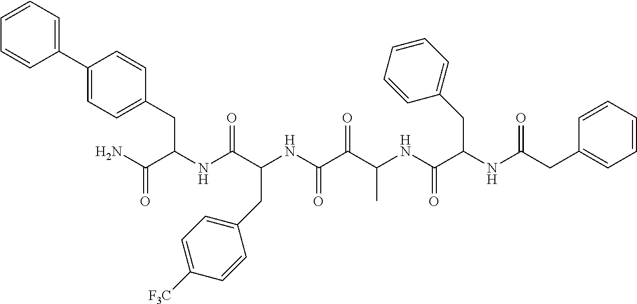 [SMILES: CC(NC(=O)C(Cc1ccccc1)NC(=O)Cc1ccccc1)C(=O)C(=O)NC(Cc1ccc(C(F)(F)F)cc1)C(=O)NC(Cc1ccc(-c2ccccc2)cc1)C(N)=O] | N-(1-((3-([1,1′-biphenyl]-4-yl)-1-amino-1- oxopropan-2-yl)amino)- 1-oxo-3-(4-(trifluoromethyl)phenyl)propan-2-yl)-2-oxo-3-(3-phenyl- 2-(2-phenylacetamido)propanamido)butanamide |
| PHELIX262 | 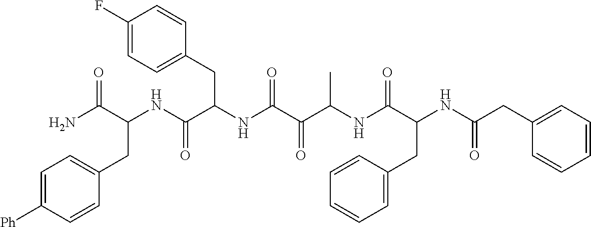 [SMILES: CC(NC(=O)C(Cc1ccccc1)NC(=O)Cc1ccccc1)C(=O)C(=O)NC(Cc1ccc(F)cc1)C(=O)NC(Cc1ccc(-c2ccccc2)cc1)C(N)=O] | N-(1-((3-([1,1′-biphenyl]-4-yl)-1-amino-1- oxopropan-2-yl)amino- 3-(4-fluorophenyl)-1-oxopropan-2-yl)-2-oxo-3-(3-phenyl-2-(2- phenylacetamido)propanamido)butanamide |
| PHELIX263 | 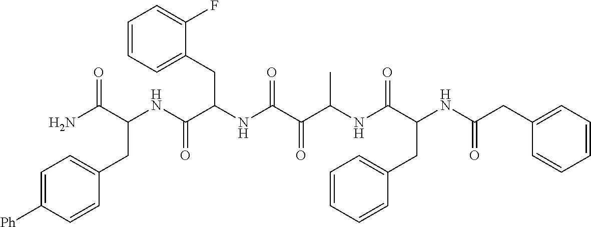 [SMILES: CC(NC(=O)C(Cc1ccccc1)NC(=O)Cc1ccccc1)C(=O)C(=O)NC(Cc1ccccc1F)C(=O)NC(Cc1ccc(-c2ccccc2)cc1)C(N)=O] | N-(1-((3-([1,1′-biphenyl]-4-yl)-1-amino-1- oxopropan-2-yl)amino)- 3-(2-fluorophenyl)-1-oxopropan-2-yl)-2-oxo-3-(3-phenyl-2-(2- phenylacetamido)propanamido)butanamide |
| PHELIX264 | 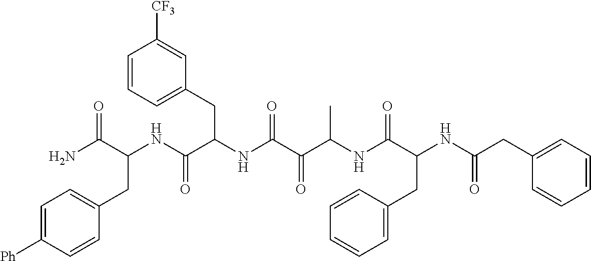 [SMILES: CC(NC(=O)C(Cc1ccccc1)NC(=O)Cc1ccccc1)C(=O)C(=O)NC(Cc1cccc(C(F)(F)F)c1)C(=O)NC(Cc1ccc(-c2ccccc2)cc1)C(N)=O] | N-(1-((3-([1,1′-biphenyl]-4-yl)-1-amino- 1-oxopropan-2-yl)amino)- 1-oxo-3-(3-(trifluoromethyl)phenyl)propan-2-yl)-2-oxo-3-(3-phenyl-2- (2-phenylacetmido)propanamido)butanamide |
| PHELIX265 | 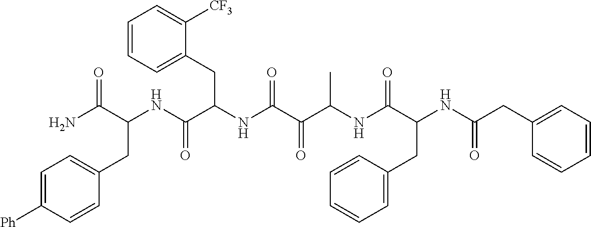 [SMILES: CC(NC(=O)C(Cc1ccccc1)NC(=O)Cc1ccccc1)C(=O)C(=O)NC(Cc1ccccc1C(F)(F)F)C(=O)NC(Cc1ccc(-c2ccccc2)cc1)C(N)=O] | N-(1-((3-([1,1′-biphenyl]-4-yl)-1-amino-1- oxopropan-2-yl)amino)- 1-oxo-3-(2-(trifluoromethyl)phenyl)propan-2-yl)-2-oxo-3-(3-phenyl-2- (2-phenylacetamido)propanamido)butanamide |
| PHELIX266 | 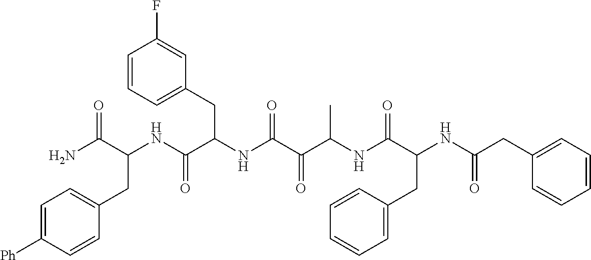 [SMILES: CC(NC(=O)C(Cc1ccccc1)NC(=O)Cc1ccccc1)C(=O)C(=O)NC(Cc1cccc(F)c1)C(=O)NC(Cc1ccc(-c2ccccc2)cc1)C(N)=O] | N-(1-((3-([1,1′-biphenyl]-4-yl)-1-amino-1- oxopropan-2-yl)amino)- 3-(3-fluorophenyl)-1-oxopropan-2-yl)-2-oxo-3-(3-phenyl-2-(2- phenylacetamido)propanamido)butanamide |
| PHELIX268 PHELIX294 | 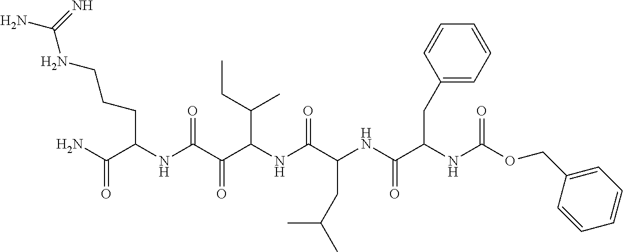 [SMILES: CCC(C)C(NC(=O)C(CC(C)C)NC(=O)C(Cc1ccccc1)NC(=O)OCc1ccccc1)C(=O)C(=O)NC(CCCNC(=N)N)C(N)=O] | benzyl (1-amino-10-(sec-butyl)-6-carbamoyl- 1-imino-13-isobutyl-8,9,12,15- tetraoxo-17-phenyl-2,7,11,14-tetraazaheptadecan-16-yl)carbamate |
| PHELIX269 | 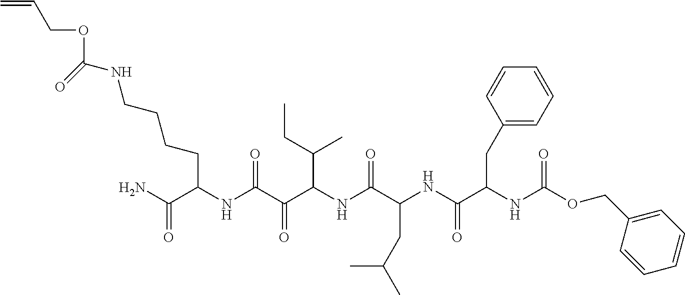 [SMILES: C=CCOC(=O)NCCCCC(NC(=O)C(=O)C(NC(=O)C(CC(C)C)NC(=O)C(Cc1ccccc1)NC(=O)OCc1ccccc1)C(C)CC)C(N)=O] | benzyl (15-(sec-butyl)-11-carbamoyl- 18-isobutyl-5,13,14,17,20- pentaoxo-22-phenyl-4-oxa-6,12,16,19-tetraazadocos-1-en-21-yl)carbamate |
| PHELIX270 | 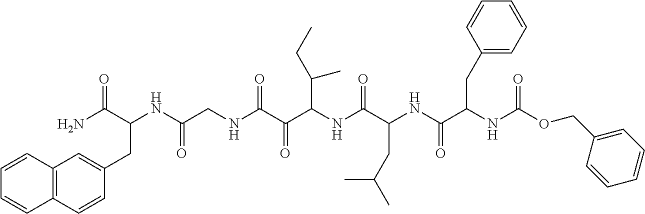 [SMILES: CCC(C)C(NC(=O)C(CC(C)C)NC(=O)C(Cc1ccccc1)NC(=O)OCc1ccccc1)C(=O)C(=O)NCC(=O)NC(Cc1ccc2ccccc2c1)C(N)=O] | benzyl (1-amino-9-(sec-butyl)-12-isobutyl-2-(naphthalen-2- ylmethyl)-1,4,7,8,11,14-hexaoxo-16-phenyl-3,6,10,13-tetraazahexadecan- 15-yl)carbamate |
| PHELIX289 | 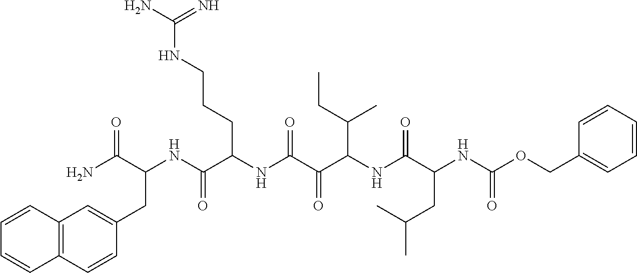 [SMILES: CCC(C)C(NC(=O)C(CC(C)C)NC(=O)OCc1ccccc1)C(=O)C(=O)NC(CCCNC(=N)N)C(=O)NC(Cc1ccc2ccccc2c1)C(N)=O] | benzyl (1-((1-((1-((1-amino-3-(naphthalen- 2-yl)-1-oxopropan-2-yl)amino)- 5-guanidino-1-oxopentan-2-yl)amino)-4-methyl-1,2-dioxohexan-3- yl)amino)-4-methyl-1-oxopentan-2-yl)carbamate |
| PHELIX290 | 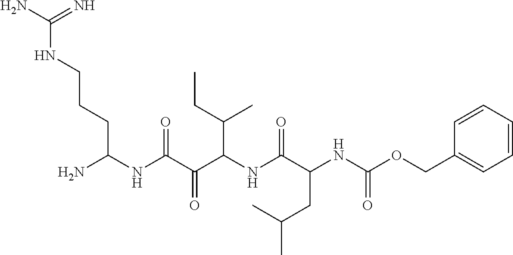 [SMILES: CCC(C)C(NC(=O)C(CC(C)C)NC(=O)OCc1ccccc1)C(=O)C(=O)NC(N)CCCNC(=N)N] | benzyl (1-((1-((1-amino-5- guanidino-1-oxopentan-2-yl)amino)-4- methyl-1,2-dioxohexan-3-yl)amino)-4-methyl-1-oxopentan-2-yl)carbamate |
| PHELIX291 | 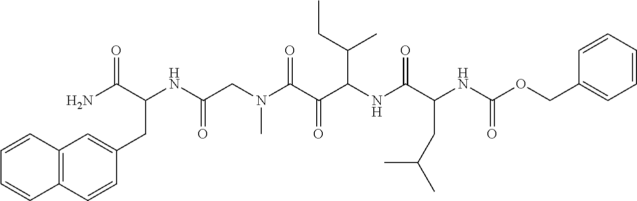 [SMILES: CCC(C)C(NC(=O)C(CC(C)C)NC(=O)OCc1ccccc1)C(=O)C(=O)N(C)CC(=O)NC(Cc1ccc2ccccc2c1)C(N)=O] | benzyl (1-((1-((2-((1-amino-3- (naphthalen-2-yl)-1-oxopropan-2-yl)amino)- 2-oxoethyl)(methyl)amino)-4-methyl-1,2-dioxohexan-3- yl)amino)-4-methyl-1-oxopentan-2-yl)carbamate |
| PHELIX292 | 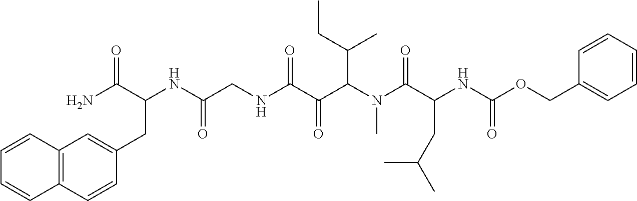 [SMILES: CCC(C)C(C(=O)C(=O)NCC(=O)NC(Cc1ccc2ccccc2c1)C(N)=O)N(C)C(=O)C(CC(C)C)NC(=O)OCc1ccccc1] | benzyl (1-((1-((2-((1-amino-3-(naphthalen- 2-yl)-1-oxopropan-2- yl)amino)-2-oxoethyl)amino)-4-methyl-1,2-dioxohexan-3-yl)(methyl)amino)- 4-methyl-1-oxopentan-2-yl)carbamate |
| PHELIX293 | 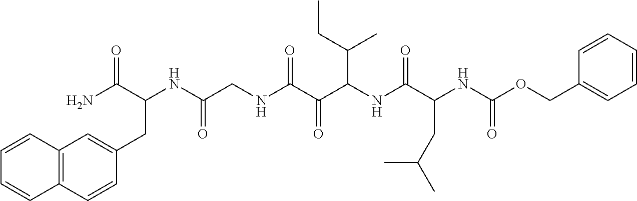 [SMILES: CCC(C)C(NC(=O)C(CC(C)C)NC(=O)OCc1ccccc1)C(=O)C(=O)NCC(=O)NC(Cc1ccc2ccccc2c1)C(N)=O] | benzyl (1-((1-((2-((1-amino-3-(naphthalen-2-yl)- 1-oxopropan-2- yl)amino)-2-oxoethyl)amino)-4-methyl-1,2-dioxohexan-3-yl)amino)- 4-methyl-1-oxopentan-2-yl)carbamate |
| PHELIX295 | 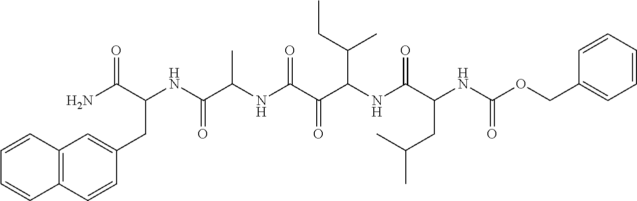 [SMILES: CCC(C)C(NC(=O)C(CC(C)C)NC(=O)OCc1ccccc1)C(=O)C(=O)NC(C)C(=O)NC(Cc1ccc2ccccc2c1)C(N)=O] | benzyl (1-((1-((1-((1-amino-3-(naphthalen-2-yl)- 1-oxopropan-2-yl)amino)- 1-oxopropan-2-yl)amino)-4-methyl-1,2-dioxohexan-3-yl)amino)- 4-methyl-1-oxopentan-2-yl)carbamate |
| PHELIX296 | 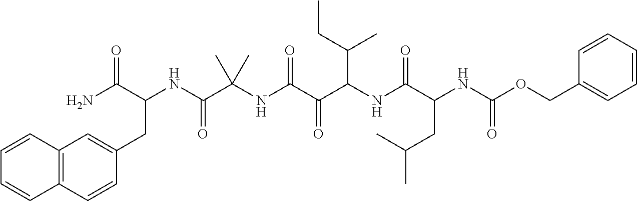 [SMILES: CCC(C)C(NC(=O)C(CC(C)C)NC(=O)OCc1ccccc1)C(=O)C(=O)NC(C)(C)C(=O)NC(Cc1ccc2ccccc2c1)C(N)=O] | benzyl (1-((1-((1-((1-amino-3-(naphthalen- 2-yl)-1-oxopropan-2-yl)amino)- 2-methyl-1-oxopropan-2-yl)amino)-4-methyl-1,2- dioxohexan-3-yl)amino)-4-methyl-1-oxopentan-2-yl)carbamate |
| PHELIX297 | 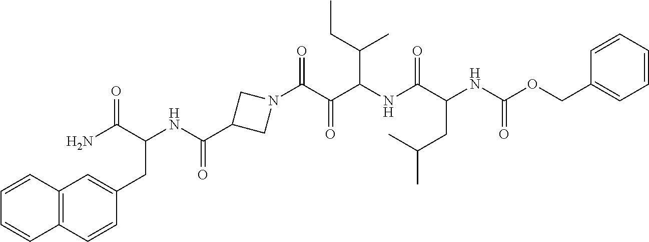 [SMILES: CCC(C)C(NC(=O)C(CC(C)C)NC(=O)OCc1ccccc1)C(=O)C(=O)N1CC(C(=O)NC(Cc2ccc3ccccc3c2)C(N)=O)C1] | benzyl (1-((1-(3-((1-amino-3-(naphthalen- 2-yl)-1-oxopropan-2- yl)carbamoyl)azetidin-1-yl)-4-methyl-1,2-dioxohexan-3-yl)amino)- 4-methyl-1-oxopentan-2-yl)carbamate |
| PHELIX298 | 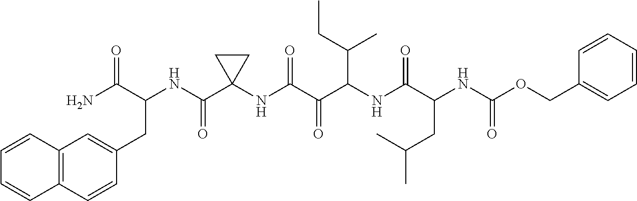 [SMILES: CCC(C)C(NC(=O)C(CC(C)C)NC(=O)OCc1ccccc1)C(=O)C(=O)NC1(C(=O)NC(Cc2ccc3ccccc3c2)C(N)=O)CC1] | benzyl (1-((1-((1-((1-amino-3-(naphthalen- 2-yl)-1-oxopropan-2-yl)carbamoyl) cyclopropyl)amino)-4-methyl-1,2-dioxohexan-3-yl)amino)- 4-methyl-1-oxopentan-2-yl)carbamate |
| PHELIX299 | 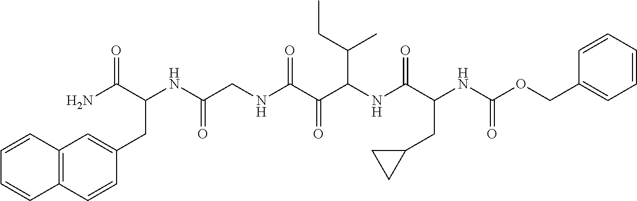 [SMILES: CCC(C)C(NC(=O)C(CC1CC1)NC(=O)OCc1ccccc1)C(=O)C(=O)NCC(=O)NC(Cc1ccc2ccccc2c1)C(N)=O] | benzyl (1-((1-((2-((1-amino-3- (naphthalen-2-yl)-1-oxopropan-2- yl)amino)-2-oxoethyl)amino)-4-methyl-1,2-dioxohexan-3-yl)amino)- 3-cyclopropyl-1-oxopropan-2-yl)carbamate |
| PHELIX300 | 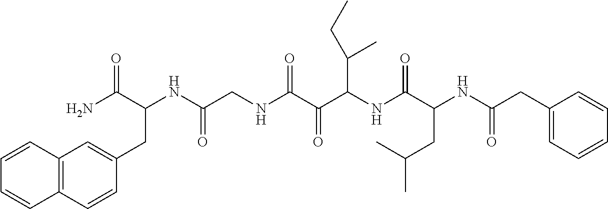 [SMILES: CCC(C)C(NC(=O)C(CC(C)C)NC(=O)Cc1ccccc1)C(=O)C(=O)NCC(=O)NC(Cc1ccc2ccccc2c1)C(N)=O] | N-(2-((1-amino-3-(naphthalen- 2-yl)-1-oxopropan-2-yl)amino)-2- oxoethyl)-4-methyl-3-(4-methyl-2-(2-phenylacetamido)pentanamido)- 2-oxohexanamide |
| PHELIX302 | 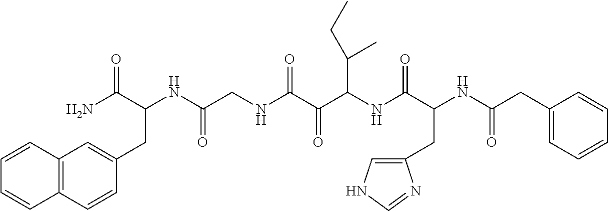 [SMILES: CCC(C)C(NC(=O)C(Cc1c[nH]cn1)NC(=O)Cc1ccccc1)C(=O)C(=O)NCC(=O)NC(Cc1ccc2ccccc2c1)C(N)=O] | 3-(3-(1H-imidazol-4-yl)-2- (2-phenylacetamido)propanamido)-N- (2-((1-amino-3-(naphthalen-2-yl)-1-oxopropan-2-yl)amino)-2- oxoethyl)-4-methyl-2-oxohexanamide |
| PHELIX303 | 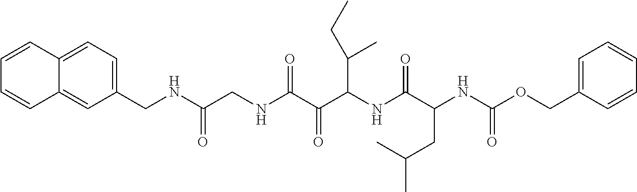 [SMILES: CCC(C)C(NC(=O)C(CC(C)C)NC(=O)OCc1ccccc1)C(=O)C(=O)NCC(=O)NCc1ccc2ccccc2c1] | benzyl (4-methyl-1-((4-methyl-1-((2-((naphthalen-2- ylmethyl)amino)-2-oxoethyl)amino)-1,2-dioxohexan-3- yl)amino)-1-oxopentan-2-yl)carbamate |
| PHELIX304 | 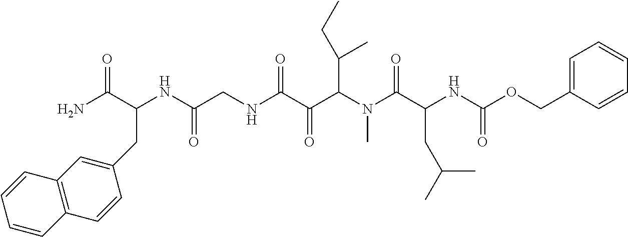 [SMILES: CCC(C)C(C(=O)C(=O)NCC(=O)NC(Cc1ccc2ccccc2c1)C(N)=O)N(C)C(=O)C(CC(C)C)NC(=O)OCc1ccccc1] | benzyl (1-((1-((2-((1-amino-3-(naphthalen- 2-yl)-1-oxopropan-2- yl)amino)-2-oxoethyl)amino)-4-methyl-1,2-dioxohexan-3-yl)amino)- 4-methyl-1-oxopentan-2-yl)carbamate |
| PHELIX305/306 | 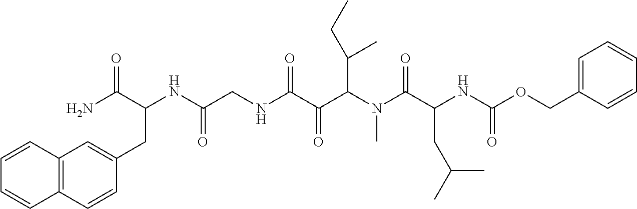 [SMILES: CCC(C)C(C(=O)C(=O)NCC(=O)NC(Cc1ccc2ccccc2c1)C(N)=O)N(C)C(=O)C(CC(C)C)NC(=O)OCc1ccccc1] | benzyl (1-((1-((2-((1-amino-3-(naphthalen- 2-yl)-1-oxopropan-2- yl)amino)-2-oxoethyl)amino)-4-methyl-1,2-dioxohexan-3-yl)(methyl)amino)- 4-methyl-1-oxopentan-2-yl)carbamate |
| PHELIX307 | 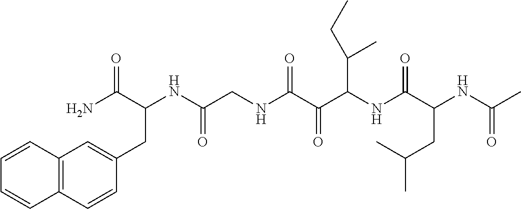 [SMILES: CCC(C)C(NC(=O)C(CC(C)C)NC(C)=O)C(=O)C(=O)NCC(=O)NC(Cc1ccc2ccccc2c1)C(N)=O] | 3-(2-acetamido-4-methylpentanamido)-N-(2-((1-amino-3- (naphthalen-2-yl)-1-oxopropan-2-yl)amino)-2-oxoethyl)-4-methyl- 2-oxohexanamide |
| PHELIX308 | 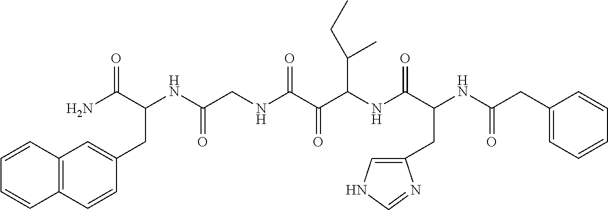 [SMILES: CCC(C)C(NC(=O)C(Cc1c[nH]cn1)NC(=O)Cc1ccccc1)C(=O)C(=O)NCC(=O)NC(Cc1ccc2ccccc2c1)C(N)=O] | 3-(3-(1H-imidazol-4-yl)-2-(2-phenylacetamido) propanamido)-N- (2-((1-amino-3-(naphthalen-2-yl)-1-oxopropan-2-yl)amino)-2-oxoethyl)- 4-methyl-2-oxohexanamide |
| PHELIX309 | 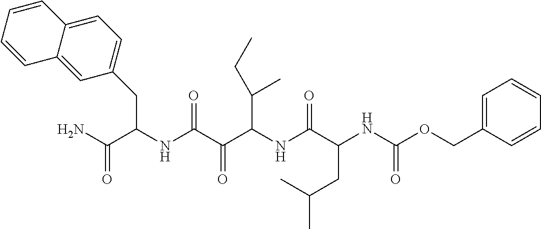 [SMILES: CCC(C)C(NC(=O)C(CC(C)C)NC(=O)OCc1ccccc1)C(=O)C(=O)NC(Cc1ccc2ccccc2c1)C(N)=O] | benzyl (1-((1-((1-amino-3-(naphthalen-2-yl)-1-oxopropan-2- yl)amino)-4-methyl-1,2-dioxohexan-3-yl)amino)-4-methyl-1-oxopentan- 2-yl)carbamate |
| PHELIX310 | 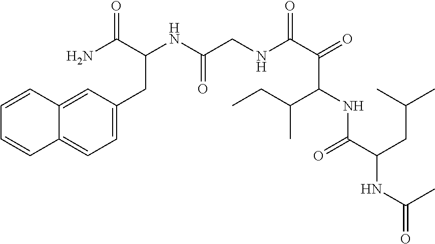 [SMILES: CCC(C)C(NC(=O)C(CC(C)C)NC(C)=O)C(=O)C(=O)NCC(=O)NC(Cc1ccc2ccccc2c1)C(N)=O] | 3-(2-acetamido)-4-methylpentanamido)-N-(2-((1-amino-3- (naphthalen-2-yl)-1-oxopropan-2-yl)amino)-2-oxoethyl)-4-methyl- 2-oxohexanamide |
| PHELIX311 | 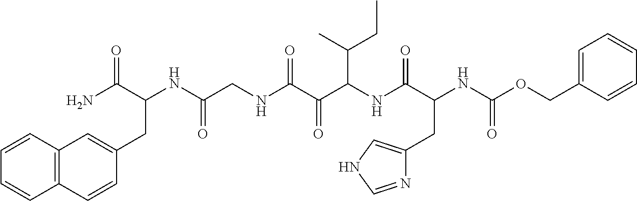 [SMILES: CCC(C)C(NC(=O)C(Cc1c[nH]cn1)NC(=O)OCc1ccccc1)C(=O)C(=O)NCC(=O)NC(Cc1ccc2ccccc2c1)C(N)=O] | benzyl (1-((1-((2-((1-amino-3-(naphthalen- 2-yl)-1-oxopropan-2- yl)amino)-2-oxoethyl)amino)-4-methyl-1,2-dioxohexan-3-yl)amino)-3- (1H-imidazol-4-yl)-1-oxopropan-2-yl)carbamate |
| PHELIX312 | 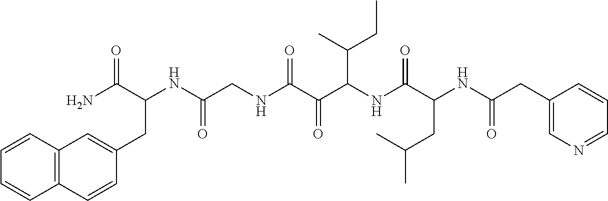 [SMILES: CCC(C)C(NC(=O)C(CC(C)C)NC(=O)Cc1cccnc1)C(=O)C(=O)NCC(=O)NC(Cc1ccc2ccccc2c1)C(N)=O] | N-(2-((1-amino-3-(naphthalen-2-yl)-1- oxopropan-2-yl)amino)-2- oxoethyl)-4-methyl-3-(4-methyl-2-(2-(pyridin-3-yl)acetamido)pentanamido)- 2-oxohexanamide |
| PHELIX313 | 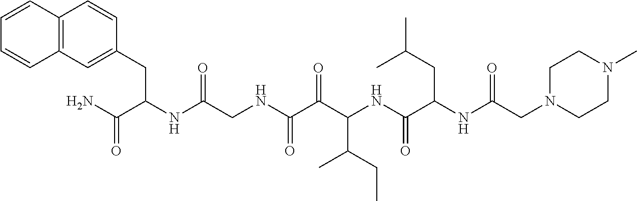 [SMILES: CCC(C)C(NC(=O)C(CC(C)C)NC(=O)CN1CCN(C)CC1)C(=O)C(=O)NCC(=O)NC(Cc1ccc2ccccc2c1)C(N)=O] | N-(2-((1-amino-3-(naphthalen-2-yl)-1- oxopropan-2-yl)amino)-2- oxoethyl)-4-methyl-3-(4-methyl-2-(2-(4-methylpiperazin-1-yl)acetamido)pentanamido)- 2-oxohexanamide |
| PHELIX314 | 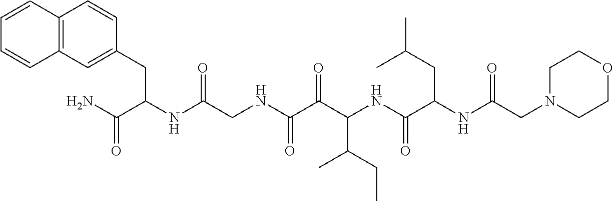 [SMILES: CCC(C)C(NC(=O)C(CC(C)C)NC(=O)CN1CCOCC1)C(=O)C(=O)NCC(=O)NC(Cc1ccc2ccccc2c1)C(N)=O] | N-(2-((1-amino-3-(naphthalen-2-yl)-1-oxopropan-2-yl)amino)- 2-oxoethyl)-4-methyl-3-(4-methyl-2-(2-morpholinoacetamido)pentanamido)- 2-oxohexanamide |
| PHELIX315 | 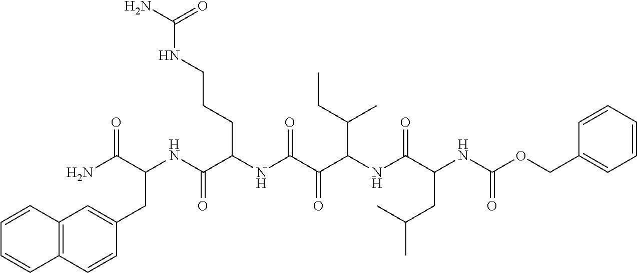 [SMILES: CCC(C)C(NC(=O)C(CC(C)C)NC(=O)OCc1ccccc1)C(=O)C(=O)NC(CCCNC(N)=O)C(=O)NC(Cc1ccc2ccccc2c1)C(N)=O] | benzyl (1-((1-((1-((1-amino-3-(naphthalen- 2-yl)-1-oxopropan-2-yl)amino)-1- oxo-5-ureidopentan-2-yl)amino)-4-methyl-1,2- dioxohexan-3-yl)amino)-4-methyl-1-oxopentan-2-yl)carbamate |
| PHELIX316 | 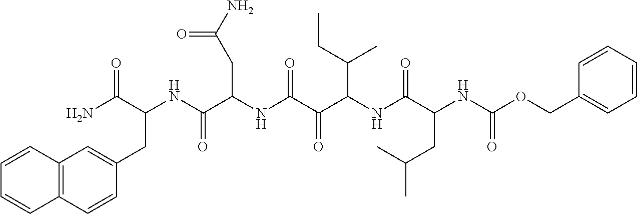 [SMILES: CCC(C)C(NC(=O)C(CC(C)C)NC(=O)OCc1ccccc1)C(=O)C(=O)NC(CC(N)=O)C(=O)NC(Cc1ccc2ccccc2c1)C(N)=O] | benzyl (1-((1-((4-amino-1-((1-amino-3-(naphthalen-2-yl)-1- oxopropan-2-yl)amino)-1,4-dioxobutan-2-yl)amino)- 4-methyl-1,2-dioxohexan- 3-yl)amino)-4-methyl-1-oxopentan-2-yl)carbamate |
| PHELIX317 | 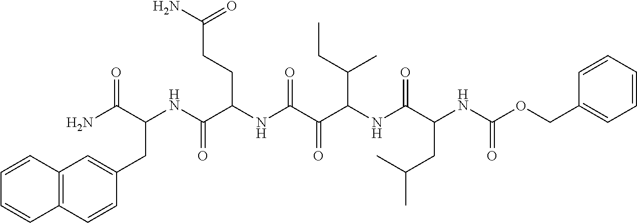 [SMILES: CCC(C)C(NC(=O)C(CC(C)C)NC(=O)OCc1ccccc1)C(=O)C(=O)NC(CCC(N)=O)C(=O)NC(Cc1ccc2ccccc2c1)C(N)=O] | benzyl (1-((1-((5-amino-1-((1-amino-3-(naphthalen-2-yl)-1- oxopropan-2-yl)amino)-1,5-dioxopentan-2-yl)amino)-4- methyl-1,2-dioxohexan-3-yl)amino)-4-methyl-1-oxopentan-2-yl)carbamate |
| PHELIX318 | 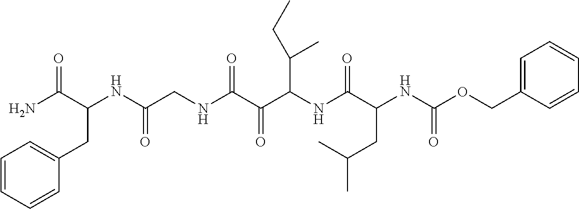 [SMILES: CCC(C)C(NC(=O)C(CC(C)C)NC(=O)OCc1ccccc1)C(=O)C(=O)NCC(=O)NC(Cc1ccccc1)C(N)=O] | benzyl (1-((1-((2-((1-amino-1-oxo-3-phenylpropan- 2-yl)amino)-2-oxoethyl)amino)- 4-methyl-1,2-dioxohexan-3-yl)amino)-4-methyl-1-oxopentan-2-yl)carbamate |
| PHELIX319 | 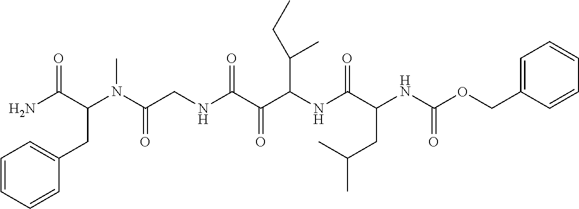 [SMILES: CCC(C)C(NC(=O)C(CC(C)C)NC(=O)OCc1ccccc1)C(=O)C(=O)NCC(=O)N(C)C(Cc1ccccc1)C(N)=O] | benzyl (1-((1-((2-((1-amino-1-oxo-3-phenylpropan-2- yl)(methyl)amino)-2-oxoethyl)amino)-4- methyl-1,2-dioxohexan-3-yl)amino)-4-methyl-1-oxopentan-2-yl)carbamate |
| PHELIX320 | 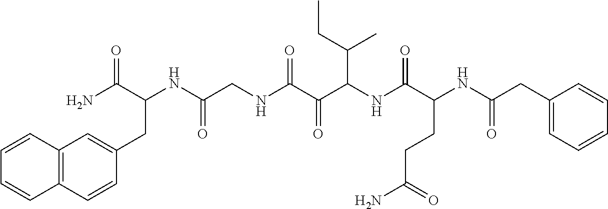 [SMILES: CCC(C)C(NC(=O)C(CCC(N)=O)NC(=O)Cc1ccccc1)C(=O)C(=O)NCC(=O)NC(Cc1ccc2ccccc2c1)C(N)=O] | N¹-(1-((2-((1-amino-3-(naphthalen-2-yl)-1-oxopropan-2- yl)amino)-2-oxoethyl)amino)-4-methyl-1,2-dioxohexan-3-yl)-2-(2- phenylacetamido)pentanediamide |
| PHELIX321 | 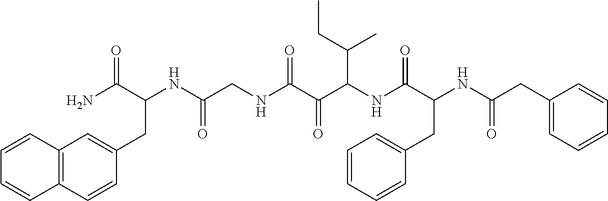 [SMILES: CCC(C)C(NC(=O)C(Cc1ccccc1)NC(=O)Cc1ccccc1)C(=O)C(=O)NCC(=O)NC(Cc1ccc2ccccc2c1)C(N)=O] | N-(2-((1-amino-3-(naphthalen-2-yl)-1-oxopropan-2- yl)amino)-2-oxoethyl)-4-methyl-2-oxo-3-(3-phenyl-2-(2- phenylacetamido)propanamido)hexanamide |
| PHELIX322 | 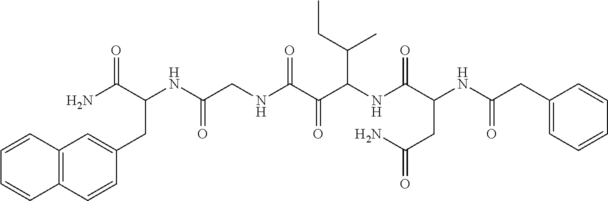 [SMILES: CCC(C)C(NC(=O)C(CC(N)=O)NC(=O)Cc1ccccc1)C(=O)C(=O)NCC(=O)NC(Cc1ccc2ccccc2c1)C(N)=O] | N¹-(1-((2-((1-amino-3-(naphthalen-2-yl)-1-oxopropan-2- yl)amino)-2-oxoethyl)amino)-4-methyl-1,2-dioxohexan-3-yl)-2-(2- phenylacetamido)succinamide |
| PHELIX323 | 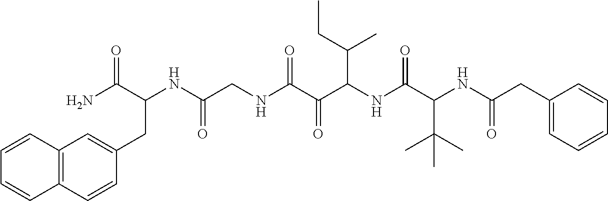 [SMILES: CCC(C)C(NC(=O)C(NC(=O)Cc1ccccc1)C(C)(C)C)C(=O)C(=O)NCC(=O)NC(Cc1ccc2ccccc2c1)C(N)=O] | N-(2-((1-amino-3-(naphthalen-2-yl)-1-oxopropan- 2-yl)amino)-2-oxoethyl)- 3-(3,3-dimethyl-2-(2-phenylacetamido)butanamido)-4-methyl-2-oxohexanamide |
| PHELIX324 | 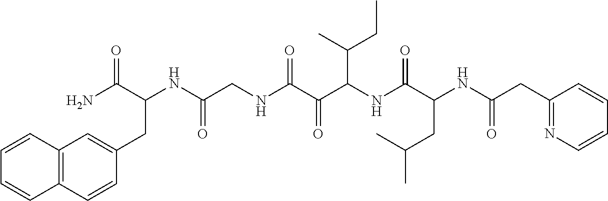 [SMILES: CCC(C)C(NC(=O)C(CC(C)C)NC(=O)Cc1ccccn1)C(=O)C(=O)NCC(=O)NC(Cc1ccc2ccccc2c1)C(N)=O] | N-(2-((1-amino-3-(naphthalen-2-yl)- 1-oxopropan-2-yl)amino)-2- oxoethyl)-4-methyl-3-(4-methyl-2-(2-(pyridin-2-yl)acetamido)pentanamido)-2- oxohexanamide |
| PHELIX325 | 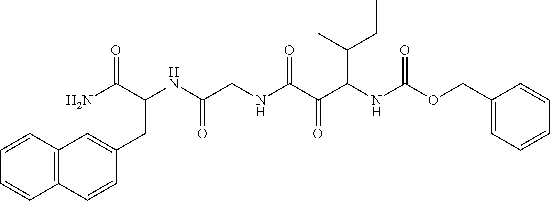 [SMILES: CCC(C)C(NC(=O)OCc1ccccc1)C(=O)C(=O)NCC(=O)NC(Cc1ccc2ccccc2c1)C(N)=O] | benzyl (1-((2-((1-amino-3-(naphthalen-2-yl)-1-oxopropan-2- yl)amino)-2-oxoethyl)amino)-4-methyl-1,2-dioxohexan-3-yl)carbamate |
| PHELIX326 | 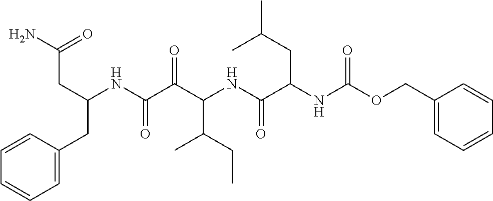 [SMILES: CCC(C)C(NC(=O)C(CC(C)C)NC(=O)OCc1ccccc1)C(=O)C(=O)NC(CC(N)=O)Cc1ccccc1] | benzyl (1-((1-((4-amino-4-oxo-1-phenylbutan-2-yl)amino)-4- methyl-1,2-dioxohexan-3-yl)amino)-4-methyl-1-oxopentan-2-yl)carbamate |
| PHELIX327 | 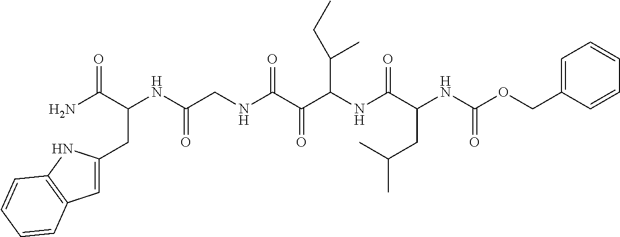 [SMILES: CCC(C)C(NC(=O)C(CC(C)C)NC(=O)OCc1ccccc1)C(=O)C(=O)NCC(=O)NC(Cc1cc2ccccc2[nH]1)C(N)=O] | benzyl (1-((1-((2-((1-amino-3-(1H-indol-2-yl)-1-oxopropan-2- yl)amino)-2-oxoethyl)amino)-4-methyl-1,2-dioxohexan-3-yl)amino)-4-methyl-1- oxopentan-2-yl)carbamate |
| PHELIX328 | 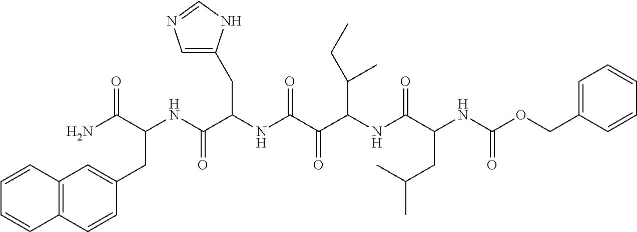 [SMILES: CCC(C)C(NC(=O)C(CC(C)C)NC(=O)OCc1ccccc1)C(=O)C(=O)NC(Cc1cnc[nH]1)C(=O)NC(Cc1ccc2ccccc2c1)C(N)=O] | benzyl (1-((1-((1-((1-amino-3-(naphthalen-2-yl)-1-oxopropan-2- yl)amino)-3-(1H-imidazol-5-yl)-1-oxopropan-2-yl)amino)-4- methyl-1,2-dioxohexan-3-yl)amino)-4-methyl-1-oxopentan-2-yl)carbamate |
| PHELIX329 PHELIX330 | 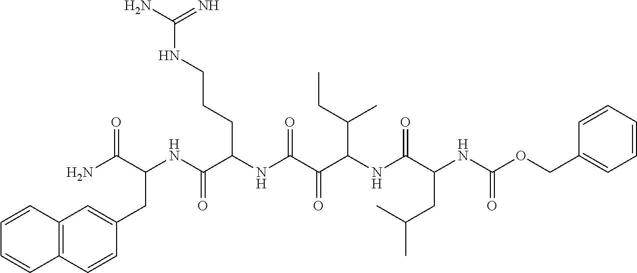 [SMILES: CCC(C)C(NC(=O)C(CC(C)C)NC(=O)OCc1ccccc1)C(=O)C(=O)NC(CCCNC(=N)N)C(=O)NC(Cc1ccc2ccccc2c1)C(N)=O] | benzyl (1-((1-((1-((1-amino-3-(naphthalen- 2-yl)-1-oxopropan-2- yl)amino)-5-guanidino-1-oxopentan-2-yl)amino)-4-methyl-1,2-dioxohexan- 3-yl)amino)-4-methyl-1-oxopentan-2-yl)carbamate |
| PHELIX331 | 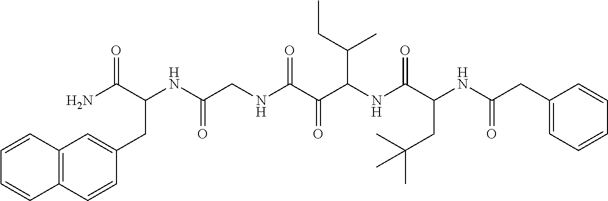 [SMILES: CCC(C)C(NC(=O)C(CC(C)(C)C)NC(=O)Cc1ccccc1)C(=O)C(=O)NCC(=O)NC(Cc1ccc2ccccc2c1)C(N)=O] | N-(2-((1-amino-3-(naphthalen-2-yl)-1-oxopropan-2-yl)amino)-2- oxoethyl)-3-(4,4-dimethyl-2-(2-phenylacetamido)pentanamido)- 4-methyl-2-oxohexanamide |
| PHELIX332 | 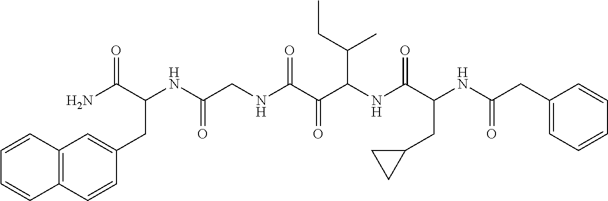 [SMILES: CCC(C)C(NC(=O)C(CC1CC1)NC(=O)Cc1ccccc1)C(=O)C(=O)NCC(=O)NC(Cc1ccc2ccccc2c1)C(N)=O] | N-(2-((1-amino-3-(naphthalen-2-yl)-1- oxopropan-2-yl)amino)-2-oxoethyl)- 3-(3-cyclopropyl-2-(2-phenylacetamido)propanamido)-4-methyl-2-oxohexanamide |
| PHELIX333 | 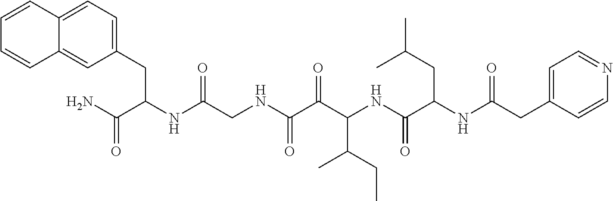 [SMILES: CCC(C)C(NC(=O)C(CC(C)C)NC(=O)Cc1ccncc1)C(=O)C(=O)NCC(=O)NC(Cc1ccc2ccccc2c1)C(N)=O] | N-(2-((1-amino-3-(naphthalen-2-yl)-1-oxopropan-2- yl)amino)-2-oxoethyl)-4-methyl-3-(4-methyl-2-(2-(pyridin-4- yl)acetamido)pentanamido)-2-oxohexanamide |
| PHELIX334 | 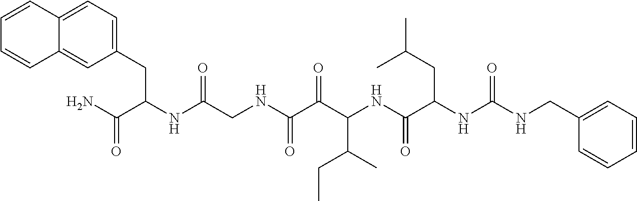 [SMILES: CCC(C)C(NC(=O)C(CC(C)C)NC(=O)NCc1ccccc1)C(=O)C(=O)NCC(=O)NC(Cc1ccc2ccccc2c1)C(N)=O] | N-(2-((1-amino-3-(naphthalen-2-yl)- 1-oxopropan-2-yl)amino)-2-oxoethyl)- 3-(2-(3-benzylureido)-4-methylpentamido)-4-methyl-2-oxohexanamide |
| PHELIX335 | 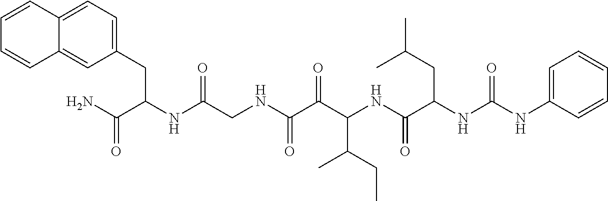 [SMILES: CCC(C)C(NC(=O)C(CC(C)C)NC(=O)Nc1ccccc1)C(=O)C(=O)NCC(=O)NC(Cc1ccc2ccccc2c1)C(N)=O] | N-(2-((1-amino-3-(naphthalen- 2-yl)-1-oxopropan-2-yl)amino)-2-oxoethyl)- 4-methyl-3-(4-methyl-2-(3-phenylureido)pentanamido)-2-oxohexanamide |
| PHELIX336 | 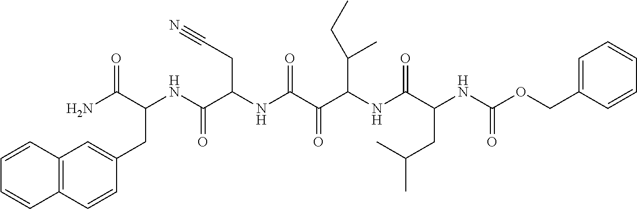 [SMILES: CCC(C)C(NC(=O)C(CC(C)C)NC(=O)OCc1ccccc1)C(=O)C(=O)NC(CC#N)C(=O)NC(Cc1ccc2ccccc2c1)C(N)=O] | benzyl (1-((1-((1-((1-amino-3- (naphthalen-2-yl)-1-oxopropan-2-yl)amino)- 3-cyano-1-oxopropan-2-yl)amino)-4-methyl-1,2- dioxohexan-3-yl)amino)-4-methyl-1-oxopentan-2-yl)carbamate |
| PHELIX337 | 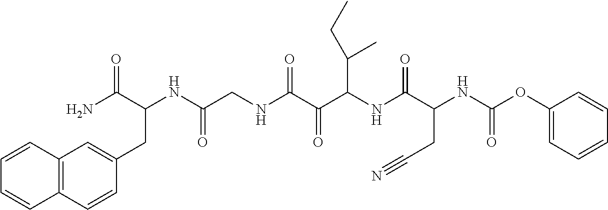 [SMILES: CCC(C)C(NC(=O)C(CC#N)NC(=O)Oc1ccccc1)C(=O)C(=O)NCC(=O)NC(Cc1ccc2ccccc2c1)C(N)=O] | N-(2-((1-amino-3-(naphthalen-2-yl)-1- oxopropan-2-yl)amino)-2- oxoethyl)-3-(3-cyano-2-(2-phenylacetamido)propanamido)-4-methyl-2-oxohexanamide |
| PHELIX338 | 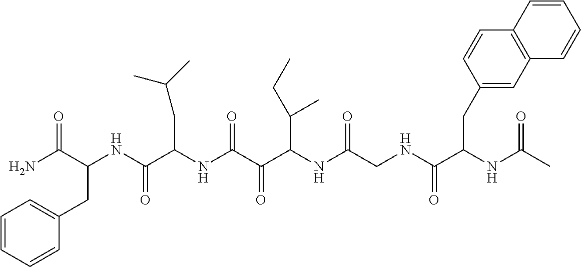 [SMILES: CCC(C)C(NC(=O)CNC(=O)C(Cc1ccc2ccccc2c1)NC(C)=O)C(=O)C(=O)NC(CC(C)C)C(=O)NC(Cc1ccccc1)C(N)=O] | 3-(2-(2-acetamido-3-(naphthalen-2- yl)propanamido)acetamido)-N-(1- ((1-amino-1-oxo-3-phenylpropan-2-yl)amino)-4-methyl-1-oxopentan-2-yl)-4-methyl-2- oxohexanamide |
| PHELIX340 PHELIX341 | 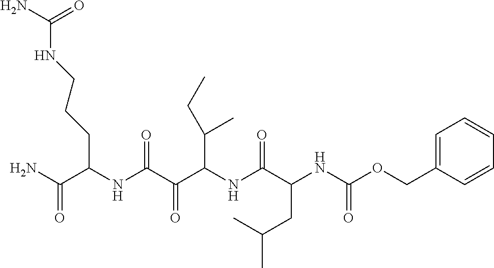 [SMILES: CCC(C)C(NC(=O)C(CC(C)C)NC(=O)OCc1ccccc1)C(=O)C(=O)NC(CCCNC(N)=O)C(N)=O] | benzyl (1-((1-((1-amino-1-oxo-5- ureidopentan-2-yl)amino)-4- methyl-1,2-dioxohexan-3-yl)amino)-4-methyl-1-oxopentan-2-yl)carbamate |
| PHELIX342 | 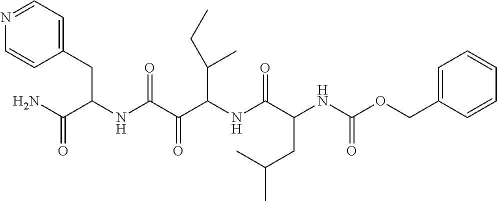 [SMILES: CCC(C)C(NC(=O)C(CC(C)C)NC(=O)OCc1ccccc1)C(=O)C(=O)NC(Cc1ccncc1)C(N)=O] | benzyl (1-((1-((1-amino-1-oxo-3-(pyridin-4-yl)propan-2- yl)amino)-4-methyl-1,2-dioxohexan-3-yl)amino)-4-methyl-1-oxopentan-2-yl)carbamate |
| PHELIX343 | 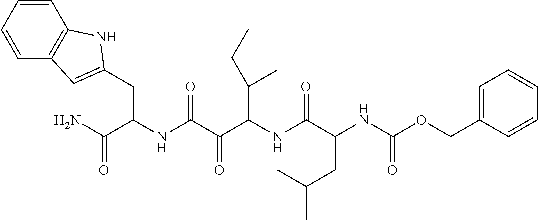 [SMILES: CCC(C)C(NC(=O)C(CC(C)C)NC(=O)OCc1ccccc1)C(=O)C(=O)NC(Cc1cc2ccccc2[nH]1)C(N)=O] | benzyl (1-((1-((1-amino-3-(1H-indol-2-yl)-1-oxopropan-2- yl)amino)-4-methyl-1,2-dioxohexan-3-yl)amino)-4-methyl-1-oxopentan-2-yl)carbamate |
| PHELIX344 | 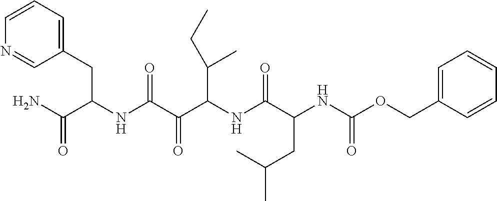 [SMILES: CCC(C)C(NC(=O)C(CC(C)C)NC(=O)OCc1ccccc1)C(=O)C(=O)NC(Cc1cccnc1)C(N)=O] | benzyl (1-((1-((1-amino-1-oxo-3-(pyridin-3-yl)propan-2- yl)amino)-4-methyl-1,2-dioxohexan-3-yl)amino)-4-ethyl-1-oxopentan-2-yl)carbamate |
| PHELIX345 | 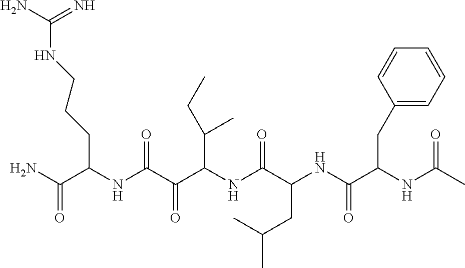 [SMILES: CCC(C)C(NC(=O)C(CC(C)C)NC(=O)C(Cc1ccccc1)NC(C)=O)C(=O)C(=O)NC(CCCNC(=N)N)C(N)=O] | 3-(2-(2-acetamido)-3-phenylpropanamido)-4- methylpentanamido)- N-(1-amino-5-guanidino-1-oxopentan-2-yl)-4-methyl-2-oxohexanamide |
| PHELIX346 | 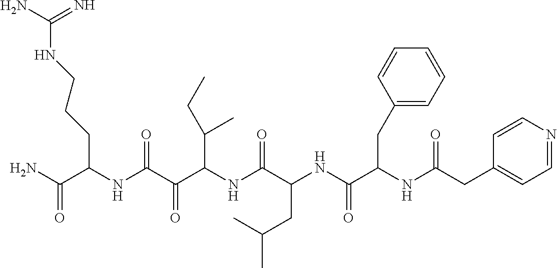 [SMILES: CCC(C)C(NC(=O)C(CC(C)C)NC(=O)C(Cc1ccccc1)NC(=O)Cc1ccncc1)C(=O)C(=O)NC(CCCNC(=N)N)C(N)=O] | N-(1-amino-5-guanidino-1-oxopentan-2-yl)-4-methyl-3-(4- methyl-2-(3-phenyl-2-(2-(pyridin-4-yl)acetamido)propanamido)pentanamido)-2- oxohexanamide |
| PHELIX348 | 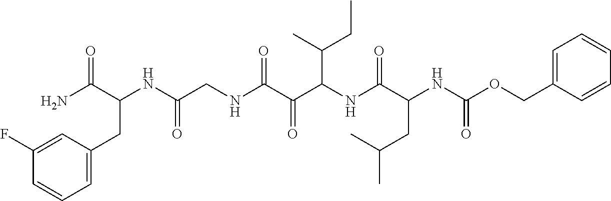 [SMILES: CCC(C)C(NC(=O)C(CC(C)C)NC(=O)OCc1ccccc1)C(=O)C(=O)NCC(=O)NC(Cc1cccc(F)c1)C(N)=O] | benzyl (1-((1-((2-((1-amino-3-(3-fluorophenyl)-1-oxopropan- 2-yl)amino)-2-oxoethyl)amino)-4-methyl-1,2-dioxohexan-3-yl)amino)-4-methyl-1- oxopentan-2-yl)carbamate |
| PHELIX349 | 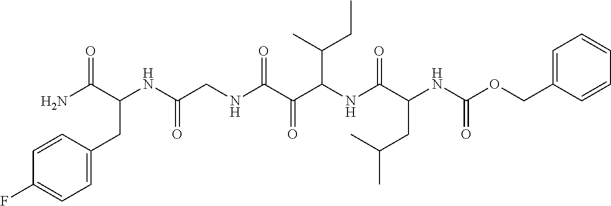 [SMILES: CCC(C)C(NC(=O)C(CC(C)C)NC(=O)OCc1ccccc1)C(=O)C(=O)NCC(=O)NC(Cc1ccc(F)cc1)C(N)=O] | benzyl (1-((1-((2-((1-amino-3-(4-fluorophenyl)-1-oxopropan-2- yl)amino)-2-oxoethyl)amino)-4-methyl-1,2-dioxohexan-3-yl)amino)-4-methyl-1- oxopentan-2-yl)carbamate |
| PHELIX350 | 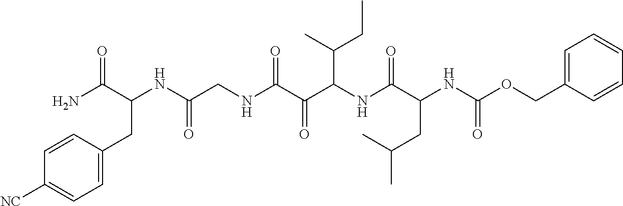 [SMILES: CCC(C)C(NC(=O)C(CC(C)C)NC(=O)OCc1ccccc1)C(=O)C(=O)NCC(=O)NC(Cc1ccc(C#N)cc1)C(N)=O] | benzyl (1-((1-((2-((1-amino-3-(4-cyanophenyl)-1-oxopropan-2- yl)amino)-2-oxoethyl)amino)-4-methyl-1,2-dioxohexan-3-yl)amino)-4-methyl-1- oxopentan-2-yl)carbamate |
| PHELIX351 | 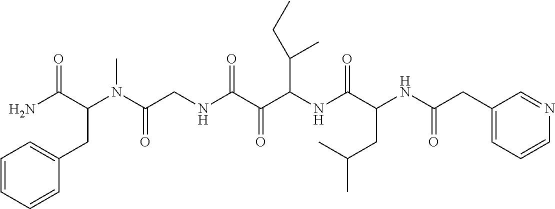 [SMILES: CCC(C)C(NC(=O)C(CC(C)C)NC(=O)Cc1cccnc1)C(=O)C(=O)NCC(=O)N(C)C(Cc1ccccc1)C(N)=O] | N-(2-((1-amino-1-oxo-3-phenylpropan-2-yl)(methyl)amino)-2- oxoethyl)-4-methyl-3-(4-methyl-2-(2-(pyridin-3-yl)acetamido)pentanamido)-2- oxohexanamide |
| PHELIX352 | 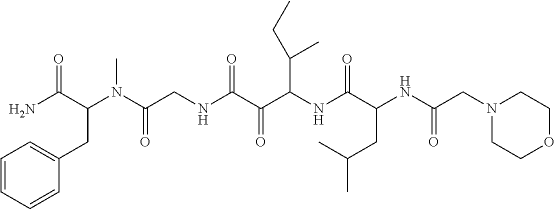 [SMILES: CCC(C)C(NC(=O)C(CC(C)C)NC(=O)CN1CCOCC1)C(=O)C(=O)NCC(=O)N(C)C(Cc1ccccc1)C(N)=O] | N-(2-((1-amino-1-oxo-3-phenylpropan-2-yl)(methyl)amino)-2- oxoethyl)-4-methyl-3-(4-methyl-2-(2-morpholinoacetamido)pentanamido)-2- oxohexanamide |
| PHELIX353 | 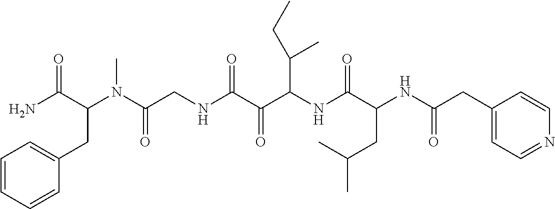 [SMILES: CCC(C)C(NC(=O)C(CC(C)C)NC(=O)Cc1ccncc1)C(=O)C(=O)NCC(=O)N(C)C(Cc1ccccc1)C(N)=O] | N-(2-((1-amino-1-oxo-3-phenylpropan-2-yl)(methyl)amino)-2- oxoethyl)-4-methyl-3-(4-methyl-2-(2-(pyridin-4-yl)acetamido)pentanamido)-2- oxohexanamide |
| PHELIX354 | 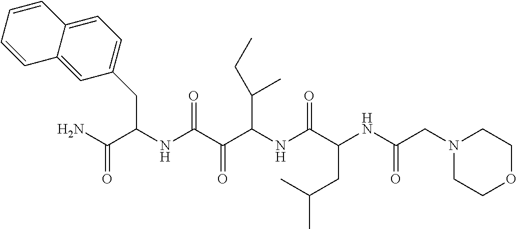 [SMILES: CCC(C)C(NC(=O)C(CC(C)C)NC(=O)CN1CCOCC1)C(=O)C(=O)NC(Cc1ccc2ccccc2c1)C(N)=O] | N-(1-amino-3-(naphthalen-2-yl)-1-oxopropan-2-yl)-4-methyl-3- (4-methyl-2-(2-morpholinoacetamido)pentanamido)-2-oxohexanamide |
| PHELIX355 | 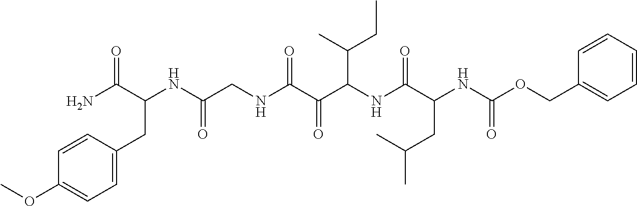 [SMILES: CCC(C)C(NC(=O)C(CC(C)C)NC(=O)OCc1ccccc1)C(=O)C(=O)NCC(=O)NC(Cc1ccc(OC)cc1)C(N)=O] | benzyl (1-((1-((2-((1-amino-3-(4- methoxyphenyl)-1-oxopropan-2- yl)amino)-2-oxoethyl)amino)-4-methyl-1,2-dioxohexan-3-yl)amino)-4-methyl-1- oxopentan-2-yl)carbamate |
| PHELIX356 | 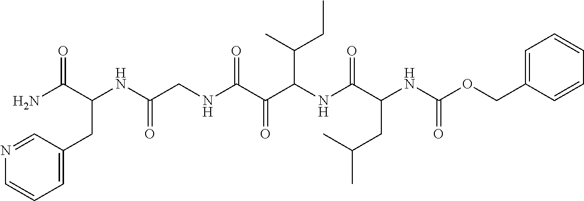 [SMILES: CCC(C)C(NC(=O)C(CC(C)C)NC(=O)OCc1ccccc1)C(=O)C(=O)NCC(=O)NC(Cc1cccnc1)C(N)=O] | benzyl (1-((1-((2-((1-amino-1-oxo-3-(pyridin-3-yl)propan-2- yl)amino)-2-oxoethyl)amino)-4-methyl-1,2-dioxohexan-3-yl)amino)-4-methyl-1- oxopentan-2-yl)carbamate |
| PHELIX357 | 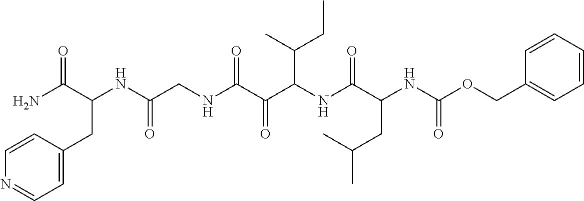 [SMILES: CCC(C)C(NC(=O)C(CC(C)C)NC(=O)OCc1ccccc1)C(=O)C(=O)NCC(=O)NC(Cc1ccncc1)C(N)=O] | benzyl (1-((1-((2-((1-amino-1-oxo-3-(pyridin-4-yl)propan-2- yl)amino)-2-oxoethyl)amino)-4-methyl-1,2-dioxohexan-3-yl)amino)-4-methyl-1- oxopentan-2-yl)carbamate |
| PHELIX358 | 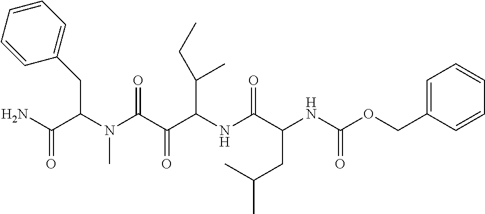 [SMILES: CCC(C)C(NC(=O)C(CC(C)C)NC(=O)OCc1ccccc1)C(=O)C(=O)N(C)C(Cc1ccccc1)C(N)=O] | benzyl (1-((1-((1-amino-1-oxo-3-phenylpropan-2- yl)(methyl)amino)-4-methyl-1,2-dioxohexan-3-yl)amino)-4-methyl-1-oxopentan-2- yl)carbamate |
| PHELIX359 | 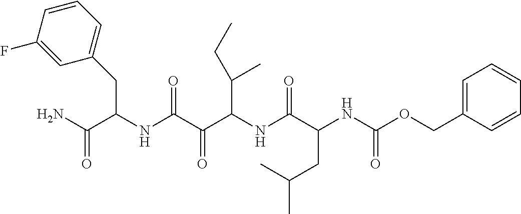 [SMILES: CCC(C)C(NC(=O)C(CC(C)C)NC(=O)OCc1ccccc1)C(=O)C(=O)NC(Cc1cccc(F)c1)C(N)=O] | benzyl (1-((1-((1-amino-3-(3-fluorophenyl)-1-oxopropan-2- yl)amino)-4-methyl-1,2-dioxohexan-3-yl)amino)-4-methyl-1-oxopentan-2-yl)carbamate |
| PHELIX360 | 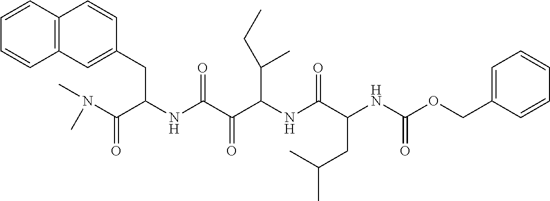 [SMILES: CCC(C)C(NC(=O)C(CC(C)C)NC(=O)OCc1ccccc1)C(=O)C(=O)NC(Cc1ccc2ccccc2c1)C(=O)N(C)C] | benzyl (1-((1-((1-(dimethylamino)-3-(naphthalen-2-yl)-1- oxopropan-2-yl)amino)-4-methyl-1,2-dioxohexan-3-yl)amino)-4-methyl-1-oxopentan-2- yl)carbamate |
| PHELIX361 | 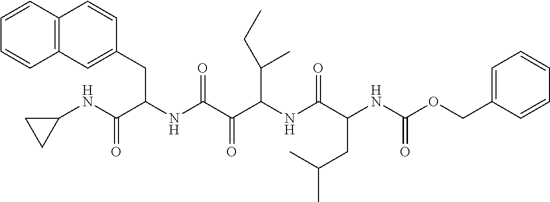 [SMILES: CCC(C)C(NC(=O)C(CC(C)C)NC(=O)OCc1ccccc1)C(=O)C(=O)NC(Cc1ccc2ccccc2c1)C(=O)NC1CC1] | benzyl (1-((1-((1-(cyclopropylamino)-3-(naphthalen-2-yl)-1- oxopropan-2-yl)amino)-4-methyl-1,2-dioxohexan-3-yl)amino)-4-methyl-1-oxopentan-2- yl)carbamate |
| PHELIX362 | 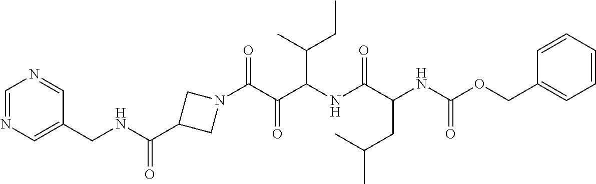 [SMILES: CCC(C)C(NC(=O)C(CC(C)C)NC(=O)OCc1ccccc1)C(=O)C(=O)N1CC(C(=O)NCc2cncnc2)C1] | benzyl (4-methyl-1-((4-methyl-1,2-dioxo-1-(3-((pyrimidin-5- ylmethyl)carbamoyl)azetidin-1-yl)hexan-3-yl)amino)-1-oxopentan-2-yl)carbamate |
| PHELIX363 | 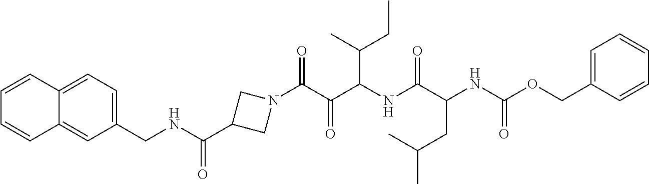 [SMILES: CCC(C)C(NC(=O)C(CC(C)C)NC(=O)OCc1ccccc1)C(=O)C(=O)N1CC(C(=O)NCc2ccc3ccccc3c2)C1] | benzyl (4-methyl-1-((4-methyl-1-(3-((naphthalen-2- ylmethyl)carbamoyl)azetidin-1-yl)-1,2-dioxohexan-3-yl)amino)-1-oxopentan-2- yl)carbamate |
| PHELIX364 | 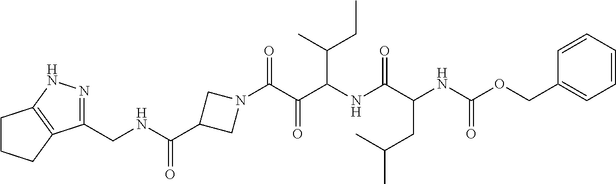 [SMILES: CCC(C)C(NC(=O)C(CC(C)C)NC(=O)OCc1ccccc1)C(=O)C(=O)N1CC(C(=O)NCc2n[nH]c3c2CCC3)C1] | benzyl (4-methyl-1-((4-methyl-1,2-dioxo-1-(3-(((1,4,5,6- tetrahydrocyclopenta[c]pyrazol-3-yl)methyl)carbamoyl)azetidin-1-yl)hexan-3-yl)amino)- 1-oxopentan-2-yl)carbamate |
| PHELIX365 | 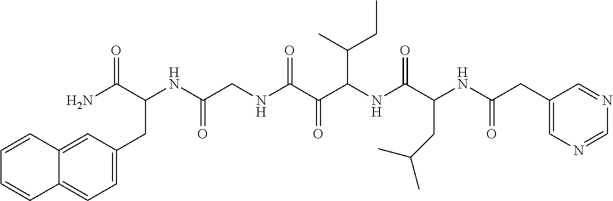 [SMILES: CCC(C)C(NC(=O)C(CC(C)C)NC(=O)Cc1cncnc1)C(=O)C(=O)NCC(=O)NC(Cc1ccc2ccccc2c1)C(N)=O] | N-(2-((1-amino-3-(naphthalen-2-yl)-1-oxopropan-2-yl)amino)- 2-oxoethyl)-4-methyl-3-(4-methyl-2-(2-(pyrimidin-5-yl)acetamido)pentanamido)-2- oxohexanamide |
| PHELIX366 | 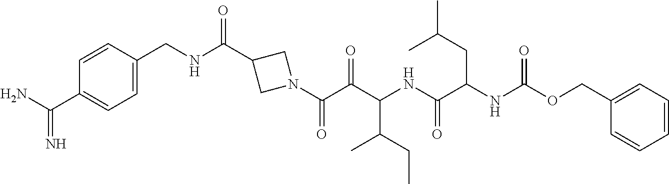 [SMILES: CCC(C)C(NC(=O)C(CC(C)C)NC(=O)OCc1ccccc1)C(=O)C(=O)N1CC(C(=O)NCc2ccc(C(=N)N)cc2)C1] | benzyl (1-((1-(3-((4-carbamimidoylbenzyl)carbamoyl)azetidin- 1-yl)-4-methyl-1,2-dioxohexan-3-yl)amino)-4-methyl-1-oxopentan-2-yl)carbamate |
| PHELIX367 | 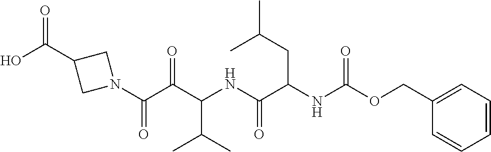 [SMILES: CC(C)CC(NC(=O)OCc1ccccc1)C(=O)NC(C(=O)C(=O)N1CC(C(=O)O)C1)C(C)C] | 1-(3-(2-(((benzyloxy)carbonyl)amino)-4-methylpentanamido)-4-methyl-2- oxohexanoyl)azetidine-3-carboxylic acid |

In treatment methods according to the disclosure, an effective amount of a pharmaceutical agent according to the disclosure is administered to a subject suffering from or diagnosed as having such a disease, disorder, or condition. An “effective amount” means an amount or dose sufficient to generally bring about the desired therapeutic benefit in patients in need of such treatment for the designated disease, disorder, or condition. Effective amounts or doses of the compounds of the present disclosure may be ascertained by routine methods such as modeling, dose escalation studies or clinical trials, and by taking into consideration routine factors, e.g., the mode or route of administration or drug delivery, the pharmacokinetics of the compound, the severity and course of the disease, disorder, or condition, the subject's previous or ongoing therapy, the subject's health status and response to drugs, and the judgment of the treating physician. An example of a dose is in the range of from about 0.001 to about 200 mg of compound per kg of subject's body weight per day, preferably about 0.05 to 100 mg/kg/day, or about 1 to 35 mg/kg/day, in single or divided dosage units (e.g., BID, TID, QID). For a 70-kg human, an illustrative range for a suitable dosage amount is from about 0.05 to about 7 g/day, or about 0.2 to about 2.5 g/day.

III. Compositions Containing the Compound

Pharmaceutical compositions useful herein, in one embodiment, contain a compound discussed above in a pharmaceutically acceptable carrier or diluent with other optional suitable pharmaceutically inert or inactive ingredients. In another embodiment, a compound described above is present in a single composition. In a further embodiment, a compound described above is combined with one or more excipients and/or other therapeutic agents as described below.

(i) Salts

The compounds discussed above may encompass tautomeric forms of the structures provided herein characterized by the bioactivity of the drawn structures. Further, the compounds may also be used in the form of salts derived from pharmaceutically or physiologically acceptable acids, bases, alkali metals and alkaline earth metals. In some embodiments, the salts are formed from amino acid side chain groups.

In one embodiment, pharmaceutically acceptable salts can be formed from organic and inorganic acids including, e.g., acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids.

In another embodiment, pharmaceutically acceptable salts may also be formed from inorganic bases, desirably alkali metal salts including, e.g., sodium, lithium, or potassium, such as alkali metal hydroxides. Examples of inorganic bases include, without limitation, sodium hydroxide, potassium hydroxide, calcium hydroxide, and magnesium hydroxide. Pharmaceutically acceptable salts may also be formed from organic bases, such as ammonium salts, mono-, di-, and trimethylammonium, mono-, di- and triethylammonium, mono-, di- and tripropylammonium, ethyldimethylammonium, benzyldimethylammonium, cyclohexylammonium, benzyl-ammonium, dibenzylammonium, piperidinium, morpholinium, pyrrolidinium, piperazinium, 1-methylpiperidinium, 4-ethylmorpholinium, 1-isopropylpyrrolidinium, 1,4-dimethylpiperazinium, 1 n-butyl piperidinium, 2-methylpiperidinium, 1-ethyl-2-methylpiperidinium, mono-, di- and triethanolammonium, ethyl diethanolammonium, n-butylmonoethanolammonium, tris(hydroxymethyl)methylammonium, phenylmono-ethanolammonium, diethanolamine, ethylenediamine, and the like. In one example, the base is selected from among sodium hydroxide, lithium hydroxide, potassium hydroxide, and mixtures thereof.

(ii) Prodrugs

The salts, as well as other compounds, can be in the form of esters, carbamates and other conventional “pro-drug” forms, which, when administered in such form, convert to the active moiety in vivo. In one embodiment, the prodrugs are esters. In another embodiment, the prodrugs are carbamates. See, e.g., B. Testa and J. Caldwell, “Prodrugs Revisited: The “Ad Hoc” Approach as a Complement to Ligand Design”, Medicinal Research Reviews, 16(3):233-241, ed., John Wiley & Sons (1996), which is incorporated by reference.

(iii) Carriers and Diluents

The pharmaceutical compositions include a compound described herein formulated neat or with one or more pharmaceutical carriers for administration, the proportion of which is determined by the solubility and chemical nature of the compound, chosen route of administration and standard pharmacological practice. The pharmaceutical carrier may be solid or liquid.

The compound may be administered to a subject by any desirable route, taking into consideration the specific condition for which it has been selected. The compound may, therefore, be delivered orally, by injection, i.e., transdermally, intravenously, subcutaneously, intramuscularly, intravenous, intra-arterial, intraperitoneal, intracavitary, or epiduraly, among others.

Although the compound may be administered alone, it may also be administered in the presence of one or more pharmaceutical carriers that are physiologically compatible. The carriers may be in dry or liquid form and must be pharmaceutically acceptable. Liquid pharmaceutical compositions are typically sterile solutions or suspensions.

When liquid carriers are utilized, they are desirably sterile liquids. Liquid carriers are typically utilized in preparing solutions, suspensions, emulsions, syrups and elixirs. In one embodiment, the compound is dissolved a liquid carrier. In another embodiment, the compound is suspended in a liquid carrier. One of skill in the art of formulations would be able to select a suitable liquid carrier, depending on the route of administration. In one embodiment, the liquid carrier includes, without limitation, water, organic solvents, oils, fats, or mixtures thereof. In another embodiment, the liquid carrier is water containing cellulose derivatives such as sodium carboxymethyl cellulose. In a further embodiment, the liquid carrier is water and/or dimethylsulfoxide. Examples of organic solvents include, without limitation, alcohols such as monohydric alcohols and polyhydric alcohols, e.g., glycols and their derivatives, among others. Examples of oils include, without limitation, fractionated coconut oil, arachis oil, corn oil, peanut oil, and sesame oil and oily esters such as ethyl oleate and isopropyl myristate.

Alternatively, the compound may be formulated in a solid carrier. In one embodiment, the composition may be compacted into a unit dose form, i.e., tablet or caplet. In another embodiment, the composition may be added to unit dose form, i.e., a capsule. In a further embodiment, the composition may be formulated for administration as a powder. The solid carrier may perform a variety of functions, i.e., may perform the functions of two or more of the excipients described below. For example, the solid carrier may also act as a flavoring agent, lubricant, solubilizer, suspending agent, filler, glidant, compression aid, binder, disintegrant, or encapsulating material. Suitable solid carriers include, without limitation, calcium phosphate, dicalcium phosphate, magnesium stearate, talc, starch, sugars (including, e.g., lactose and sucrose), cellulose (including, e.g., microcrystalline cellulose, methyl cellulose, sodium carboxymethyl cellulose), polyvinylpyrrolidine, low melting waxes, ion exchange resins, and kaolin. The solid carrier can contain other suitable excipients, including those described below.

Examples of excipients which may be combined with the compound include, without limitation, adjuvants, antioxidants, binders, buffers, coatings, coloring agents, compression aids, diluents, disintegrants, emulsifiers, emollients, encapsulating materials, fillers, flavoring agents, glidants, granulating agents, lubricants, metal chelators, osmo-regulators, pH adjusters, preservatives, solubilizers, sorbents, stabilizers, sweeteners, surfactants, suspending agents, syrups, thickening agents, or viscosity regulators. See, the excipients described in the “Handbook of Pharmaceutical Excipients”, 5^{th} Edition, Eds.: Rowe, Sheskey, and Owen, APhA Publications (Washington, D.C.), Dec. 14, 2005, which is incorporated herein by reference.

IV. Methods of Using the Compound

The compounds discussed herein are useful for inhibiting calpain activity, cathepsin activity, or both.

In some embodiments, the compounds are useful in inhibiting calpain activity and, thus, are useful in treating conditions, disorder or disease associated with calpain activity. The compounds may act as an antagonist to the calpain receptor, agonist to the calpain receptor or combination thereof. In other embodiments, methods of treating a calpain-mediated disorder in a subject are provided and comprise administering to the subject a therapeutically effective amount of one or more compound described herein to the subject.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

The terms “patient” or “subject” as used herein refer to a mammalian animal. In one embodiment, the patient or subject is a human. In another embodiment, the patient or subject is a veterinary or farm animal, a domestic animal or pet, or animal normally used for clinical research. In a further embodiment, the subject or patient has a disorder, disease or conditions associated with calpain activity. In yet other embodiments, the subject or patient has a disorder, disease, or condition associated with cathepsin activity.

A variety calpain-related conditions, diseases, and disorders can be treated using the compounds discussed herein. Such diseases include, without limitation, Alzheimer's disease, atherosclerosis, bacterial infection, cancer, cataract formation, diabetes, fibrotic disease, halogen/acid exposure injury, ischemia, muscular dystrophy, neurological injury, parasite infection, reperfusion injury, or Wolfram's syndrome. In some embodiments, the calpain-related condition, disease, or disorder is a neurological injury. In other embodiments, the calpain-related condition, disease, or disorder is a neurological injury such as a traumatic brain injury, stroke, Parkinson's disease, spinal cord injury, or combination thereof. In further embodiments, the condition, disease, or disorder is a fibrotic disease. In still other embodiments, the condition, disease or disorder is a fibrotic disease that is pulmonary fibrosis, scleroderma, cardiac fibrosis, renal fibrosis, liver fibrosis including nonalcoholic steatohepatitis (NASH), Crohn's disease, strabismus, or hypertrophic scarring.

In other embodiments, the compounds are useful in inhibiting cathepsin activity and, thus, are useful in treating conditions, disorder or disease associated with cathepsin activity. The compounds may act as an antagonist to the cathepsin receptor, agonist to the cathepsin receptor or combination thereof. In further embodiments, the compounds are useful in inhibiting cathepsin-B, cathepsin-L, cathepsin-S, cathepsin-L activity, or a combination thereof. In other embodiments, methods of treating a cathepsin-mediated disorder in a subject are provided and comprise administering to the subject a therapeutically effective amount of one or more compound described herein to the subject. In yet further embodiments, methods of treating a cathepsin-B, cathepsin-L, cathepsin-S, or cathepsin-L mediated disorder in a subject are provided and comprise administering to the subject a therapeutically effective amount of one or more compound to the subject. In still other embodiments, the compounds are useful in treating a cathepsin-L or cathepsin-S mediated disorder. In further embodiments, the compounds are useful in treating a cathepsin mediated disorder that is chronic pain, a coronavirus infection, Ebola virus infection, a Filovirus infection, marburg virus syndrome, or osteoporosis. In other embodiments, the compounds are useful in treating a cathepsin mediated disorder that is corona virus infection. In yet further embodiments, the compounds are useful in treating a cathepsin mediated disorder that is a middle-east respiratory syndrome (MERS).

The term “cancer” as used herein, refers to neoplastic cells in a patient which have abnormal cell group and invade or have the potential to invade one or more body parts of the patient. In one embodiment, the cancer is a neuroendocrine cancer. In another embodiment, the cancer is of the adrenal gland, appendix, bladder, blood, brain, bone, breast, bronchus, central nervous system, cervix, chest, colon, esophagus, eye, gallbladder, head, intestines, kidney, larynx, liver, lung, lymph nodes, mouth, neck, ovaries, pancreas, pharynx, pituitary, prostate, rectum, skin, stomach, testicles, throat, thymus, thyroid, uterus, urinary tract, or vagina, or is a leukemia. In a further embodiment, the cancer is breast cancer.

Delivery forms of the pharmaceutical compositions containing one or more dosage units of the active agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the disclosure can be provided in the form of tablets or capsules, or as a solution, emulsion, or suspension. To prepare the oral compositions, the compounds may be formulated to yield a dosage of, e.g., from about 0.05 to about 100 mg/kg daily, or from about 0.05 to about 35 mg/kg daily, or from about 0.1 to about 10 mg/kg daily. For example, a total daily dosage of about 5 mg to 5 g daily may be accomplished by dosing once, twice, three, or four times per day.

Oral tablets may include a compound according to the disclosure mixed with pharmaceutically acceptable excipients such as inert diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, compounds of the disclosure may be mixed with a solid, semi-solid, or liquid diluent. Soft gelatin capsules may be prepared by mixing the compound of the disclosure with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this disclosure may also be administered by non-oral routes. For example, the compositions may be formulated for rectal administration as a suppository. For parenteral use, including intravenous, intramuscular, intraperitoneal, or subcutaneous routes, the compounds of the disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compounds of the disclosure may utilize a patch formulation to affect transdermal delivery.

Compounds of the disclosure may alternatively be administered in methods of this disclosure by inhalation, via the nasal or oral routes, e.g., in a spray formulation also containing a suitable carrier.

V. Kits Containing the Compound

Also provided herein are kits or packages of pharmaceutical formulations containing one or more compounds of formula (I), (II), (III), (IV), and (V) (or salts thereof), or pharmaceutical compositions described herein. The kits may be organized to indicate a single formulation or combination of formulations to be taken at each desired time. The composition may also be sub-divided to contain appropriate quantities of one or more compound of formula (I), (II), (III), (IV), and (V). For example, the unit dosage can be packaged compositions, e.g., packeted powders, vials, ampoules, prefilled syringes or sachets containing liquids.

Suitably, the kit contains packaging or a container with the one or more compound of formula (I), (II), (III), (IV), and (V) formulated for the desired delivery route. Suitably, the kit contains instructions on dosing and an insert regarding the one or more compound of formula (I), (II), (III), (IV), and (V). Optionally, the kit may further contain instructions for monitoring circulating levels of product and materials for performing such assays including, e.g., reagents, well plates, containers, markers or labels, and the like. Such kits are readily packaged in a manner suitable for treatment of a desired indication. For example, the kit may also contain instructions for use of the delivery device. Other suitable components to include in such kits will be readily apparent to one of skill in the art, taking into consideration the desired indication and the delivery route. The doses are repeated daily, weekly, or monthly, for a predetermined length of time or as prescribed.

The one or more compound of formula (I), (II), (III), (IV), and (V) or composition described herein can be a single dose or for continuous or periodic discontinuous administration. For continuous administration, a package or kit can include the compound in each dosage unit (e.g., solution, lotion, tablet, pill, or other unit described above or utilized in drug delivery). When the one or more compound of formula (I), (II), (III), (IV), and (V) is to be delivered with periodic discontinuation, a package or kit can include placebos during periods when the one or more compound of formula (I), (II), (III), (IV), and (V) is not delivered. When varying concentrations of a composition, of the components of the composition, or of relative ratios of the one or more compound of formula (I), (II), (III), (IV), and (V) or other agents within a composition over time is desired, a package or kit may contain a sequence of dosage units, so varying.

A number of packages or kits are known in the art for the use in dispensing pharmaceutical agents for oral use. In one embodiment, the package has indicators for each period. In another embodiment, the package is a labeled blister package, dial dispenser package, or bottle.

The packaging means of a kit may itself be geared for administration, such as an inhalant, syringe, pipette, eye dropper, or other such like apparatus, from which the formulation may be applied to an infected area of the body, such as the lungs, injected into a subject, or even applied to and mixed with the other components of the kit.

The one or more compound of formula (I), (II), (III), (IV), and (V) or composition of these kits also may be provided in dried or lyophilized forms. When reagents or components are provided as a dried form, reconstitution generally is by the addition of a suitable solvent. It is envisioned that the solvent also may be provided in another packaging means.

The kits may include a means for containing the vials in close confinement for commercial sale such as, e.g., injection or blow-molded plastic containers into which the desired vials are retained.

Irrespective of the number or type of packages, the kits also may include, or be packaged with a separate instrument for assisting with the injection/administration or placement of the ultimate complex composition within the body of an animal. Such an instrument may be an inhalant, syringe, pipette, forceps, measuring spoon, eye dropper or any such medically approved delivery means. Other instrumentation includes devices that permit the reading or monitoring of reactions in vitro.

In one embodiment, a pharmaceutical kit is provided and contains one or more compound of formula (I), (II), (III), (IV), and (V). The one or more compound of formula (I), (II), (III), (IV), and (V) may be in the presence or absence of one or more of the carriers or excipients described above. The kit may optionally contain an additional active agent as described above and/or instructions for administering the additional active agent and the one or more compound of formula (I), (II), (III), (IV), and (V) to a subject.

In a further embodiment, a pharmaceutical kit is provided and contains an additional active agent in a first dosage unit, one or more compound of formula (I), (II), (III), (IV), and (V) selected from those described herein in a second dosage unit, and one or more of the carriers or excipients described above in a third dosage unit. The kit may optionally contain instructions for administering the additional active agent and/or one or more compound of formula (I), (II), (III), (IV), and (V) to a subject.

The following Examples are provided to illustrate some of the concepts described within this disclosure. While each Example is considered to provide specific individual embodiments of composition, methods of preparation and use, none of the Examples should be considered to limit the more general embodiments described herein.

In the following examples, efforts have been made to ensure accuracy with respect to numbers used (e.g. amounts, temperature, etc.) but some experimental error and deviation should be accounted for. Unless indicated otherwise, temperature is in degrees C., pressure is at or near atmospheric.

### EXAMPLES

Exemplary compounds useful in methods of the disclosure will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

### Example A

### Synthesis Procedure for Hydroxy Acid

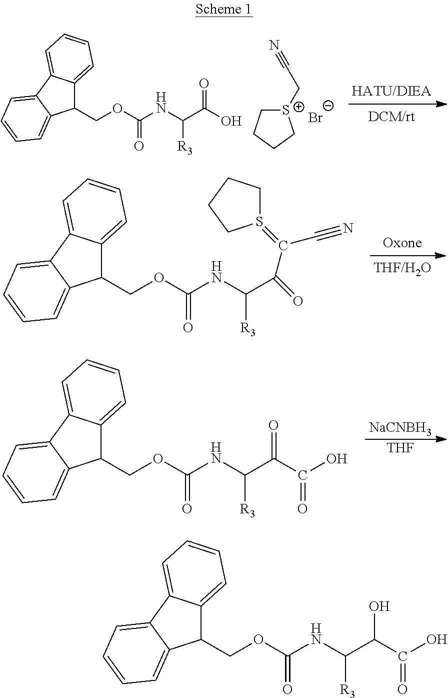

Step 1: Dissolved Fmoc-amino acid (14.15 mmol, 5 g) in 50 mL dichloromethane. Next, HATU (15.56 mmol, 5.92 g) and DIEA (42.4 mmol, 7.41 mL) were added. Lastly, thiophene acetonitrile (18.39 mmol, 3.83 g) was added and the mixture was stirred at room temperature overnight. The reaction mixture was poured over sat. NH₄Cl, mixed, and separated. The aqueous layer was extracted 2×75 mL with DCM. The combined organic layers were washed once with sat. NaCl. The organic layer was dried over Na₂SO₄, filtered, and solvent removed under reduced pressure. The crude material was purified by flash chromatography eluting with EA:acetone. LC/MS corresponds to correct mass.

Step 2: The ylide (14.15 mmol) from Step 1 was dissolved in 1:1 THF:H₂O (150 mL) and oxone (28.3 mmol, 17.38 g) was added. The mixture was stirred at room temperature for 1 hour. Reaction mixture was poured over 100 mL 1N HCl then extracted with 3×100 mL DCM. The combined organic layers were washed once with sat. NaCl. The organic layer was dried over Na₂SO₄, filtered, and solvent removed under reduced pressure. The crude material was used without purification for the next step of the synthesis.

Step 3: Ketoacid (13.11 mmol, 5 g) from Step 2 was dissolved in 40 mL THF then sodium cyanoborohydride (8.65 mmol, 0.544 g) was added. The mixture was stirred at room temperature for 1 hour. The reaction was quenched with 5 mL water followed by 40 mL 1M HCl. The mixture was extracted 2×50 mL DCM. The combined organic layers were washed once with sat. NaCl. The organic layer was dried over Na₂SO₄, filtered, and solvent removed under reduced pressure. The crude material was used without purification for peptide synthesis.

### Example B

### General Peptide Synthesis Procedure for Method A, Scheme 2

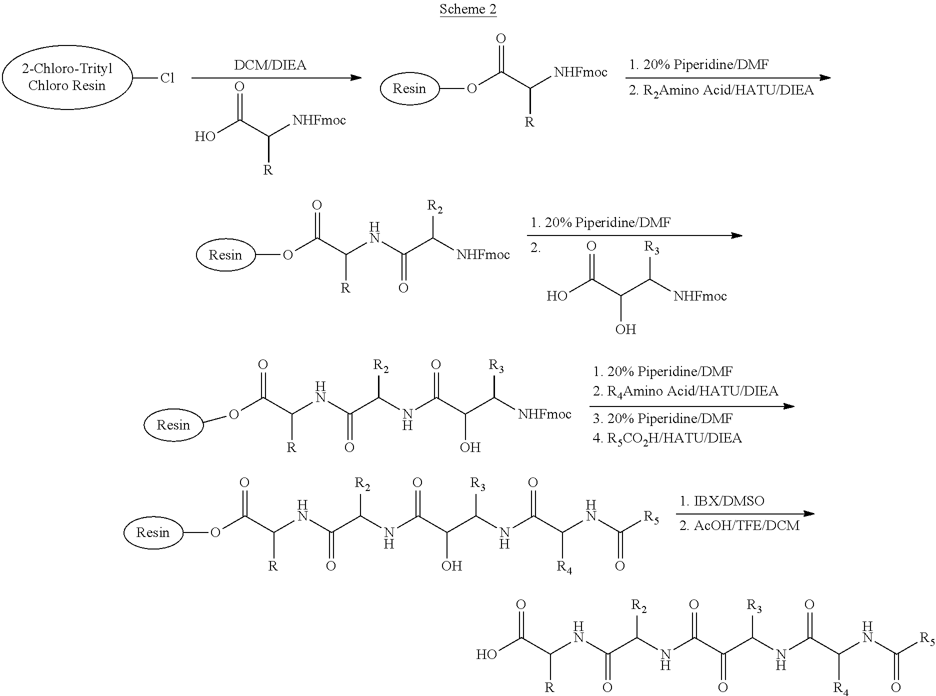

Step 1 Load first amino acid onto the 2-chlorotrityl chloride resin: 2-Chlorotrityl chloride resin (0.1 g., 1.1 meq/g, 0.11 mmol) was pre-swelled by mixing in 3 mL dichloromethane for 30 minutes at room temperature in a 10 mL fritted synthesis vial. Solvent was drained off and resin was washed with additional 3 mL of DCM. Resin was mixed with 3 mL DCM, Fmoc-amino acid (0.275 mmol), and DIEA (5.5 mmol) at rt for 1 hour. Half of the reaction volume was drained off and 2 mL methanol was added to cap any unreacted resin end groups. Mixture was stirred at rt for 30 minutes. Solvents/reagent were drained off and resin was rinsed with additional methanol (3-4 mL) followed by DCM (3-4 mL) then DMF (3-4 mL).

Step 2 Automated microwave assisted peptide synthesis on Biotage® Initiator+ Alstra™: Synthesizer was set up with the following reagents: DMF, NMP, 20% piperidine in DMF, 1.5 M DIEA in NMP, 0.5 M HATU in DMF, and desired Fmoc-amino acids/Fmoc-amino acid derivatives/end capping reagents (0.5 M in 1:1 DMF:NMP). The vial containing the resin from Step 1 was placed in the synthesizer. The instrument was programmed with the following sequence for Fmoc piperidine deprotection and subsequent HATU amino acid coupling. The resin was initially swelled in 4.5 mL DMF for 20 min. at 70° C. followed by draining of solvent. Deprotection sequence: a) 3.0 mL 20% Piperidine in DMF for 3 min. at 70° C. b) Reagents were drained. c) 3.0 mL 20% piperidine in DMF for 3 min. at 70° C. d) Reagents were drained and resin washed with 4.5 mL DMF. Coupling sequence: a) Reaction vial was filled with 5 equivalents Fmoc-amino acid (or reagent), 0.98 equiv. HATU, 1.5 mL DMF and 2 equiv. DIEA. Mixture heated at 75° C. for 5 min. b) Reagents were drained and resin washed with 4.5 mL DMF. The deprotection and coupling sequences were repeated for each amino acid or derivative until peptide was the desired length.

Step 3 Oxidation of alcohol on resin: After synthesis was complete on the Biotage, the resin was rinsed with DMF followed by DCM and finally DMSO. The resin was mixed with 3 mL DMSO and 5 equiv. 2-iodoxybenzoic acid (IBX) and stirred at rt at least 8 hours. Solvents/reagent were drained off and resin was rinsed with 2-3 mL DMSO followed by 5 mL DCM.

Step 4 Cleavage of peptide from resin: Cleavage cocktail of 1:1:8 AcOH:TFE:DCM (3 mL) was added to the resin and stirred at rt for 1.5 hours. Solvents/reagent were drained off and resin was rinsed with 5 mL DCM. Hexane (25 mL) was added to the solution and the solvents were removed under reduced pressure.

Step 5 Purification of final product: After confirming desired product was synthesized by analytical LC/MS, the general purification was using Water system prep-LC/MS using X-Select Peptide CSH.C18 080 Prep Column, 130 Angstrom, 5 μm, 19 mm×150 mm, with elution solvent A Water with 0.5% Formic acid and solvent B Acetonitrile with 0.5% Formic acid.

### Example C

### General Peptide Synthesis Procedure for Method B, Scheme 3

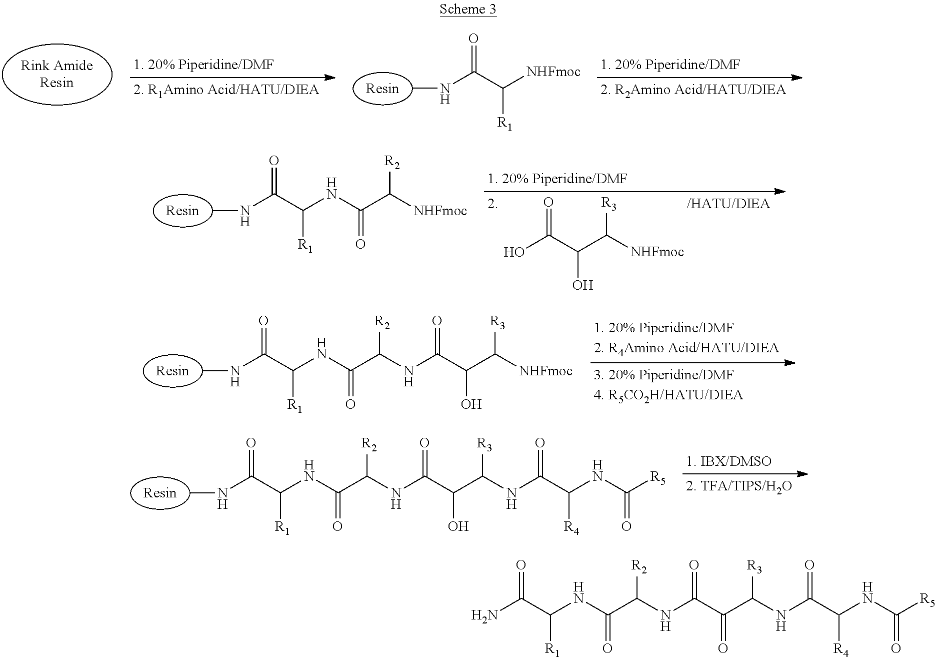

Step 1 Automated microwave assisted peptide synthesis on Biotage® Initiator+ Alstra™: Synthesizer was set up with the following reagents: DMF, NMP, 20% piperidine in DMF, 1.5 M DIEA in NMP, 0.5 M HATU in DMF, and desired Fmoc-amino acids/Fmoc-amino acid derivatives/end capping reagents (0.5 M in 1:1 DMF:NMP). The vial containing the Rink amide MBHE resin (with Nle) (0.2 g, 0.1 mmol) was placed in the synthesizer. The instrument was programmed with the following sequence for Fmoc piperidine deprotection and subsequent HATU amino acid coupling. The resin was initially swelled in 4.5 mL DMF for 20 min. at 70° C. followed by draining of solvent. Deprotection sequence: a) 3.0 mL 20% Piperidine in DMF for 3 min. at 70° C. b) Reagents were drained. c) 3.0 mL 20% piperidine in DMF for 3 min. at 70° C. d) Reagents were drained and resin washed with 4.5 mL DMF. Coupling sequence: a) Reaction vial was filled with 5 equivalents Fmoc-amino acid (or reagent), 0.98 equiv. HATU, 1.5 mL DMF and 2 equiv. DIEA. Mixture heated at 75° C. for 5 min. b)

Reagents were drained and resin washed with 4.5 mL DMF. The deprotection and coupling sequences were repeated for each amino acid or derivative until peptide was the desired length.

Step 2 Oxidation of alcohol on resin: After synthesis was complete on the Biotage, the resin was rinsed with DMF followed by DCM and finally DMSO. The resin was mixed with 3 mL DMSO and 5 equiv. 2-iodoxybenzoic acid (IBX) and stirred at rt at least 8 hours. Solvents/reagent were drained off and resin was rinsed with 2-3 mL DMSO followed by 5 mL DCM.

Step 3 Cleavage of peptide from resin: Cleavage cocktail of 9:0.5:0.5 TFA:TIPS:H₂O (3 mL) was added to the resin and stirred at rt for 1.5 hours. Solvents/reagent were drained off and resin was rinsed with 5 mL DCM.

Step 4 Purification of final product: After confirming desired product was synthesized by analytical LC/MS, the general purification was using Water system prep-LC/MS using X-Select Peptide CSH.C18 080 Prep Column, 130 Angstrom, 5 μm, 19 mm×150 mm, with elution solvents A=Water with 0.5% Formic acid and B=Acetonitrile with 0.5% Formic acid.

### Example D

### General Peptide Synthesis Procedure for Method C

Step 1 Automated microwave assisted peptide synthesis on Biotage® Initiator+ Alstra™: Synthesizer was set up with the following reagents: DMF, NMP, 20% piperidine in DMF, 1.5 M DIEA in NMP, 0.5 M HATU in DMF, and desired Fmoc-amino acids/Fmoc-amino acid derivatives/end capping reagents (0.5 M in 1:1 DMF:NMP). The vial containing the Rink amide MBHE resin (with Nle) (0.2 g, 0.1 mmol) was placed in the synthesizer. The instrument was programmed with the following sequence for Fmoc piperidine deprotection and subsequent HATU amino acid coupling. The resin was initially swelled in 4.5 mL DMF for 20 min. at 70° C. followed by draining of solvent. Deprotection sequence: a) 3.0 mL 20% Piperidine in DMF for 3 min. at 70° C. b) Reagents were drained. c) 3.0 mL 20% piperidine in DMF for 3 min. at 70° C. d) Reagents were drained and resin washed with 4.5 mL DMF. Coupling sequence: a) Reaction vial was filled with 5 equivalents Fmoc-amino acid (or reagent), 0.98 equiv. HATU, 1.5 mL DMF and 2 equiv. DIEA. Mixture heated at 75° C. for 5 min. b) Reagents were drained and resin washed with 4.5 mL DMF. The deprotection and coupling sequences were repeated for each amino acid or derivative until peptide was the desired length.

Step 2 Cleavage of peptide from resin: Cleavage cocktail of 9:0.5:0.5 TFA:TIPS:H₂O (3 mL) was added to the resin and stirred at rt for 1.5 hours. Solvents/reagent were drained off and resin was rinsed with 5 mL DCM. TFA was recovered under vacuum to yield oil

Step 3 Oxidation of Alcohol: Oil from above step was dissolved in 3 mL DCM and then 1.5 eq Dess-Martin-Periodinane reagent was added. Reaction mixture stirred for 2 hrs at RT and diluted with 10 ml DCM. Then the reaction mixture was quenched with sodium thiosulphate (1M) and saturated sodium bicarbonate solutions. The organic layer was separated, dried over sodium sulfate and evaporated under vacuum to afford crude ketoamide product.

Step 4 Purification of final product: After confirming desired product was synthesized by analytical LC/MS, the general purification was using Water system prep-LC/MS using X-Select Peptide CSH.C18 080 Prep Column, 130 Angstrom, 5 μm, 19 mm×150 mm, with elution solvents A=Water with 0.5% Formic acid and B=Acetonitrile with 0.5% Formic acid.

### Example E

### General Peptide Synthesis Procedure for Method D

Step 1 Load first amino acid onto the 2-chlorotrityl chloride resin: 2-Chlorotrityl chloride resin (0.1 g., 1.1 meq/g, 0.11 mmol) was pre-swelled by mixing in 3 mL dichloromethane for 30 minutes at room temperature in a 10 mL fritted synthesis vial. Solvent was drained off and resin was washed with additional 3 mL of DCM. Resin was mixed with 3 mL DCM, Fmoc-amino acid (0.275 mmol), and DIEA (5.5 mmol) at rt for 1 hour. Half of the reaction volume was drained off and 2 mL methanol was added to cap any unreacted resin end groups. Mixture was stirred at rt for 30 minutes. Solvents/reagent were drained off and resin was rinsed with additional methanol (3-4 mL) followed by DCM (3-4 mL) then DMF (3-4 mL).

Step 2 Automated microwave assisted peptide synthesis on Biotage® Initiator+ Alstra™: Synthesizer was set up with the following reagents: DMF, NMP, 20% piperidine in DMF, 1.5 M DIEA in NMP, 0.5 M HATU in DMF, and desired Fmoc-amino acids/Fmoc-amino acid derivatives/end capping reagents (0.5 M in 1:1 DMF:NMP).

The vial containing the resin from Step 1 is placed in the synthesizer. The instrument was programmed with the following sequence for Fmoc piperidine deprotection and subsequent HATU amino acid coupling. The resin was initially swelled in 4.5 mL DMF for 20 min. at 70° C. followed by draining of solvent. Deprotection sequence: a) 3.0 mL 20% Piperidine in DMF for 3 min. at 70° C. b) Reagents were drained. c) 3.0 mL 20% piperidine in DMF for 3 min. at 70° C. d) Reagents were drained and resin washed with 4.5 mL DMF. Coupling sequence: a) Reaction vial was filled with 5 equivalents Fmoc-amino acid (or reagent), 0.98 equiv. HATU, 1.5 mL DMF and 2 equiv. DIEA. Mixture heated at 75° C. for 5 min. b) Reagents were drained and resin washed with 4.5 mL DMF. The deprotection and coupling sequences were repeated for each amino acid or derivative until peptide was the desired length.

Step 3 Oxidation of alcohol on resin: After synthesis was complete on the Biotage, the resin is rinsed with DMF followed by DCM and finally DMSO. The resin was mixed with 3 mL DMSO and 5 equiv. 2-iodoxybenzoic acid (IBX) and stirred at rt at least 8 hours. Solvents/reagent were drained off and resin was rinsed with 2-3 mL DMSO followed by 5 mL DCM.

Step 4 Cleavage of peptide from resin: Cleavage cocktail of 1:1:8 AcOH:TFE:DCM (3 mL) was added to the resin and stirred at rt for 1.5 hours. Solvents/reagent were drained off and resin was rinsed with 5 mL DCM. Hexane (25 mL) was added to the solution and the solvents were removed under reduced pressure.

Step 5 Coupling reaction. Obtained acid dissolved in 2.0 ml DMF and to which was added desired amine (1.2), HATU and DIPEA (3.0). The reaction stirred at RT overnight. Reaction diluted with water and extracted with DCM. The organic layer was dried over sodium sulfate and evaporated under vacuum to yield crude product which was purified by HPLC.

Step 6 Purification of final product: After confirming desired product was synthesized by analytical LC/MS, the general purification was using Water system prep-LC/MS using X-Select Peptide CSH.C18 080 Prep Column, 130 Angstrom, 5 μm, 19 mm×150 mm, with elution solvent A Water with 0.5% Formic acid and solvent B Acetonitrile with 0.5% Formic acid.

### Example 1

### 2-methyl-2-(3-methyl-2-(2-oxo-4-phenyl-3-(2-phenylacetamido)butanamido)pentanamido)propanoic acid

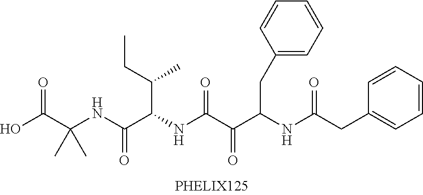 [SMILES: CC[C@H](C)[C@H](NC(=O)C(=O)C(Cc1ccccc1)NC(=O)Cc1ccccc1)C(=O)NC(C)(C)C(=O)O]

Synthesized by Method A. Molecular Weight: 509.6 LC/MS: 510.2 (M+H)⁺

### Example 2

### 2-methyl-2-((2S,3S)-3-methyl-2-(2-oxo-3-(2-phenylacetamido)butanamido)pentanamido)propanoic acid

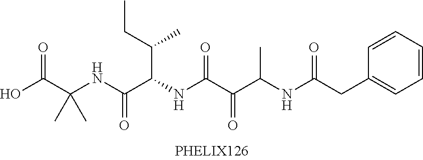 [SMILES: CC[C@H](C)[C@H](NC(=O)C(=O)C(C)NC(=O)Cc1ccccc1)C(=O)NC(C)(C)C(=O)O]

Synthesized by Method A. Molecular Weight: 433.5 LC/MS: 434.2 (M+H)⁺

### Example 3

### 2-methyl-2-((2S,3S)-3-methyl-2-(2-oxo-4-(2-phenylacetamido)butanamido)pentanamido)propanoic acid

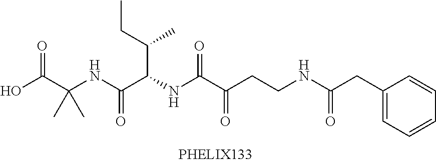 [SMILES: CC[C@H](C)[C@H](NC(=O)C(=O)CCNC(=O)Cc1ccccc1)C(=O)NC(C)(C)C(=O)O]

Synthesized by Method A. Molecular Weight: 433.5 LC/MS: 434.2 (M+H)⁺

### Example 4

### (2-oxo-3-(2-phenylacetamido)butanoyl)-L-isoleucyl-L-proline

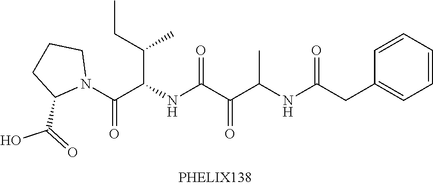 [SMILES: CC[C@H](C)[C@H](NC(=O)C(=O)C(C)NC(=O)Cc1ccccc1)C(=O)N1CCC[C@H]1C(=O)O]

Synthesized by Method A. Molecular Weight: 445.5 LC/MS: 446.2 (M+H)⁺

### Example 5

### 2-methyl-2-((2S,3S)-3-methyl-2-(3-methyl-2-oxo-3-(2-phenylacetamido)butanamido)pentanamido)propanoic acid

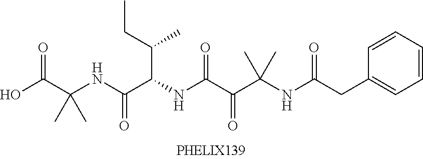 [SMILES: CC[C@H](C)[C@H](NC(=O)C(=O)C(C)(C)NC(=O)Cc1ccccc1)C(=O)NC(C)(C)C(=O)O]

Synthesized by Method A. Molecular Weight: 447.5 LC/MS: 448.2 (M+H)⁺

### Example 6

### (5S,12S)-12-benzyl-5-((S)-sec-butyl)-2,2,9-trimethyl-4,7,8,11,14-pentaoxo-15-phenyl-3,6,10,13-tetraazapentadecanoic acid

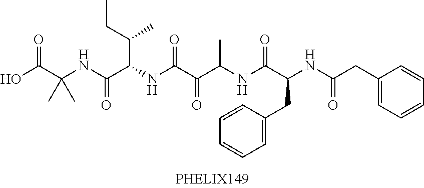 [SMILES: CC[C@H](C)[C@H](NC(=O)C(=O)C(C)NC(=O)[C@H](Cc1ccccc1)NC(=O)Cc1ccccc1)C(=O)NC(C)(C)C(=O)O]

Synthesized by Method A. Molecular Weight: 580.7 LC/MS: 581.3 (M+H)⁺

### Example 7

### (5S,12S)-9,12-dibenzyl-5-((S)-sec-butyl)-2,2-dimethyl-4,7,8,11,14-pentaoxo-15-phenyl-3,6,10,13-tetraazapentadecanoic acid

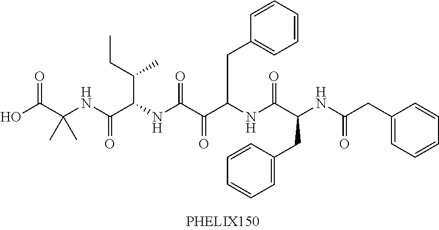 [SMILES: CC[C@H](C)[C@H](NC(=O)C(=O)C(Cc1ccccc1)NC(=O)[C@H](Cc1ccccc1)NC(=O)Cc1ccccc1)C(=O)NC(C)(C)C(=O)O]

Synthesized by Method A. Molecular Weight: 656.8 LC/MS: 657.3 (M+H)⁺

### Example 8

### (2-oxo-3-((S)-3-phenyl-2-(2-phenylacetamido)propanamido)butanoyl)-L-isoleucylproline

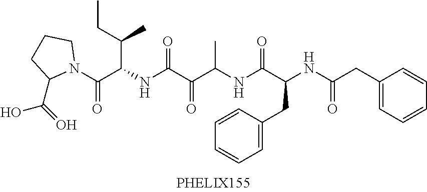 [SMILES: CC[C@@H](C)[C@H](NC(=O)C(=O)C(C)NC(=O)[C@H](Cc1ccccc1)NC(=O)Cc1ccccc1)C(=O)N1CCCC1C(=O)O]

Synthesized by Method A. Molecular Weight: 592.7 LC/MS: 593.3 (M+H)⁺

### Example 9

### (2-oxo-4-phenyl-3-((S)-3-phenyl-2-(2-phenylacetamido)propanamido)butanoyl)-L-isoleucylproline

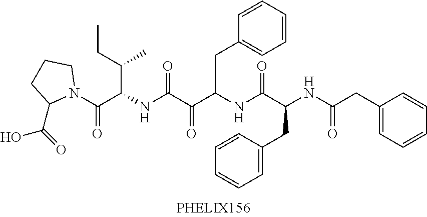 [SMILES: CC[C@H](C)[C@H](NC(=O)C(=O)C(Cc1ccccc1)NC(=O)[C@H](Cc1ccccc1)NC(=O)Cc1ccccc1)C(=O)N1CCCC1C(=O)O]

Synthesized by Method A. Molecular Weight: 668.8 LC/MS: 669.3 (M+H)⁺

### Example 10

### (5S,12S)-5,12-dibenzyl-2,2,9-trimethyl-4,7,8,11,14-pentaoxo-15-phenyl-3,6,10,13-tetraazapentadecanoic acid

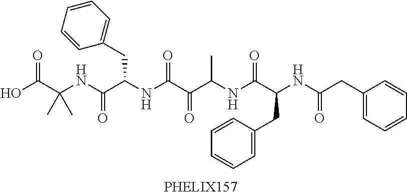 [SMILES: CC(NC(=O)[C@H](Cc1ccccc1)NC(=O)Cc1ccccc1)C(=O)C(=O)N[C@@H](Cc1ccccc1)C(=O)NC(C)(C)C(=O)O]

Synthesized by Method A. Molecular Weight: 614.7 LC/MS: 615.2 (M+H)⁺

### Example 11

### (5S,12S)-12-benzyl-2,2,9-trimethyl-5-(naphthalen-2-ylmethyl)-4,7,8,11,14-pentaoxo-15-phenyl-3,6,10,13-tetraazapentadecanoic acid

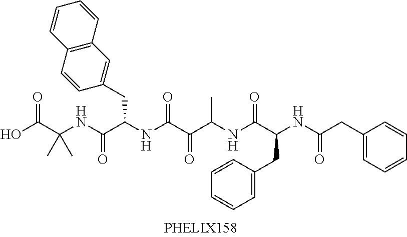 [SMILES: CC(NC(=O)[C@H](Cc1ccccc1)NC(=O)Cc1ccccc1)C(=O)C(=O)N[C@@H](Cc1ccc2ccccc2c1)C(=O)NC(C)(C)C(=O)O]

Synthesized by Method A. Molecular Weight: 664.8 LC/MS: 665.3 (M+H)⁺

### Example 12

### (5S,12S)-15-([1,1′-biphenyl]-4-yl)-12-benzyl-5-((S)-sec-butyl)-2,2,9-trimethyl-4,7,8,11,14-pentaoxo-3,6,10,13-tetraazapentadecanoic acid

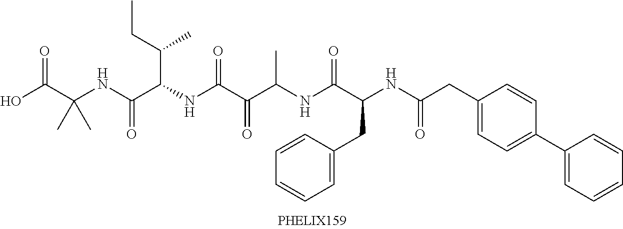 [SMILES: CC[C@H](C)[C@H](NC(=O)C(=O)C(C)NC(=O)[C@H](Cc1ccccc1)NC(=O)Cc1ccc(-c2ccccc2)cc1)C(=O)NC(C)(C)C(=O)O]

Synthesized by Method A. Molecular Weight: 656.8 LC/MS: 657.3(M+H)⁺

### Example 13

### (5S,12S)-12-benzyl-5-((S)-sec-butyl)-2,2,9,9-tetramethyl-4,7,8,11,14-pentaoxo-15-phenyl-3,6,10,13-tetraazapentadecanoic acid

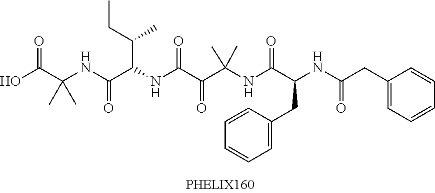

Synthesized by method A. Molecular Weight: 594.71 Observed molecular weight (M+H)⁺ 595.85

### Example 14

### (5S,12S)-12-benzyl-5-((S)-sec-butyl)-2,2,9-trimethyl-15-(naphthalen-2-yl)-4,7,8,11,14-pentaoxo-3,6,10,13-tetraazapentadecanoic acid

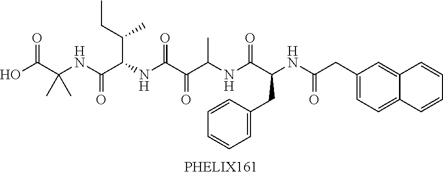 [SMILES: CC[C@H](C)[C@H](NC(=O)C(=O)C(C)NC(=O)[C@H](Cc1ccccc1)NC(=O)Cc1ccc2ccccc2c1)C(=O)NC(C)(C)C(=O)O]

Synthesized by Method A. Molecular Weight: 630.7 LC/MS: 631.3 (M+H)⁺

### Example 15

### (5S,12S)-12-benzyl-2,2,9-trimethyl-5-(naphthalen-1-ylmethyl)-4,7,8,11,14-pentaoxo-15-phenyl-3,6,10,13-tetraazapentadecanoic acid

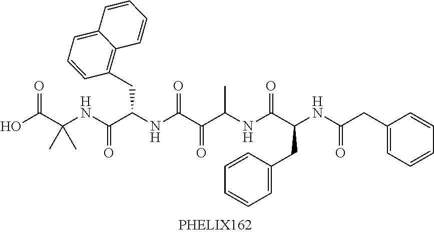 [SMILES: CC(NC(=O)[C@H](Cc1ccccc1)NC(=O)Cc1ccccc1)C(=O)C(=O)N[C@@H](Cc1cccc2ccccc12)C(=O)NC(C)(C)C(=O)O]

Synthesized by Method A. Molecular Weight: 664.8 LC/MS: 665.3 (M+H)⁺

### Example 16

### (5S,12S)-12-benzyl-5-((S)-sec-butyl)-2,2,9-trimethyl-15-(naphthalen-1-yl)-4,7,8,11,14-pentaoxo-3,6,10,13-tetraazapentadecanoic acid

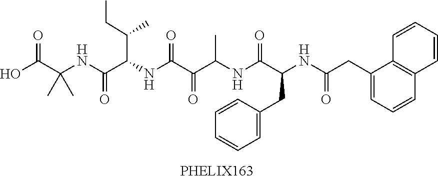 [SMILES: CC[C@H](C)[C@H](NC(=O)C(=O)C(C)NC(=O)[C@H](Cc1ccccc1)NC(=O)Cc1cccc2ccccc12)C(=O)NC(C)(C)C(=O)O]

Synthesized by Method A. Molecular Weight: 630.7 LC/MS: 631.3 (M+H)⁺

### Example 17

### (13S)-13-((S)-sec-butyl)-9,16,16-trimethyl-3,7,10,11,14-pentaoxo-1-phenyl-2-oxa-4,8,12,15-tetraazaheptadecan-17-oic acid

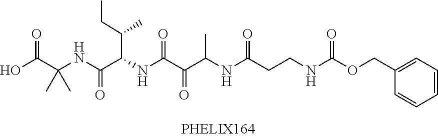 [SMILES: CC[C@H](C)[C@H](NC(=O)C(=O)C(C)NC(=O)CCNC(=O)OCc1ccccc1)C(=O)NC(C)(C)C(=O)O]

Synthesized by Method A. Molecular Weight: 520.6 LC/MS: 521.3 (M+H)⁺

### Example 18

### (5S,12S)-12-([1,1′-biphenyl]-4-ylmethyl)-5-((S)-sec-butyl)-2,2,9-trimethyl-4,7,8,11,14-pentaoxo-15-phenyl-3,6,10,13-tetraazapentadecanoic acid

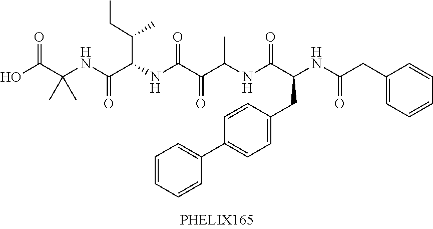 [SMILES: CC[C@H](C)[C@H](NC(=O)C(=O)C(C)NC(=O)[C@H](Cc1ccc(-c2ccccc2)cc1)NC(=O)Cc1ccccc1)C(=O)NC(C)(C)C(=O)O]

Synthesized by Method A. Molecular Weight: 656.8 LC/MS: 657.3 (M+H)⁺

### Example 19

### (5S,12S)-12-benzyl-5-(cyclopropylmethyl)-2,2,9-trimethyl-4,7,8,11,14-pentaoxo-15-phenyl-3,6,10,13-tetraazapentadecanoic acid

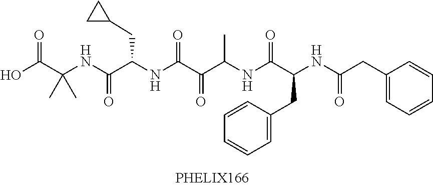 [SMILES: CC(NC(=O)[C@H](Cc1ccccc1)NC(=O)Cc1ccccc1)C(=O)C(=O)N[C@@H](CC1CC1)C(=O)NC(C)(C)C(=O)O]

Synthesized by method A. Molecular Weight: 578.67. Observed molecular weight (M+H)⁺ 579.27

### Example 20

### (5S,12S)-5-((S)-sec-butyl)-12-(4-(tert-butyl)benzyl)-2,2,9-trimethyl-4,7,8,11,14-pentaoxo-15-phenyl-3,6,10,13-tetraazapentadecanoic acid

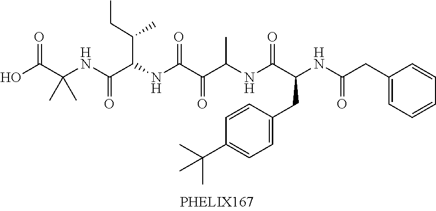

Synthesized by method A. Molecular Weight: 636.79. Observed molecular weight (M+H)⁺ 637.98

### Example 21

### (5S,12S)-5-((S)-sec-butyl)-2,2,9-trimethyl-4,7,8,11,14-pentaoxo-15-phenyl-12-(4-(trifluoromethyl)benzyl)-3,6,10,13-tetraazapentadecanoic acid

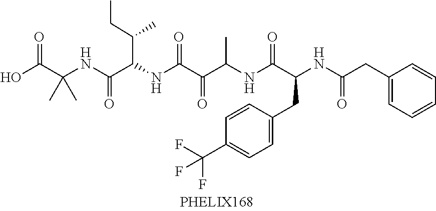

Synthesized by method A. Molecular Weight: 648.68 Observed molecular weight (M+H)⁺ 649.26

### Example 22

### (5S,12S)-12-(4-bromobenzyl)-5-((S)-sec-butyl)-2,2,9-trimethyl-4,7,8,11,14-pentaoxo-15-phenyl-3,6,10,13-tetraazapentadecanoic acid

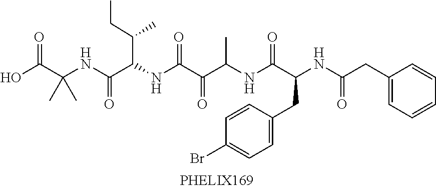

Synthesized by method A. Molecular Weight: 659.58. Observed molecular weight (M+H)⁺ 661.18

### Example 23

### (5S,12S)-5-((S)-sec-butyl)-2,2,9-trimethyl-4,7,8,11,14-pentaoxo-15-phenyl-12-(3-(trifluoromethyl)benzyl)-3,6,10,13-tetraazapentadecanoic acid

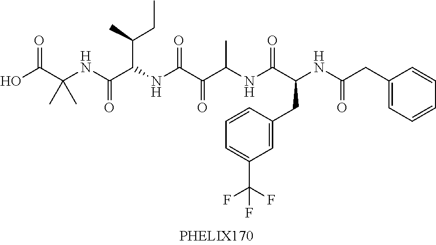

Synthesized by method A. Molecular Weight: 648.68 Observed molecular weight (M+H)⁺ 649.01

### Example 24

### (5S,12S)-5-((S)-sec-butyl)-12-isobutyl-2,2,9-trimethyl-4,7,8,11,14-pentaoxo-15-phenyl-3,6,10,13-tetraazapentadecanoic acid

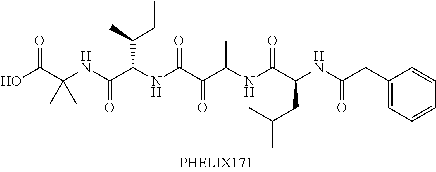 [SMILES: CC[C@H](C)[C@H](NC(=O)C(=O)C(C)NC(=O)[C@H](CC(C)C)NC(=O)Cc1ccccc1)C(=O)NC(C)(C)C(=O)O]

Synthesized by method A. Molecular Weight: 546.67. Observed molecular weight (M+H)⁺ 548.30

### Example 25

### (5S,12S)-5-((S)-sec-butyl)-12-(4-fluorobenzyl)-2,2,9-trimethyl-4,7,8,11,14-pentaoxo-15-phenyl-3,6,10,13-tetraazapentadecanoic acid

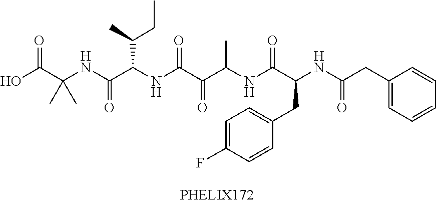 [SMILES: CC[C@H](C)[C@H](NC(=O)C(=O)C(C)NC(=O)[C@H](Cc1ccc(F)cc1)NC(=O)Cc1ccccc1)C(=O)NC(C)(C)C(=O)O]

Synthesized by method A. Molecular Weight: 598.67 Observed molecular weight (M+H)⁺ 598.88

### Example 26

### (5S,12S)-5-((S)-sec-butyl)-2,2,9-trimethyl-4,7,8,11,14-pentaoxo-15-phenyl-12-(thiazol-5-ylmethyl)-3,6,10,13-tetraazapentadecanoic acid

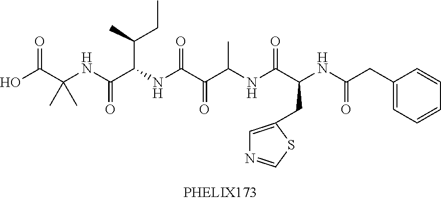 [SMILES: CC[C@H](C)[C@H](NC(=O)C(=O)C(C)NC(=O)[C@H](Cc1cncs1)NC(=O)Cc1ccccc1)C(=O)NC(C)(C)C(=O)O]

Synthesized by Method A. Molecular Weight: 587.8 LC/MS: 588.2 (M+H)⁺

### Example 27

### (4R)-3-((2-oxo-4-phenyl-3-((S)-3-phenyl-2-(2-phenylacetamido)propanamido)butanoyl)-L-isoleucyl)thiazolidine-4-carboxylic acid

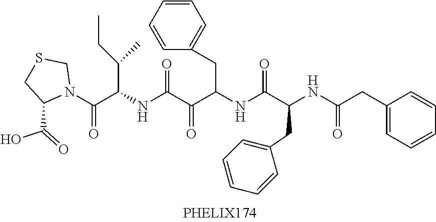 [SMILES: CC[C@H](C)[C@H](NC(=O)C(=O)C(Cc1ccccc1)NC(=O)[C@H](Cc1ccccc1)NC(=O)Cc1ccccc1)C(=O)N1CSC[C@H]1C(=O)O]

Synthesized by Method A. Molecular Weight: 610.7 LC/MS: 611.2 (M+H)⁺

### Example 28

### (5S,12S)-5-((S)-sec-butyl)-2,2,9-trimethyl-12-(3-methylbenzyl)-4,7,8,11,14-pentaoxo-15-phenyl-3,6,10,13-tetraazapentadecanoic acid

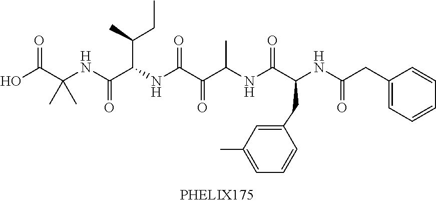 [SMILES: CC[C@H](C)[C@H](NC(=O)C(=O)C(C)NC(=O)[C@H](Cc1cccc(C)c1)NC(=O)Cc1ccccc1)C(=O)NC(C)(C)C(=O)O]

Synthesized by Method A. Molecular Weight: 594.7 LC/MS: 595.3 (M+H)⁺

### Example 29

### (5S,12S)-5-((S)-sec-butyl)-12-(3-cyanobenzyl)-2,2,9-trimethyl-4,7,8,11,14-pentaoxo-15-phenyl-3,6,10,13-tetraazapentadecanoic acid

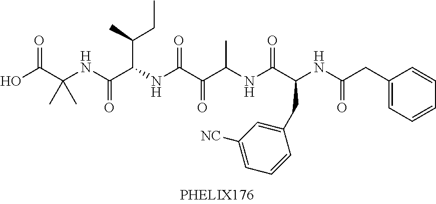 [SMILES: CC[C@H](C)[C@H](NC(=O)C(=O)C(C)NC(=O)[C@H](Cc1cccc(C#N)c1)NC(=O)Cc1ccccc1)C(=O)NC(C)(C)C(=O)O]

Synthesized by Method A. Molecular Weight: 606.7 LC/MS: 608.2 (M+H)⁺

### Example 30

### (5S,12S)-12-benzyl-5-(cyclohexylmethyl)-2,2,9-trimethyl-4,7,8,11,14-pentaoxo-15-phenyl-3,6,10,13-tetraazapentadecanoic acid

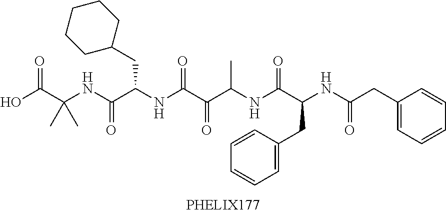 [SMILES: CC(NC(=O)[C@H](Cc1ccccc1)NC(=O)Cc1ccccc1)C(=O)C(=O)N[C@@H](CC1CCCCC1)C(=O)NC(C)(C)C(=O)O]

Synthesized by method A. Molecular Weight: 620.75 Observed molecular weight (M+H)⁺ 622.34

### Example 31

### (2-oxo-3-((S)-3-phenyl-2-(2-phenylacetamido)propanamido)butanoyl)-L-phenylalanyl-L-proline

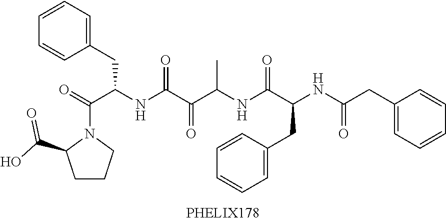 [SMILES: CC(NC(=O)[C@H](Cc1ccccc1)NC(=O)Cc1ccccc1)C(=O)C(=O)N[C@@H](Cc1ccccc1)C(=O)N1CCC[C@H]1C(=O)O]

Synthesized by method A. Molecular Weight: 626.71. Observed molecular weight (M+H)⁺ 627.32

### Example 32

### methyl (5S,12S)-12-benzyl-5-((S)-sec-butyl)-2,2,9-trimethyl-4,7,8,11,14-pentaoxo-15-phenyl-3,6,10,13-tetraazapentadecanoate

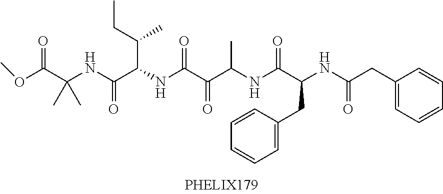 [SMILES: CC[C@H](C)[C@H](NC(=O)C(=O)C(C)NC(=O)[C@H](Cc1ccccc1)NC(=O)Cc1ccccc1)C(=O)NC(C)(C)C(=O)OC]

Synthesized by Method A followed esterification by (trimethylsilyl)diazomethane. Molecular Weight: 594.7 LC/MS: 595.3 (M+H)⁺

### Example 33

### (5S,12S)-12-benzyl-5-(4-(tert-butyl)benzyl)-2,2,9-trimethyl-4,7,8,11,14-pentaoxo-15-phenyl-3,6,10,13-tetraazapentadecanoic acid

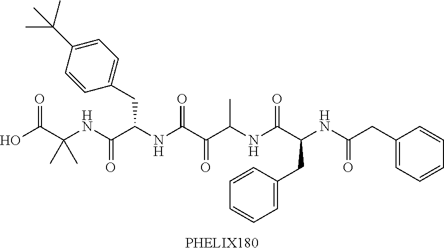 [SMILES: CC(NC(=O)[C@H](Cc1ccccc1)NC(=O)Cc1ccccc1)C(=O)C(=O)N[C@@H](Cc1ccc(C(C)(C)C)cc1)C(=O)NC(C)(C)C(=O)O]

Synthesized by method A. Molecular Weight: 670.81 Observed molecular weight (M+H)⁺ 672.35

### Example 34

### (2S)-N-(4-(((S)-1-((1-amino-2-methyl-1-oxopropan-2-yl)amino)-1-oxopropan-2-yl)amino)-3,4-dioxobutan-2-yl)-4-methyl-2-(2-phenylacetamido)pentanamide

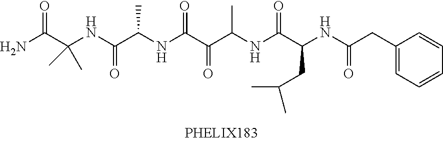 [SMILES: CC(C)C[C@H](NC(=O)Cc1ccccc1)C(=O)NC(C)C(=O)C(=O)N[C@@H](C)C(=O)NC(C)(C)C(N)=O]

Synthesized by Method B. Molecular Weight: 503.6 LC/MS: 504.3 (M+H)⁺

### Example 35

### benzyl ((2S)-1-((4-((2-(((S)-1-amino-3-(naphthalen-2-yl)-1-oxopropan-2-yl)amino)-2-oxoethyl)amino)-3,4-dioxobutan-2-yl)amino)-4-methyl-1-oxopentan-2-yl)carbamate

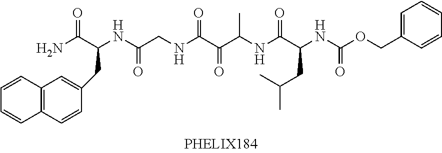 [SMILES: CC(C)C[C@H](NC(=O)OCc1ccccc1)C(=O)NC(C)C(=O)C(=O)NCC(=O)N[C@@H](Cc1ccc2ccccc2c1)C(N)=O]

Synthesized by Method B. Molecular Weight: 617.7 LC/MS: 618.3 (M+H)⁺

### Example 36

### benzyl ((2S)-1-((4-((3-(((S)-1-amino-3-(3-cyanophenyl)-1-oxopropan-2-yl)amino)-3-oxopropyl)amino)-3,4-dioxobutan-2-yl)amino)-4-methyl-1-oxopentan-2-yl)carbamate

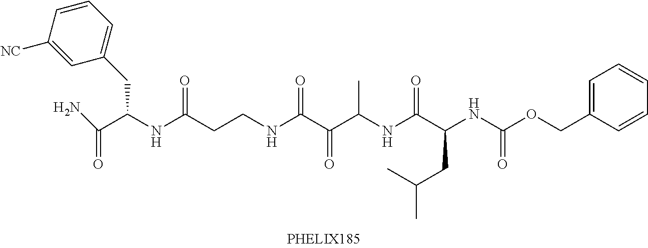 [SMILES: CC(C)C[C@H](NC(=O)OCc1ccccc1)C(=O)NC(C)C(=O)C(=O)NCCC(=O)N[C@@H](Cc1cccc(C#N)c1)C(N)=O]

Synthesized by Method B. Molecular Weight: 606.7 LC/MS: 607.3 (M+H)⁺

### Example 37

### benzyl ((2S)-1-((4-(4-(((S)-1-amino-1-oxo-3-phenylpropan-2-yl)carbamoyl)piperidin-1-yl)-3,4-dioxobutan-2-yl)amino)-4-methyl-1-oxopentan-2-yl)carbamate

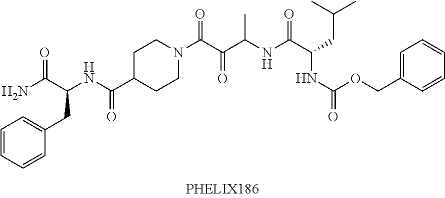 [SMILES: CC(C)C[C@H](NC(=O)OCc1ccccc1)C(=O)NC(C)C(=O)C(=O)N1CCC(C(=O)N[C@@H](Cc2ccccc2)C(N)=O)CC1]

Synthesized by method B. Molecular Weight: 621.74 Observed molecular weight (M+H)⁺ 623.03

### Example 38

### benzyl ((2S)-1-((4-(3-(((S)-1-amino-1-oxo-3-phenylpropan-2-yl)carbamoyl)azetidin-1-yl-3,4-dioxobutan-2-yl)amino)-4-methyl-1-oxopentan-2-yl)carbamate

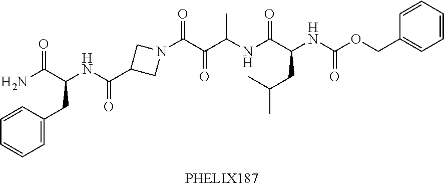 [SMILES: CC(C)C[C@H](NC(=O)OCc1ccccc1)C(=O)NC(C)C(=O)C(=O)N1CC(C(=O)N[C@@H](Cc2ccccc2)C(N)=O)C1]

Synthesized by method B. Molecular Weight: 593.68 Observed molecular weight (M+H)⁺ 594.34

### Example 39

### benzyl ((2S)-1-((4-(((S)-1-amino-1-oxo-3-(pyridin-4-yl)propan-2-yl)amino)-3,4-dioxobutan-2-yl)amino)-4-methyl-1-oxopentan-2-yl)carbamate

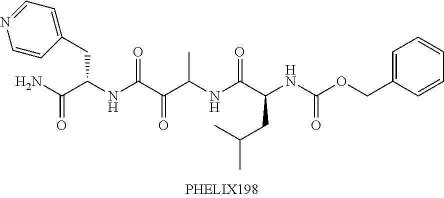 [SMILES: CC(C)C[C@H](NC(=O)OCc1ccccc1)C(=O)NC(C)C(=O)C(=O)N[C@@H](Cc1ccncc1)C(N)=O]

Synthesized by Method B. Molecular Weight: 511.6 LC/MS: 512.3 (M+H)⁺

### Example 40

### (2S)-2-((S)-3-(allyloxy)-2-(2-phenylacetamido)propanamido)-N-(1-(allyloxy)-4-((2-(((S)-1-amino-3-(naphthalen-2-yl)-1-oxopropan-2-yl)amino)-2-oxoethyl)amino)-3,4-dioxobutan-2-yl)-4-methylpentanamide

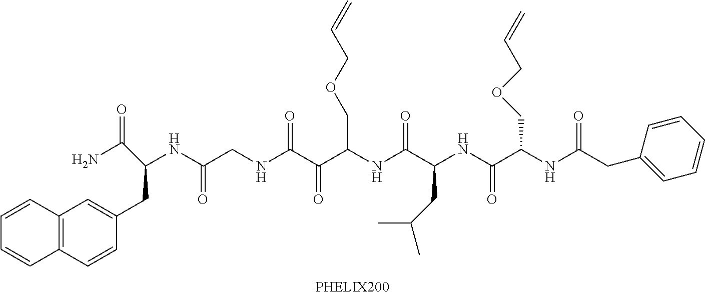 [SMILES: C=CCOCC(NC(=O)[C@H](CC(C)C)NC(=O)[C@H](COCC=C)NC(=O)Cc1ccccc1)C(=O)C(=O)NCC(=O)N[C@@H](Cc1ccc2ccccc2c1)C(N)=O]

Synthesized by Method B. Molecular Weight: 784.9 LC/MS: 785.5 (M+H)⁺

### Example 41

### (2S)-N-(1-(allyloxy)-4-((2-(((S)-1-amino-3-(naphthalen-2-yl)-1-oxopropan-2-yl)amino)-2-oxoethyl)amino)-3,4-dioxobutan-2-yl)-2-((S)-3-(4-(allyloxy)phenyl)-2-(2-phenylacetamido)propanamido)-4-methylpentanamide

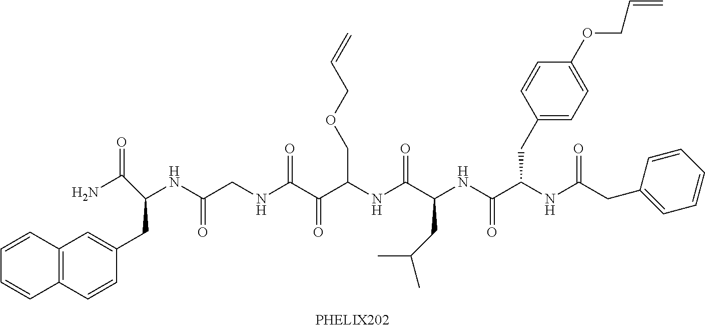 [SMILES: C=CCOCC(NC(=O)[C@H](CC(C)C)NC(=O)[C@H](Cc1ccc(OCC=C)cc1)NC(=O)Cc1ccccc1)C(=O)C(=O)NCC(=O)N[C@@H](Cc1ccc2ccccc2c1)C(N)=O]

Synthesized by Method B. Molecular Weight: 861.0 LC/MS: 861.5 (M+H)⁺

### Example 42

### (2S)-1-((2-oxo-4-phenyl-3-((S)-3-phenyl-2-(2-phenylacetamido)propanamido)butanoyl)-L-isoleucyl)pyrrolicline-2-carboxamide

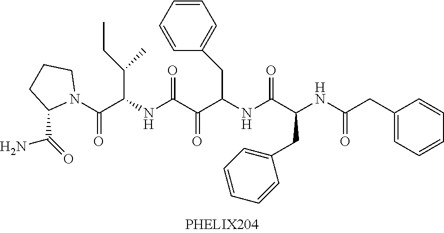 [SMILES: CC[C@H](C)[C@H](NC(=O)C(=O)C(Cc1ccccc1)NC(=O)[C@H](Cc1ccccc1)NC(=O)Cc1ccccc1)C(=O)N1CCC[C@H]1C(N)=O]

Synthesized by Method B. Molecular Weight: 667.8 LC/MS: 668.4 (M+H)⁺

### Example 43

### N-((S)-1-((1-amino-2-methyl-1-oxopropan-2-yl)amino)-1-oxo-3-phenylpropan-2-yl)-3-((S)-3-(naphthalen-1-yl)-2-(2-phenylacetamido)propanamido)-2-oxobutanamide

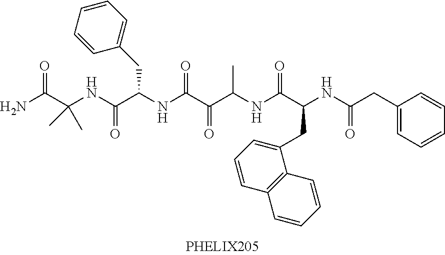 [SMILES: CC(NC(=O)[C@H](Cc1cccc2ccccc12)NC(=O)Cc1ccccc1)C(=O)C(=O)N[C@@H](Cc1ccccc1)C(=O)NC(C)(C)C(N)=O]

Synthesized by Method B. Molecular Weight: 663.8 LC/MS: 664.3 (M+H)⁺

### Example 44

### N-((S)-1-((1-amino-2-methyl-1-oxopropan-2-yl)amino)-1-oxo-3-phenylpropan-2-yl)-3-((S)-3-(naphthalen-2-yl)-2-(2-phenylacetamido)propanamido)-2-oxobutanamide

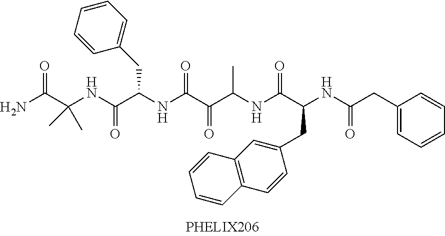 [SMILES: CC(NC(=O)[C@H](Cc1ccc2ccccc2c1)NC(=O)Cc1ccccc1)C(=O)C(=O)N[C@@H](Cc1ccccc1)C(=O)NC(C)(C)C(N)=O]

Synthesized by Method B. Molecular Weight: 663.8 LC/MS: 664.3 (M+H)⁺

### Example 45

### N-((S)-1-((1-amino-2-methyl-1-oxopropan-2-yl)amino)-1-oxo-3-phenylpropan-2-yl)-3-((S)-3-(2-fluorophenyl)-2-(2-phenylacetamido)propanamido)-2-oxobutanamide

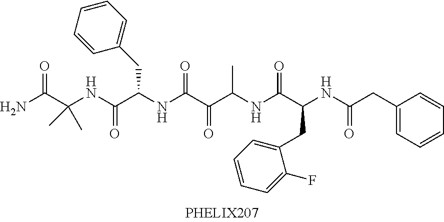 [SMILES: CC(NC(=O)[C@H](Cc1ccccc1F)NC(=O)Cc1ccccc1)C(=O)C(=O)N[C@@H](Cc1ccccc1)C(=O)NC(C)(C)C(N)=O]

Synthesized by Method B. Molecular Weight: 631.7 LC/MS: 632.3 (M+H)⁺

### Example 46

### N-((S)-1-((1-amino-2-methyl-1-oxopropan-2-yl)amino)-1-oxo-3-phenylpropan-2-yl)-3-((S)-3-(4-fluorophenyl)-2-(2-phenylacetamido)propanamido)-2-oxobutanamide

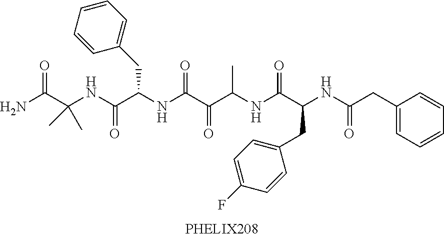 [SMILES: CC(NC(=O)[C@H](Cc1ccc(F)cc1)NC(=O)Cc1ccccc1)C(=O)C(=O)N[C@@H](Cc1ccccc1)C(=O)NC(C)(C)C(N)=O]

Synthesized by Method B. Molecular Weight: 631.7 LC/MS: 632.3 (M+H)⁺

### Example 47

### N-((S)-1-((1-amino-2-methyl-1-oxopropan-2-yl)amino)-1-oxo-3-phenylpropan-2-yl)-2-oxo-3-((S)-2-(2-phenylacetamido)-3-(3-(trifluoromethyl)phenyl)propanamido)butanamide

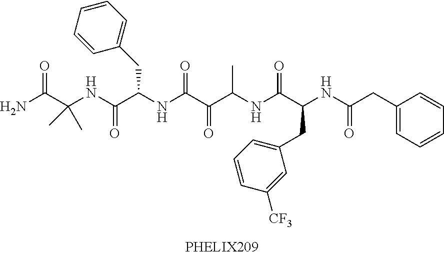 [SMILES: CC(NC(=O)[C@H](Cc1cccc(C(F)(F)F)c1)NC(=O)Cc1ccccc1)C(=O)C(=O)N[C@@H](Cc1ccccc1)C(=O)NC(C)(C)C(N)=O]

Synthesized by Method B. Molecular Weight: 681.7 LC/MS: 682.3 (M+H)⁺

### Example 48

### N-((S)-1-((1-amino-2-methyl-1-oxopropan-2-yl)amino)-1-oxo-3-phenylpropan-2-yl)-2-oxo-3-((S)-2-(2-phenylacetamido)-3-(pyridin-3-yl)propanamido)butanamide

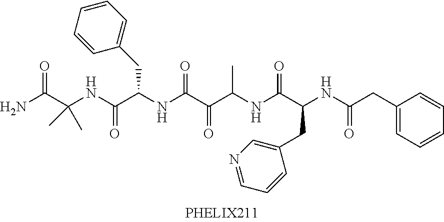 [SMILES: CC(NC(=O)[C@H](Cc1cccnc1)NC(=O)Cc1ccccc1)C(=O)C(=O)N[C@@H](Cc1ccccc1)C(=O)NC(C)(C)C(N)=O]

Synthesized by Method B. Molecular Weight: 614.7 LC/MS: 615.2 (M+H)⁺

### Example 49

### N-((S)-1-((1-amino-2-methyl-1-oxopropan-2-yl)amino)-1-oxo-3-phenylpropan-2-yl)-3-((S)-3-(3-fluorophenyl)-2-(2-phenylacetamido)propanamido)-2-oxobutanamide

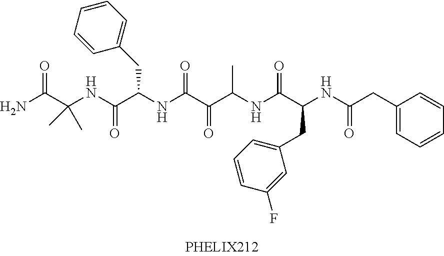 [SMILES: CC(NC(=O)[C@H](Cc1cccc(F)c1)NC(=O)Cc1ccccc1)C(=O)C(=O)N[C@@H](Cc1ccccc1)C(=O)NC(C)(C)C(N)=O]

Synthesized by Method B. Molecular Weight: 631.7 LC/MS: 632.3 (M+H)⁺

### Example 50

### N-((S)-1-(((S)-3-([1,1′-biphenyl]-4-yl)-1-amino-1-oxopropan-2-yl)amino)-1-oxo-3-phenylpropan-2-yl)-2-oxo-3-((S)-3-phenyl-2-(2-phenylacetamido)propanamido)butanamide

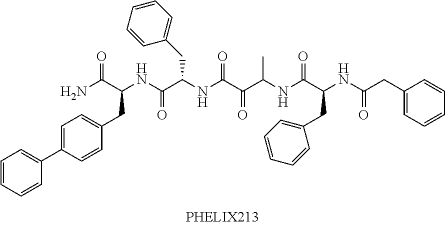 [SMILES: CC(NC(=O)[C@H](Cc1ccccc1)NC(=O)Cc1ccccc1)C(=O)C(=O)N[C@@H](Cc1ccccc1)C(=O)N[C@@H](Cc1ccc(-c2ccccc2)cc1)C(N)=O]

Synthesized by Method B. Molecular Weight: 751.9 LC/MS: 752.4 (M+H)⁺

### Example 51

### (2S)-N-(1-amino-2-methyl-1-oxopropan-2-yl)-4-methyl-2-(2-oxo-3-((S)-3-phenyl-2-(2-phenylacetamido)propanamido)butanamido)pentanamide

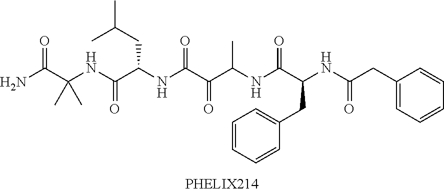 [SMILES: CC(C)C[C@H](NC(=O)C(=O)C(C)NC(=O)[C@H](Cc1ccccc1)NC(=O)Cc1ccccc1)C(=O)NC(C)(C)C(N)=O]

Synthesized by Method B. Molecular Weight: 579.31. LC/MS: 580.13 (M+1)⁺

### Example 52

### N-((S)-1-(((S)-1-amino-3-(naphthalen-2-yl)-1-oxopropan-2-yl)amino)-1-oxo-3-phenylpropan-2-yl)-2-oxo-3-((S)-3-phenyl-2-(2-phenylacetamido)propanamido)butanamide

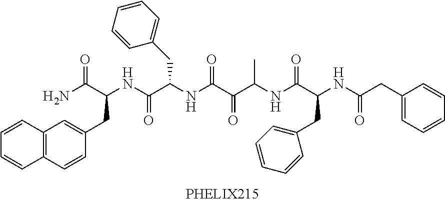 [SMILES: CC(NC(=O)[C@H](Cc1ccccc1)NC(=O)Cc1ccccc1)C(=O)C(=O)N[C@@H](Cc1ccccc1)C(=O)N[C@@H](Cc1ccc2ccccc2c1)C(N)=O]

Synthesized by Method B. Molecular Weight: 725.9 LC/MS: 726.3 (M+H)⁺

### Example 53

### (5S,12S)-12-benzyl-5-((S)-sec-butyl)-2,2,9-trimethyl-4,7,8,11,14-pentaoxo-3,6,10,13-tetraazapentadecanoic acid

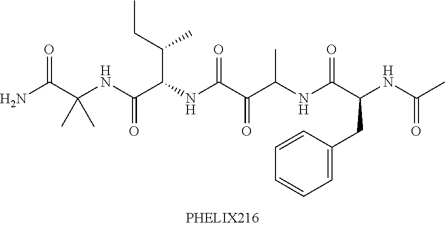 [SMILES: CC[C@H](C)[C@H](NC(=O)C(=O)C(C)NC(=O)[C@H](Cc1ccccc1)NC(C)=O)C(=O)NC(C)(C)C(N)=O]

Synthesized by method B. Molecular Weight: 504.58 Observed molecular weight (M+H)⁺ 505.23

### Example 54

### 1-((2S,3S)-3-methyl-2-(2-oxo-3-((S)-3-phenyl-2-(2-phenylacetamido)propanamido)-butanamido)pentanamido)cyclopentane-1-carboxylic acid

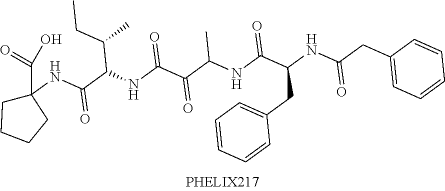

Synthesized by method A. Molecular Weight: 606.72 Observed molecular weight (M+H)⁺ 607.33

### Example 55

### (5S,13S)-13-benzyl-5-((S)-sec-butyl)-2,2-dimethyl-4,7,8,12,15-pentaoxo-16-phenyl-3,6,11,14-tetraazahexadecanoic acid

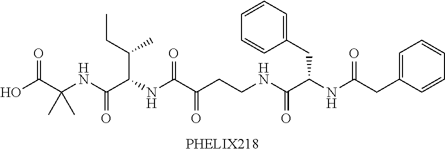 [SMILES: CC[C@H](C)[C@H](NC(=O)C(=O)CCNC(=O)[C@H](Cc1ccccc1)NC(=O)Cc1ccccc1)C(=O)NC(C)(C)C(=O)O]

Synthesized by Method A. Molecular Weight: 580.7 LC/MS: 581.3 (M+H)⁺

### Example 56

### N-((S)-1-(((S)-1-amino-1-oxo-3-phenylpropan-2-yl)amino)-1-oxo-3-phenylpropan-2-yl)-2-oxo-3-((S)-2-(2-phenylacetamido)-3-(pyridin-2-yl)propanamido)butanamide

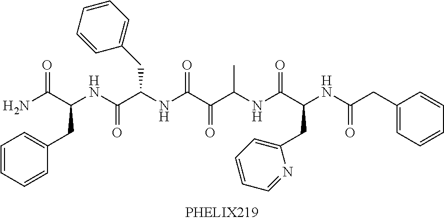 [SMILES: CC(NC(=O)[C@H](Cc1ccccn1)NC(=O)Cc1ccccc1)C(=O)C(=O)N[C@@H](Cc1ccccc1)C(=O)N[C@@H](Cc1ccccc1)C(N)=O]

Synthesized by Method B. Molecular Weight: 614.7 LC/MS: 615.2 (M+H)⁺

### Example 57

### N-((S)-1-(((S)-1-amino-3-(naphthalen-1-yl)-1-oxopropan-2-yl)amino)-1-oxo-3-phenylpropan-2-yl)-2-oxo-3-((S)-3-phenyl-2-(2-phenylacetamido)propanamido)butanamide

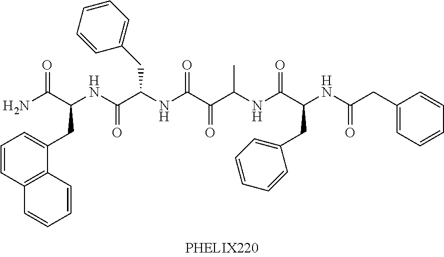 [SMILES: CC(NC(=O)[C@H](Cc1ccccc1)NC(=O)Cc1ccccc1)C(=O)C(=O)N[C@@H](Cc1ccccc1)C(=O)N[C@@H](Cc1cccc2ccccc12)C(N)=O]

Synthesized by Method B. Molecular Weight: 725.9 LC/MS: 726.4 (M+H)⁺

### Example 58

### N-((S)-1-(((S)-1-amino-3-(4-(tert-butyl)phenyl)-1-oxopropan-2-yl)amino)-1-oxo-3-phenylpropan-2-yl)-2-oxo-3-((S)-3-phenyl-2-(2-phenylacetamido)propanamido)butanamide

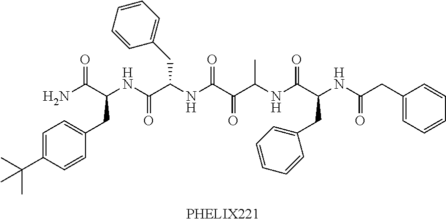 [SMILES: CC(NC(=O)[C@H](Cc1ccccc1)NC(=O)Cc1ccccc1)C(=O)C(=O)N[C@@H](Cc1ccccc1)C(=O)N[C@@H](Cc1ccc(C(C)(C)C)cc1)C(N)=O]

Synthesized by Method B. Molecular Weight: 731.9 LC/MS: 732.4 (M+H)⁺

### Example 59

### N-((2S)-1-((4-(((S)-1-((1-amino-2-methyl-1-oxopropan-2-yl)amino)-1-oxo-3-phenylpropan-2-yl)amino)-3,4-dioxobutan-2-yl)amino)-1-oxo-3-phenylpropan-2-yl)-3-fluorobenzamide

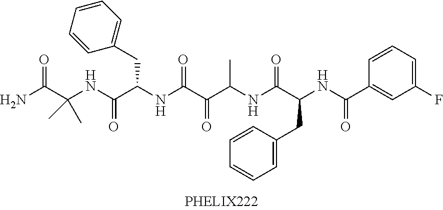 [SMILES: CC(NC(=O)[C@H](Cc1ccccc1)NC(=O)c1cccc(F)c1)C(=O)C(=O)N[C@@H](Cc1ccccc1)C(=O)NC(C)(C)C(N)=O]

Synthesized by method B. Molecular Weight: 617.68 Observed molecular weight (M+H)⁺ 618.17

### Example 60

### N-((2S)-1-((4-(((S)-1-((1-amino-2-methyl-1-oxopropan-2-yl)amino)-1-oxo-3-phenylpropan-2-yl)amino)-3,4-dioxobutan-2-yl)amino)-1-oxo-3-phenylpropan-2-yl)benzamide

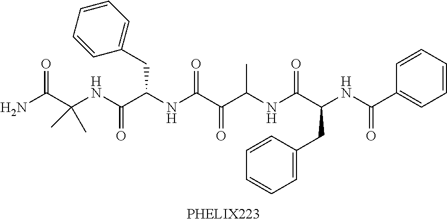 [SMILES: CC(NC(=O)[C@H](Cc1ccccc1)NC(=O)c1ccccc1)C(=O)C(=O)N[C@@H](Cc1ccccc1)C(=O)NC(C)(C)C(N)=O]

Synthesized by method B. Molecular Weight: 599.69 Observed molecular weight (M+H)⁺ 600.26

### Example 61

### N-((2S)-1-((4-(((S)-1-((1-amino-2-methyl-1-oxopropan-2-yl)amino)-1-oxo-3-phenylpropan-2-yl)amino)-3,4-dioxobutan-2-yl)amino)-1-oxo-3-phenylpropan-2-yl)-2-fluorobenzamide

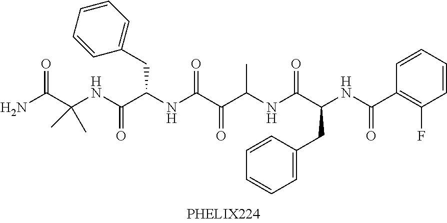 [SMILES: CC(NC(=O)[C@H](Cc1ccccc1)NC(=O)c1ccccc1F)C(=O)C(=O)N[C@@H](Cc1ccccc1)C(=O)NC(C)(C)C(N)=O]

Synthesized by method B. Molecular Weight: 617.68 Observed molecular weight (M+H)⁺ 618.24

### Example 62

### N-((S)-1-((1-amino-2-methyl-1-oxopropan-2-yl)amino)-1-oxo-3-phenylpropan-2-yl)-2-oxo-3-((S)-3-phenyl-2-(2-(m-tolyl)acetamido)propanamido)butanamide

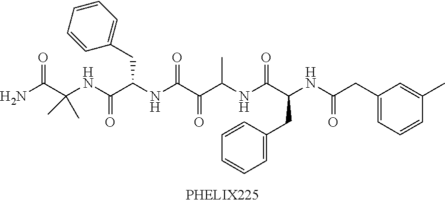 [SMILES: Cc1cccc(CC(=O)N[C@@H](Cc2ccccc2)C(=O)NC(C)C(=O)C(=O)N[C@@H](Cc2ccccc2)C(=O)NC(C)(C)C(N)=O)c1]

Synthesized by method B. Molecular Weight: 627.74 Observed molecular weight (M+H)⁺ 628.33

### Example 63

### N-((S)-1-((1-amino-2-methyl-1-oxopropan-2-yl)amino)-1-oxo-3-phenylpropan-2-yl)-2-oxo-3-((S)-3-phenyl-2-(2-(o-tolyl)acetamido)propanamido)butanamide

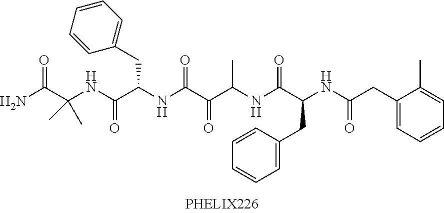 [SMILES: Cc1ccccc1CC(=O)N[C@@H](Cc1ccccc1)C(=O)NC(C)C(=O)C(=O)N[C@@H](Cc1ccccc1)C(=O)NC(C)(C)C(N)=O]

Synthesized by method B. Molecular Weight: 627.74 Observed molecular weight (M+H)⁺ 628.26

### Example 64

### N-((S)-1-((1-amino-2-methyl-1-oxopropan-2-yl)amino)-1-oxo-3-phenylpropan-2-yl)-3-((S)-2-(2-(3-fluorophenyl)acetamido)-3-phenylpropanamido)-2-oxobutanamide

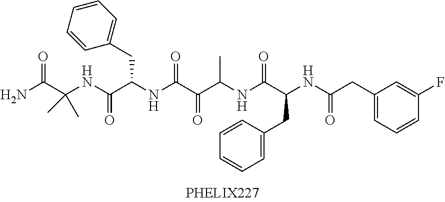 [SMILES: CC(NC(=O)[C@H](Cc1ccccc1)NC(=O)Cc1cccc(F)c1)C(=O)C(=O)N[C@@H](Cc1ccccc1)C(=O)NC(C)(C)C(N)=O]

Synthesized by method B. Molecular Weight: 631.71 Observed molecular weight (M+H)⁺ 632.24

### Example 65

### benzyl ((2S)-1-((1-(allyloxy)-4-(((S)-3-(allyloxy)-1-(((S)-1-amino-3-(naphthalen-2-yl)-1-oxopropan-2-yl)amino)-1-oxopropan-2-yl)amino)-3,4-dioxobutan-2-yl)amino)-4-methyl-1-oxopentan-2-yl)carbamate

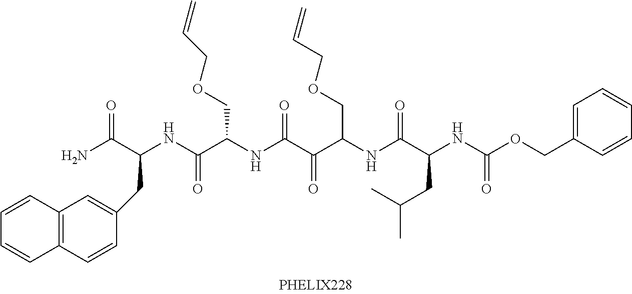 [SMILES: C=CCOCC(NC(=O)[C@H](CC(C)C)NC(=O)OCc1ccccc1)C(=O)C(=O)N[C@@H](COCC=C)C(=O)N[C@@H](Cc1ccc2ccccc2c1)C(N)=O]

Synthesized by Method B. Molecular Weight: 800.9. LC/MS: 801.4 (M+H)⁺

### Example 66

### N-((S)-1-((2-methyl-1-oxo-1-(phenethylamino)propan-2-yl)amino)-1-oxo-3-phenylpropan-2-yl)-2-oxo-3-((S)-3-phenyl-2-(2-phenylacetamido)propanamido)butanamide

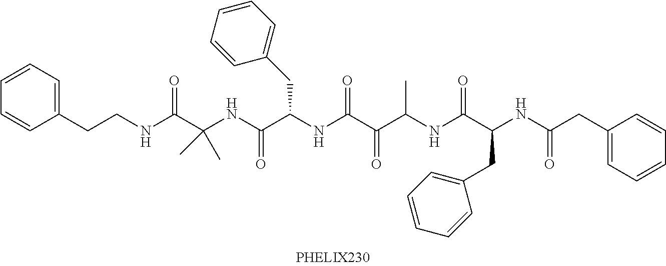 [SMILES: CC(NC(=O)[C@H](Cc1ccccc1)NC(=O)Cc1ccccc1)C(=O)C(=O)N[C@@H](Cc1ccccc1)C(=O)NC(C)(C)C(=O)NCCc1ccccc1]

Synthesized by Method D. Molecular Weight: 717.9 LC/MS: 718.4(M+H)⁺

### Example 67

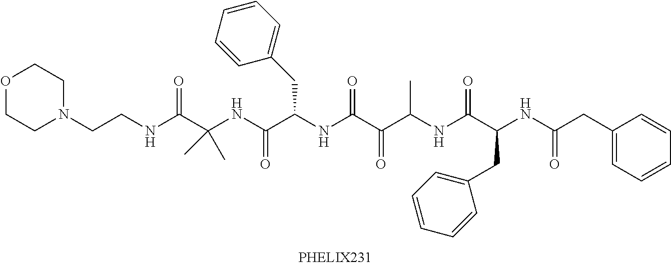 [SMILES: CC(NC(=O)[C@H](Cc1ccccc1)NC(=O)Cc1ccccc1)C(=O)C(=O)N[C@@H](Cc1ccccc1)C(=O)NC(C)(C)C(=O)NCCN1CCOCC1]

Synthesized by Method D. Molecular Weight: 726.9 LC/MS: 727.4 (M+H)⁺

### Example 68

### N-((S)-1-((1-amino-2-methyl-1-oxopropan-2-yl)amino)-3-(1H-indol-2-yl)-1-oxopropan-2-yl)-2-oxo-3-((S)-3-phenyl-2-(2-phenylacetamido)propanamido)butanamide

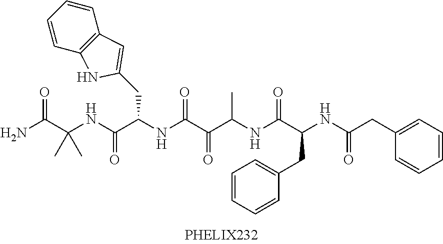 [SMILES: CC(NC(=O)[C@H](Cc1ccccc1)NC(=O)Cc1ccccc1)C(=O)C(=O)N[C@@H](Cc1cc2ccccc2[nH]1)C(=O)NC(C)(C)C(N)=O]

Synthesized by Method B. Molecular Weight: 652.30. LC/MS: 653.14 (M+1)⁺

### Example 69

### (4S)-N-((S)-1-((1-amino-2-methyl-1-oxopropan-2-yl)amino)-1-oxo-3-phenylpropan-2-yl)-4-methyl-2-oxo-3-((S)-3-phenyl-2-(2-phenylacetamido)propanamido)hexanamide

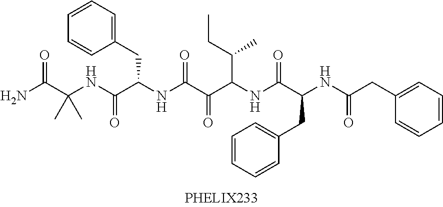 [SMILES: CC[C@H](C)C(NC(=O)[C@H](Cc1ccccc1)NC(=O)Cc1ccccc1)C(=O)C(=O)N[C@@H](Cc1ccccc1)C(=O)NC(C)(C)C(N)=O]

Synthesized by Method B. Molecular Weight: 655.34. LC/MS: 655.81 (M+1)⁺

### Example 70

### (2S)-N-(1-amino-2-methyl-1-oxopropan-2-yl)-5-guanidino-2-(2-oxo-3-((S)-3-phenyl-2-(2-phenylacetamido)propanamido)butanamido)pentanamide

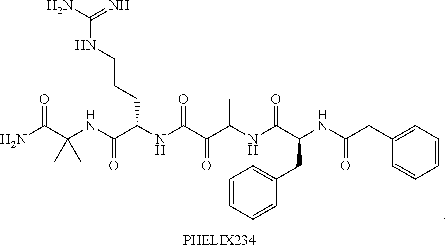 [SMILES: CC(NC(=O)[C@H](Cc1ccccc1)NC(=O)Cc1ccccc1)C(=O)C(=O)N[C@@H](CCCNC(=N)N)C(=O)NC(C)(C)C(N)=O ; "."]

Synthesized by Method B. Molecular Weight: 622.7. LC/MS: 622.94 (M+1)⁺

### Example 71

### N-((S)-1-((1-amino-2-methyl-1-oxopropan-2-yl)amino)-1-oxo-3-phenylpropan-2-yl)-3-((S)-2-(3-methylbutanamido)-3-phenylpropanamido)-2-oxobutanamide

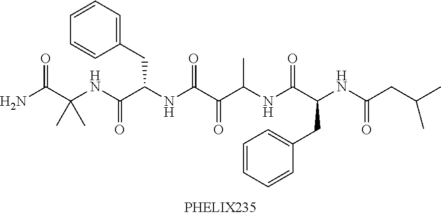 [SMILES: CC(C)CC(=O)N[C@@H](Cc1ccccc1)C(=O)NC(C)C(=O)C(=O)N[C@@H](Cc1ccccc1)C(=O)NC(C)(C)C(N)=O]

Synthesized by method B. Molecular Weight: 579.70 Observed molecular weight (M+H)⁺ 580.34

### Example 72

### N-((2S)-1-((4-(((S)-1-((1-amino-2-methyl-1-oxopropan-2-yl)amino)-1-oxo-3-phenylpropan-2-yl)amino)-3,4-dioxobutan-2-yl)amino)-1-oxo-3-phenylpropan-2-yl)pentanamide

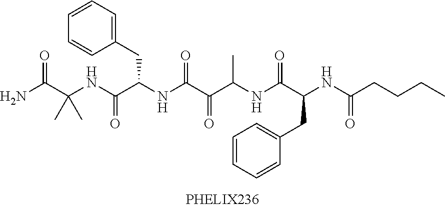 [SMILES: CCCCC(=O)N[C@@H](Cc1ccccc1)C(=O)NC(C)C(=O)C(=O)N[C@@H](Cc1ccccc1)C(=O)NC(C)(C)C(N)=O]

Synthesized by method B. Molecular Weight: 579.70 Observed molecular weight (M+H)⁺ 580.46

### Example 73

### N-((S)-1-((1-amino-2-methyl-1-oxopropan-2-yl)amino)-1-oxo-3-phenylpropan-2-yl)-3-((S)-2-(2,2-diphenylacetamido)-3-phenylpropanamido)-2-oxobutanamide

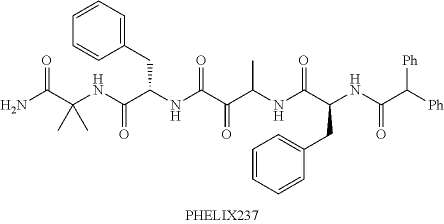 [SMILES: CC(NC(=O)[C@H](Cc1ccccc1)NC(=O)C(c1ccccc1)c1ccccc1)C(=O)C(=O)N[C@@H](Cc1ccccc1)C(=O)NC(C)(C)C(N)=O]

Synthesized by method B. Molecular Weight: 689.81 Observed molecular weight (M+H)⁺ 691.33

### Example 74

### benzyl ((5S,15S)-5-benzyl-12,17-dimethyl-4,7,10,11,14-pentaoxo-1-phenyl-3,6,9,13-tetraazaoctadecan-15-yl)carbamate

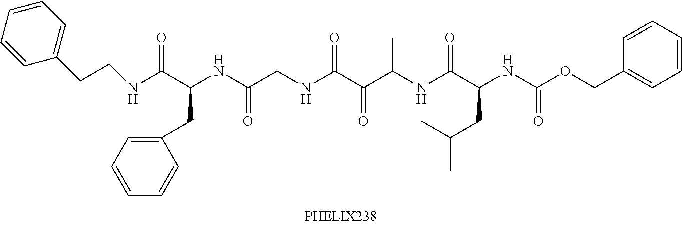 [SMILES: CC(C)C[C@H](NC(=O)OCc1ccccc1)C(=O)NC(C)C(=O)C(=O)NCC(=O)N[C@@H](Cc1ccccc1)C(=O)NCCc1ccccc1]

Synthesized by Method D. Molecular Weight: 671.8 LC/MS: 672.3 (M+H)⁺

### Example 75

### N-((S)-3-([1,1′-biphenyl]-4-yl)-1-((1-amino-2-methyl-1-oxopropan-2-yl)amino)-1-oxopropan-2-yl)-2-oxo-3-((S)-3-phenyl-2-(2-phenylacetamido)propanamido)butanamide

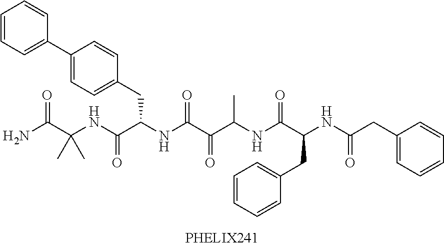 [SMILES: CC(NC(=O)[C@H](Cc1ccccc1)NC(=O)Cc1ccccc1)C(=O)C(=O)N[C@@H](Cc1ccc(-c2ccccc2)cc1)C(=O)NC(C)(C)C(N)=O]

Synthesized by Method B. Molecular Weight: 689.81. LC/MS: 691.96 (M+1)⁺

### Example 76

### (2S)-N-(1-amino-2-methyl-1-oxopropan-2-yl)-2-(2-oxo-3-((S)-3-phenyl-2-(2-phenylacetamido)propanamido)butanamido)hexanamide

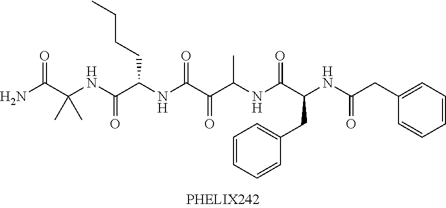 [SMILES: CCCC[C@H](NC(=O)C(=O)C(C)NC(=O)[C@H](Cc1ccccc1)NC(=O)Cc1ccccc1)C(=O)NC(C)(C)C(N)=O]

Synthesized by Method B. Molecular Weight: 579.7. LC/MS: 580.27 (M+1)⁺

### Example 77

### (2S)-N-(1-amino-2-methyl-1-oxopropan-2-yl)-2-(2-oxo-3-((S)-3-phenyl-2-(2-phenylacetamido)propanamido)butanamido)pentanamide

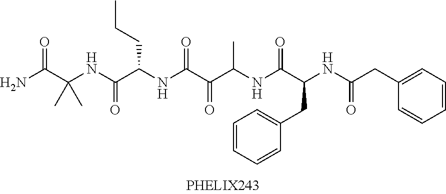 [SMILES: CCC[C@H](NC(=O)C(=O)C(C)NC(=O)[C@H](Cc1ccccc1)NC(=O)Cc1ccccc1)C(=O)NC(C)(C)C(N)=O]

Synthesized by Method B. Molecular Weight: 565.7. LC/MS: 566.26 (M+1)⁺

### Example 78

### N-((S)-1-(((S)-3-([1,1′-biphenyl]-4-yl)-1-amino-1-oxopropan-2-yl)amino)-1-oxo-3-phenylpropan-2-yl)-2-oxo-3-((R)-3-phenyl-2-(2-phenylacetamido)propanamido)butanamide

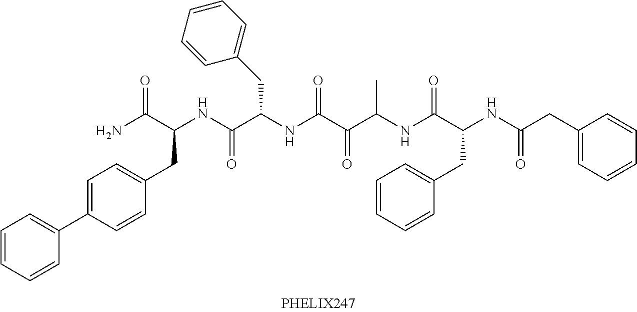 [SMILES: CC(NC(=O)[C@@H](Cc1ccccc1)NC(=O)Cc1ccccc1)C(=O)C(=O)N[C@@H](Cc1ccccc1)C(=O)N[C@@H](Cc1ccc(-c2ccccc2)cc1)C(N)=O]

Synthesized by Method B. Molecular Weight: 751.9 LC/MS: 752.3 (M+H)⁺

### Example 79

### N-((R)-1-(((S)-3-([1,1′-biphenyl]-4-yl)-1-amino-1-oxopropan-2-yl)amino)-1-oxo-3-phenylpropan-2-yl)-2-oxo-3-((S)-3-phenyl-2-(2-phenylacetamido)propanamido)butanamide

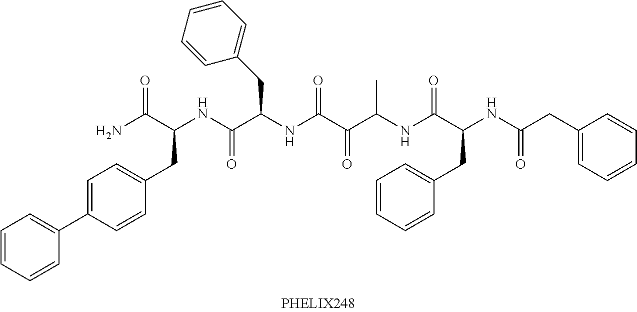 [SMILES: CC(NC(=O)[C@H](Cc1ccccc1)NC(=O)Cc1ccccc1)C(=O)C(=O)N[C@H](Cc1ccccc1)C(=O)N[C@@H](Cc1ccc(-c2ccccc2)cc1)C(N)=O]

Synthesized by Method B. Molecular Weight: 751.9 LC/MS: 752.2 (M+H)⁺

### Example 80

### N-((S)-1-((1-amino-2-methyl-1-oxopropan-2-yl)amino)-1-oxo-3-phenylpropan-2-yl)-2-oxo-3-((S)-3-phenyl-2-(2-phenylacetamido)propanamido)butanamide

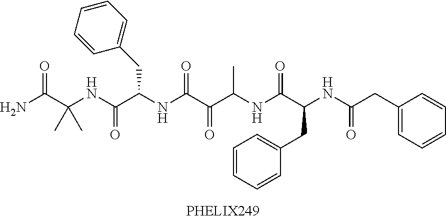 [SMILES: CC(NC(=O)[C@H](Cc1ccccc1)NC(=O)Cc1ccccc1)C(=O)C(=O)N[C@@H](Cc1ccccc1)C(=O)NC(C)(C)C(N)=O]

Synthesized by Method B. Molecular Weight: 613.7 LC/MS: 614.2 (M+H)⁺

### Example 81

### (2S)-N-(4-((2-(((S)-1-amino-3-(naphthalen-2-yl)-1-oxopropan-2-yl)amino)-2-oxoethyl)amino)-3,4-dioxobutan-2-yl)-2-(2,2-diphenylacetamido)-4-methylpentanamide

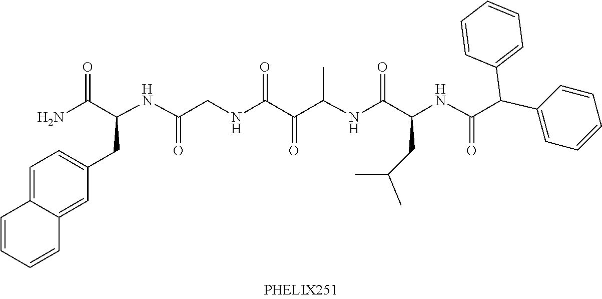 [SMILES: CC(C)C[C@H](NC(=O)C(c1ccccc1)c1ccccc1)C(=O)NC(C)C(=O)C(=O)NCC(=O)N[C@@H](Cc1ccc2ccccc2c1)C(N)=O]

Synthesized by Method B. Molecular Weight: 677.8 LC/MS: 678.5 (M+H)⁺

### Example 82

### (5S,12S,15S)-15-(((S)-1-amino-5-guanidino-1-oxopentan-2-yl)carbamoyl)-8-benzyl-5-isobutyl-12-methyl-3,6,9,10,13-pentaoxo-1-phenyl-2-oxa-4,7,11,14-tetraazaoctadecan-18-oic acid

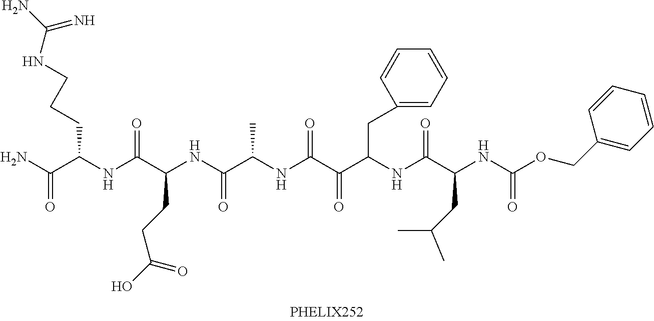 [SMILES: CC(C)C[C@H](NC(=O)OCc1ccccc1)C(=O)NC(Cc1ccccc1)C(=O)C(=O)N[C@@H](C)C(=O)N[C@@H](CCC(=O)O)C(=O)N[C@@H](CCCNC(=N)N)C(N)=O]

Synthesized by Method B. Molecular Weight: 795.9 LC/MS: 796.7 (M+H)⁺

### Example 83

### (2S)-2-(2-([1,1′-biphenyl]-4-yl)acetamido)-N-(4-((2-(((S)-1-amino-3-(naphthalen-2-yl)-1-oxopropan-2-yl)amino)-2-oxoethyl)amino)-3,4-dioxobutan-2-yl)-4-methylpentanamide

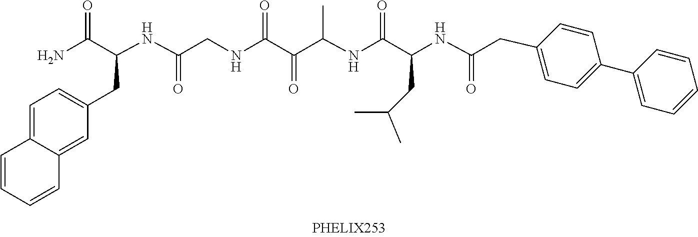 [SMILES: CC(C)C[C@H](NC(=O)Cc1ccc(-c2ccccc2)cc1)C(=O)NC(C)C(=O)C(=O)NCC(=O)N[C@@H](Cc1ccc2ccccc2c1)C(N)=O]

Synthesized by Method B. Molecular Weight: 677.8 LC/MS: 678.5 (M+H)⁺

### Example 84

### benzyl ((6S,13S,16S)-1-amino-10-benzyl-6-carbamoyl-1-imino-13-isobutyl-8,9,12,15-tetraoxo-17-phenyl-2,7,11,14-tetraazaheptadecan-16-yl)carbamate

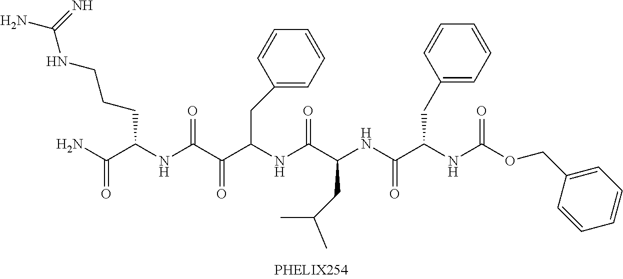 [SMILES: CC(C)C[C@H](NC(=O)[C@H](Cc1ccccc1)NC(=O)OCc1ccccc1)C(=O)NC(Cc1ccccc1)C(=O)C(=O)N[C@@H](CCCNC(=N)N)C(N)=O]

Synthesized by Method B. Molecular Weight: 742.9 LC/MS: 743.6 (M+H)⁺

### Example 85

### benzyl ((2S)-1-((4-((2-(((S)-1-amino-3-(naphthalen-2-yl)-1-oxopropan-2-yl)amino)-2-oxoethyl)amino)-3,4-dioxo-1-phenylbutan-2-yl)amino)-4-methyl-1-oxopentan-2-yl)carbamate

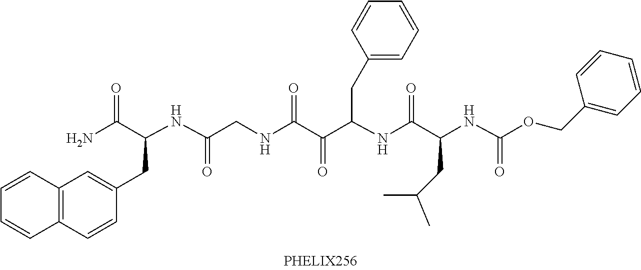 [SMILES: CC(C)C[C@H](NC(=O)OCc1ccccc1)C(=O)NC(Cc1ccccc1)C(=O)C(=O)NCC(=O)N[C@@H](Cc1ccc2ccccc2c1)C(N)=O]

Synthesized by Method B. Molecular Weight: 693.8 LC/MS: 694.5 (M+H)⁺

### Example 86

### benzyl ((2S)-1-(((4S)-1-((2-(((S)-1-amino-3-(naphthalen-2-yl)-1-oxopropan-2-yl)amino)-2-oxoethyl)amino)-4-methyl-1,2-dioxohexan-3-yl)amino)-4-methyl-1-oxopentan-2-yl)carbamate

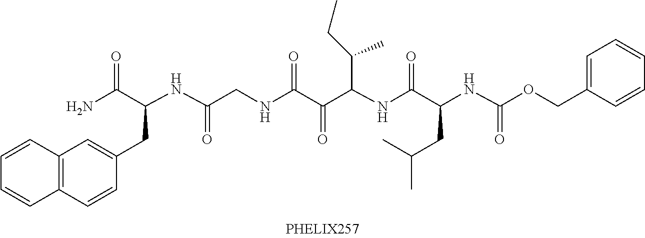 [SMILES: CC[C@H](C)C(NC(=O)[C@H](CC(C)C)NC(=O)OCc1ccccc1)C(=O)C(=O)NCC(=O)N[C@@H](Cc1ccc2ccccc2c1)C(N)=O]

Synthesized by Method B. Molecular Weight: 659.8. LC/MS: 660.4 (M+H)⁺

### Example 87

### benzyl ((2S,12S,15S)-1-amino-12-isobutyl-9-methyl-2-(naphthalen-2-ylmethyl)-1,4,7,8,11,14-hexaoxo-16-phenyl-3,6,10,13-tetraazahexadecan-15-yl)carbamate

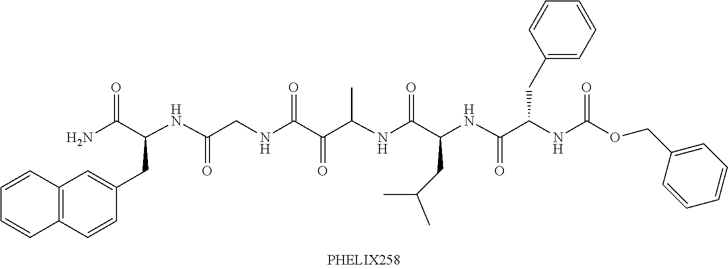 [SMILES: CC(C)C[C@H](NC(=O)[C@H](Cc1ccccc1)NC(=O)OCc1ccccc1)C(=O)NC(C)C(=O)C(=O)NCC(=O)N[C@@H](Cc1ccc2ccccc2c1)C(N)=O]

Synthesized by Method B. Molecular Weight: 764.9 LC/MS: 765.5 (M+H)⁺

### Example 88

### N-((S)-1-(((S)-3-([1,1′-biphenyl]-4-yl)-1-amino-1-oxopropan-2-yl)amino)-3-cyclohexyl-1-oxopropan-2-yl)-2-oxo-3-((S)-3-phenyl-2-(2-phenylacetamido)propanamido)butanamide

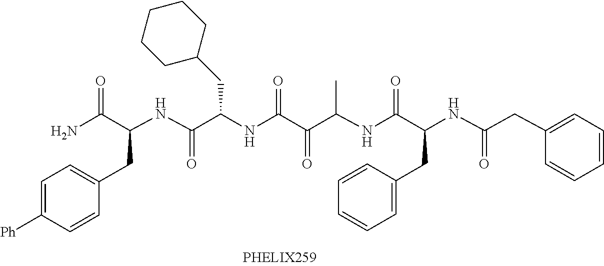 [SMILES: CC(NC(=O)[C@H](Cc1ccccc1)NC(=O)Cc1ccccc1)C(=O)C(=O)N[C@@H](CC1CCCCC1)C(=O)N[C@@H](Cc1ccc(-c2ccccc2)cc1)C(N)=O]

Synthesized by method B. Molecular Weight: 757.93 Observed molecular weight (M+H)⁺ 758.65

### Example 89

### N-((S)-1-(((S)-3-([1,1′-biphenyl]-4-yl)-1-amino-1-oxopropan-2-yl)amino)-3-cyclopropyl-1-oxopropan-2-yl)-2-oxo-3-((S)-3-phenyl-2-(2-phenylacetamido)propanamido)butanamide

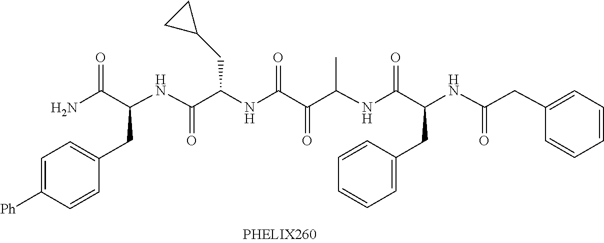 [SMILES: CC(NC(=O)[C@H](Cc1ccccc1)NC(=O)Cc1ccccc1)C(=O)C(=O)N[C@@H](CC1CC1)C(=O)N[C@@H](Cc1ccc(-c2ccccc2)cc1)C(N)=O]

Synthesized by method B. Molecular Weight: 715.85 Observed molecular weight (M+H)⁺ 716.52

### Example 90

### N-((S)-1-(((S)-3-([1,1′-biphenyl]-4-yl)-1-amino-1-oxopropan-2-yl)amino)-1-oxo-3-(4-(trifluoromethyl)phenyl)propan-2-yl)-2-oxo-3-((S)-3-phenyl-2-(2phenylacetamido)propanamido)butanamide

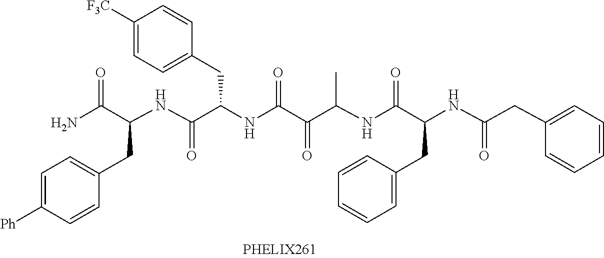 [SMILES: CC(NC(=O)[C@H](Cc1ccccc1)NC(=O)Cc1ccccc1)C(=O)C(=O)N[C@@H](Cc1ccc(C(F)(F)F)cc1)C(=O)N[C@@H](Cc1ccc(-c2ccccc2)cc1)C(N)=O]

Synthesized by method B. Molecular Weight: 819.88 Observed molecular weight (M+H)⁺ 820.65

### Example 91

### N-((S)-1-(((S)-3-([1,1′-biphenyl]-4-yl)-1-amino-1-oxopropan-2-yl)amino)-3-(4-fluorophenyl)-1-oxopropan-2-yl)-2-oxo-3-((S)-3-phenyl-2-(2-phenylacetamido)propanamido)butanamide

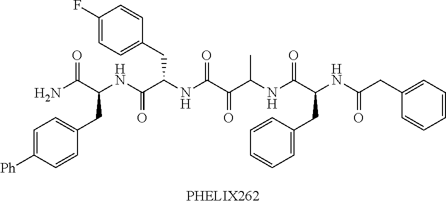 [SMILES: CC(NC(=O)[C@H](Cc1ccccc1)NC(=O)Cc1ccccc1)C(=O)C(=O)N[C@@H](Cc1ccc(F)cc1)C(=O)N[C@@H](Cc1ccc(-c2ccccc2)cc1)C(N)=O]

Synthesized by method B. Molecular Weight: 769.87 Observed molecular weight (M+H)⁺ 770.63

### Example 92

### N-((S)-1-(((S)-3-([1,1′-biphenyl]-4-yl)-1-amino-1-oxopropan-2-yl)amino)-3-(2-fluorophenyl)-1-oxopropan-2-yl)-2-oxo-3-((S)-3-phenyl-2-(2-phenylacetamido)propanamido)butanamide

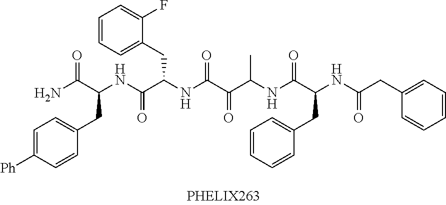 [SMILES: CC(NC(=O)[C@H](Cc1ccccc1)NC(=O)Cc1ccccc1)C(=O)C(=O)N[C@@H](Cc1ccccc1F)C(=O)N[C@@H](Cc1ccc(-c2ccccc2)cc1)C(N)=O]

Synthesized by method B. Molecular Weight: 769.87 Observed molecular weight (M+H)⁺ 770.57

### Example 93

### N-((S)-1-(((S)-3-([1,1′-biphenyl]-4-yl)-1-amino-1-oxopropan-2-yl)amino)-1-oxo-3-(3-(trifluoromethyl)phenyl)propan-2-yl)-2-oxo-3-((S)-3-phenyl-2-(2-phenylacetamido)propanamido)butanamide

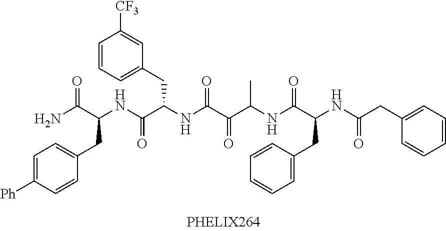 [SMILES: CC(NC(=O)[C@H](Cc1ccccc1)NC(=O)Cc1ccccc1)C(=O)C(=O)N[C@@H](Cc1cccc(C(F)(F)F)c1)C(=O)N[C@@H](Cc1ccc(-c2ccccc2)cc1)C(N)=O]

Synthesized by method B. Molecular Weight: 819.88 Observed molecular weight (M+H)⁺ 820.65

### Example 94

### N-((S)-1-(((S)-3-([1,1′-biphenyl]-4-yl)-1-amino-1-oxopropan-2-yl)amino)-1-oxo-3-(2-(trifluoromethyl)phenyl)propan-2-yl)-2-oxo-3-((S)-3-phenyl-2-(2-phenylacetamido)propanamido)butanamide

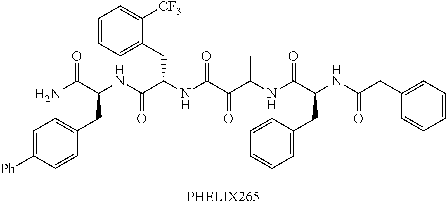 [SMILES: CC(NC(=O)[C@H](Cc1ccccc1)NC(=O)Cc1ccccc1)C(=O)C(=O)N[C@@H](Cc1ccccc1C(F)(F)F)C(=O)N[C@@H](Cc1ccc(-c2ccccc2)cc1)C(N)=O]

Synthesized by method B. Molecular Weight: 819.88 Observed molecular weight (M+H)⁺ 820.59

### Example 95

### N-((S)-1-(((S)-3-([1,1′-biphenyl]-4-yl)-1-amino-1-oxopropan-2-yl)amino)-3-(3-fluorophenyl)-1-oxopropan-2-yl)-2-oxo-3-((S)-3-phenyl-2-(2-phenylacetamido)propanamido)butanamide

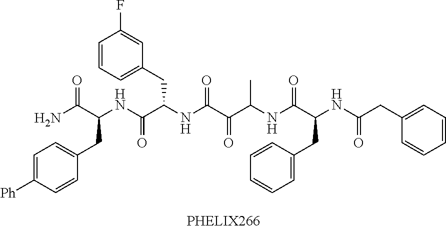 [SMILES: CC(NC(=O)[C@H](Cc1ccccc1)NC(=O)Cc1ccccc1)C(=O)C(=O)N[C@@H](Cc1cccc(F)c1)C(=O)N[C@@H](Cc1ccc(-c2ccccc2)cc1)C(N)=O]

Synthesized by method B. Molecular Weight: 769.87 Observed molecular weight (M+H)⁺ 770.54

### Example 96

### benzyl ((6S,13S,16S)-1-amino-10-((S)-sec-butyl)-6-carbamoyl-1-imino-13-isobutyl-8,9,12,15-tetraoxo-17-phenyl-2,7,11,14-tetraazaheptadecan-16-yl)carbamate

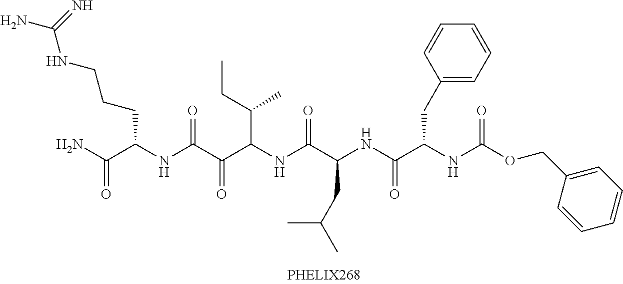 [SMILES: CC[C@H](C)C(NC(=O)[C@H](CC(C)C)NC(=O)[C@H](Cc1ccccc1)NC(=O)OCc1ccccc1)C(=O)C(=O)N[C@@H](CCCNC(=N)N)C(N)=O]

Synthesized by Method B. Molecular Weight: 708.9 LC/MS: 710.1 (M+H)⁺

### Example 97

### benzyl ((11S,18S,21S)-15-((S)-sec-butyl)-11-carbamoyl-18-isobutyl-5,13,14,17,20-pentaoxo-22-phenyl-4-oxa-6,12,16,19-tetraazadocos-1-en-21-yl)carbamate

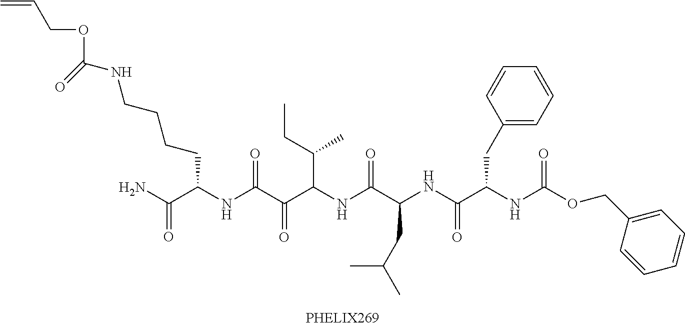 [SMILES: C=CCOC(=O)NCCCC[C@H](NC(=O)C(=O)C(NC(=O)[C@H](CC(C)C)NC(=O)[C@H](Cc1ccccc1)NC(=O)OCc1ccccc1)[C@@H](C)CC)C(N)=O]

Synthesized by Method B. Molecular Weight: 764.9 LC/MS: 765.6 (M+H)⁺

### Example 98

### benzyl ((2S,12S,15S)-1-amino-9-((S)-sec-butyl)-12-isobutyl-2-(naphthalen-2-ylmethyl)-1,4,7,8,11,14-hexaoxo-16-phenyl-3,6,10,13-tetraazahexadecan-15-yl)carbamate

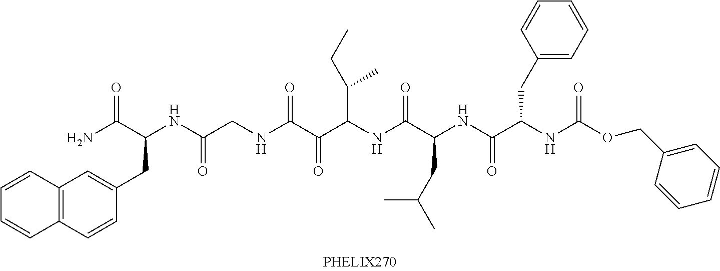 [SMILES: CC[C@H](C)C(NC(=O)[C@H](CC(C)C)NC(=O)[C@H](Cc1ccccc1)NC(=O)OCc1ccccc1)C(=O)C(=O)NCC(=O)N[C@@H](Cc1ccc2ccccc2c1)C(N)=O]

Synthesized by Method B. Molecular Weight: 807.0 LC/MS: 807.6 (M+H)⁺

### Example 99

### benzyl ((2S)-1-(((4S)-1-(((S)-1-(((S)-1-amino-3-(naphthalen-2-yl)-1-oxopropan-2-yl)amino)-5-guanidino-1-oxopentan-2-yl)amino)-4-methyl-1,2-dioxohexan-3-yl)amino)-4-methyl-1-oxopentan-2-yl)carbamate

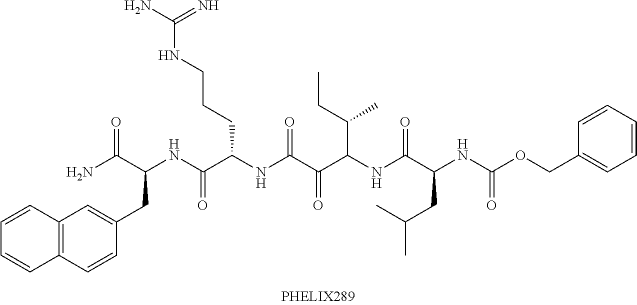 [SMILES: CC[C@H](C)C(NC(=O)[C@H](CC(C)C)NC(=O)OCc1ccccc1)C(=O)C(=O)N[C@@H](CCCNC(=N)N)C(=O)N[C@@H](Cc1ccc2ccccc2c1)C(N)=O]

Synthesized by Method B. Molecular Weight: 758.9 LC/MS: 760.1 (M+H)⁺

### Example 100

### benzyl ((2S)-1-(((4S)-1-(((S)-1-amino-5-guanidino-1-oxopentan-2-yl)amino)-4-methyl-1,2-dioxohexan-3-yl)amino)-4-methyl-1-oxopentan-2-yl)carbamate

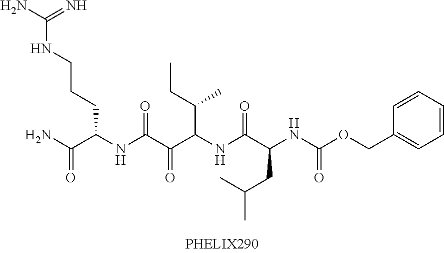 [SMILES: CC[C@H](C)C(NC(=O)[C@H](CC(C)C)NC(=O)OCc1ccccc1)C(=O)C(=O)N[C@@H](CCCNC(=N)N)C(N)=O]

Synthesized by Method B. Molecular Weight: 561.7 LC/MS: 562.9 (M+H)⁺

### Example 101

### benzyl ((2S)-1-(((4S)-1-((2-(((S)-1-amino-3-(naphthalen-2-yl)-1-oxopropan-2-yl)amino)-2-oxoethyl)(methyl)amino)-4-methyl-1,2-dioxohexan-3-yl)amino)-4-methyl-1-oxopentan-2-yl)carbamate

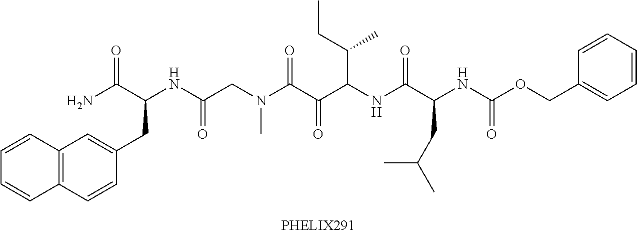 [SMILES: CC[C@H](C)C(NC(=O)[C@H](CC(C)C)NC(=O)OCc1ccccc1)C(=O)C(=O)N(C)CC(=O)N[C@@H](Cc1ccc2ccccc2c1)C(N)=O]

Synthesized by Method B. Molecular Weight: 673.8 LC/MS: 674.6 (M+H)⁺

### Example 102

### benzyl ((2S)-1-(((4S)-1-((2-(((S)-1-amino-3-(naphthalen-2-yl)-1-oxopropan-2-yl)amino)-2-oxoethyl)amino)-4-methyl-1,2-dioxohexan-3-yl)(methyl)amino)-4-methyl-1-oxopentan-2-yl)carbamate

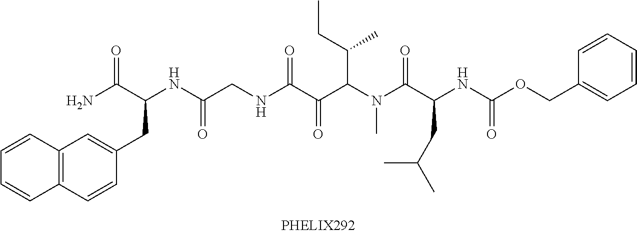 [SMILES: CC[C@H](C)C(C(=O)C(=O)NCC(=O)N[C@@H](Cc1ccc2ccccc2c1)C(N)=O)N(C)C(=O)[C@H](CC(C)C)NC(=O)OCc1ccccc1]

Synthesized by Method B. Molecular Weight: 673.8 LC/MS: 674.6 (M+H)⁺

### Example 103

### benzyl ((2R)-1-(((4S)-1-((2-(((S)-1-amino-3-(naphthalen-2-yl)-1-oxopropan-2-yl)amino)-2-oxoethyl)amino)-4-methyl-1,2-dioxohexan-3-yl)amino)-4-methyl-1-oxopentan-2-yl)carbamate

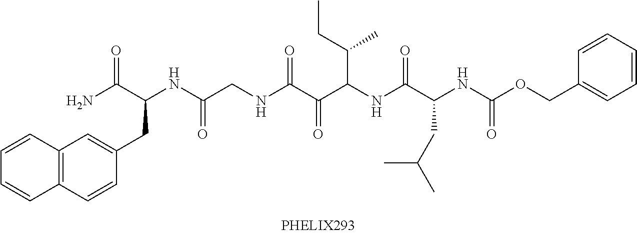 [SMILES: CC[C@H](C)C(NC(=O)[C@@H](CC(C)C)NC(=O)OCc1ccccc1)C(=O)C(=O)NCC(=O)N[C@@H](Cc1ccc2ccccc2c1)C(N)=O]

Synthesized by Method B. Molecular Weight: 659.8 LC/MS: 660.5 (M+H)⁺

### Example 104

### benzyl ((6R,13S,16S)-1-amino-10-((S)-sec-butyl)-6-carbamoyl-1-imino-13-isobutyl-8,9,12,15-tetraoxo-17-phenyl-2,7,11,14-tetraazaheptadecan-16-yl)carbamate

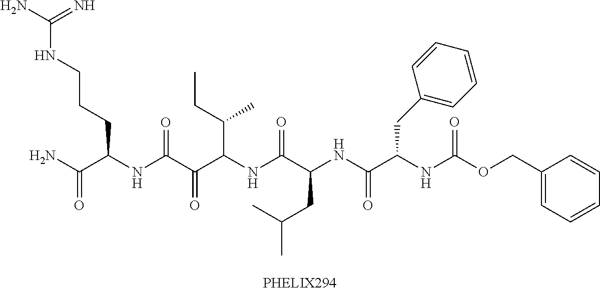 [SMILES: CC[C@H](C)C(NC(=O)[C@H](CC(C)C)NC(=O)[C@H](Cc1ccccc1)NC(=O)OCc1ccccc1)C(=O)C(=O)N[C@H](CCCNC(=N)N)C(N)=O]

Synthesized by Method B. Molecular Weight: 708.9 LC/MS: 710.2 (M+H)⁺

### Example 105

### benzyl ((2S)-1-(((4S)-1-(((S)-1-(((S)-1-amino-3-(naphthalen-2-yl)-1-oxopropan-2-yl)amino)-1-oxopropan-2-yl)amino)-4-methyl-1,2-dioxohexan-3-yl)amino)-4-methyl-1-oxopentan-2-yl)carbamate

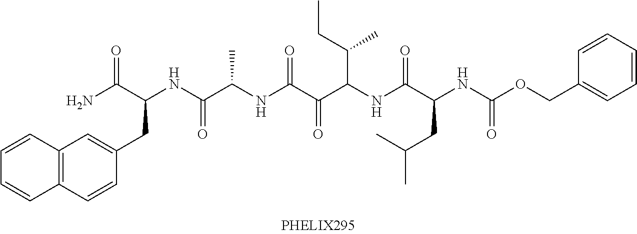 [SMILES: CC[C@H](C)C(NC(=O)[C@H](CC(C)C)NC(=O)OCc1ccccc1)C(=O)C(=O)N[C@@H](C)C(=O)N[C@@H](Cc1ccc2ccccc2c1)C(N)=O]

Synthesized by Method B. Molecular Weight: 673.8. LC/MS: 674.40 (M+1)⁺

### Example 106

### benzyl ((2S)-1-(((4S)-1-((1-(((S)-1-amino-3-(naphthalen-2-yl)-1-oxopropan-2-yl)amino)-2-methyl-1-oxopropan-2-yl)amino)-4-methyl-1,2-dioxohexan-3-yl)amino)-4-methyl-1-oxopentan-2-yl)carbamate

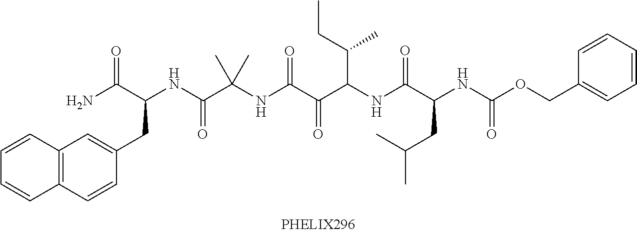 [SMILES: CC[C@H](C)C(NC(=O)[C@H](CC(C)C)NC(=O)OCc1ccccc1)C(=O)C(=O)NC(C)(C)C(=O)N[C@@H](Cc1ccc2ccccc2c1)C(N)=O]

Synthesized by Method B. Molecular Weight: 687.8. LC/MS: 688.26 (M+1)⁺

### Example 107

### benzyl ((2S)-1-(((4S)-1-(3-(((S)-1-amino-3-(naphthalen-2-yl)-1-oxopropan-2-yl)carbamoyl)azetidin-1-yl)-4-methyl-1,2-dioxohexan-3-yl)amino)-4-methyl-1-oxopentan-2-yl)carbamate

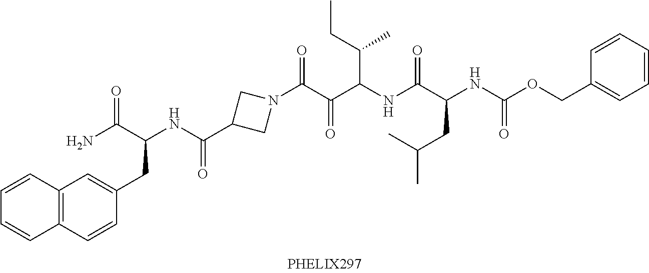 [SMILES: CC[C@H](C)C(NC(=O)[C@H](CC(C)C)NC(=O)OCc1ccccc1)C(=O)C(=O)N1CC(C(=O)N[C@@H](Cc2ccc3ccccc3c2)C(N)=O)C1]

Synthesized by Method B. Molecular Weight: 685.82. LC/MS: 686.30 (M+1)⁺

### Example 108

### benzyl ((2S)-1-(((4S)-1-((1-(((S)-1-amino-3-(naphthalen-2-yl)-1-oxopropan-2-yl)carbamoyl)cyclopropyl)amino)-4-methyl-1,2-dioxohexan-3-yl)amino)-4-methyl-1-oxopentan-2-yl)carbamate

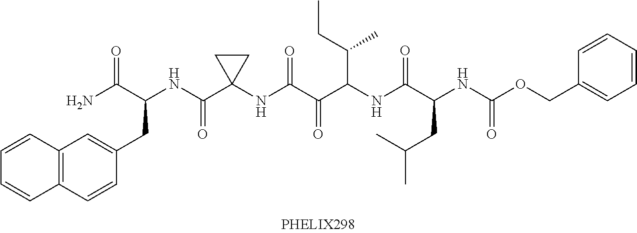 [SMILES: CC[C@H](C)C(NC(=O)[C@H](CC(C)C)NC(=O)OCc1ccccc1)C(=O)C(=O)NC1(C(=O)N[C@@H](Cc2ccc3ccccc3c2)C(N)=O)CC1]

Synthesized by Method B. Molecular Weight: 685.8. LC/MS: 686.30 (M+1)⁺

### Example 109

### benzyl ((2S)-1-(((4S)-1-((2-(((S)-1-amino-3-(naphthalen-2-yl)-1-oxopropan-2-yl)amino)-2-oxoethyl)amino)-4-methyl-1,2-dioxohexan-3-yl)amino)-3-cyclopropyl-1-oxopropan-2-yl)carbamate

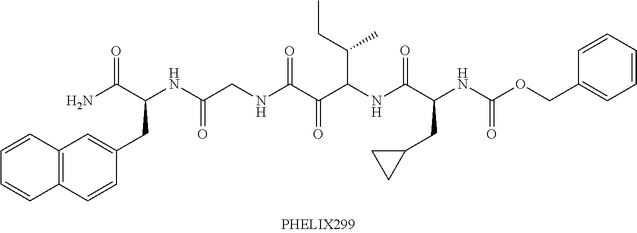 [SMILES: CC[C@H](C)C(NC(=O)[C@H](CC1CC1)NC(=O)OCc1ccccc1)C(=O)C(=O)NCC(=O)N[C@@H](Cc1ccc2ccccc2c1)C(N)=O]

Synthesized by Method B. Molecular Weight: 657.8. LC/MS: 658.44 (M+1)⁺

### Example 110

### (4S)-N-(2-(((S)-1-amino-3-(naphthalen-2-yl)-1-oxopropan-2-yl)amino)-2-oxoethyl)-4-methyl-3-((S)-4-methyl-2-(2-phenylacetamido)pentanamido)-2-oxohexanamide

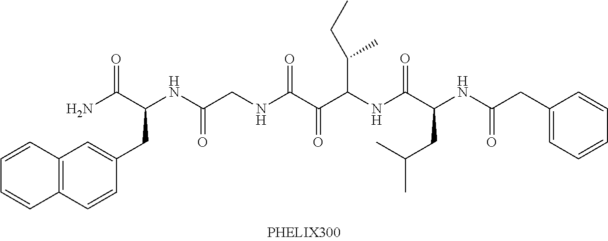 [SMILES: CC[C@H](C)C(NC(=O)[C@H](CC(C)C)NC(=O)Cc1ccccc1)C(=O)C(=O)NCC(=O)N[C@@H](Cc1ccc2ccccc2c1)C(N)=O]

Synthesized by method B. Molecular Weight: 643.79 Observed molecular weight (M+H)⁺ 644.54

### Example 111

### benzyl ((4S,14S)-11-((S)-sec-butyl)-2,16-dimethyl-4-(naphthalen-2-ylmethyl)-3,6,9,10,13-pentaoxo-2,5,8,12-tetraazaheptadecan-14-yl)carbamate

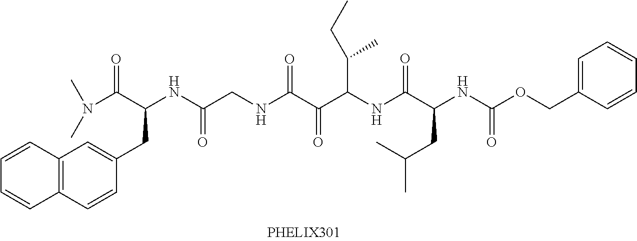 [SMILES: CC[C@H](C)C(NC(=O)[C@H](CC(C)C)NC(=O)OCc1ccccc1)C(=O)C(=O)NCC(=O)N[C@@H](Cc1ccc2ccccc2c1)C(=O)N(C)C]

Synthesized by method B. Molecular Weight: 687.84 Observed molecular weight (M+H)⁺ 688.65

### Example 112

### (4S)-3-(3-(1H-imidazol-4-yl)-2-(2-phenylacetamido)propanamido)-N-(2-(((S)-1-amino-3-(naphthalen-2-yl)-1-oxopropan-2-yl)amino)-2-oxoethyl)-4-methyl-2-oxohexanamide

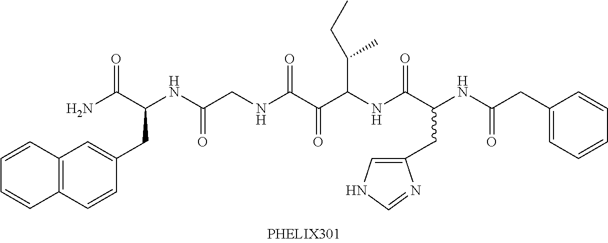 [SMILES: CC[C@H](C)C(NC(=O)C(Cc1c[nH]cn1)NC(=O)Cc1ccccc1)C(=O)C(=O)NCC(=O)N[C@@H](Cc1ccc2ccccc2c1)C(N)=O]

Synthesized by method B. Molecular Weight: 667.77 Observed molecular weight (M+H)⁺ 668.55

### Example 113

### benzyl ((2S)-4-methyl-1-(((4S)-4-methyl-1-((2-((naphthalen-2-ylmethyl)amino)-2-oxoethyl)amino)-1,2-dioxohexan-3-yl)amino)-1-oxopentan-2-yl)carbamate

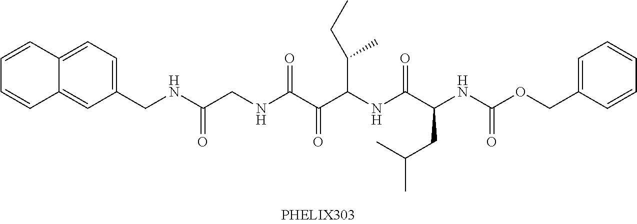 [SMILES: CC[C@H](C)C(NC(=O)[C@H](CC(C)C)NC(=O)OCc1ccccc1)C(=O)C(=O)NCC(=O)NCc1ccc2ccccc2c1]

Synthesized by Method D. Molecular Weight: 602.7 LC/MS: 603.4 (M+H)⁺

### Example 114

### benzyl ((2S)-1-(((4S)-1-((2-(((R)-1-amino-3-(naphthalen-2-yl)-1-oxopropan-2-yl)amino)-2-oxoethyl)amino)-4-methyl-1,2-dioxohexan-3-yl)amino)-4-methyl-1-oxopentan-2-yl)carbamate

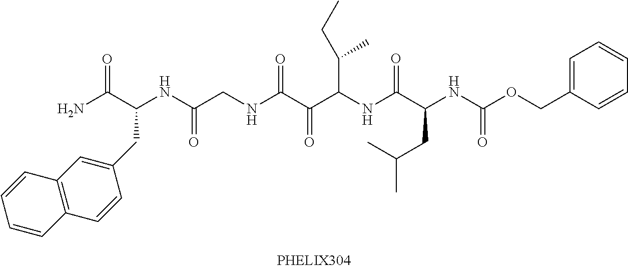 [SMILES: CC[C@H](C)C(NC(=O)[C@H](CC(C)C)NC(=O)OCc1ccccc1)C(=O)C(=O)NCC(=O)N[C@H](Cc1ccc2ccccc2c1)C(N)=O]

Synthesized by Method B. Molecular Weight: 659.8 LC/MS: 660.5 (M+H)⁺

### Example 115

### benzyl ((2S)-1-(((4S)-1-((2-(((S)-1-amino-3-(naphthalen-2-yl)-1-oxopropan-2-yl)amino)-2-oxoethyl)amino)-4-methyl-1,2-dioxohexan-3-yl)(methyl)amino)-4-methyl-1-oxopentan-2-yl)carbamate

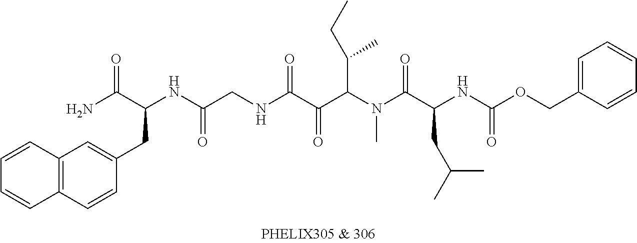 [SMILES: CC[C@H](C)C(C(=O)C(=O)NCC(=O)N[C@@H](Cc1ccc2ccccc2c1)C(N)=O)N(C)C(=O)[C@H](CC(C)C)NC(=O)OCc1ccccc1]

Synthesized by Method B. Molecular Weight: 673.8 LC/MS: 674.5 (M+H)⁺

### Example 116

### (4S)-3-((S)-2-acetamido-4-methylpentanamido)-N-(2-(((S)-1-amino-3-(naphthalen-2-yl)-1-oxopropan-2-yl)amino)-2-oxoethyl)-4-methyl-2-oxohexanamide

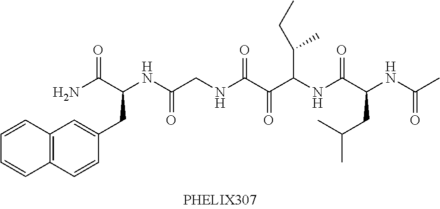 [SMILES: CC[C@H](C)C(NC(=O)[C@H](CC(C)C)NC(C)=O)C(=O)C(=O)NCC(=O)N[C@@H](Cc1ccc2ccccc2c1)C(N)=O]

Synthesized by Method B. Molecular Weight: 714.9 LC/MS: 715.6 (M+H)⁺

### Example 117

### (4S)-3-(3-(1H-imidazol-4-yl)-2-(2-phenylacetamido)propanamido)-N-(2-(((S)-1-amino-3-(naphthalen-2-yl)-1-oxopropan-2-yl)amino)-2-oxoethyl)-4-methyl-2-oxohexanamide

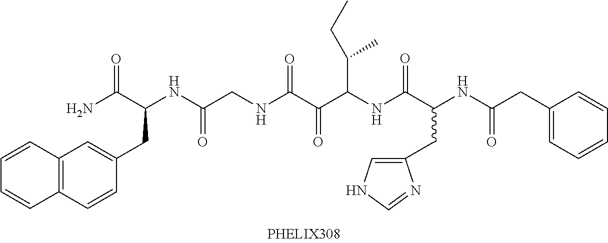 [SMILES: CC[C@H](C)C(NC(=O)C(Cc1c[nH]cn1)NC(=O)Cc1ccccc1)C(=O)C(=O)NCC(=O)N[C@@H](Cc1ccc2ccccc2c1)C(N)=O]

Synthesized by method B. Molecular Weight: 667.77 Observed molecular weight (M+H)⁺ 669.49

### Example 118

### benzyl ((2S)-1-(((4S)-1-(((S)-1-amino-3-(naphthalen-2-yl)-1-oxopropan-2-yl)amino)-4-methyl-1,2-dioxohexan-3-yl)amino)-4-methyl-1-oxopentan-2-yl)carbamate

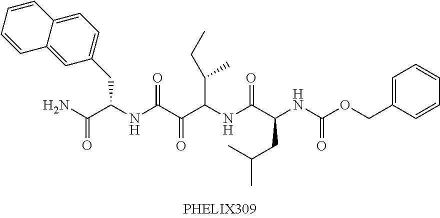 [SMILES: CC[C@H](C)C(NC(=O)[C@H](CC(C)C)NC(=O)OCc1ccccc1)C(=O)C(=O)N[C@@H](Cc1ccc2ccccc2c1)C(N)=O]

Synthesized by Method B. Molecular Weight: 602.7 LC/MS: 603.5(M+H)⁺

### Example 119

### (4S)-3-((S)-2-acetamido-4-methylpentanamido)-N-(2-(((S)-1-amino-3-(naphthalen-2-yl)-1-oxopropan-2-yl)amino)-2-oxoethyl)-4-methyl-2-oxohexanamide

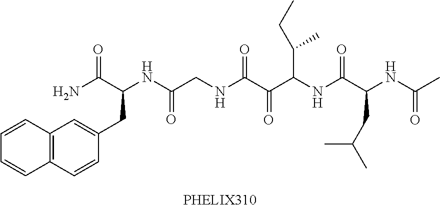 [SMILES: CC[C@H](C)C(NC(=O)[C@H](CC(C)C)NC(C)=O)C(=O)C(=O)NCC(=O)N[C@@H](Cc1ccc2ccccc2c1)C(N)=O]

Synthesized by method B. Molecular Weight: 567.69 Observed molecular weight (M+H)⁺ 568.60

### Example 120

### benzyl ((2S)-1-(((4S)-1-((2-(((S)-1-amino-3-(naphthalen-2-yl)-1-oxopropan-2-yl)amino)-2-oxoethyl)amino)-4-methyl-1,2-dioxohexan-3-yl)amino)-3-(1H-imidazol-4-yl)-1-oxopropan-2-yl)carbamate

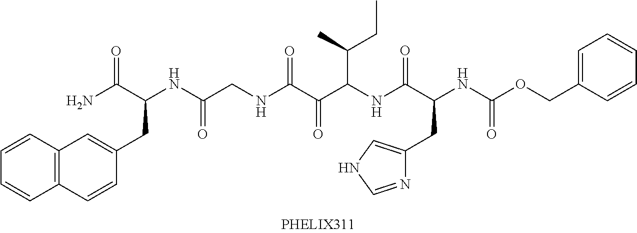 [SMILES: CC[C@H](C)C(NC(=O)[C@H](Cc1c[nH]cn1)NC(=O)OCc1ccccc1)C(=O)C(=O)NCC(=O)N[C@@H](Cc1ccc2ccccc2c1)C(N)=O]

Synthesized by method C. Molecular Weight: 683.77 Observed molecular weight (M+H)⁺ 684.62

### Example 121

### (4S)-N-(2-(((S)-1-amino-3-(naphthalen-2-yl)-1-oxopropan-2-yl)amino)-2-oxoethyl)-4-methyl-3-((S)-4 methyl-2-(2-(pyridin-3-yl)acetamido)pentanamido)-2-oxohexanamide

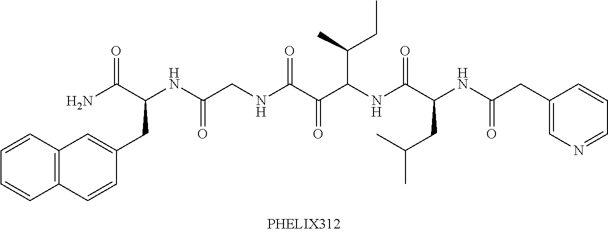 [SMILES: CC[C@H](C)C(NC(=O)[C@H](CC(C)C)NC(=O)Cc1cccnc1)C(=O)C(=O)NCC(=O)N[C@@H](Cc1ccc2ccccc2c1)C(N)=O]

Synthesized by method C. Molecular Weight: 644.77

### Example 122

### (4S)-N-(2-(((S)-1-amino-3-(naphthalen-2-yl)-1-oxopropan-2-yl)amino)-2-oxoethyl)-4-methyl-3-((S)-4-methyl-2-(2-(4-methylpiperazin-1-yl)acetamido)pentanamido)-2-oxohexanamide

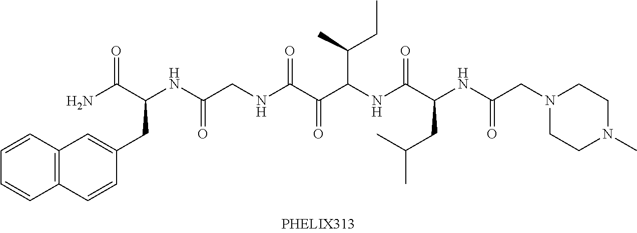 [SMILES: CC[C@H](C)C(NC(=O)[C@H](CC(C)C)NC(=O)CN1CCN(C)CC1)C(=O)C(=O)NCC(=O)N[C@@H](Cc1ccc2ccccc2c1)C(N)=O]

Synthesized by method B. Molecular Weight: 665.84 Observed molecular weight (M+H)⁺ 666.72

### Example 123

### (4S)-N-(2-(((S)-1-amino-3-(naphthalen-2-yl)-1-oxopropan-2-yl)amino)-2-oxoethyl)-4-methyl-3-((S)-4-methyl-2-(2-morpholinoacetamido)pentanamido)-2-oxohexanamide

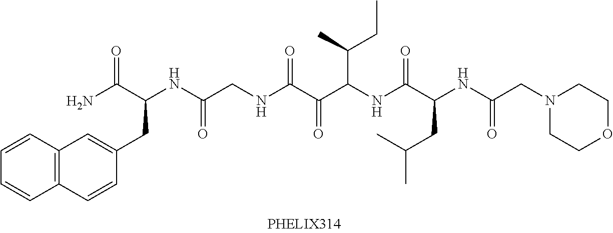 [SMILES: CC[C@H](C)C(NC(=O)[C@H](CC(C)C)NC(=O)CN1CCOCC1)C(=O)C(=O)NCC(=O)N[C@@H](Cc1ccc2ccccc2c1)C(N)=O]

Synthesized by method B. Molecular Weight: 652.79 Observed molecular weight (M+H)⁺ 653.61

### Example 124

### benzyl ((2S)-1-(((4S)-1-(((S)-1-(((S)-1-amino-3-(naphthalen-2-yl)-1-oxopropan-2-yl)amino)-1-oxo-5-ureidopentan-2-yl)amino)-4-methyl-1,2-dioxohexan-3-yl)amino)-4-methyl-1-oxopentan-2-yl)carbamate

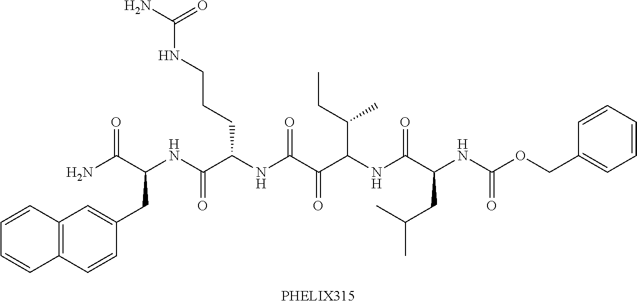 [SMILES: CC[C@H](C)C(NC(=O)[C@H](CC(C)C)NC(=O)OCc1ccccc1)C(=O)C(=O)N[C@@H](CCCNC(N)=O)C(=O)N[C@@H](Cc1ccc2ccccc2c1)C(N)=O]

Synthesized by Method B. Molecular Weight: 759.9 LC/MS: 760.6 (M+H)⁺

### Example 125: benzyl ((2S)-1-(((4S)-1-(((S)-4-amino-1-(((S)-1-amino-3-(naphthalen-2-yl)-1-oxopropan-2-yl)amino)-1,4-dioxobutan-2-yl)amino)-4-methyl-1,2-dioxohexan-3-yl)amino)-4-methyl-1-oxopentan-2-yl)carbamate

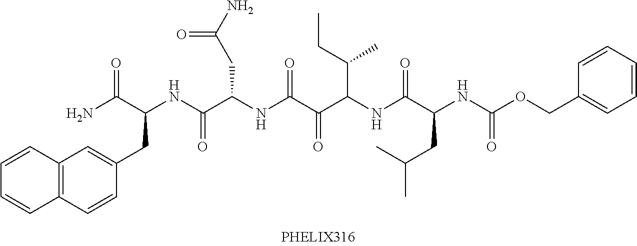 [SMILES: CC[C@H](C)C(NC(=O)[C@H](CC(C)C)NC(=O)OCc1ccccc1)C(=O)C(=O)N[C@@H](CC(N)=O)C(=O)N[C@@H](Cc1ccc2ccccc2c1)C(N)=O]

Synthesized by Method B. Molecular Weight: 716.8 LC/MS: 717.6 (M+H)⁺

### Example 126

### benzyl ((2S)-1-(((4S)-1-(((S)-5-amino-1-(((S)-1-amino-3-(naphthalen-2-yl)-1-oxopropan-2-yl)amino)-1,5-dioxopentan-2-yl)amino)-4-methyl-1,2-dioxohexan-3-yl)amino)-4-methyl-1-oxopentan-2-yl)carbamate

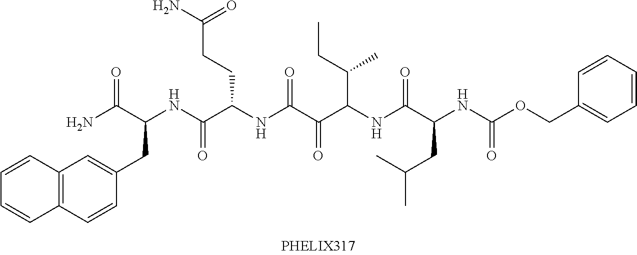 [SMILES: CC[C@H](C)C(NC(=O)[C@H](CC(C)C)NC(=O)OCc1ccccc1)C(=O)C(=O)N[C@@H](CCC(N)=O)C(=O)N[C@@H](Cc1ccc2ccccc2c1)C(N)=O]

Synthesized by Method B. Molecular Weight: 730.8 LC/MS: 731.6 (M+H)⁺

### Example 127

### benzyl ((2S)-1-(((4S)-1-((2-(((S)-1-amino-1-oxo-3-phenylpropan-2-yl)amino)-2-oxoethyl)amino)-4-methyl-1,2-dioxohexan-3-yl)amino)-4-methyl-1-oxopentan-2-yl)carbamate

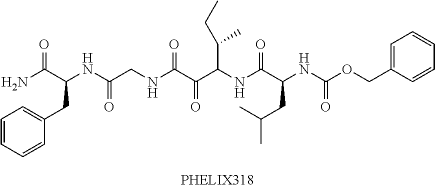 [SMILES: CC[C@H](C)C(NC(=O)[C@H](CC(C)C)NC(=O)OCc1ccccc1)C(=O)C(=O)NCC(=O)N[C@@H](Cc1ccccc1)C(N)=O]

Synthesized by Method B. Molecular Weight: 609.7 LC/MS: 610.5 (M+H)⁺

### Example 128

### benzyl ((2S)-1-(((4S)-1-((2-(((S)-1-amino-1-oxo-3-phenylpropan-2-yl)(methyl)amino)-2-oxoethyl)amino)-4-methyl-1,2-dioxohexan-3-yl)amino)-4-methyl-1-oxopentan-2-yl)carbamate

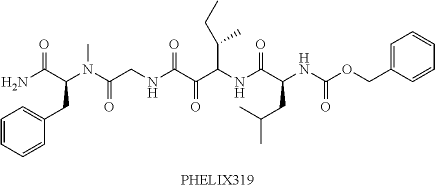 [SMILES: CC[C@H](C)C(NC(=O)[C@H](CC(C)C)NC(=O)OCc1ccccc1)C(=O)C(=O)NCC(=O)N(C)[C@@H](Cc1ccccc1)C(N)=O]

Synthesized by Method B. Molecular Weight: 623.8 LC/MS: 624.5 (M+H)⁺

### Example 129

### (2S)-N¹-((4S)-1-((2-(((S)-1-amino-3-(naphthalen-2-yl)-1-oxopropan-2-yl)amino)-2-oxoethyl)amino)-4-methyl-1,2-dioxohexan-3-yl)-2-(2-phenylacetamido)pentanediamide

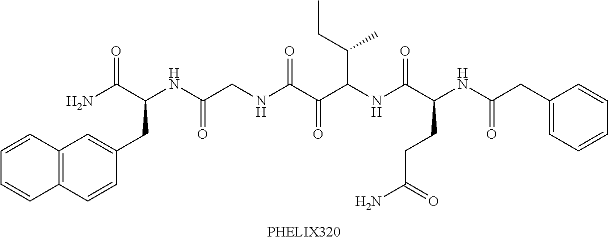 [SMILES: CC[C@H](C)C(NC(=O)[C@H](CCC(N)=O)NC(=O)Cc1ccccc1)C(=O)C(=O)NCC(=O)N[C@@H](Cc1ccc2ccccc2c1)C(N)=O]

Synthesized by Method B. Molecular Weight: 658.8. LC/MS: 659.35 (M+1)⁺

### Example 130

### (4S)-N-(2-(((S)-1-amino-3-(naphthalen-2-yl)-1-oxopropan-2-yl)amino)-2-oxoethyl)-4-methyl-2-oxo-3-((S)-3-phenyl-2-(2-phenylacetamido)propanamido)hexanamide

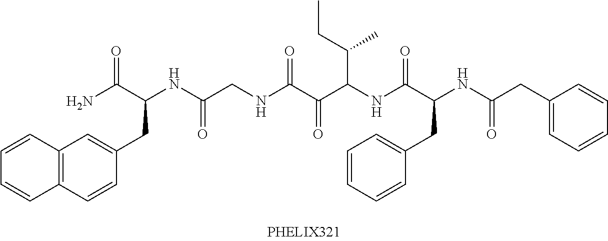 [SMILES: CC[C@H](C)C(NC(=O)[C@H](Cc1ccccc1)NC(=O)Cc1ccccc1)C(=O)C(=O)NCC(=O)N[C@@H](Cc1ccc2ccccc2c1)C(N)=O]

Synthesized by Method B. Molecular Weight: 677.8. LC/MS: 678.32 (M+1)⁺

### Example 131

### (2S)-N¹-((4S)-1-((2-(((S)-1-amino-3-(naphthalen-2-yl)-1-oxopropan-2-yl)amino)-2-oxoethyl)amino)-4-methyl-1,2-dioxohexan-3-yl)-2-(2-phenylacetamido)succinamide

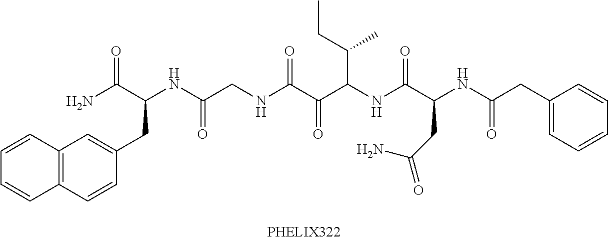 [SMILES: CC[C@H](C)C(NC(=O)[C@H](CC(N)=O)NC(=O)Cc1ccccc1)C(=O)C(=O)NCC(=O)N[C@@H](Cc1ccc2ccccc2c1)C(N)=O]

Synthesized by Method B. Molecular Weight: 644.30. LC/MS: 645.27 (M+1)⁺

### Example 132

### (4S)-N-(2-(((S)-1-amino-3-(naphthalen-2-yl)-1-oxopropan-2-yl)amino)-2-oxoethyl)-3-((S)-3,3-dimethyl-2-(2-phenylacetamido)butanamido)-4-methyl-2-oxohexanamide

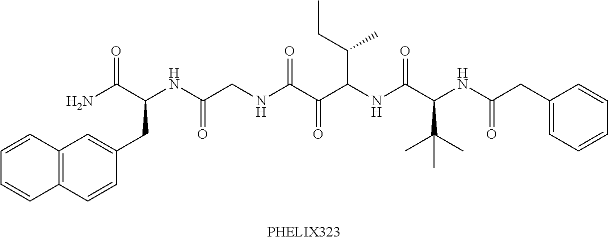 [SMILES: CC[C@H](C)C(NC(=O)[C@@H](NC(=O)Cc1ccccc1)C(C)(C)C)C(=O)C(=O)NCC(=O)N[C@@H](Cc1ccc2ccccc2c1)C(N)=O]

Synthesized by Method B. Molecular Weight: 643.8. LC/MS: 644.29 (M+1)⁺

### Example 133

### (4S)-N-(2-(((S)-1-amino-3-(naphthalen-2-yl)-1-oxopropan-2-yl)amino)-2-oxoethyl)-4-methyl-3-((S)-4-methyl-2-(2-(pyridin-2-yl)acetamido)pentanamido)-2-oxohexanamide

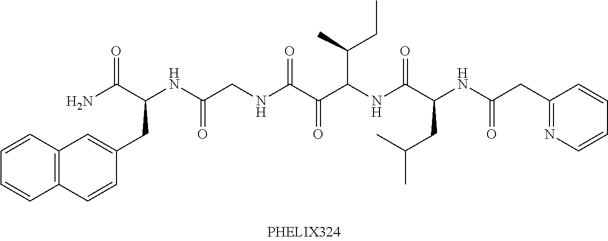 [SMILES: CC[C@H](C)C(NC(=O)[C@H](CC(C)C)NC(=O)Cc1ccccn1)C(=O)C(=O)NCC(=O)N[C@@H](Cc1ccc2ccccc2c1)C(N)=O]

Synthesized by method C. Molecular Weight: 644.77 Observed molecular weight (M+H)⁺ 645.42

### Example 134

### benzyl ((4S)-1-((2-(((S)-1-amino-3-(naphthalen-2-yl)-1-oxopropan-2-yl)amino)-2-oxoethyl)amino)-4-methyl-1,2-dioxohexan-3-yl)carbamate

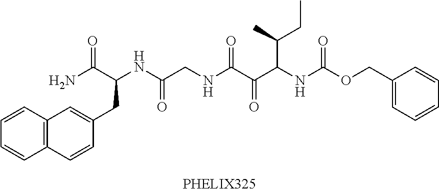 [SMILES: CC[C@H](C)C(NC(=O)OCc1ccccc1)C(=O)C(=O)NCC(=O)N[C@@H](Cc1ccc2ccccc2c1)C(N)=O]

Synthesized by method B. Molecular Weight: 546.62 Observed molecular weight (M+H)⁺ 547.56

### Example 135

### benzyl ((2S)-1-(((4S)-1-(((S)-4-amino-4-oxo-1-phenylbutan-2-yl)amino)-4-methyl-1,2-dioxohexan-3-yl)amino)-4-methyl-1-oxopentan-2-yl)carbamate

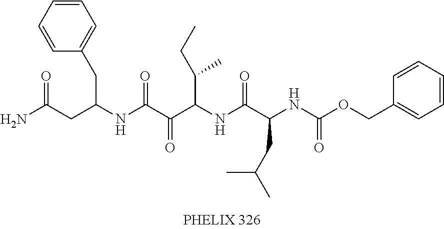 [SMILES: CC[C@H](C)C(NC(=O)[C@H](CC(C)C)NC(=O)OCc1ccccc1)C(=O)C(=O)NC(CC(N)=O)Cc1ccccc1]

Synthesized by method B. Molecular Weight: 566.70 Observed molecular weight (M+H)⁺ 567.53

### Example 136

### benzyl ((2S)-1-(((4S)-1-((2-(((S)-1-amino-3-(1H-indol-2-yl)-1-oxopropan-2-yl)amino)-2-oxoethyl)amino)-4-methyl-1,2-dioxohexan-3-yl)amino)-4-methyl-1-oxopentan-2-yl)carbamate

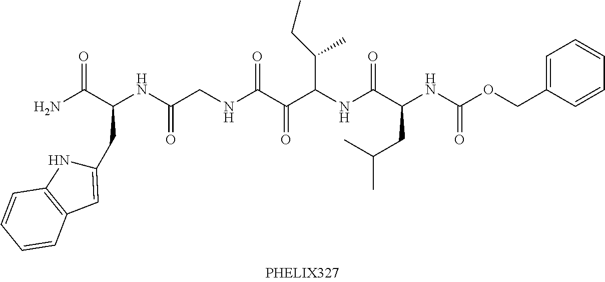 [SMILES: CC[C@H](C)C(NC(=O)[C@H](CC(C)C)NC(=O)OCc1ccccc1)C(=O)C(=O)NCC(=O)N[C@@H](Cc1cc2ccccc2[nH]1)C(N)=O]

Synthesized by Method B. Molecular Weight: 648.8 LC/MS: 649.5 (M+H)⁺

### Example 137

### benzyl ((2S)-1-(((4S)-1-(((S)-1-(((S)-1-amino-3-(naphthalen-2-yl)-1-oxopropan-2-yl)amino)-3-(1H-imidazol-5-yl)-1-oxopropan-2-yl)amino)-4-methyl-1,2-dioxohexan-3-yl)amino)-4-methyl-1-oxopentan-2-yl)carbamate

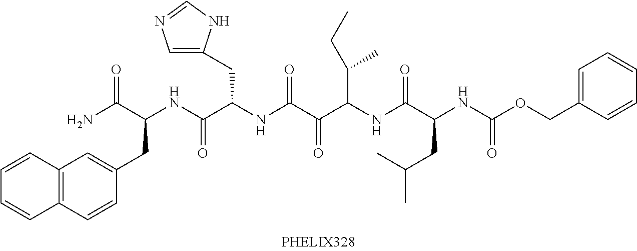 [SMILES: CC[C@H](C)C(NC(=O)[C@H](CC(C)C)NC(=O)OCc1ccccc1)C(=O)C(=O)N[C@@H](Cc1cnc[nH]1)C(=O)N[C@@H](Cc1ccc2ccccc2c1)C(N)=O]

Synthesized by Method B. Molecular Weight:739.9 LC/MS: 740.9 (M+H)⁺

### Example 138

### benzyl ((2S)-1-(((4S)-1-(((S)-1-(((R)-1-amino-3-(naphthalen-2-yl)-1-oxopropan-2-yl)amino)-5-guanidino-1-oxopentan-2-yl)amino)-4-methyl-1,2-dioxohexan-3-yl)amino)-4-methyl-1-oxopentan-2-yl)carbamate

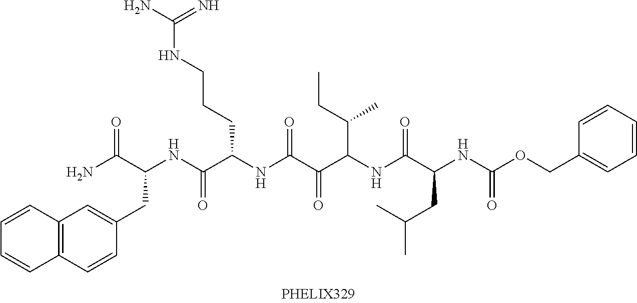 [SMILES: CC[C@H](C)C(NC(=O)[C@H](CC(C)C)NC(=O)OCc1ccccc1)C(=O)C(=O)N[C@@H](CCCNC(=N)N)C(=O)N[C@H](Cc1ccc2ccccc2c1)C(N)=O]

Synthesized by Method B. Molecular Weight: 758.9 LC/MS: 760.3 (M+H)⁺

### Example 139

### benzyl ((2S)-1-(((4S)-1-(((R)-1-(((S)-1-amino-3-(naphthalen-2-yl)-1-oxopropan-2-yl)amino)-5-guanidino-1-oxopentan-2-yl)amino)-4-methyl-1,2-dioxohexan-3-yl)amino)-4-methyl-1-oxopentan-2-yl)carbamate

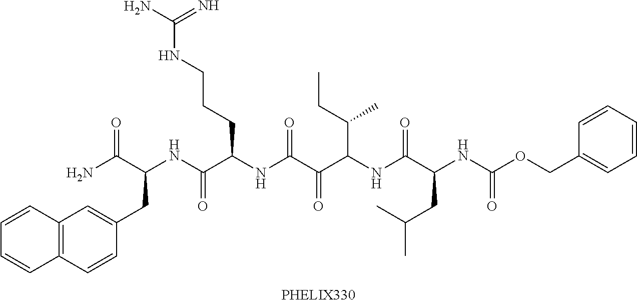 [SMILES: CC[C@H](C)C(NC(=O)[C@H](CC(C)C)NC(=O)OCc1ccccc1)C(=O)C(=O)N[C@H](CCCNC(=N)N)C(=O)N[C@@H](Cc1ccc2ccccc2c1)C(N)=O]

Synthesized by Method B. Molecular Weight: 758.9 LC/MS: 760.3 (M+H)⁺

### Example 140

### (4S)-N-(2-(((S)-1-amino-3-(naphthalen-2-yl)-1-oxopropan-2-yl)amino)-2-oxoethyl)-3-((S)-4,4-dimethyl-2-(2-phenylacetamido)pentanamido)-4-methyl-2-oxohexanamide

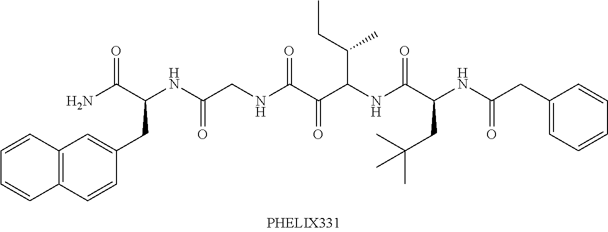 [SMILES: CC[C@H](C)C(NC(=O)[C@H](CC(C)(C)C)NC(=O)Cc1ccccc1)C(=O)C(=O)NCC(=O)N[C@@H](Cc1ccc2ccccc2c1)C(N)=O]

Synthesized by Method B. Molecular Weight: 657.8. LC/MS: 658.30 (M+1)⁺

### Example 141

### (4S)-N-(2-(((S)-1-amino-3-(naphthalen-2-yl)-1-oxopropan-2-yl)amino)-2-oxoethyl)-3-((S)-3-cyclopropyl-2-(2-phenylacetamido)propanamido)-4-methyl-2-oxohexanamide

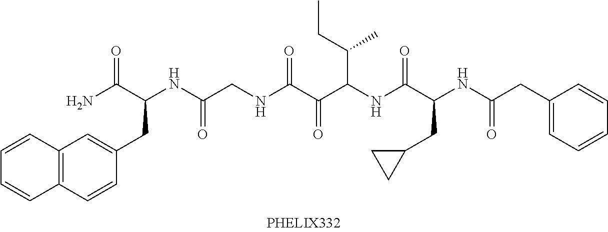 [SMILES: CC[C@H](C)C(NC(=O)[C@H](CC1CC1)NC(=O)Cc1ccccc1)C(=O)C(=O)NCC(=O)N[C@@H](Cc1ccc2ccccc2c1)C(N)=O]

Synthesized by Method B. Molecular Weight: 641.8. LC/MS: 642.33 (M+1)⁺

### Example 142

### (4S)-N-(2-(((S)-1-amino-3-(naphthalen-2-yl)-1-oxopropan-2-yl)amino)-2-oxoethyl)-4-methyl-3-((S)-4-methyl-2-(2-(pyridin-4-yl)acetamido)pentanamido)-2-oxohexanamide

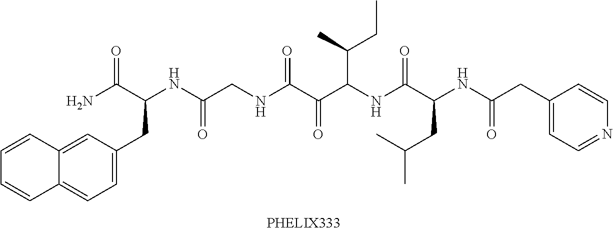 [SMILES: CC[C@H](C)C(NC(=O)[C@H](CC(C)C)NC(=O)Cc1ccncc1)C(=O)C(=O)NCC(=O)N[C@@H](Cc1ccc2ccccc2c1)C(N)=O]

Synthesized by method C. Molecular Weight: 644.77 Observed molecular weight (M+H)⁺ 646.49

### Example 143

### (4S)-N-(2-(((S)-1-amino-3-(naphthalen-2-yl)-1-oxopropan-2-yl)amino)-2-oxoethyl)-3-((S)-2-(3-benzylureido)-4-methylpentanamido)-4-methyl-2-oxohexanamide

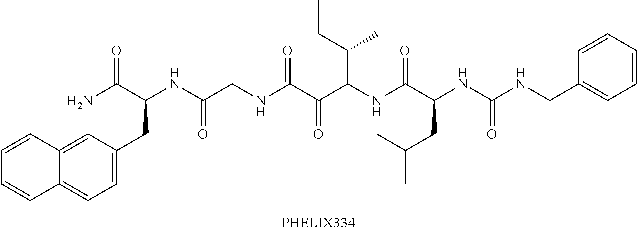 [SMILES: CC[C@H](C)C(NC(=O)[C@H](CC(C)C)NC(=O)NCc1ccccc1)C(=O)C(=O)NCC(=O)N[C@@H](Cc1ccc2ccccc2c1)C(N)=O]

Synthesized by method B. Molecular Weight: 658.80 Observed molecular weight (M+H)⁺ 659.66

### Example 144

### (4S)-N-(2-(((S)-1-amino-3-(naphthalen-2-yl)-1-oxopropan-2-yl)amino)-2-oxoethyl)-4-methyl-3-((S)-4-methyl-2-(3-phenylureido)pentanamido)-2-oxohexanamide

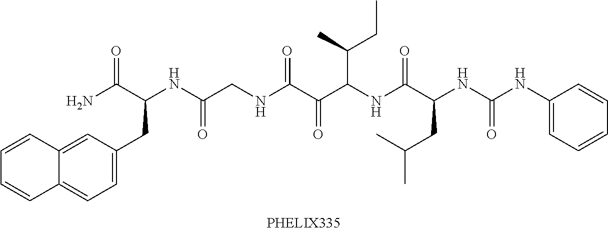 [SMILES: CC[C@H](C)C(NC(=O)[C@H](CC(C)C)NC(=O)Nc1ccccc1)C(=O)C(=O)NCC(=O)N[C@@H](Cc1ccc2ccccc2c1)C(N)=O]

Synthesized by method B. Molecular Weight: 644.77 Observed molecular weight (M+H)⁺ 645.55

### Example 145

### benzyl ((2S)-1-(((4S)-1-(((S)-1-(((S)-1-amino-3-(naphthalen-2-yl)-1-oxopropan-2-yl)amino)-3-cyano-1-oxopropan-2-yl)amino)-4-methyl-1,2-dioxohexan-3-yl)amino)-4-methyl-1-oxopentan-2-yl)carbamate

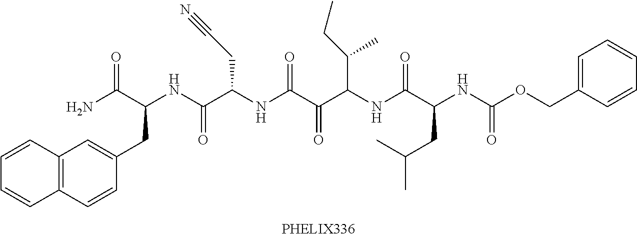 [SMILES: CC[C@H](C)C(NC(=O)[C@H](CC(C)C)NC(=O)OCc1ccccc1)C(=O)C(=O)N[C@@H](CC#N)C(=O)N[C@@H](Cc1ccc2ccccc2c1)C(N)=O]

Synthesized by Method B. Molecular Weight: 698.8 LC/MS: 699.7 (M+H)⁺

### Example 146

### (4S)-N-(2-(((S)-1-amino-3-(naphthalen-2-yl)-1-oxopropan-2-yl)amino)-2-oxoethyl)-3-((S)-3-cyano-2-(2-phenylacetamido)propanamido)-4-methyl-2-oxohexanamide

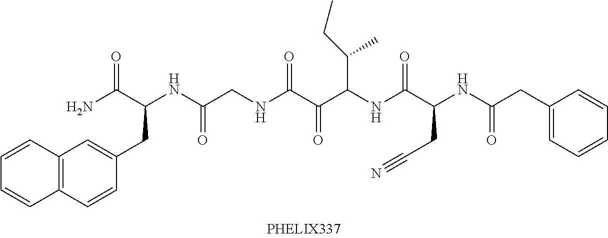 [SMILES: CC[C@H](C)C(NC(=O)[C@H](CC#N)NC(=O)Cc1ccccc1)C(=O)C(=O)NCC(=O)N[C@@H](Cc1ccc2ccccc2c1)C(N)=O]

Synthesized by Method B. Molecular Weight: 626.7 LC/MS: 627.5 (M+H)⁺

### Example 147

### (4S)-3-(2-((S)-2-acetamido-3-(naphthalen-2-yl)propanamido)acetamido)-N-((S)-1-(((S)-1-amino-1-oxo-3-phenylpropan-2-yl)amino)-4-methyl-1-oxopentan-2-yl)-4-methyl-2-oxohexanamide

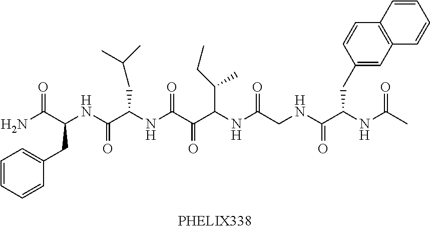 [SMILES: CC[C@H](C)C(NC(=O)CNC(=O)[C@H](Cc1ccc2ccccc2c1)NC(C)=O)C(=O)C(=O)N[C@@H](CC(C)C)C(=O)N[C@@H](Cc1ccccc1)C(N)=O]

Synthesized by Method B. Molecular Weight: 714.9 LC/MS: 715.6 (M+H)⁺

### Example 148

### benzyl ((6R,13S,16S)-1-amino-10-benzyl-6-carbamoyl-1-imino-13-isobutyl-8,9,12,15-tetraoxo-17-phenyl-2,7,11,14-tetraazaheptadecan-16-yl)carbamate

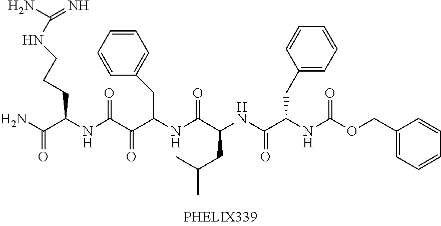 [SMILES: CC(C)C[C@H](NC(=O)[C@H](Cc1ccccc1)NC(=O)OCc1ccccc1)C(=O)NC(Cc1ccccc1)C(=O)C(=O)N[C@H](CCCNC(=N)N)C(N)=O]

Synthesized by Method B. Molecular Weight: 742.9 LC/MS: 743.9 (M+H)⁺

### Example 149

### benzyl ((2S)-1-(((4S)-1-(((S)-1-amino-1-oxo-5-ureidopentan-2-yl)amino)-4-methyl-1,2-dioxohexan-3-yl)amino)-4-methyl-1-oxopentan-2-yl)carbamate

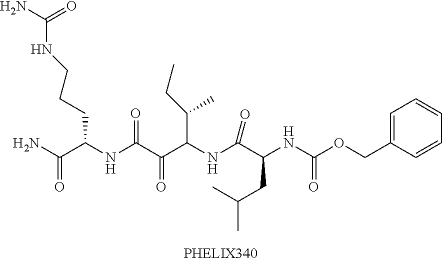 [SMILES: CC[C@H](C)C(NC(=O)[C@H](CC(C)C)NC(=O)OCc1ccccc1)C(=O)C(=O)N[C@@H](CCCNC(N)=O)C(N)=O]

Synthesized by Method B. Molecular Weight: 709.9 LC/MS: 710.7 (M+H)⁺

### Example 150

### benzyl ((2S)-1-(((4S)-1-(((R)-1-amino-1-oxo-5-ureidopentan-2-yl)amino)-4-methyl-1,2-dioxohexan-3-yl)amino)-4-methyl-1-oxopentan-2-yl)carbamate

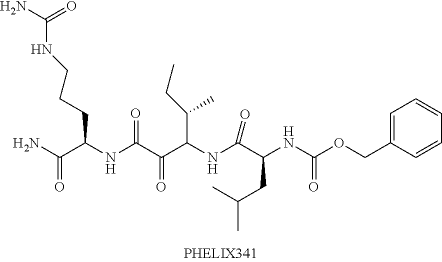 [SMILES: CC[C@H](C)C(NC(=O)[C@H](CC(C)C)NC(=O)OCc1ccccc1)C(=O)C(=O)N[C@H](CCCNC(N)=O)C(N)=O]

Synthesized by Method B. Molecular Weight: 709.9 LC/MS: 710.7 (M+H)⁺

### Example 151

### benzyl ((2S)-1-(((4S)-1-(((S)-1-amino-1-oxo-3-(pyridin-4-yl)propan-2-yl)amino)-4-methyl-1,2- dioxohexan-3-yl)amino)-4-methyl-1-oxopentan-2-yl)carbamate

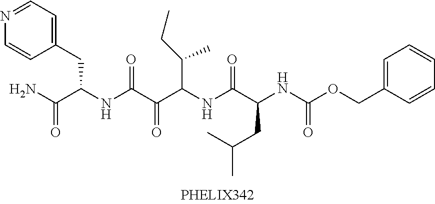 [SMILES: CC[C@H](C)C(NC(=O)[C@H](CC(C)C)NC(=O)OCc1ccccc1)C(=O)C(=O)N[C@@H](Cc1ccncc1)C(N)=O]

Synthesized by Method C. Molecular Weight: 553.8 LC/MS: 554.5(M+H)⁺

### Example 152

### benzyl ((2S)-1-(((4S)-1-(((S)-1-amino-3-(1H-indol-2-yl)-1-oxopropan-2-yl)amino)-4-methyl-1,2-dioxohexan-3-yl)amino)-4-methyl-1-oxopentan-2-yl)carbamate

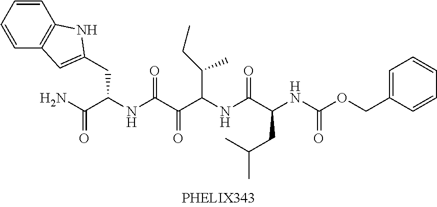 [SMILES: CC[C@H](C)C(NC(=O)[C@H](CC(C)C)NC(=O)OCc1ccccc1)C(=O)C(=O)N[C@@H](Cc1cc2ccccc2[nH]1)C(N)=O]

Synthesized by Method B. Molecular Weight: 591.7 LC/MS: 592.5 (M+H)⁺

### Example 153

### benzyl ((2S)-1-(((4S)-1-(((S)-1-amino-1-oxo-3-(pyridin-3-yl)propan-2-yl)amino)-4-methyl-1,2-dioxohexan-3-yl)amino)-4-methyl-1-oxopentan-2-yl)carbamate

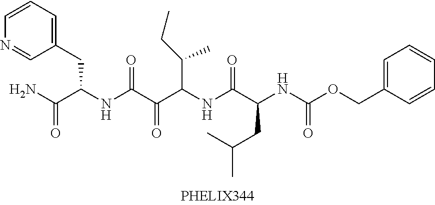 [SMILES: CC[C@H](C)C(NC(=O)[C@H](CC(C)C)NC(=O)OCc1ccccc1)C(=O)C(=O)N[C@@H](Cc1cccnc1)C(N)=O]

Synthesized by Method C. Molecular Weight: 553.7 LC/MS: 554.7 (M+H)⁺

### Example 154

### (4S)-3-((S)-2-((S)-2-acetamido-3-phenylpropanamido)-4-methylpentanamido)-N-((R)-1-amino-5-guanidino-1-oxopentan-2-yl)-4-methyl-2-oxohexanamide

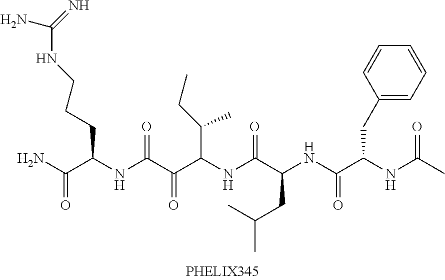 [SMILES: CC[C@H](C)C(NC(=O)[C@H](CC(C)C)NC(=O)[C@H](Cc1ccccc1)NC(C)=O)C(=O)C(=O)N[C@H](CCCNC(=N)N)C(N)=O]

Synthesized by Method B. Molecular Weight: 616.8 LC/MS: 617.9 (M+H)⁺

### Example 155

### (4S)-N-((R)-1-amino-5-guanidino-1-oxopentan-2-yl)-4-methyl-3-((S)-4-methyl-2-((S)-3-phenyl-2-(2-(pyridin-4-yl)acetamido)propanamido)pentanamido)-2-oxohexanamide

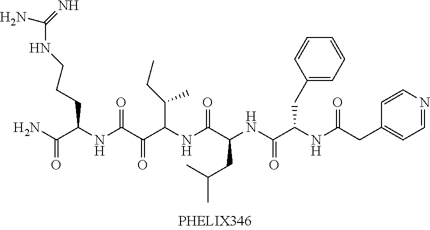 [SMILES: CC[C@H](C)C(NC(=O)[C@H](CC(C)C)NC(=O)[C@H](Cc1ccccc1)NC(=O)Cc1ccncc1)C(=O)C(=O)N[C@H](CCCNC(=N)N)C(N)=O]

Synthesized by Method C. Molecular Weight: 693.9 LC/MS: 694.7 (M+H)⁺

### Example 156

### benzyl ((2S)-1-(((4S)-1-((2-(((S)-1-amino-3-(3-fluorophenyl)-1-oxopropan-2-yl)amino)-2-oxoethyl)amino)-4-methyl-1,2-dioxohexan-3-yl)amino)-4-methyl-1-oxopentan-2-yl)carbamate

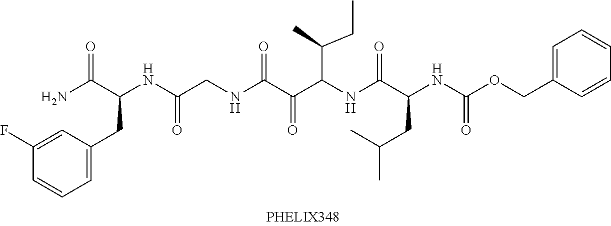 [SMILES: CC[C@H](C)C(NC(=O)[C@H](CC(C)C)NC(=O)OCc1ccccc1)C(=O)C(=O)NCC(=O)N[C@@H](Cc1cccc(F)c1)C(N)=O]

### Synthesized by method B. Molecular Weight: 627.71 Observed molecular weight (M+H)⁺ 628.66

### Example 157

### benzyl ((2S)-1-(((4S)-1-((2-(((S)-1-amino-3-(4-fluorophenyl)-1-oxopropan-2-yl)amino)-2-oxoethyl)amino)-4-methyl-1,2-dioxohexan-3-yl)amino)-4-methyl-1-oxopentan-2-yl)carbamate

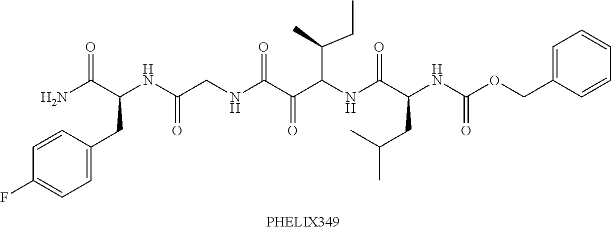 [SMILES: CC[C@H](C)C(NC(=O)[C@H](CC(C)C)NC(=O)OCc1ccccc1)C(=O)C(=O)NCC(=O)N[C@@H](Cc1ccc(F)cc1)C(N)=O]

Synthesized by method B. Molecular Weight: 627.71 Observed molecular weight (M+H)⁺ 628.60

### Example 158

### benzyl ((2S)-1-(((4S)-1-((2-(((S)-1-amino-3-(4-cyanophenyl)-1-oxopropan-2-yl)amino)-2-oxoethyl)amino)-4-methyl-1,2-dioxohexan-3-yl)amino)-4-methyl-1-oxopentan-2-yl)carbamate

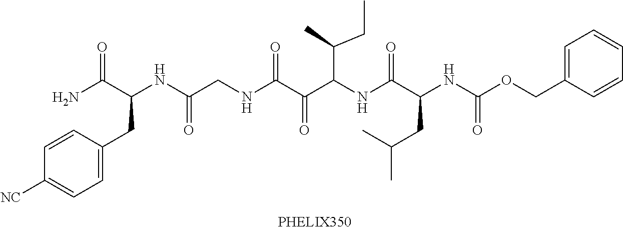 [SMILES: CC[C@H](C)C(NC(=O)[C@H](CC(C)C)NC(=O)OCc1ccccc1)C(=O)C(=O)NCC(=O)N[C@@H](Cc1ccc(C#N)cc1)C(N)=O]

Synthesized by method B. Molecular Weight: 634.73 Observed molecular weight (M+H)⁺ 635.53

### Example 159

### (4S)-N-(2-(((S)-1-amino-1-oxo-3-phenylpropan-2-yl)(methyl)amino)-2-oxoethyl)-4-methyl-3-((S)-4-methyl-2-(2-(pyridin-3-yl)acetamido)pentanamido)-2-oxohexanamide

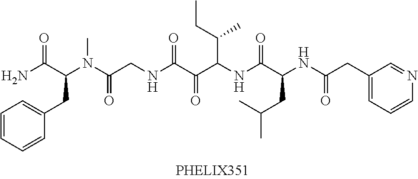 [SMILES: CC[C@H](C)C(NC(=O)[C@H](CC(C)C)NC(=O)Cc1cccnc1)C(=O)C(=O)NCC(=O)N(C)[C@@H](Cc1ccccc1)C(N)=O]

Synthesized by Method C. Molecular Weight: 608.7 LC/MS: 609.6 (M+H)⁺

### Example 160

### (4S)-N-(2-(((S)-1-amino-1-oxo-3-phenylpropan-2-yl)(methyl)amino)-2-oxoethyl)-4-methyl-3-((S)-4-methyl-2-(2-morpholinoacetamido)pentanamido)-2-oxohexanamide

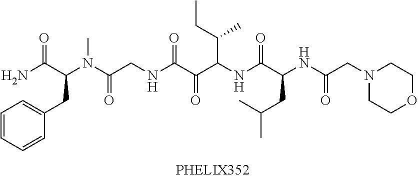 [SMILES: CC[C@H](C)C(NC(=O)[C@H](CC(C)C)NC(=O)CN1CCOCC1)C(=O)C(=O)NCC(=O)N(C)[C@@H](Cc1ccccc1)C(N)=O]

Synthesized by Method C. Molecular Weight: 616.8 LC/MS: 617.9 (M+H)⁺

### Example 161

### (4S)-N-(2-(((S)-1-amino-1-oxo-3-phenylpropan-2-yl)(methyl)amino)-2-oxoethyl)-4-methyl-3-((S)-4-methyl-2-(2-(pyridin-4-yl)acetamido)pentanamido)-2-oxohexanamide

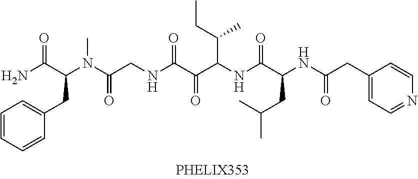 [SMILES: CC[C@H](C)C(NC(=O)[C@H](CC(C)C)NC(=O)Cc1ccncc1)C(=O)C(=O)NCC(=O)N(C)[C@@H](Cc1ccccc1)C(N)=O]

Synthesized by Method C. Molecular Weight: 608.7 LC/MS: 609.5 (M+H)⁺

### Example 162

### (4S)-N-((S)-1-amino-3-(naphthalen-2-yl)-1-oxopropan-2-yl)-4-methyl-3-((S)-4-methyl-2-(2-morpholinoacetamido)pentanamido)-2-oxohexanamide

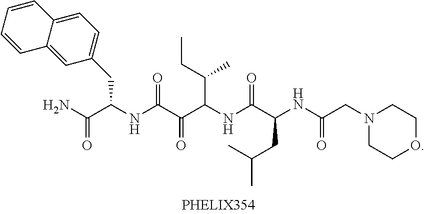 [SMILES: CC[C@H](C)C(NC(=O)[C@H](CC(C)C)NC(=O)CN1CCOCC1)C(=O)C(=O)N[C@@H](Cc1ccc2ccccc2c1)C(N)=O]

Synthesized by Method C. Molecular Weight: 595.7 LC/MS: 596.6 (M+H)⁺

### Example 163

### benzyl ((2S)-1-(((4S)-1-((2-(((S)-1-amino-3-(4-methoxyphenyl)-1-oxopropan-2-yl)amino)-2-oxoethyl)amino)-4-methyl-1,2-dioxohexan-3-yl)amino)-4-methyl-1-oxopentan-2-yl)carbamate

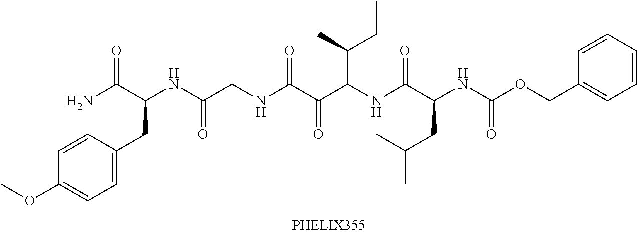 [SMILES: CC[C@H](C)C(NC(=O)[C@H](CC(C)C)NC(=O)OCc1ccccc1)C(=O)C(=O)NCC(=O)N[C@@H](Cc1ccc(OC)cc1)C(N)=O]

Synthesis by method B. Molecular Weight: 639.75 Observed molecular weight (M+H)⁺ 640.63

### Example 164

### benzyl ((2S)-1-(((4S)-1-((2-(((S)-1-amino-1-oxo-3-(pyridin-3-yl)propan-2-yl)amino)-2-oxoethyl)amino)-4-methyl-1,2-dioxohexan-3-yl)amino)-4-methyl-1-oxopentan-2-yl)carbamate

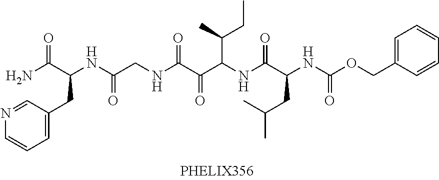 [SMILES: CC[C@H](C)C(NC(=O)[C@H](CC(C)C)NC(=O)OCc1ccccc1)C(=O)C(=O)NCC(=O)N[C@@H](Cc1cccnc1)C(N)=O]

Synthesis by method C. Molecular Weight: 610.71 Observed molecular weight (M+H)⁺ 612.40

### Example 165

### benzyl ((2S)-1-(((4S)-1-((2-(((S)-1-amino-1-oxo-3-(pyridin-4-yl)propan-2-yl)amino)-2-oxoethyl)amino)-4-methyl-1,2-dioxohexan-3-yl)amino)-4-methyl-1-oxopentan-2-yl)carbamate

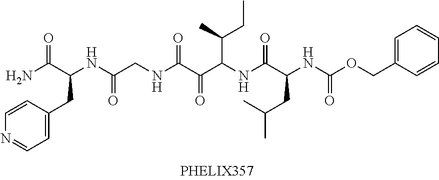 [SMILES: CC[C@H](C)C(NC(=O)[C@H](CC(C)C)NC(=O)OCc1ccccc1)C(=O)C(=O)NCC(=O)N[C@@H](Cc1ccncc1)C(N)=O]

Synthesis by method C. Molecular Weight: 610.71 Observed molecular weight (M+H)⁺ 611.64

### Example 166

### benzyl ((2S)-1-(((4S)-1-(((S)-1-amino-1-oxo-3-phenylpropan-2-yl)(methyl)amino)-4-methyl-1,2-dioxohexan-3-yl)amino)-4-methyl-1-oxopentan-2-yl)carbamate

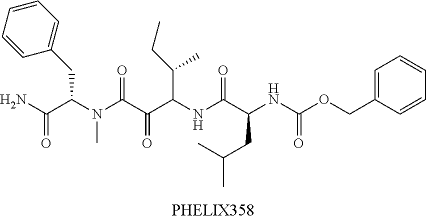 [SMILES: CC[C@H](C)C(NC(=O)[C@H](CC(C)C)NC(=O)OCc1ccccc1)C(=O)C(=O)N(C)[C@@H](Cc1ccccc1)C(N)=O]

Synthesized by Method B. Molecular Weight: 566.7 LC/MS: 567.5 (M+H)⁺

### Example 167

### benzyl ((2S)-1-(((4S)-1-(((S)-1-amino-3-(3-fluorophenyl)-1-oxopropan-2-yl)amino)-4-methyl-1,2-dioxohexan-3-yl)amino)-4-methyl-1-oxopentan-2-yl)carbamate

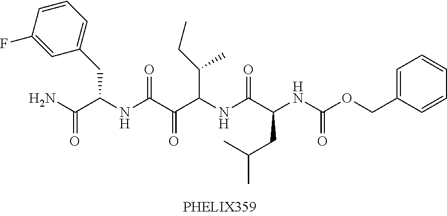 [SMILES: CC[C@H](C)C(NC(=O)[C@H](CC(C)C)NC(=O)OCc1ccccc1)C(=O)C(=O)N[C@@H](Cc1cccc(F)c1)C(N)=O]

Synthesized by Method B. Molecular Weight: 570.7 LC/MS: 571.5 (M+H)⁺

### Example 168

### benzyl ((2S)-1-(((4S)-1-(((S)-1-(dimethylamino)-3-(naphthalen-2-yl)-1-oxopropan-2-yl)amino)-4-methyl-1,2-dioxohexan-3-yl)amino)-4-methyl-1-oxopentan-2-yl)carbamate

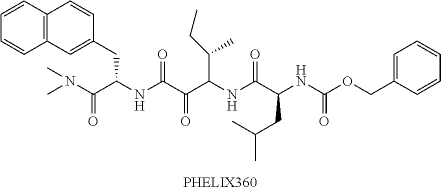 [SMILES: CC[C@H](C)C(NC(=O)[C@H](CC(C)C)NC(=O)OCc1ccccc1)C(=O)C(=O)N[C@@H](Cc1ccc2ccccc2c1)C(=O)N(C)C]

Synthesized by Method D. Molecular Weight: 630.8 LC/MS: 629.6 (M+H)⁺

### Example 169

### benzyl ((2S)-1-(((4S)-1-(((S)-1-(cyclopropylamino)-3-(naphthalen-2-yl)-1-oxopropan-2-yl)amino)-4-methyl-1,2-dioxohexan-3-yl)amino)-4-methyl-1-oxopentan-2-yl)carbamate

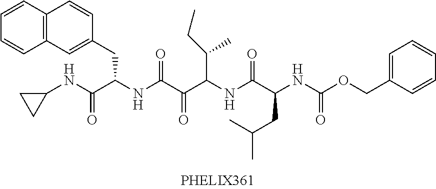 [SMILES: CC[C@H](C)C(NC(=O)[C@H](CC(C)C)NC(=O)OCc1ccccc1)C(=O)C(=O)N[C@@H](Cc1ccc2ccccc2c1)C(=O)NC1CC1]

Synthesized by Method D. Molecular Weight: 642.8 LC/MS: 641.6 (M+H)⁺

### Example 170

### benzyl ((2S)-4-methyl-1-(((4S)-4-methyl-1,2-dioxo-1-(3((pyrimidinylmethyl)carbamoyl)azetidin-1-yl)hexan-3-yl)amino)-1-oxopentan-2-yl)carbamate

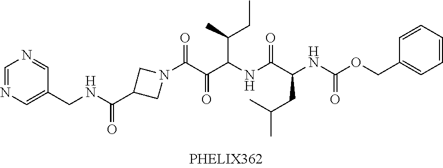 [SMILES: CC[C@H](C)C(NC(=O)[C@H](CC(C)C)NC(=O)OCc1ccccc1)C(=O)C(=O)N1CC(C(=O)NCc2cncnc2)C1]

Synthesized by method D. Molecular Weight: 580.69 Observed molecular weight (M+H)⁺ 581.65

### Example 171

### benzyl ((2S)-4-methyl-1-(((4S)-4-methyl-1-(3-((naphthalen-2-ylmethyl)carbamoyl)azetidin-1-yl)-1,2-dioxohexan-3-yl)amino)-1-oxopentan-2-yl)carbamate

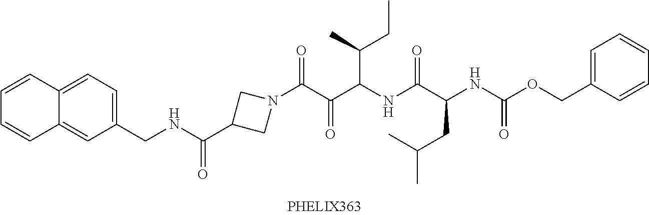 [SMILES: CC[C@H](C)C(NC(=O)[C@H](CC(C)C)NC(=O)OCc1ccccc1)C(=O)C(=O)N1CC(C(=O)NCc2ccc3ccccc3c2)C1]

Synthesized by method D. Molecular Weight: 628.77 Observed molecular weight (M+H)⁺ 629.54

### Example 172

### benzyl ((2S)-4-methyl-1-(((4S)-4-methyl-1,2-dioxo-1-(3-(((1,4,5,6-tetrahydrocyclopenta[c]pyrazol-3-yl)methyl)carbamoyl)azetidin-1-yl)hexan-3-yl)amino)-1-oxopentan-2-yl)carbamate

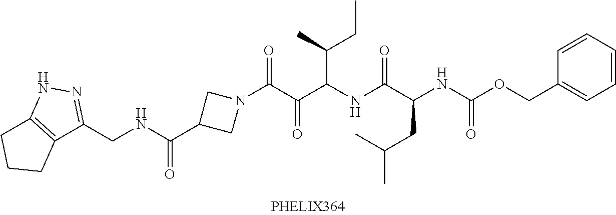 [SMILES: CC[C@H](C)C(NC(=O)[C@H](CC(C)C)NC(=O)OCc1ccccc1)C(=O)C(=O)N1CC(C(=O)NCc2n[nH]c3c2CCC3)C1]

Synthesized by method D. Molecular Weight: 608.74 Observed molecular weight (M+H)⁺ 609.69

### Example 173

### (4S)-N-(2-(((S)-1-amino-3-(naphthalen-2-yl)-1-oxopropan-2-yl)amino)-2-oxoethyl)-4-methyl-3-((S)-4-methyl-2-(2-(pyrimidin-5-yl)acetamido)pentanamido)-2-oxohexanamide

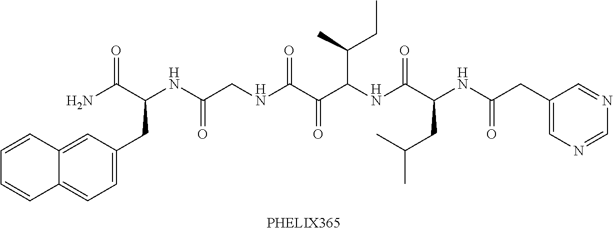 [SMILES: CC[C@H](C)C(NC(=O)[C@H](CC(C)C)NC(=O)Cc1cncnc1)C(=O)C(=O)NCC(=O)N[C@@H](Cc1ccc2ccccc2c1)C(N)=O]

Synthesized by method C. Molecular Weight: 645.76 Observed molecular weight (M+H)⁺ 646.68

### Example 174

### benzyl ((2S)-1-(((4S)-1-(3-((4-carbamimidoylbenzyl)carbamoyl)azetidin-1-yl)-4-methyl-1,2-dioxohexan-3-yl)amino)-4-methyl-1-oxopentan-2-yl)carbamate

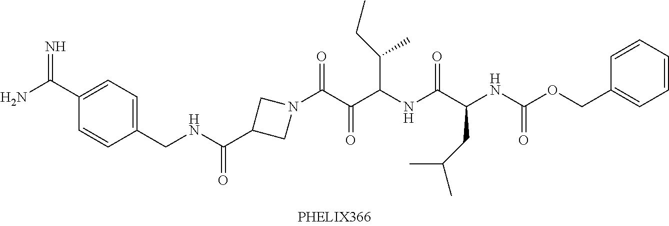 [SMILES: CC[C@H](C)C(NC(=O)[C@H](CC(C)C)NC(=O)OCc1ccccc1)C(=O)C(=O)N1CC(C(=O)NCc2ccc(C(=N)N)cc2)C1]

Synthesized by method C. Molecular Weight: 620.75 Observed molecular weight (M+H)⁺ 621.54

### Example 175

### 1-((4S)-3-((S)-2-(((benzyloxy)carbonyl)amino)-4-methylpentanamido)-4-methyl-2-oxohexanoyl)azetidine-3-carboxylic acid

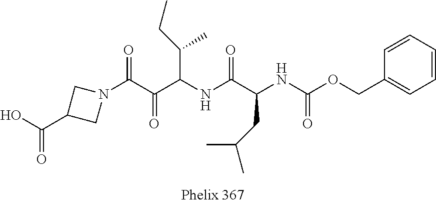 [SMILES: CC[C@H](C)C(NC(=O)[C@H](CC(C)C)NC(=O)OCc1ccccc1)C(=O)C(=O)N1CC(C(=O)O)C1]

Synthesized by method A. Molecular Weight: 489.57 Observed molecular weight (M+H)⁺ 490.28

### Example 176

### Enzyme Assays

A. Inhibitor Dilutions

Purified peptide was dissolved in DMSO to a concentration of 10 mM then further diluted to 100 μM in DMSO. Stock plates were established with inhibitors at ten concentrations with top concentrations of 10 mM, 1 mM, 100 μM, or 10 μM which were then subsequently halved in DMSO down to bottom concentrations of 9.8 μM, 0.98 μM, 98 nM, or 9.8 nM respectively in DMSO. Alternatively, the stock plate was established with a top inhibitor concentration of 100 μM diluted by thirds with ten concentrations down to a bottom concentration of 65.1 nM in DMSO. Two wells contained DMSO only for controls.

B. Calpain Assay

Enzyme was dissolved to 25 nM in 50 mM Tris Buffer (pH 7.5), 100 mM NaCl, 1 mM EGTA, 0.015% Brij-35, and 5 mM DTT. Substrate, H-Glu(Edans)-Pro-Leu-Phe-Ala-Glu-Arg-Lys(Dabcyl)-OH, was diluted to 5 μM in buffer with enzyme. Enzyme/Buffer/Substrate solution was aliquoted 267 uL per well in one row (12 wells). Using a multichannel pipette 3 μL inhibitor was taken from the stock plate and added to each well, a 1:100 dilution. Final concentrations of inhibitor was a top concentration of 100 μM, 10 μM, 1 μM, or 0.1 μM which which were then subsequently halved in down to a bottom concentration of 0.098 μM, 0.0098 μM, 0.98 nM, or 0.098 nM respectively. Ten concentrations in all. Two controls were measured, 0 μM inhibitor and no calcium (effectively dead enzyme). A multichannel pipette was then used to distribute 90 μL to three wells per concentration. The reaction was initiated with an injection of 10 μL of 50 mM CaCl2 (final concentration of 5 mM CaCl2) resulting in a final volume of 100 μL per well. Kinetic measurements were taken for 30 min. with Ex/Em 340/520 nm.

GraphPad Prism was used for all data analysis. Data was plotted as a RFU vs. time graph. Velocity (RFU/sec) was calculated from a straight line fit of the straight portion of the curve. Velocity was converted to μM/sec. Data was then plotted as velocity (μM/sec) vs. inhibitor concentration and analyzed using the Morrison equation that was standard in GraphPad Prism to calculate a Ki.

C. Cathepsin L Assay

Enzyme was dissolved to 3 nM in 50 mM Sodium Acetate Buffer (pH 5.5), 2.5 mM EGTA, 0.01% Triton X-100, and 5 mM DTT. Substrate, Z-Phe-Arg-Amc, was diluted to 50 μM in buffer. Enzyme/Buffer solution was aliquoted 267 uL per well in one row (12 wells). Using a multichannel pipette 3 μL inhibitor was taken from the stock plate and added to each well, a 1:100 dilution resulting in ten concentrations of inhibitor and two controls, 0 μM inhibitor and dead enzyme. The enzyme/inhibitor was incubated at room temperature for 30 min. A multichannel pipette was then used to distribute 90 μL to three wells per concentration. The reaction was initiated with an injection of 10 μL of 50 mM Z-FR-Amc substrate resulting in a final volume of 100 μL per well. Kinetic measurements were taken for 30 min. with Ex/Em 380/460 nm.

GraphPad Prism was used for all data analysis. Data was plotted as a RFU vs. time graph. Velocity (RFU/sec) was calculated from a straight line fit of the straight portion of the curve. Velocity was converted to μM/sec. Data was then plotted as velocity (μM/sec) vs. inhibitor concentration and analyzed using the Morrison equation that was standard in GraphPad Prism to calculate a Ki.

D. Cathepsin B Assay

Enzyme was dissolved to 5 nM in 50 mM Potassium Phosphate Buffer (pH 6.0), 2.5 mM EGTA, 0.01% Triton X-100, and 5 mM DTT. Substrate, Z-Phe-Arg-Amc, was diluted to 50 μM in buffer. Enzyme/Buffer solution was aliquoted 267 uL per well in one row (12 wells). Using a multichannel pipette 3 μL inhibitor was taken from the stock plate and added to each well, a 1:100 dilution resulting in ten concentrations of inhibitor and two controls, 0 μM inhibitor and dead enzyme. The enzyme/inhibitor was incubated at room temperature for 30 min. A multichannel pipette was then used to distribute 90 μL to three wells per concentration. The reaction was initiated with an injection of 10 μL of 50 mM Z-FR-Amc substrate resulting in a final volume of 100 μL per well. Kinetic measurements were taken for 30 min. with Ex/Em 380/460 nm.

GraphPad Prism was used for all data analysis. Data was plotted as a RFU vs. time graph. Velocity (RFU/sec) was calculated from a straight line fit of the straight portion of the curve. Velocity was converted to μM/sec. Data was then plotted as velocity (μM/sec) vs. inhibitor concentration and analyzed using the Morrison equation that was standard in GraphPad Prism to calculate a Ki.

| | Cat B | Cat L | Cat S | Cat K | Calpain |
|---|---|---|---|---|---|
| PHELIX # | (μM) | (μM) | (μM) | (μM) | (μM) |
| | 85.9500 | 22.92000 | | | |
| PHELIX130 | 531.6000 | | | | |
| PHELIX132 | 702.1000 | 133.90000 | | | |
| PHELIX133 | NI | 209.60000 | | | |
| PHELIX137 | 331.4000 | 55.65000 | | | |
| PHELIX138 | 27.7500 | 69.85000 | | | |
| PHELIX139 | 1128.0000 | 57.44000 | | | |
| PHELIX149 | 0.0318 | 0.81620 | | | >100 |
| PHELIX150 | 0.0303 | 0.03925 | | | 5.10700 |
| PHELIX155 | 0.0215 | 0.29990 | | | |
| PHELIX156 | 0.0102 | 0.00158 | | | 9.84600 |
| PHELIX157 | 0.0028 | 0.05617 | | | 34.56000 |
| PHELIX158 | 0.4394 | 0.14150 | | | |
| PHELIX159 | 0.0414 | 2.45200 | | | 16.65000 |
| PHELIX160 | 111.2000 | 63.44000 | | | 51.00000 |
| PHELIX161 | 0.0549 | 1.99100 | | | |
| PHELIX162 | 0.0300 | 0.40230 | | | |
| PHELIX163 | 0.0485 | 0.42860 | | | |
| PHELIX164 | 34.6200 | >1 | | | >100 |
| PHELIX165 | 0.0736 | 0.45270 | | | |
| PHELIX166 | 0.0081 | 0.58560 | | | >100 |
| PHELIX167 | 0.2730 | 2.59500 | | | |
| PHELIX168 | 0.5189 | 1.26600 | | | |
| PHELIX169 | 0.0698 | 0.31930 | | | |
| PHELIX170 | 0.0220 | 1.19200 | | | 59.68000 |
| PHELIX171 | 0.5602 | 0.63190 | | | 3.89800 |
| PHELIX172 | 0.0746 | 0.51950 | | | |
| PHELIX173 | 0.7199 | 3.33300 | | | >100 |
| PHELIX174 | 0.1575 | 0.46060 | | | |
| PHELIX175 | 0.0069 | 0.10830 | | | |
| PHELIX176 | 0.0378 | 2.68900 | | | |
| PHELIX177 | 0.0012 | 0.09222 | | | |
| PHELIX178 | 0.0067 | 0.03855 | | | |
| PHELIX179 | 17.7900 | 0.88120 | | | |
| PHELIX180 | 0.1969 | 0.12710 | | | |
| PHELIX183 | 7.7430 | 0.19470 | | | 0.15340 |
| PHELIX184 | 0.6408 | 0.00485 | 0.00100 | 0.0005 | 0.01027 |
| PHELIX185 | 2.1010 | 0.10220 | | | 0.06838 |
| PHELIX186 | 59.9800 | 4.31700 | | | 18.09000 |
| PHELIX187 | 0.0479 | 0.05356 | 0.00204 | 0.0038 | 0.04546 |
| PHELIX198 | 7.2990 | 0.03389 | | | 0.02200 |
| PHELIX200 | 0.0881 | 0.00151 | | | 0.00657 |
| PHELIX202 | 0.6920 | 0.02396 | | | 0.02529 |
| PHELIX204 | 0.2772 | 0.00001 | 0.00044 | 0.0093 | 0.20450 |
| PHELIX205 | 0.9770 | 0.07039 | 0.22900 | 6.4130 | 12.49000 |
| PHELIX206 | 0.1202 | 0.00652 | 0.00396 | >10 | >10 |
| PHELIX207 | 0.1874 | 0.01499 | 0.00260 | 0.4295 | >10 |
| PHELIX208 | 0.4200 | 0.01537 | 0.00373 | 1.9170 | >10 |
| PHELIX209 | 0.2344 | 0.06628 | 0.03990 | >10 | >10 |
| PHELIX210 | 24.2300 | 2.38300 | 6.58600 | >10 | >10 |
| PHELIX211 | 0.5316 | 0.21750 | | | >10 |
| PHELIX212 | 0.2313 | 0.02033 | 0.01370 | 1.2040 | >10 |
| PHELIX213 | 22.9800 | 0.00006 | 0.00079 | 0.0798 | >10 |
| PHELIX214 | 5.7910 | 0.28070 | 0.03979 | 2.4590 | 85.38000 |
| PHELIX215 | 9.2400 | 0.00018 | 0.00048 | 0.0222 | >10 |
| PHELIX216 | 2939.0000 | >1000 | | | >100 |
| PHELIX217 | 3974.0000 | 1082.00000 | | | >100 |
| PHELIX218 | >100000 | >1000 | | | |
| PHELIX219 | 7.5160 | 2.91500 | | | >10 |
| PHELIX220 | 5.1010 | 0.00005 | 0.00022 | 0.0219 | >10 |
| PHELIX221 | 3.5660 | 0.00002 | 0.00019 | 0.0136 | >10 |
| PHELIX222 | 1.8300 | 0.02503 | 0.01769 | 3.3150 | >10 |
| PHELIX223 | 0.2458 | 0.01990 | 0.00444 | 0.2811 | >10 |
| PHELIX224 | 0.4223 | 0.04572 | 0.03319 | | >10 |
| PHELIX225 | 0.2987 | 0.01865 | 0.00812 | 0.2540 | >10 |
| PHELIX226 | 0.2732 | 0.00954 | | 1.7660 | >10 |
| PHELIX227 | 0.1712 | 0.05893 | 0.01631 | 0.5359 | >10 |
| PHELIX228 | 0.2761 | 0.00008 | | | 0.02531 |
| PHELIX230 | 0.2356 | 0.00678 | 0.01147 | 0.3101 | >10 |
| PHELIX231 | 2.3010 | 0.01374 | 0.19960 | 4.8880 | 3.78900 |
| PHELIX232 | 19.8700 | 0.21320 | 0.14700 | 1.8450 | >10 |
| PHELIX233 | 0.2233 | 0.00809 | 0.00255 | 0.3545 | >10 |
| PHELIX234 | >10 | 0.12880 | 0.07808 | 1.0820 | >10 |
| PHELIX235 | 0.6440 | 0.23610 | 0.02890 | | >10 |
| PHELIX236 | >10 | 1.72300 | 0.25960 | | >10 |
| PHELIX237 | 0.0211 | 0.00336 | 0.00080 | 0.2263 | >10 |
| PHELIX238 | 0.3128 | 0.00388 | 0.02025 | 0.0277 | 0.09115 |
| PHELIX241 | >10 | 1.35600 | 2.73800 | | >10 |
| PHELIX242 | >10 | 7.51600 | 0.55540 | | >10 |
| PHELIX243 | >10 | 5.57900 | | | >10 |
| PHELIX247 | >10 | 6.97100 | 2.35800 | 4.7880 | >10 |
| PHELIX248 | >10 | 0.04659 | 0.00172 | >10 | >10 |
| PHELIX249 | 0.3489 | 0.10100 | 0.00681 | 0.7358 | >10 |
| PHELIX251 | 0.1061 | 0.00140 | | | 0.02500 |
| PHELIX252 | >10 | 0.00503 | | | 0.01697 |
| PHELIX253 | 1.1720 | 0.02153 | | | 0.02138 |
| PHELIX254 | 0.0432 | 0.00071 | | | 0.00342 |
| PHELIX256 | 0.2402 | 0.00147 | | | 0.01221 |
| PHELIX257 | 0.7276 | 0.00181 | | | 0.00092 |
| PHELIX258 | 0.4203 | 0.00614 | | | 0.00190 |
| PHELIX259 | >10 | 0.00193 | | | >10 |
| PHELIX260 | 0.0178 | 0.00027 | | | >10 |
| PHELIX261 | >10 | 0.00248 | | | >10 |
| PHELIX262 | 0.4352 | 0.00004 | | | >10 |
| PHELIX263 | 0.5995 | 0.00006 | | | >10 |
| PHELIX264 | >10 | 0.00191 | | | >10 |
| PHELIX265 | >10 | 0.30180 | | | >10 |
| PHELIX266 | >10 | 0.00014 | | | >10 |
| PHELIX268 | 0.1708 | 0.00095 | | | 0.01057 |
| PHELIX269 | 0.7634 | 0.00413 | | | 0.00434 |
| PHELIX270 | >10 | 0.00182 | | | 0.00053 |
| PHELIX289 | | <0.0002 | | | 0.02053 |
| PHELIX290 | | 0.00270 | | | 0.00727 |
| PHELIX291 | | 5.90900 | | | >10 |
| PHELIX292 | | 0.03708 | | | 4.30000 |
| PHELIX293 | | 0.23000 | | | 1.19400 |
| PHELIX294 | | 0.00253 | | | 0.00049 |
| PHELIX295 | | 0.00084 | | | 0.00205 |
| PHELIX296 | | 1.71900 | | | 1.55200 |
| PHELIX297 | 0.2351 | 0.04242 | | | 0.03994 |
| PHELIX298 | | 0.31540 | | | 2.57200 |
| PHELIX299 | | 0.01226 | | | 0.00483 |
| PHELIX300 | | 0.00002 | | | 0.00081 |
| PHELIX301 | 0.0109 | 0.00020 | | | 0.00539 |
| PHELIX302 | | 0.38880 | | | 0.20060 |
| PHELIX303 | | 0.00114 | | | 0.00603 |
| PHELIX304 | | 0.00060 | | | 0.00824 |
| PHELIX305 | | 0.91200 | | | 9.45300 |
| PHELIX306 | | 0.07757 | | | 0.44650 |
| PHELIX307 | | 0.00035 | | | 0.00261 |
| PHELIX308 | | 0.95600 | | | 1.51300 |
| PHELIX309 | >10 | 0.00976 | | | 0.00134 |
| PHELIX310 | | 0.01129 | | | 0.00550 |
| PHELIX311 | | 0.10640 | | | 0.30860 |
| PHELIX312 | | 0.00265 | | | 0.00081 |
| PHELIX313 | | <0.0001 | | | 0.00991 |
| PHELIX314 | 0.3865 | 0.00306 | | | 0.00244 |
| PHELIX315 | >10 | 0.00047 | | | 0.00148 |
| PHELIX316 | | 0.00333 | | | 0.00801 |
| PHELIX317 | | 0.00137 | | | 0.01983 |
| PHELIX318 | | 0.00382 | | | 0.00163 |
| PHELIX319 | 0.4564 | 0.00028 | | | 0.00068 |
| PHELIX320 | | 0.49500 | | | 0.03864 |
| PHELIX321 | | <0.0001 | | | 0.08390 |
| PHELIX322 | | 0.10080 | | | 0.07102 |
| PHELIX323 | | 0.27290 | | | 2.37 ± 1.046 |
| PHELIX324 | | 0.00132 | | | 0.00815 |
| PHELIX325 | | 0.23940 | | | 1.21100 |
| PHELIX326 | | 0.06015 | | | 0.70210 |
| PHELIX327 | | 0.00933 | | | 0.00998 |
| PHELIX328 | | 0.00252 | | | 0.03513 |
| PHELIX329 | | 0.00267 | | | 0.11050 |
| PHELIX330 | | 0.00291 | | | 0.02142 |
| PHELIX331 | | 0.00167 | | | 0.00926 |
| PHELIX332 | | 0.00115 | | | 0.00237 |
| PHELIX333 | | 0.00059 | | | 0.00098 |
| PHELIX334 | | 0.00043 | | | 0.00067 |
| PHELIX335 | | <0.00015 | | | 0.00145 |
| PHELIX336 | | 0.00404 | | | 0.03361 |
| PHELIX337 | | 0.00848 | | | 0.22440 |
| PHELIX338 | | 2.87900 | | | NI |
| PHELIX339 | | | | | 0.00218 |
| PHELIX340 | | 0.00388 | | | 0.01711 |
| PHELIX341 | | 0.02792 | | | 0.02336 |
| PHELIX342 | | 0.03133 | | | 0.02024 |
| PHELIX343 | | 0.00353 | | | 0.03412 |
| PHELIX344 | | 0.02061 | | | 0.02666 |
| PHELIX345 | | 0.01133 | | | 0.03706 |
| PHELIX346 | | 0.00328 | | | 0.00171 |
| PHELIX348 | | 0.00276 | | | 0.00168 |
| PHELIX349 | | 0.00211 | | | 0.00227 |
| PHELIX350 | | 0.00420 | | | 0.00345 |
| PHELIX351 | | 0.00045 | | | 0.00030 |
| PHELIX352 | | 0.00048 | | | 0.00193 |
| PHELIX353 | | 0.00055 | | | 0.00303 |
| PHELIX354 | | 0.01959 | | | 0.01006 |
| PHELIX355 | | 0.00157 | | | 0.00171 |
| PHELIX356 | | 0.01117 | | | 0.00917 |
| PHELIX357 | | 0.00439 | | | 0.01548 |
| PHELIX358 | | >1 | | | NI |
| PHELIX359 | | 0.00073 | | | 0.00203 |
| PHELIX360 | | 0.02914 | | | 0.04406 |
| PHELIX361 | | 0.02375 | | | 0.04154 |
| PHELIX362 | | 0.15710 | | | 0.63020 |
| PHELIX363 | | 0.01937 | | | 0.17850 |
| PHELIX364 | | 0.11730 | | | 0.03205 |
| PHELIX365 | | 0.01795 | | | 0.00621 |
| PHELIX366 | | 0.13660 | | | 0.44190 |
| PHELIX367 | | 0.90550 | | | >1 |

| | EBOV WT | | MARV WT | Fibrosis-Sirius | Fibrosis-SMA |
|---|---|---|---|---|---|
| | (μM) (Ebola | MERS WT | (μM) (Marburg | Red Collagen Assay | Elisa |
| PHELIX # | Wild Type) | (μM) | Wild Type) | (μM) | (μM) |
| PHELIX183 | 29.1300 | 407.33 | | | |
| PHELIX184 | 1.8700 | 7.20 | 3.34 | | |
| PHELIX185 | 13.3500 | NI | | | |
| PHELIX186 | 57.5000 | NI | | | |
| PHELIX187 | 3.6600 | 56.00 | 13.48 | | |
| PHELIX198 | 4.0300 | NI | | | |
| PHELIX200 | 6.4000 | NI | | | |
| PHELIX202 | NI | NI | | | |
| PHELIX204 | 0.1300 | 1.80 | 0.42 | | |
| PHELIX205 | 21.8700 | NI | | | |
| PHELIX206 | 2.5100 | 12.57 | 4.68 | | |
| PHELIX207 | 8.5900 | 194.57 | | | |
| PHELIX208 | 8.1500 | 184.34 | | | |
| PHELIX209 | 7.8700 | >10 | | | |
| PHELIX210 | >10 | >10 | | | |
| PHELIX211 | 208.6300 | NI | | | |
| PHELIX212 | 7.4100 | 138.53 | | | |
| PHELIX213 | 0.0200 | 1.35 | 0.13 | | |
| PHELIX214 | 24.1500 | NI | | | |
| PHELIX215 | 0.0200 | 0.99 | 0.16 | | |
| PHELIX219 | NI | NI | | | |
| PHELIX220 | 0.0600 | 3.15 | | | |
| PHELIX221 | 0.0300 | 1.32 | | | |
| PHELIX222 | 5.1900 | 151.28 | | | |
| PHELIX223 | 5.6100 | 35.33 | | | |
| PHELIX224 | 11.0200 | NI | | | |
| PHELIX225 | 6.1700 | 84.20 | | | |
| PHELIX226 | 5.0900 | 113.66 | | | |
| PHELIX227 | 16.7700 | NI | | | |
| PHELIX228 | 3.0207 | | | | |
| PHELIX247 | | >30 | | | |
| PHELIX248 | | >30 | | | |
| PHELIX249 | | >30 | | | |
| PHELIX251 | | >30 | | | |
| PHELIX252 | | >30 | | | |
| PHELIX253 | | >30 | | | |
| PHELIX254 | | >30 | | 3.4 | NI |
| PHELIX256 | | 4.97 | | | |
| PHELIX257 | | 5.08 | | 3.5 | 3 |
| PHELIX259 | | 1.10 | | | |
| PHELIX260 | | 1.81 | | | |
| PHELIX261 | | 2.56 | | | |
| PHELIX263 | | 0.99 | | | |
| PHELIX264 | | 2.68 | | | |
| PHELIX265 | | 2.20 | | | |
| PHELIX266 | | 0.78 | | | |
| PHELIX268 | | >30 | | | |
| PHELIX269 | | >30 | | NI | NI |
| PHELIX270 | | >30 | | 3.1 | NI |
| PHELIX294 | | | | NI | NI |
| PHELIX297 | | | | 3.8 | 3.4 |
| PHELIX299 | | | | NI | NI |
| PHELIX300 | | | | 4.5 | NI |
| PHELIX301 | | | | 2.9 | 1.1 |
| PHELIX309 | | | | 2.7 | 2 |
| PHELIX310 | | | | NI | NI |
| PHELIX312 | | | | NI | NI |
| PHELIX314 | | | | NI | 1.2 |
| PHELIX315 | | | | NI | NI |
| PHELIX319 | | | | 11.7 | NI |

What is claimed:

1. A compound comprising an α-ketoamide linkage that is terminated on each end by an amino acid.

2. The compound of claim 2, which is of Formula (I): 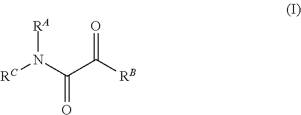
wherein:
R^{A} is H or C₁₋₆alkyl; and
R^{B} and R^{C} are, independently, an amino acid.

3. A compound of Formula (II): 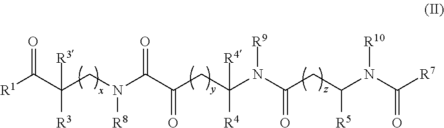
wherein:
x, y, and z are, independently, 0 or 1;
R¹ is:
absent,
OH,
NH₂,
optionally substituted heterocyclyl,
NH-(optionally substituted C₁₋₆alkyl),
N-(optionally substituted C₁₋₆alkyl)₂,
NH-(optionally substituted C₁₋₆alkyl-optionally substituted aryl)C(O)NH₂,
NH-(optionally substituted C₁₋₆alkyl-optionally substituted aryl)C(O)N(optionally substituted C₁₋₆alkyl)₂,
NH-(optionally substituted C₁₋₆alkyl-optionally substituted aryl)C(O)NH-optionally substituted C₁₋₆alkyl-optionally substituted aryl,
NH-(optionally substituted C₃₋₈cycloalkyl),
NH-(optionally substituted C₁₋₆alkyl-optionally substituted aryl),
NH-(optionally substituted C₁₋₆alkyl-optionally substituted heterocyclyl),
NH-(optionally substituted C₁₋₆alkyl)₂C(O)NH-(optionally substituted C₁₋₆alkyl)-optionally substituted heterocyclyl,
NH-(optionally substituted C₁₋₆alkyl)C(O)NH-(optionally substituted C₁₋₆alkyl)-(optionally substituted aryl),
NH-(optionally substituted C₁₋₆alkyl)C(O)NH-(optionally substituted C₁₋₆alkyl)-(optionally substituted aryl),
NH-(optionally substituted C₁₋₆alkoxy)-(optionally substituted C₂₋₆alkenyl)-C(O)NH(optionally substituted C₁₋₆alkylC(O)NH₂)-optionally substituted C₁₋₆alkyl-optionally substituted aryl, or
NHCH(optionally substituted C₁₋₆alkyl)(C(O)NH(C(O)NH₂)(optionally substituted C₁₋₆alkyl-optionally substituted guanidinyl);
R³ and R^{3′} are, independently:
H,
optionally substituted C₁₋₆alkyl,
optionally substituted C₁₋₆alkyl-optionally substituted aryl,
optionally substituted C₁₋₆alkyl-optionally substituted C₃₋₈cycloalkyl,
optionally substituted C₁₋₆alkyl-optionally substituted heteroaryl,
optionally substituted C₁₋₆alkyl-guanidinyl, or
optionally substituted C₁₋₆alkyl-NHC(O)(optionally substituted C₂₋₆alkenyl);
or R³ and R^{3′} are fused to form a 3 to 8-membered ring;
R⁴, R^{4′}, and R⁵ are, independently:
H,
optionally substituted C₁₋₆alkyl,
optionally substituted C₁₋₆alkoxy-optionally substituted C₂₋₆alkenyl,
optionally substituted C₁₋₆alkyl-optionally substituted aryl,
optionally substituted C₁₋₆alkyl-optionally substituted C₃₋₈cycloalkyl, or
optionally substituted C₁₋₆alkyl-optionally substituted heteroaryl;
R⁷ is:
absent;
optionally substituted C₁₋₆alkyl,
optionally substituted C₁₋₆oxyalkyl-optionally substituted aryl,
optionally substituted C₁₋₆alkyl-optionally substituted aryl,
optionally substituted C₁₋₆alkyl-optionally substituted aryl-NHC(O)-(optionally substituted C₁₋₆oxyalkyl-optionally substituted aryl),
optionally substituted aryl,
(optionally substituted C₁₋₆alkyl)-NHC(O)-optionally substituted C₁₋₆alkyl,
(optionally substituted C₁₋₆alkoxy)-(optionally substituted C₂₋₆alkenyl)-NHC(O)-optionally substituted C₁₋₆alkyl-optionally substituted aryl;
optionally substituted C₁₋₆aminoalkyl; or
optionally substituted C₁₋₆alkyl-optionally substituted aryl-optionally substituted C₁₋₆oxyalkyl-optionally substituted C₂₋₆alkenyl)NHC(O)(optionally substituted C₁₋₆alkyl)-(optionally substituted aryl);
R⁸, R⁹, and R¹⁰ are, independently, H or optionally substituted C₁₋₆alkyl;
or a pharmaceutically acceptable salt thereof.

4. The compound of claim 3, wherein R¹ is OH or NH₂.

5. The compound of claim 3, wherein R¹ is NH(optionally substituted C₁₋₆alkyl) such as NH(methyl), NH(ethyl), NH(propyl), NH(butyl), NH(pentyl), or NH(hexyl) or N(optionally substituted C₁₋₆alkyl)₂ such as N(methyl)₂, N(ethyl)₂, N(propyl)₂, N(butyl)₂, N(pentyl)₂, or N(hexyl)₂.

6. The compound of claim 3, wherein R¹ is NH(optionally substituted C₁₋₆alkyl-optionally substituted aryl) or NH(optionally substituted C₁₋₆alkyl-optionally substituted heterocyclyl).

7. The compound of claim 3, wherein R¹ is NH(optionally substituted C₁₋₆alkyl) substituted with C(O)OH, C(O)O(C₁₋₆alkyl), or C(O)NH₂.

8. The compound of claim 3, wherein R¹ is NH(optionally substituted C₃₋₈cycloalkyl) such as NH(cyclopentyl).

9. The compound of claim 3, wherein R¹ is heteroaryl such as pyrrolidinyl or thiazolyl.

10. The compound of claim 3, wherein R¹ is NH(optionally substituted C₁₋₆alkyl-optionally substituted aryl)C(O)NH₂ such as NH(benzyl)C(O)NH₂.

11. The compound of claim 3, wherein R¹ is NH-(optionally substituted C₁₋₆alkyl)₂C(O)NH-(optionally substituted C₁₋₆alkyl-optionally substituted heterocyclyl) such as NH—(C₁₋₆alkyl)₂C(O)NH—C₁₋₆alkylmorpholinyl.

12. The compound of claim 3, wherein R¹ is NH-(optionally substituted C₁₋₆alkyl)C(O)NH-optionally substituted C₁₋₆alkyl-optionally substituted aryl such as NH—(C₁₋₆alkyl)C(O)NH-benzyl or NH—(C₁₋₆alkyl)C(O)NH—CH₂CH₂-phenyl.

13. The compound of claim 3, wherein R¹ is NH-(optionally substituted C₁₋₆alkyl-optionally substituted aryl)C(O)N(optionally substituted C₁₋₆alkyl)₂ such as NH—(C₁₋₆alkyl-aryl)C(O)N(methyl)₂.

14. The compound of claim 3, wherein R¹ is NH-(optionally substituted C₁₋₆alkyl-optionally substituted aryl)C(O)NH-(optionally substituted C₁₋₆alkyl-optionally substituted aryl) such as —NH—(C₁₋₆alkyl-aryl)C(O)NH-benzyl or —NH—(C₁₋₆alkyl-aryl)C(O)NH—CH₂CH₂-phenyl.

15. The compound of claim 3, wherein R¹ is NH-(optionally substituted C₁₋₆alkoxy-optionally substituted C₂₋₆alkenyl)-C(O)NH(optionally substituted C₁₋₆alkylC(O)NH₂)-(optionally substituted C₁₋₆alkyl-optionally substituted aryl).

16. The compound of claim 3, wherein R¹ is NHCH(optionally substituted C₁₋₆alkyl)(C(O)NH(C(O)NH₂)(optionally substituted C₁₋₆alkyl-guanidinyl).

17. The compound of any one of claims 3-15, wherein R³ is H or optionally substituted C₁₋₆alkyl such as methyl, ethyl, propyl, butyl, pentyl, or hexyl.

18. The compound of any one of claims 3-15, wherein R³ is optionally substituted C₁₋₆alkyl-optionally substituted aryl such as phenyl or naphthyl.

19. The compound of claim 17, wherein the C₁₋₆alkyl is substituted with guanidinyl or citrulline.

20. The compound of any one of claims 3-15, wherein R³ is optionally substituted C₁₋₆alkyl-optionally substituted heteroaryl such as pyridyl or indolyl.

21. The compound of any one of claims 3-15, wherein R³ is optionally substituted C₁₋₆alkyl-optionally substituted C₃₋₈cycloalkyl such as C₁₋₆alkylcyclopropyl, C₁₋₆alkylcyclobutyl, C₁₋₆alkylcyclopentyl, or C₁₋₆alkylcyclohexyl.

22. The compound of any one of claims 3-15, wherein R³ is optionally substituted C₁₋₆alkyl-NHC(O)O(optionally substituted C₂₋₆alkenyl).

23. The compound of any one of claims 3-15, wherein R³ and R^{3′} are joined to from an optionally substituted ring such as cyclopropyl, cyclobutyl, cyclopentyl, or cyclohexyl.

24. The compound of any one of claims 3-23, wherein R⁴ is H or optionally substituted C₁₋₆alkyl such as methyl, ethyl, propyl, butyl, pentyl, or hexyl.

25. The compound of any one of claims 3-23, wherein R⁴ is optionally substituted C₁₋₆alkyl-optionally substituted aryl such as C₁₋₆alkylphenyl.

26. The compound of any one of claims 3-23, wherein R⁴ is optionally substituted C₁₋₆alkoxy-optionally substituted C₂₋₆alkenyl.

27. The compound of any one of claims 3-26, wherein R⁵ is H or optionally substituted C₁₋₆alkyl such as methyl, ethyl, propyl, butyl, pentyl, or hexyl.

28. The compound of any one of claims 3-26, wherein R⁵ is optionally substituted C₁₋₆alkyl-optionally substituted aryl such as C₁₋₆alkylphenyl.

29. The compound of any one of claims 3-26, wherein R⁵ is optionally substituted C₁₋₆alkyl-optionally substituted C₃₋₈cycloalkyl such as C₁₋₆alkylcyclopropyl.

30. The compound of any one of claims 3-26, wherein R⁵ is optionally substituted C₁₋₆alkyl-optionally substituted heteroaryl such as C₁₋₆alkylthiazolyl or C₁₋₆alkylpyridinyl.

31. The compound of any one of claims 3-30, wherein R⁷ is absent.

32. The compound of any one of claims 3-30, wherein R⁷ is optionally substituted C₁₋₆alkyl such as methyl, ethyl, propyl, butyl, pentyl, or hexyl.

33. The compound of any one of claims 3-30, wherein R⁷ is optionally substituted C₁₋₆alkyl-optionally substituted aryl such as C₁₋₆alkylphenyl, C₁₋₆alkyl-bi-phenyl or C₁₋₆alkylnaphthyl.

34. The compound of any one of claims 3-30, wherein R⁷ is optionally substituted C₁₋₆oxyalkyl-optionally substituted aryl such as C₁₋₆oxyalkylphenyl.

35. The compound of any one of claims 3-30, wherein R⁷ is optionally substituted aryl such as phenyl.

36. The compound of any one of claims 3-30, wherein R⁷ is -(optionally substituted C₁₋₆alkyl)NHC(O)(optionally substituted C₁₋₆alkyl).

37. The compound of any one of claims 3-30, wherein R⁷ is -(optionally substituted C₁₋₆alkoxy)-(optionally substituted C₂₋₆alkenyl)-NHC(O)-optionally substituted C₁₋₆alkyl-optionally substituted aryl such as —(C₁₋₆alkoxy)-(C₂₋₆alkenyl)-NHC(O)benzyl, —CH(CH₂OCH₂—C₂₋₆alkenyl)-NHC(O)benzyl, or —(C₁₋₆alkoxy)-(CH═CH₂)—NHC(O)benzyl.

38. The compound of any one of claims 3-30, wherein R⁷ is optionally substituted-C₁₋₆alkyl-optionally substituted aryl-NHC(O)-(optionally substituted C₁₋₆oxyalkyl-optionally substituted aryl) such as —C₁₋₆alkylphenyl-NHC(O)—(C₁₋₆oxyalkylphenyl).

39. The compound of any one of claims 3-30, wherein R⁷ is optionally substituted C₁₋₆alkyl-optionally substituted aryl-optionally substituted C₁₋₆oxyalkyl-optionally substituted C₂₋₆alkenyl)NHC(O)-(optionally substituted C₁₋₆alkyl)-(optionally substituted aryl).

40. The compound of any one of claims 3-39, wherein R⁸, R⁹, and R¹⁰ are H.

41. The compound of any one of claims 3-39, wherein R⁸, R⁹, and R¹⁰ are optionally substituted C₁₋₆alkyl.

42. The compound of any one of claims 3-41, which is of Formula (III):

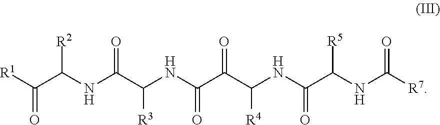

43. The compound of any one of claims 3-41, which is of Formula (IV): 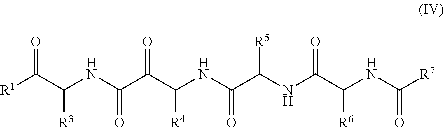
wherein, R⁶ is optionally substituted C₁₋₆alkyl.

44. The compound of any one of claims 3-41, which is of Formula (V): 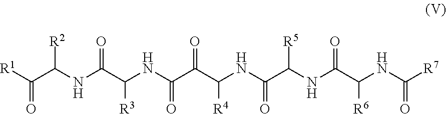
wherein, R⁶ is optionally substituted C₁₋₆alkyl- optionally substituted C₂₋₆alkenyloxy, optionally substituted C₁₋₆alkyl- optionally substituted aryl- optionally substituted C₂₋₆alkenyloxy, or optionally substituted C₁₋₆alkyl- optionally substituted aryl such as benzyl.

45. The compound of any one of claims 1-3, that is any of:
2-methyl-2-(3-methyl-2-(2-oxo-4-phenyl-3-(2-phenylacetamido)butanamido)pentanamido)propanoic acid;
2-methyl-2-(3-methyl-2-(2-oxo-3-(2-phenylacetamido)butanamido)pentanamido)propanoic acid;
2-methyl-2-(3-methyl-2-(2-oxo-4-(2-phenylacetamido)butanamido)pentanamido)propanoic acid;
(2-oxo-3-(2-phenylacetamido)butanoyl)isoleucylproline;
2-methyl-2-(3-methyl-2-(3-methyl-2-oxo-3-(2-phenylacetamido)butanamido)pentanamido)propanoic acid;
12-benzyl-5-(sec-butyl)-2,2,9-trimethyl-4,7,8,11,14-pentaoxo-15-phenyl-3,6,10,13-tetraazapentadecanoic acid;
9,12-dibenzyl-5-(sec-butyl)-2,2-dimethyl-4,7,8,11,14-pentaoxo-15-phenyl-3,6,10,13-tetraazapentadecanoic acid;
(2-oxo-3-(3-phenyl-2-(2-phenylacetamido)propanamido)butanoyl)isoleucylproline;
(2-oxo-4-phenyl-3-(3-phenyl-2-(2-phenylacetamido)propanamido)butanoyl)isoleucylproline;
5,12-dibenzyl-2,2,9-trimethyl-4,7,8,11,14-pentaoxo-15-phenyl-3,6,10,13-tetraazapentadecanoic acid;
12-benzyl-2,2,9-trimethyl-5-(naphthalen-2-ylmethyl)-4,7,8,11,14-pentaoxo-15-phenyl-3,6,10,13-tetraazapentadecanoic acid;
15-([1,1′-biphenyl]-4-yl)-12-benzyl-5-(sec-butyl)-2,2,9-trimethyl-4,7,8,11,14-pentaoxo-3,6,10,13-tetraazapentadecanoic acid;
12-benzyl-5-(sec-butyl)-2,2,9,9-tetramethyl-4,7,8,11,14-pentaoxo-15-phenyl-3,6,10,13-tetraazapentadecanoic acid;
12-benzyl-5-(sec-butyl)-2,2,9-trimethyl-15-(naphthalen-2-yl)-4,7,8,11,14-pentaoxo-3,6,10,13-tetraazapentadecanoic acid;
12-benzyl-2,2,9-trimethyl-5-(naphthalen-1-ylmethyl)-4,7,8,11,14-pentaoxo-15-phenyl-3,6,10,13-tetraazapentadecanoic acid;
12-benzyl-5-(sec-butyl)-2,2,9-trimethyl-15-(naphthalen-1-yl)-4,7,8,11,14-pentaoxo-3,6,10,13-tetraazapentadecanoic acid;
13-(sec-butyl)-9,16,16-trimethyl-3,7,10,11,14-pentaoxo-1-phenyl-2-oxa-4,8,12,15-tetraazaheptadecan-17-oic acid;
12-([1,1′-biphenyl]-4-ylmethyl)-5-(sec-butyl)-2,2,9-trimethyl-4,7,8,11,14-pentaoxo-15-phenyl-3,6,10,13-tetraazapentadecanoic acid;
12-benzyl-5-(cyclopropylmethyl)-2,2,9-trimethyl-4,7,8,11,14-pentaoxo-15-phenyl-3,6,10,13-tetraazapentadecanoic acid;
5-(sec-butyl)-12-(4-(tert-butyl)benzyl)-2,2,9-trimethyl-4,7,8,11,14-pentaoxo-15-phenyl-3,6,10,13-tetraazapentadecanoic acid;
5-(sec-butyl)-2,2,9-trimethyl-4,7,8,11,14-pentaoxo-15-phenyl-12-(4-(trifluoromethyl)benzyl)-3,6,10,13-tetraazapentadecanoic acid;
12-(4-bromobenzyl)-5-(sec-butyl)-2,2,9-trimethyl-4,7,8,11,14-pentaoxo-15-phenyl-3,6,10,13-tetraazapentadecanoic acid;
5-(sec-butyl)-2,2,9-trimethyl-4,7,8,11,14-pentaoxo-15-phenyl-12-(3-(trifluoromethyl)benzyl)-3,6,10,13-tetraazapentadecanoic acid;
5-(sec-butyl)-12-isobutyl-2,2,9-trimethyl-4,7,8,11,14-pentaoxo-15-phenyl-3,6,10,13-tetraazapentadecanoic acid;
5-(sec-butyl)-12-(4-fluorobenzyl)-2,2,9-trimethyl-4,7,8,11,14-pentaoxo-15-phenyl-3,6,10,13-tetraazapentadecanoic acid;
(12S)-5-(sec-butyl)-2,2,9-trimethyl-4,7,8,11,14-pentaoxo-15-phenyl-12-(thiazol-5-ylmethyl)-3,6,10,13-tetraazapentadecanoic acid;
3-((2-oxo-4-phenyl-3-(3-phenyl-2-(2-phenylacetamido)propanamido)butanoyl)isoleucyl)thiazolidine-4-carboxylic acid;
5-(sec-butyl)-2,2,9-trimethyl-12-(3-methylbenzyl)-4,7,8,11,14-pentaoxo-15-phenyl-3,6,10,13-tetraazapentadecanoic acid;
5-(sec-butyl)-12-(3 -cyanobenzyl)-2,2,9-trimethyl-4,7,8,11,14-pentaoxo-15-phenyl-3,6,10,13-tetraazapentadecanoic acid;
12-benzyl-5-(cyclohexylmethyl)-2,2,9-trimethyl-4,7,8,11,14-pentaoxo-15-phenyl-3,6,10,13-tetraazapentadecanoic acid;
(2-oxo-3-(3-phenyl-2-(2-phenylacetamido)propanamido)butanoyl)phenylalanylproline;
methyl 12-benzyl-5-(sec-butyl)-2,2,9-trimethyl-4,7,8,11,14-pentaoxo-15-phenyl-3,6,10,13-tetraazapentadecanoate;
12-benzyl-5-(4-(tert-butyl)benzyl)-2,2,9-trimethyl-4,7,8,11,14-pentaoxo-15-phenyl-3,6,10,13-tetraazapentadecanoic acid;
N-(4-((1-((1-amino-2-methyl-1-oxopropan-2-yl)amino)-1-oxopropan-2-yl)amino)-3,4-dioxobutan-2-yl)-4-methyl-2-(2-phenylacetamido)pentanamide;
benzyl (1-((4-((2-((1-amino-3-(naphthalen-2-yl)-1-oxopropan-2-yl)amino)-2-oxoethyl)amino)-3,4-dioxobutan-2-yl)amino)-4-methyl-1-oxopentan-2-yl)carbamate;
benzyl (1-((4-((3-((1-amino-3-(3-cyanophenyl)-1-oxopropan-2-yl)amino)-3-oxopropyl)amino)-3,4-dioxobutan-2-yl)amino)-4-methyl-1-oxopentan-2-yl)carbamate;
benzyl (1-((4-(4-((1-amino-1-oxo-3-phenylpropan-2-yl)carbamoyl)piperidin-1-yl)-3,4-dioxobutan-2-yl)amino)-4-methyl-1-oxopentan-2-yl)carbamate;
benzyl (1-((4-(3-((1-amino-1-oxo-3-phenylpropan-2-yl)carbamoyl)azetidin-1-yl)-3,4-dioxobutan-2-yl)amino)-4-methyl-1-oxopentan-2-yl)carbamate;
benzyl (1-((4-((1-amino-1-oxo-3-(pyridin-4-yl)propan-2-yl)amino)-3,4-dioxobutan-2-yl)amino)-4-methyl-1-oxopentan-2-yl)carbamate;
2-(3-(allyloxy)-2-(2-phenylacetamido)propanamido)-N-(1-(allyloxy)-4-((2-((1-amino-3-(naphthalen-2-yl)-1-oxopropan-2-yl)amino)-2-oxoethyl)amino)-3,4-dioxobutan-2-yl)-4-methylpentanamide;
N-(1-(allyloxy)-4-((2-((1-amino-3-(naphthalen-2-yl)-1-oxopropan-2-yl)amino)-2-oxoethyl)amino)-3,4-dioxobutan-2-yl)-2-(3-(4-(allyloxy)phenyl)-2-(2-phenylacetamido)propanamido)-4-methylpentanamide;
1-((2-oxo-4-phenyl-3-(3 -phenyl-2-(2-phenylacetamido)propanamido)butanoyl)isoleucyl)pyrrolidine-2-carboxamide;
N-(1-((1-amino-2-methyl-1-oxopropan-2-yl)amino)-1-oxo-3-phenylpropan-2-yl)-3-(3-(naphthalen-1-yl)-2-(2-phenylacetamido)propanamido)-2-oxobutanamide;
N-(1-((1-amino-2-methyl-1-oxopropan-2-yl)amino)-1-oxo-3-phenylpropan-2-yl)-3-(3-(naphthalen-2-yl)-2-(2-phenylacetamido)propanamido)-2-oxobutanamide;
N-(1-((1-amino-2-methyl-1-oxopropan-2-yl)amino)-1-oxo-3-phenylpropan-2-yl)-3-(3-(2-fluorophenyl)-2-(2-phenylacetamido)propanamido)-2-oxobutanamide;
N-(1-((1-amino-2-methyl-1-oxopropan-2-yl)amino)-1-oxo-3-phenylpropan-2-yl)-3-(3-(4-fluorophenyl)-2-(2-phenylacetamido)propanamido)-2-oxobutanamide;
N-(1-((1-amino-2-methyl-1-oxopropan-2-yl)amino)-1-oxo-3-phenylpropan-2-yl)-2-oxo-3-(2-(2-phenylacetamido)-3-(3-(trifluoromethyl)phenyl)propanamido)butanamide;
N-(1-((1-amino-2-methyl-1-oxopropan-2-yl)amino)-1-oxo-3-phenylpropan-2-yl)-2-oxo-3-(2-(2-phenylacetamido)-3-(pyridin-3-yl)propanamido)butanamide;
N-(1-((1-amino-2-methyl-1-oxopropan-2-yl)amino)-1-oxo-3-phenylpropan-2-yl)-3-(3-(3-fluorophenyl)-2-(2-phenylacetamido)propanamido)-2-oxobutanamide;
N-(1-((3-([1,1′-biphenyl]-4-yl)-1-amino-1-oxopropan-2-yl)amino)-1-oxo-3-phenylpropan-2-yl)-2-oxo-3-(3-phenyl-2-(2-phenylacetamido)propanamido)butanamide;
N-(1-amino-2-methyl-1-oxopropan-2-yl)-4-methyl-2-(2-oxo-3-(3-phenyl-2-(2-phenylacetamido)propanamido)butanamido)pentanamide;
N-(1-((1-amino-3-(naphthalen-2-yl)-1-oxopropan-2-yl)amino)-1-oxo-3-phenylpropan-2-yl)-2-oxo-3-(3-phenyl-2-(2-phenylacetamido)propanamido)butanamide;
1-(3-methyl-2-(2-oxo-3-(3-phenyl-2-(2-phenylacetamido)propanamido)butanamido)pentanamido)cyclopentane-1-carboxylic acid;
13-benzyl-5-(sec-butyl)-2,2-dimethyl-4,7,8,12,15-pentaoxo-16-phenyl-3,6,11,14-tetraazahexadecanoic acid;
N-(1-((1-amino-1-oxo-3-phenylpropan-2-yl)amino)-1-oxo-3-phenylpropan-2-yl)-2-oxo-3-(2-(2-phenylacetamido)-3-(pyridin-2-yl)propanamido)butanamide;
N-(1-((1-amino-3-(4-(tert-butyl)phenyl)-1-oxopropan-2-yl)amino)-1-oxo-3-phenylpropan-2-yl)-2-oxo-3-(3-phenyl-2-(2-phenylacetamido)propanamido)butanamide;
N-(1-((4-((1-((1-amino-2-methyl-1-oxopropan-2-yl)amino)-1-oxo-3-phenylpropan-2-yl)amino)-3,4-dioxobutan-2-yl)amino)-1-oxo-3-phenylpropan-2-yl)-3-fluorobenzamide;
N-(1-((4-((1-((1-amino-2-methyl-1-oxopropan-2-yl)amino)-1-oxo-3-phenylpropan-2-yl)amino)-3,4-dioxobutan-2-yl)amino)-1-oxo-3-phenylpropan-2-yl)benzamide;
N-(1-((4-((1-((1-amino-2-methyl-1-oxopropan-2-yl)amino)-1-oxo-3-phenylpropan-2-yl)amino)-3,4-dioxobutan-2-yl)amino)-1-oxo-3-phenylpropan-2-yl)-2-fluorobenzamide;
N-(1-((1-amino-2-methyl-1-oxopropan-2-yl)amino)-1-oxo-3-phenylpropan-2-yl)-2-oxo-3-(3-phenyl-2-(2-(m-tolyl)acetamido)propanamido)butanamide;
N-(1-((1-amino-2-methyl-1-oxopropan-2-yl)amino)-1-oxo-3-phenylpropan-2-yl)-2-oxo-3-(3-phenyl-2-(2-(o-tolyl)acetamido)propanamido)butanamide;
N-(1-((1-amino-2-methyl-1-oxopropan-2-yl)amino)-1-oxo-3-phenylpropan-2-yl)-3-(2-(2-(3-fluorophenyl)acetamido)-3-phenylpropanamido)-2-oxobutanamide;
benzyl (1-((1-(allyloxy)-4-((3-(allyloxy)-1-((1-amino-3-(naphthalen-2-yl)-1-oxopropan-2-yl)amino)-1-oxopropan-2-yl)amino)-3,4-dioxobutan-2-yl)amino)-4-methyl-1-oxopentan-2-yl)carbamate;
N-(1-((2-methyl-1-oxo-1-(phenethylamino)propan-2-yl)amino)-1-oxo-3-phenylpropan-2-yl)-2-oxo-3-(3-phenyl-2-(2-phenylacetamido)propanamido)butanamide;
N-(1-((2-methyl-1-((2-morpholinoethyl)amino)-1-oxopropan-2-yl)amino)-1-oxo-3-phenylpropan-2-yl)-2-oxo-3-(3-phenyl-2-(2-phenylacetamido)propanamido)butanamide;
N-(1-((1-amino-2-methyl-1-oxopropan-2-yl)amino)-3-(1H-indol-2-yl)-1-oxopropan-2-yl)-2-oxo-3-(3-phenyl-2-(2-phenylacetamido)propanamido)butanamide;
N-(1-((1-amino-2-methyl-1-oxopropan-2-yl)amino)-1-oxo-3-phenylpropan-2-yl)-4-methyl-2-oxo-3-(3-phenyl-2-(2-phenylacetamido)propanamido)hexanamide;
N-(1-amino-2-methyl-1-oxopropan-2-yl)-5-guanidino-2-(2-oxo-3-(3-phenyl-2-(2-phenylacetamido)propanamido)butanamido)pentanamide;
N-(1-((1-amino-2-methyl-1-oxopropan-2-yl)amino)-1-oxo-3-phenylpropan-2-yl)-3-(2-(3-methylbutanamido)-3-phenylpropanamido)-2-oxobutanamide;
N-(1-((4-((1-((1-amino-2-methyl-1-oxopropan-2-yl)amino)-1-oxo-3-phenylpropan-2-yl)amino)-3,4-dioxobutan-2-yl)amino)-1-oxo-3-phenylpropan-2-yl)pentanamide;
N-((S)-1-((1-amino-2-methyl-1-oxopropan-2-yl)amino)-1-oxo-3-phenylpropan-2-yl)-3-((S)-2-(2,2-diphenylacetamido)-3-phenylpropanamido)-2-oxobutanamide;
benzyl (5-benzyl-12,17-dimethyl-4,7,10,11,14-pentaoxo-1-phenyl-3,6,9,13-tetraazaoctadecan-15-yl)carbamate;
N-(3-([1,1′-biphenyl]-4-yl)-1-((1-amino-2-methyl-1-oxopropan-2-yl)amino)-1-oxopropan-2-yl)-2-oxo-3-(3-phenyl-2-(2-phenylacetamido)propanamido)butanamide;
N-(1-amino-2-methyl-1-oxopropan-2-yl)-2-(2-oxo-3-(3-phenyl-2-(2-phenylacetamido)propanamido)butanamido)hexanamide;
N-(1-amino-2-methyl-1-oxopropan-2-yl)-2-(2-oxo-3-(3-phenyl-2-(2-phenylacetamido)propanamido)butanamido)pentanamide;
N-(1-((1-amino-2-methyl-1-oxopropan-2-yl)amino)-1-oxo-3-phenylpropan-2-yl)-2-oxo-3-(3-phenyl-2-(2-phenylacetamido)propanamido)butanamide;
N-(4-((2-((1-amino-3-(naphthalen-2-yl)-1-oxopropan-2-yl)amino)-2-oxoethyl)amino)-3,4-dioxobutan-2-yl)-2-(2,2-diphenylacetamido)-4-methylpentanamide;
15-((1-amino-5-guanidino-1-oxopentan-2-yl) carbamoyl)-8-benzyl-5-isobutyl-12-methyl-3,6,9,10,13-pentaoxo-1-phenyl-2-oxa-4,7,11,14-tetraazaoctadecan-18-oic acid;
2-(2-([1,1′-biphenyl]-4-yl)acetamido)-N-(4-((2-((1-amino-3-(naphthalen-2-yl)-1-oxopropan-2-yl)amino)-2-oxoethyl)amino)-3,4-dioxobutan-2-yl)-4-methylpentanamide;
benzyl (1-amino-10-benzyl-6-carbamoyl-1-imino-13-isobutyl-8,9,12,15-tetraoxo-17-phenyl-2,7,11,14-tetraazaheptadecan-16-yl)carbamate;
benzyl (1-((4-((2-((1-amino-3-(naphthalen-2-yl)-1-oxopropan-2-yl)amino)-2-oxoethyl)amino)-3,4-dioxo-1-phenylbutan-2-yl)amino)-4-methyl-1-oxopentan-2-yl)carbamate;
benzyl (1-((1-((2-((1-amino-3-(naphthalen-2-yl)-1-oxopropan-2-yl)amino)-2-oxoethyl)amino)-4-methyl-1,2-dioxohexan-3-yl)amino)-4-methyl-1-oxopentan-2-yl)carbamate;
benzyl (1-amino-12-isobutyl-9-methyl-2-(naphthalen-2-ylmethyl)-1,4,7,8,11,14-hexaoxo-16-phenyl-3,6,10,13-tetraazahexadecan-15-yl)carbamate;
N-(1-((3-([1,1′-biphenyl]-4-yl)-1-amino-1-oxopropan-2-yl)amino)-3-cyclohexyl-1-oxopropan-2-yl)-2-oxo-3-(3-phenyl-2-(2-phenylacetamido)propanamido)butanamide;
N-(1-((3-([1,1′-biphenyl]-4-yl)-1-amino-1-oxopropan-2-yl)amino)-3-cyclopropyl-1-oxopropan-2-yl)-2-oxo-3-(3-phenyl-2-(2-phenylacetamido)propanamido)butanamide;
N-(1-((3-([1,1′-biphenyl]-4-yl)-1-amino-1-oxopropan-2-yl)amino)-1-oxo-3-(4-(trifluoromethyl)phenyl)propan-2-yl)-2-oxo-3-(3-phenyl-2-(2-phenylacetamido)propanamido)butanamide;
N-(1-((3-([1,1′-biphenyl]-4-yl)-1-amino-1-oxopropan-2-yl)amino)-3-(4-fluorophenyl)-1-oxopropan-2-yl)-2-oxo-3-(3-phenyl-2-(2-phenylacetamido)propanamido)butanamide;
N-(1-((3-([1,1′-biphenyl]-4-yl)-1-amino-1-oxopropan-2-yl)amino)-3-(2-fluorophenyl)-1-oxopropan-2-yl)-2-oxo-3-(3-phenyl-2-(2-phenylacetamido)propanamido)butanamide;
N-(1-((3-([1,1′-biphenyl]-4-yl)-1-amino-1-oxopropan-2-yl)amino)-1-oxo-3-(3-(trifluoromethyl)phenyl)propan-2-yl)-2-oxo-3-(3-phenyl-2-(2-phenylacetamido)propanamido)butanamide;
N-(1-((3-([1,1′-biphenyl]-4-yl)-1-amino-1-oxopropan-2-yl)amino)-1-oxo-3-(2-(trifluoromethyl)phenyl)propan-2-yl)-2-oxo-3-(3-phenyl-2-(2-phenylacetamido)propanamido)butanamide;
N-(1-((3-([1,1′-biphenyl]-4-yl)-1-amino-1-oxopropan-2-yl)amino)-3-(3-fluorophenyl)-1-oxopropan-2-yl)-2-oxo-3-(3-phenyl-2-(2-phenylacetamido)propanamido)butanamide;
benzyl (1-amino-10-(sec-butyl)-6-carbamoyl-1-imino-13-isobutyl-8,9,12,15-tetraoxo-17-phenyl-2,7,11,14-tetraazaheptadecan-16-yl)carbamate;
benzyl (15-(sec-butyl)-11-carbamoyl-18-isobutyl-5,13,14,17,20-pentaoxo-22-phenyl-4-oxa-6,12,16,19-tetraazadocos-1-en-21-yl)carbamate;
benzyl (1-amino-9-(sec-butyl)-12-isobutyl-2-(naphthalen-2-ylmethyl)-1,4,7,8,11,14-hexaoxo-16-phenyl-3,6,10,13-tetraazahexadecan-15-yl)carbamate;
benzyl (1-((1-((1-((1-amino-3-(naphthalen-2-yl)-1-oxopropan-2-yl)amino)-5-guanidino-1-oxopentan-2-yl)amino)-4-methyl-1,2-dioxohexan-3-yl)amino)-4-methyl-1-oxopentan-2-yl)carbamate;
benzyl (1-((1-((1-amino-5-guanidino-1-oxopentan-2-yl)amino)-4-methyl-1,2-dioxohexan-3-yl)amino)-4-methyl-1-oxopentan-2-yl)carbamate;
benzyl (1-((1-((2-((1-amino-3-(naphthalen-2-yl)-1-oxopropan-2-yl)amino)-2-oxoethyl)(methyl)amino)-4-methyl-1,2-dioxohexan-3-yl)amino)-4-methyl-1-oxopentan-2-yl)carbamate;
benzyl (1-((1-((2-((1-amino-3-(naphthalen-2-yl)-1-oxopropan-2-yl)amino)-2-oxoethyl)amino)-4-methyl-1,2-dioxohexan-3-yl)(methyl)amino)-4-methyl-1-oxopentan-2-yl)carbamate;
benzyl (1-((1-((1-((1-amino-3-(naphthalen-2-yl)-1-oxopropan-2-yl)amino)-1-oxopropan-2-yl)amino)-4-methyl-1,2-dioxohexan-3-yl)amino)-4-methyl-1-oxopentan-2-yl)carbamate;
benzyl (1-((1-((1-((1-amino-3-(naphthalen-2-yl)-1-oxopropan-2-yl)amino)-2-methyl-1-oxopropan-2-yl)amino)-4-methyl-1,2-dioxohexan-3-yl)amino)-4-methyl-1-oxopentan-2-yl)carbamate;
benzyl (1-((1-(3-((1-amino-3-(naphthalen-2-yl)-1-oxopropan-2-yl)carbamoyl)azetidin-1-yl)-4-methyl-1,2-dioxohexan-3-yl)amino)-4-methyl-1-oxopentan-2-yl)carbamate;
benzyl (1-((1-((1-((1-amino-3-(naphthalen-2-yl)-1-oxopropan-2-yl)carbamoyl)cyclopropyl)amino)-4-methyl-1,2-dioxohexan-3-yl)amino)-4-methyl-1-oxopentan-2-yl)carbamate;
benzyl (1-((1-((2-((1-amino-3-(naphthalen-2-yl)-1-oxopropan-2-yl)amino)-2-oxoethyl)amino)-4-methyl-1,2-dioxohexan-3-yl)amino)-3-cyclopropyl-1-oxopropan-2-yl)carbamate;
N-(2-((1-amino-3-(naphthalen-2-yl)-1-oxopropan-2-yl)amino)-2-oxoethyl)-4-methyl-3-(4-methyl-2-(2-phenylacetamido)pentanamido)-2-oxohexanamide;
3-(3-(1H-imidazol-4-yl)-2-(2-phenylacetamido)propanamido)-N-(2-((1-amino-3-(naphthalen-2-yl)-1-oxopropan-2-yl)amino)-2-oxoethyl)-4-methyl-2-oxohexanamide;
benzyl (4-methyl-1-((4-methyl-1-((2-((naphthalen-2-ylmethyl)amino)-2-oxoethyl)amino)-1,2-dioxohexan-3-yl)amino)-1-oxopentan-2-yl)carbamate;
3-(2-acetamido-4-methylpentanamido)-N-(2-((1-amino-3-(naphthalen-2-yl)-1-oxopropan-2-yl)amino)-2-oxoethyl)-4-methyl-2-oxohexanamide;
benzyl (1-((1-((1-amino-3-(naphthalen-2-yl)-1-oxopropan-2-yl)amino)-4-methyl-1,2-dioxohexan-3-yl)amino)-4-methyl-1-oxopentan-2-yl)carbamate;
benzyl (1-((1-((2-((1-amino-3-(naphthalen-2-yl)-1-oxopropan-2-yl)amino)-2-oxoethyl)amino)-4-methyl-1,2-dioxohexan-3-yl)amino)-3-(1H-imidazol-4-yl)-1-oxopropan-2-yl)carbamate;
N-(2-((1-amino-3-(naphthalen-2-yl)-1-oxopropan-2-yl)amino)-2-oxoethyl)-4-methyl-3-(4-methyl-2-(2-(pyridin-3-yl)acetamido)pentanamido)-2-oxohexanamide;
N-(2-((1-amino-3-(naphthalen-2-yl)-1-oxopropan-2-yl)amino)-2-oxoethyl)-4-methyl-3-(4-methyl-2-(2-(4-methylpiperazin-1-yl)acetamido)pentanamido)-2-oxohexanamide;
N-(2-((1-amino-3-(naphthalen-2-yl)-1-oxopropan-2-yl)amino)-2-oxoethyl)-4-methyl-3-(4-methyl-2-(2-morpholinoacetamido)pentanamido)-2-oxohexanamide;
benzyl (1-((1-((1-((1-amino-3-(naphthalen-2-yl)-1-oxopropan-2-yl)amino)-1-oxo-5-ureidopentan-2-yl)amino)-4-methyl-1,2-dioxohexan-3-yl)amino)-4-methyl-1-oxopentan-2-yl)carbamate;
benzyl (1-((1-((4-amino-1-((1-amino-3-(naphthalen-2-yl)-1-oxopropan-2-yl)amino)-1,4-dioxobutan-2-yl)amino)-4-methyl-1,2-dioxohexan-3-yl)amino)-4-methyl-1-oxopentan-2-yl)carbamate;
benzyl (1-((1-((5-amino-1-((1-amino-3-(naphthalen-2-yl)-1-oxopropan-2-yl)amino)-1,5-dioxopentan-2-yl)amino)-4-methyl-1,2-dioxohexan-3-yl)amino)-4-methyl-1-oxopentan-2-yl)carbamate;
benzyl (1-((1-((2-((1-amino-1-oxo-3-phenylpropan-2-yl)amino)-2-oxoethyl)amino)-4-methyl-1,2-dioxohexan-3-yl)amino)-4-methyl-1-oxopentan-2-yl)carbamate;
benzyl (1-((1-((2-((1-amino-1-oxo-3-phenylpropan-2-yl)(methyl)amino)-2-oxoethyl)amino)-4-methyl-1,2-dioxohexan-3-yl)amino)-4-methyl-1-oxopentan-2-yl)carbamate;
N¹-(1-((2-((1-amino-3-(naphthalen-2-yl)-1-oxopropan-2-yl)amino)-2-oxoethyl)amino)-4-methyl-1,2-dioxohexan-3-yl)-2-(2-phenylacetamido)pentanediamide;
N-(2-((1-amino-3-(naphthalen-2-yl)-1-oxopropan-2-yl)amino)-2-oxoethyl)-4-methyl-2-oxo-3-(3-phenyl-2-(2-phenylacetamido)propanamido)hexanamide;
N¹-(1-((2-((1-amino-3-(naphthalen-2-yl)-1-oxopropan-2-yl)amino)-2-oxoethyl)amino)-4-methyl-1,2-dioxohexan-3-yl)-2-(2-phenylacetamido)succinamide;
N-(2-((1-amino-3-(naphthalen-2-yl)-1-oxopropan-2-yl)amino)-2-oxoethyl)-3-(3,3-dimethyl-2-(2-phenylacetamido)butanamido)-4-methyl-2-oxohexanamide;
N-(2-((1-amino-3-(naphthalen-2-yl)-1-oxopropan-2-yl)amino)-2-oxoethyl)-4-methyl-3-(4-methyl-2-(2-(pyridin-2-yl)acetamido)pentanamido)-2-oxohexanamide;
benzyl (1-((2-((1-amino-3-(naphthalen-2-yl)-1-oxopropan-2-yl)amino)-2-oxoethyl)amino)-4-methyl-1,2-dioxohexan-3-yl)carbamate;
benzyl (1-((1-((4-amino-4-oxo-1-phenylbutan-2-yl)amino)-4-methyl-1,2-dioxohexan-3-yl)amino)-4-methyl-1-oxopentan-2-yl)carbamate;
benzyl (1-((1-((2-((1-amino-3-(1H-indol-2-yl)-1-oxopropan-2-yl)amino)-2-oxoethyl)amino)-4-methyl-1,2-dioxohexan-3-yl)amino)-4-methyl-1-oxopentan-2-yl)carbamate;
benzyl (1-((1-((1-((1-amino-3-(naphthalen-2-yl)-1-oxopropan-2-yl)amino)-3-(1H-imidazol-5-yl)-1-oxopropan-2-yl)amino)-4-methyl-1,2-dioxohexan-3-yl)amino)-4-methyl-1-oxopentan-2-yl)carbamate;
N-(2-((1-amino-3-(naphthalen-2-yl)-1-oxopropan-2-yl)amino)-2-oxoethyl)-3-(4,4-dimethyl-2-(2-phenylacetamido)pentanamido)-4-methyl-2-oxohexanamide;
N-(2-((1-amino-3-(naphthalen-2-yl)-1-oxopropan-2-yl)amino)-2-oxoethyl)-3-(3-cyclopropyl-2-(2-phenylacetamido)propanamido)-4-methyl-2-oxohexanamide;
N-(2-((1-amino-3-(naphthalen-2-yl)-1-oxopropan-2-yl)amino)-2-oxoethyl)-4-methyl-3-(4-methyl-2-(2-(pyridin-4-yl)acetamido)pentanamido)-2-oxohexanamide;
N-(2-((1-amino-3-(naphthalen-2-yl)-1-oxopropan-2-yl)amino)-2-oxoethyl)-3-(2-(3-benzylureido)-4-methylpentanamido)-4-methyl-2-oxohexanamide;
N-(2-((1-amino-3-(naphthalen-2-yl)-1-oxopropan-2-yl)amino)-2-oxoethyl)-4-methyl-3-(4-methyl-2-(3-phenylureido)pentanamido)-2-oxohexanamide;
benzyl (1-((1-((1-((1-amino-3-(naphthalen-2-yl)-1-oxopropan-2-yl)amino)-3-cyano-1-oxopropan-2-yl)amino)-4-methyl-1,2-dioxohexan-3-yl)amino)-4-methyl-1-oxopentan-2-yl)carbamate;
N-(2-((1-amino-3-(naphthalen-2-yl)-1-oxopropan-2-yl)amino)-2-oxoethyl)-3-(3-cyano-2-(2-phenylacetamido)propanamido)-4-methyl-2-oxohexanamide;
3-(2-(2-acetamido-3-(naphthalen-2-yl)propanamido)acetamido)-N-(1-((1-amino-1-oxo-3-phenylpropan-2-yl)amino)-4-methyl-1-oxopentan-2-yl)-4-methyl-2-oxohexanamide;
benzyl (1-((1-((1-amino-1-oxo-5-ureidopentan-2-yl)amino)-4-methyl-1,2-dioxohexan-3-yl)amino)-4-methyl-1-oxopentan-2-yl)carbamate;
benzyl (1-((1-((1-amino-1-oxo-3-(pyridin-4-yl)propan-2-yl)amino)-4-methyl-1,2-dioxohexan-3-yl)amino)-4-methyl-1-oxopentan-2-yl)carbamate;
benzyl (1-((1-((1-amino-3-(1H-indol-2-yl)-1-oxopropan-2-yl)amino)-4-methyl-1,2-dioxohexan-3-yl)amino)-4-methyl-1-oxopentan-2-yl)carbamate;
benzyl (1-((1-((1-amino-1-oxo-3-(pyridin-3-yl)propan-2-yl)amino)-4-methyl-1,2-dioxohexan-3-yl)amino)-4-methyl-1-oxopentan-2-yl)carbamate;
3-(2-(2-acetamido-3-phenylpropanamido)-4-methylpentanamido)-N-(1-amino-5-guanidino-1-oxopentan-2-yl)-4-methyl-2-oxohexanamide;
N-(1-amino-5-guanidino-1-oxopentan-2-yl)-4-methyl-3-(4-methyl-2-(3-phenyl-2-(2-(pyridin-4-yl)acetamido)propanamido)pentanamido)-2-oxohexanamide;
benzyl (1-((1-((2-((1-amino-3-(3-fluorophenyl)-1-oxopropan-2-yl)amino)-2-oxoethyl)amino)-4-methyl-1,2-dioxohexan-3-yl)amino)-4-methyl-1-oxopentan-2-yl)carbamate;
benzyl (1-((1-((2-((1-amino-3-(4-fluorophenyl)-1-oxopropan-2-yl)amino)-2-oxoethyl)amino)-4-methyl-1,2-dioxohexan-3-yl)amino)-4-methyl-1-oxopentan-2-yl)carbamate;
benzyl (1-((1-((2-((1-amino-3-(4-cyanophenyl)-1-oxopropan-2-yl)amino)-2-oxoethyl)amino)-4-methyl-1,2-dioxohexan-3-yl)amino)-4-methyl-1-oxopentan-2-yl)carbamate;
N-(2-((1-amino-1-oxo-3-phenylpropan-2-yl)(methyl)amino)-2-oxoethyl)-4-methyl-3-(4-methyl-2-(2-(pyridin-3-yl)acetamido)pentanamido)-2-oxohexanamide;
N-(2-((1-amino-1-oxo-3-phenylpropan-2-yl)(methyl)amino)-2-oxoethyl)-4-methyl-3-(4-methyl-2-(2-morpholinoacetamido)pentanamido)-2-oxohexanamide;
N-(2-((1-amino-1-oxo-3-phenylpropan-2-yl)(methyl)amino)-2-oxoethyl)-4-methyl-3-(4-methyl-2-(2-(pyridin-4-yl)acetamido)pentanamido)-2-oxohexanamide;
N-(1-amino-3-(naphthalen-2-yl)-1-oxopropan-2-yl)-4-methyl-3-(4-methyl-2-(2-morpholinoacetamido)pentanamido)-2-oxohexanamide;
benzyl (1-((1-((2-((1-amino-3-(4-methoxyphenyl)-1-oxopropan-2-yl)amino)-2-oxoethyl)amino)-4-methyl-1,2-dioxohexan-3-yl)amino)-4-methyl-1-oxopentan-2-yl)carbamate;
benzyl (1-((1-((2-((1-amino-1-oxo-3-(pyridin-3-yl)propan-2-yl)amino)-2-oxoethyl)amino)-4-methyl-1,2-dioxohexan-3-yl)amino)-4-methyl-1-oxopentan-2-yl)carbamate;
benzyl (1-((1-((2-((1-amino-1-oxo-3-(pyridin-4-yl)propan-2-yl)amino)-2-oxoethyl)amino)-4-methyl-1,2-dioxohexan-3-yl)amino)-4-methyl-1-oxopentan-2-yl)carbamate;
benzyl (1-((1-((1-amino-1-oxo-3-phenylpropan-2-yl)(methyl)amino)-4-methyl-1,2-dioxohexan-3-yl)amino)-4-methyl-1-oxopentan-2-yl)carbamate;
benzyl (1-((1-((1-amino-3-(3-fluorophenyl)-1-oxopropan-2-yl)amino)-4-methyl-1,2-dioxohexan-3-yl)amino)-4-methyl-1-oxopentan-2-yl)carbamate;
benzyl (1-((1-((1-(dimethylamino)-3-(naphthalen-2-yl)-1-oxopropan-2-yl)amino)-4-methyl-1,2-dioxohexan-3-yl)amino)-4-methyl-1-oxopentan-2-yl)carbamate;
benzyl (1-((1-((1-(cyclopropylamino)-3-(naphthalen-2-yl)-1-oxopropan-2-yl)amino)-4-methyl-1,2-dioxohexan-3-yl)amino)-4-methyl-1-oxopentan-2-yl)carbamate;
benzyl (4-methyl-1-((4-methyl-1,2-dioxo-1-(3-((pyrimidin-5-ylmethyl)carbamoyl)azetidin-1-yl)hexan-3-yl)amino)-1-oxopentan-2-yl)carbamate;
benzyl (4-methyl-1-((4-methyl-1-(3-((naphthalen-2-ylmethyl)carbamoyl)azetidin-1-yl)-1,2-dioxohexan-3-yl)amino)-1-oxopentan-2-yl)carbamate;
benzyl (4-methyl-1-((4-methyl-1,2-dioxo-1-(3-(((1,4,5,6-tetrahydrocyclopenta[c]pyrazol-3-yl)methyl)carbamoyl)azetidin-1-yl)hexan-3-yl)amino)-1-oxopentan-2-yl)carbamate;
N-(2-((1-amino-3-(naphthalen-2-yl)-1-oxopropan-2-yl)amino)-2-oxoethyl)-4-methyl-3-(4-methyl-2-(2-(pyrimidin-5-yl)acetamido)pentanamido)-2-oxohexanamide;
benzyl (1-((1-(3-((4-carbamimidoylbenzyl)carbamoyl)azetidin-1-yl)-4-methyl-1,2-dioxohexan-3-yl)amino)-4-methyl-1-oxopentan-2-yl)carbamate;
1-(3-(2-(((benzyloxy)carbonyl)amino)-4-methylpentanamido)-4-methyl-2-oxohexanoyl)azetidine-3-carboxylic acid;
or a pharmaceutically acceptable salt thereof.

47. A method of inhibiting calpain activity, comprising administering the compound of any one or more compounds of claims 1-46 to a patient in need thereof.

48. A method of treating a calpain-mediated disorder in a subject, comprising administering to the subject a therapeutically effective amount of the compound of any one of claims 1-46 to said subject.

49. The method of claim 48, wherein the calpain-mediated disorder is Alzheimer's disease, atherosclerosis, bacterial infection, cancer, cataract formation, diabetes, fibrotic disease, halogen/acid exposure injury, ischemia, muscular dystrophy, neurological injury, parasite infection, reperfusion injury, osteoarthritis, or Wolfram's syndrome.

50. The method of claim 49, wherein the neurological injury is traumatic brain injury, stroke, Parkinson's disease, or spinal cord injury.

51. The method of claim 49, wherein the disorder is fibrotic disease.

52. The method of claim 51, wherein the fibrotic disease is pulmonary fibrosis, scleroderma, cardiac fibrosis, renal fibrosis, liver fibrosis including nonalcoholic steatohepatitis (NASH), Crohn's disease, strabismus, or hypertrophic scarring.

53. A method of inhibiting cathepsin-B, cathepsin-L, cathepsin-S, or cathepsin-L activity, comprising administering the compound of any one of claims 1-44 to a patient in need thereof.

54. A method of treating a cathepsin-B, cathepsin-L, cathepsin-S, or cathepsin-L mediated disorder in a subject, comprising administering to the subject a therapeutically effective amount of the compound of any one of claims 1-46 to said subject.

55. The method of claim 54, which treats a cathepsin-L or cathepsin-S mediated disorder.

56. The method of claim 54 or 55, wherein the disorder is chronic pain, a coronavirus infection, Ebola virus infection, a filovirus infection, marburg virus syndrome or osteoporosis.

57. The method of claim 56, wherein the corona virus infection is a middle east respiratory syndrome (MERS).
